(19) United States
(12) Patent Application Publication
(10) Pub. No.: US 2024/0190970 A1
(43) Pub. Date: Jun. 13, 2024

(54) ANTI-GALECTIN-9 ANTIBODIES AND THERAPEUTIC USES THEREOF

(71) Applicant: PURETECH LYT, INC., Boston, MA (US)

(72) Inventors: Aleksandra FILIPOVIC, London (GB); Eric ELENKO, Boston, MA (US); Heather PADEN, Boston, MA (US); Christopher KORTH, Boston, MA (US)

(73) Assignee: PURETECH LYT, INC., Boston, MA (US)

(21) Appl. No.: 18/556,387

(22) PCT Filed: Apr. 29, 2022
(86) PCT No.: PCT/US2022/027127

§ 371 Date: Oct. 20, 2023

Related U.S. Application Data
(60) Provisional application No. 63/182,521, filed on Apr. 30, 2021.
(60) Provisional application No. 63/193,357, filed on May 26, 2021.
(60) Provisional application No. 63/313,879, filed on Feb. 25, 2022.

(51) Int. Cl. C07K 16/28; A61K 39/00; A61P 35/00

(52) U.S. Cl. CPC C07K 16/2851; A61P 35/00; C07K 16/2818; A61K 2039/505; A61K 2039/545; C07K 2317/92

(57) ABSTRACT

Disclosed herein are methods for treating solid tumors (e.g., pancreatic ductal adenocarcinoma (PDAC), colorectal cancer (CRC), hepatocellular carcinoma (HCC), cholangiocarcinoma (CAA), renal cell carcinoma (RCC), urothelial, head and neck, breast cancer, lung cancer, or other gastrointestinal solid tumors), using an anti-Galectin-9) antibody, e.g., as a monotherapy or as a combined therapy with an immune checkpoint inhibitor.

### CROSS REFERENCE TO RELATED APPLICATIONS

This application claims the benefit under 35 U.S.C. § 119(e) of U.S. Provisional Application No. 63/182,521, filed Apr. 30, 2021, U.S. Provisional Application No. 63/193,357, filed May 26, 2021, and U.S. Provisional Application No. 63/313,879, filed Feb. 25, 2022, the contents of each of which are incorporated by reference herein in their entirety.

### SEQUENCE LISTING

The instant application contains a Sequence Listing which has been filed electronically in ASCII format and is hereby incorporated by reference in its entirety. Said ASCII copy, created on Apr. 26, 2022, is named 112174-0211-NP009WO1_SEQ.txt and is 89,119 bytes in size.

### BACKGROUND OF INVENTION

The immune system holds remarkable potential to recognize and destroy cancer cells, but the complex network governing tumor immune escape is an obstacle to broadly effective immune modulation (Martinez-Bosch N, et al., Immune Evasion in Pancreatic Cancer: From Mechanisms to Therapy. Cancers (Basel). 2018; 10 (1)). Approved immuno-oncology (IO) agents deliver incremental survival improvements to many tumor types (e.g., melanoma, lung, renal, bladder cancer, some colon cancers etc.), and are being rapidly integrated as standard of care in addition to and in conjunction with surgery, chemotherapy, and radiotherapy. However, there is still a major gap in the treatment and survivorship of multiple other aggressive malignancies. For example, metastatic pancreatic ductal adenocarcinoma (PDAC or PDA)), cholangiocarcinoma (CCA) and colorectal cancer (CRC) still have 5-year survival rates of <9%, <5% and <15%, respectively. These gastrointestinal tumors are very aggressive, many patients have advanced-stage disease at presentation, and the effectiveness of approved immunotherapies is suboptimal (Rizvi, et al., Cholangiocarcinoma—evolving concepts and therapeutic strategies; Nat Rev Clin Oncol. 2018; 15(2):95-111; Kalyan, et al., Updates on immunotherapy for colorectal cancer; J Gastrointest Oncol. 2018; 9(1): 160-169).

The success of first-generation checkpoint inhibitors (anti-PD-1, anti-PD-L1, and anti-CTLA4) has led to an explosion of new IO clinical trial efficacy and differentiation (Holl et al., Examining Peripheral and Tumor Cellular Immunome in Patients with Cancer; Front Immunol. 2019; 10:1767). However, among successes, there have also been many unfortunate development failures, consequently, there is still a need for more novel and efficacious treatments.

Galectin-9 is a tandem-repeat lectin consisting of two carbohydrate recognition domains (CRDs) and was discovered and described for the first time in 1997 in patients suffering from Hodgkin's lymphoma (HL) (Tureci et al., J. Biol. Chem. 1997, 272, 6416-6422). Three isoforms exist and can be located within the cell or extracellularly. Elevated Galectin-9 levels have been in observed a wide range of cancers, including melanoma, Hodgkin's lymphoma, hepatocellular, pancreatic, gastric, colon and clear cell renal cell cancers (Wdowiak et al. Int. J. Mol. Sci. 2018, 19, 210). In renal cancer, patients with high Galectin-9 expression showed more advanced progression of the disease with larger tumor size (Kawashima et al.; BJU Int. 2014; 113:320-332). In melanoma, Galectin-9 was expressed in 57% of tumors and was significantly increased in the plasma of patients with advanced melanoma compared to healthy controls (Enninga et al., Melanoma Res. 2016 October; 26(5): 429-441). A number of studies have shown utility for Galectin-9 as a prognostic marker, and more recently as a potential new drug target (Enninga et al., 2016; Kawashima et al. BJU Int 2014; 113: 320-332; Kageshita et al., Int J Cancer. 2002 Jun. 20; 99(6):809-16, and references therein).

Galectin-9 has been described to play an important role in in a number of cellular processes such as adhesion, cancer cell aggregation, apoptosis, and chemotaxis. Recent studies have shown a role for Galectin-9 in immune modulation in support of the tumor, e.g., through negative regulation of Th1 type responses, Th2 polarization and polarization of macrophages to the M2 phenotype. This work also includes studies that have shown that Galectin-9 participates in direct inactivation of T cells through interactions with the T-cell immunoglobulin and mucin protein 3 (TIM-3) receptor (Dardalhon et al., J Immunol., 2010, 185, 1383-1392; Sanchez-Fueyo et al., Nat Immunol., 2003, 4, 1093-1101).

Galectin-9 has also been found to play a role in polarizing T cell differentiation into tumor suppressive phenotypes), as well as promoting tolerogenic macrophage programming and adaptive immune suppression (Daley et al., Nat Med., 2017, 23, 556-567). In mouse models of pancreatic ductal adenocarcinoma (PDAC), blockade of the checkpoint interaction between Galectin-9 and the receptor Dectin-1 found on innate immune cells in the tumor microenvironment (TME) has been shown to increase anti-tumor immune responses in the TME and to slow tumor progression (Daley et al., Nat Med., 2017, 23, 556-567). Galectin-9 also has been found to bind to CD206, a surface marker of M2 type macrophages, resulting in a reduced secretion of CVL22 (MDC), a macrophage derived chemokine which has been associated with longer survival and lower recurrence risk in lung cancer (Enninga et al, J Pathol. 2018 August; 245(4):468-477).

### SUMMARY OF INVENTION

The present disclosure is based, at least in part, on the development of treatment regimen for solid tumors (e.g., metastatic solid tumors) such as pancreatic ductal adenocarcinoma (PDAC), colorectal cancer (CRC), hepatocellular carcinoma (HCC), cholangiocarcinoma (CAA), renal cell carcinoma (RCC), urothelial, head and neck, breast cancer, lung cancer, or other GI solid tumors, involving an antibody capable of binding to human Galectin-9, either alone or in combination with a checkpoint inhibitor such as an anti-PD-1 antibody. Alternatively or in addition, the present disclosure is based, at least in part, on the unexpectedly discovery that an anti-Galectin 9 antibody G9.2-17 (IgG4) has a quicker clearance rate in human subjects as compared with other antibody therapeutics. Accordingly, a treatment regimen comprising a dosing schedule of once every week was developed to ensure a suitable plasma concentration, e.g., a therapeutic systemic exposure level, of the anti-Galectin 9 antibody for achieving therapeutic effects.

Accordingly, provided herein is a method for treating a solid tumor, the method comprising administering to a subject in need thereof (e.g., a human patient having the target solid tumor) an effective amount of an antibody that binds human Galectin-9 (anti-Galectin-9 antibody). The anti-Galectin-9 antibody may be administered to the subject at a dose of about 0.2 mg/kg to about 32 mg/kg once every week to once every six weeks, e.g., 0.2 mg/kg, 0.63 mg/kg, 2 mg/kg, 6.3 mg/kg, 10 mg/kg, or 16 mg/kg once every week to once every six weeks. In some embodiments, any of the anti-Galectin-9 antibodies disclosed herein may be administered to the subject by intravenous infusion.

In some embodiments, the anti-Galectin-9 antibody (e.g., G9.2-17 (IgG4)) may be administered to the subject at a dose of 0.2 mg/kg, 0.63 mg/kg, 2 mg/kg, 6.3 mg/kg, 10 mg/kg, or 16 mg/kg once every two weeks to once every four weeks. In some examples, the anti-Galectin-9 antibody may be administered to the subject once every two weeks. In specific embodiments, the anti-Galectin-9 antibody (e.g., G9.2-17 (IgG4)) is administered to the subject at a dose of 10 mg/kg or 16 mg/kg once every two weeks to once every four weeks (e.g., once every two weeks).

Alternatively, the anti-Gal-9 antibody such as G9.2-17 (IgG4) may be administered to a subject at a dose of about 650 mg to about 1120 mg once every 2-6 weeks, for example, once every 2 weeks, once every 3 weeks, or once every 4 weeks. In some examples, the anti-Gal-9 antibody is administered to a subject at a dose of about 650 mg to about 700 mg once every 2-6 weeks, for example, once every 2 weeks, once every 3 weeks, or once every 4 weeks. In other examples, the anti-Gal-9 antibody is administered to a subject at a dose of about 1040 mg to about 1120 mg once every 2-6 weeks, for example, once every 2 weeks, once every 3 weeks, or once every 4 weeks.

In some embodiments, the anti-Galectin-9 antibody (e.g., G9.2-17 (IgG4)) is administered to the subject at a dose of 0.2 mg/kg, 0.63 mg/kg, 2 mg/kg, 6.3 mg/kg, 10 mg/kg, or 16 mg/kg once every week. In specific embodiments, the anti-Galectin-9 antibody (e.g., G9.2-17 (IgG4)) is administered to the subject at a dose of 10 mg/kg or 16 mg/kg once every week. Alternatively, the anti-Gal-9 antibody disclosed herein, such as G9.2-17 (IgG4). may be administered to the subject at a dose of about 650 mg to about 1120 mg once every week. For example, the anti-Gal-9 antibody can be administered to the subject at a dose of 10 mg/kg once every week or at a flat dose of about 650-700 mg once every week. Alternatively, the anti-Galectin-9 antibody can be administered to the subject at a dose of 16 mg/kg once every week or at a flat dose of about 1040-1120 mg once every week.

In some embodiments, the anti-Galectin-9 antibody may comprise:
(a) a light chain comprising a light chain variable region (V_{L}), which comprises a light chain (LC) complementarity determining region 1 (CDR1) comprising the amino acid sequence of SEQ ID NO: 1, a LC complementarity determining region 2 (CDR2) comprising the amino acid sequence of SEQ ID NO: 2, and a LC complementarity determining region 3 (CDR3) comprising the amino acid sequence of SEQ ID NO: 3 and
(b) a heavy chain comprising a heavy chain variable region (V_{H}), which comprises a heavy chain (HC) complementarity determining region 1 (CDR1) comprising the amino acid sequence of SEQ ID NO: 4, a HC complementarity determining region 2 (CDR2) comprising the amino acid sequence of SEQ ID NO: 5, and a HC complementarity determining region 3 (CDR3) comprising the amino acid sequence of SEQ ID NO: 6.

In some examples, the V_{L} of the anti-Galectin-9 antibody comprises the amino acid sequence of SEQ ID NO: 8. Alternatively or in addition, the V_{H} of the anti-Galectin-9 antibody comprises the amino acid sequence of SEQ ID NO: 7.

In some instances, the anti-Galectin-9 antibody is a full-length antibody, for example, an IgG1 or IgG4 molecule. In some examples, the anti-Galectin-9 antibody is a human IgG4 molecule. Such an IgG4 molecule may have a modified Fc region relative to the wildtype human IgG4 counterpart. In some examples, the modified Fc region comprises the amino acid sequence of SEQ ID NO: 14. In specific examples, the anti-Galectin-9 antibody comprises a heavy chain comprising the amino acid sequence of SEQ ID NO: 19 and a light chain comprising the amino acid sequence of SEQ ID NO: 15. Such an anti-Galectin-9 antibody may be G9.2-17 (IgG4) as disclosed herein.

In some embodiments, the solid tumor to be treated by any of the methods disclosed herein may be pancreatic ductal adenocarcinoma (PDAC), colorectal cancer (CRC), hepatocellular carcinoma (HCC), cholangiocarcinoma (CAA), renal cell carcinoma (RCC), urothelial, head and neck, breast cancer, lung cancer, or other GI solid tumors. In some instances, the solid tumor is a metastatic tumor. In some embodiments, the method comprises administering to a subject having a solid tumor, e.g., PDAC, CRC, HCC, or CCA an effective amount of an antibody that binds human Galectin-9 (referred to herein as an anti-Gal 9 antibody or anti-Galectin-9 antibody). In some instances, the subject has one or more of the following features: (i) has no resectable cancer; (ii) has no infection by SARS-COV-2; (iii) has no active brain or leptomeningeal metastasis; and (iv) has unresectable metastatic cancer, which is adenocarcinoma, optionally squamous cell carcinoma.

In some embodiments, the subject is free of other anti-cancer therapy concurrently with the anti-Galectin-9 antibody. Alternatively, the method may further comprise administering to the subject an immune checkpoint inhibitor. In some examples, the immune checkpoint inhibitor is an antibody that binds PD-1. Examples include pembrolizumab, nivolumab, tislelizumab, dostarlimab, or cemiplimab. In some instances, the subject is free of exposure to any anti-PD-1 or anti-PD-L1 agent in any prior lines of therapy, free of microstatellite instability (MSI-H) and/or deficient mismatch repair (dMMR), or a combination thereof.

In one example, the antibody that binds PD-1 is nivolumab. In some instances, nivolumab is administered to the subject at a dose of 240 mg once every two weeks. In another example, the antibody that binds PD-1 is tislelizumab. In some instances, tislelizumab is administered intravenously at a dose of about 200 mg once every 3 weeks or at a dose of about 400 mg every six weeks.

In some embodiments, the anti-Galectin-9 antibody is administered to the subject at a dose of about 0.2 mg/kg to about 32 mg/kg (e.g., about 3 mg/kg to about 15 mg/kg or about 2 mg/kg to about 16 mg/kg or a higher dose level, or about 0.2 mg/kg to about 15 mg/kg, or about 0.2 to about 16 mg/kg or a higher dose level) once every 2-3 weeks. In some embodiments, the anti-Galectin-9 antibody is administered to the subject at a dose selected from 0.2 mg/kg, 0.63 mg/kg, 2 mg/kg, 4 mg/kg, 6 mg/kg, 6.3 mg/kg, 8 mg/kg, 10 mg/kg, 12 mg/kg, or 16 mg/kg or higher dose level In some embodiments, the anti-Galectin-9 antibody is administered to the subject at a dose selected from 2 mg/kg, 4 mg/kg, 8 mg/kg, 12 mg/kg, or 16 mg/kg or a higher dose level. In some embodiments, the anti-Galectin-9 antibody is administered to the subject at a dose selected from 0.2 mg/kg, 0.63 mg/kg, 2 mg/kg, 4 mg/kg, 6 mg/kg, 6.3 mg/kg, 10 mg/kg, or 16 mg/kg or a higher dose level. In some embodiments, the antibody is administered once every 2 weeks. In some embodiments, the anti-Galectin-9 antibody is administered to the subject at a dose selected from 2 mg/kg, 4 mg/kg, 8 mg/kg, 12 mg/kg, or 16 mg/kg or a higher dose level once every 2 weeks. In some embodiments, the anti-Galectin-9 antibody is administered to the subject at a dose selected from 0.2 mg/kg, 0.63 mg/kg, 2 mg/kg, 4 mg/kg, 6 mg/kg, 6.3 mg/kg, 10 mg/kg, or 16 mg/kg or a higher dose level once every 2 weeks. In some embodiments, the anti-Galectin-9 antibody is administered once every 2 weeks for one cycle, once every 2 weeks for two cycles, once every 2 weeks for 3 cycles, once every 2 weeks for 4 cycles, or once every 2 weeks for more than 4 cycles. In some embodiments, the duration of treatment is 0-3 months, 3-6 months, 12-24 months or longer. In some embodiments, the duration of treatment is 12-24 months or longer. In some embodiments, the cycles extend for a duration of 3 months to 6 months, or 6 months to 12 months or 12 months to 24 months or longer. In some embodiments, the cycle length is modified, e.g., temporarily or permanently to a longer duration, e.g., 3 weeks or 4 weeks. In some embodiments, the anti-Galectin-9 antibody is administered to the subject by intravenous infusion. In some embodiments, the cancer is metastatic cancer, including a metastatic cancer of any of the above-mentioned cancers. In some embodiments, the method of treatment comprising administering the anti-Galectin-9 antibody does not include any other concurrent anti-cancer therapy.

In some embodiments, the method of treatment employing the anti-Galectin-9 antibody includes another concurrent anti-cancer therapy. Thus, in some embodiments, the method of treatment employing the anti-Galectin-9 antibody further comprises administering to the subject an immune checkpoint inhibitor. In some embodiments, the immune checkpoint inhibitor is an antibody that binds PD-1, for example, pembrolizumab, nivolumab, tislelizumab, dostarlimab or cemiplimab. In some embodiments, the antibody that binds PD-1 is nivolumab, which is administered to the subject at a dose of 240 mg once every two weeks. In some embodiments, the antibody that binds PD-1 is nivolumab, which is administered to the subject at a dose of about 240 mg every two weeks or about 480 mg once every 4 weeks. In some embodiments, the antibody that binds PD-1 is pembrolizumab, which is administered at a dose of 200 mg once every 3 weeks. In some embodiments, the antibody that binds PD-1 is cemiplimab, which is administered at a dose of about 350 mg once every 3 weeks. In some embodiments, the antibody that binds PD-1 is tislelizumab, which is administered at a dose of about 200 mg once every 3 weeks or about 400 mg every six weeks. In some embodiments, the antibody that binds PD-1 is dostarlimab, which is administered at a dose of about 500 mg once every 3 weeks or at a dose of about 1000 mg every six weeks. In some embodiments, the immune checkpoint inhibitor is administered by intravenous infusion.

In some instances, the subject is (v) free of exposure to any anti-PD-1 or anti-PD-L1 agent in any prior lines of therapy, free of microsatellite instability (MSI-H) and/or deficient mismatch repair (dMMR), or a combination thereof. In some instances, the subject has microsatellite instability (MSI-H) and/or deficient mismatch repair (dMMR), or a combination thereof.

In some examples, the checkpoint inhibitor is administered to the subject on a day when the subject receives the anti-Galectin 9 antibody. Alternatively, the checkpoint inhibitor and the anti-Galectin 9 antibody are administered to the subject on two consecutive days. For example, the administration of the checkpoint inhibitor is performed prior to the administration of the anti-Galectin 9 antibody or vice versa.

In some embodiments, the subject has undergone one or more prior anti-cancer therapies. In some examples, the one or more prior anti-cancer therapies comprise chemotherapy, immunotherapy, radiation therapy, a therapy involving a biologic agent, or a combination thereof. In some instances, the subject has progressed disease through the one or more prior anti-cancer therapies or is resistant to the one or more prior therapies.

In some instances, the subject is a human patient having an elevated level of Galectin-9 relative to a control value. For example, the human patient has an elevated serum or plasma level of Galectin-9 relative to the control value. In some examples, the human patient has cancer cells expressing Galectin-9. Alternatively, or in addition, the human patient has immune cells expressing Galectin-9. In some examples, the cancer cells are in tumor organoids derived from the human patient. In some embodiments, the control value is based on a value obtained from a healthy human subject.

Any of the methods disclosed herein may further comprise monitoring occurrence of adverse effects in the subject. In some examples, the method may further comprise reducing the dose of the anti-Galectin-9 antibody, the dose of the checkpoint inhibitor, or both when an adverse effect is observed.

In some embodiments, the subject is administered multiple doses of the anti-Galectin 9 antibody and a later dose is higher than an earlier dose.

Also within the scope of the present disclosure are pharmaceutical compositions for use in treating a solid tumor (e.g., those described herein and including metastatic solid tumors), and uses of any of the anti-Galectin-9 antibodies for manufacturing a medicament for treating the solid tumor, either taken alone or in combination with a checkpoint inhibitor such as any of the anti-PD-1 antibodies disclosed herein.

The details of one or more embodiments of the invention are set forth in the description below. Other features or advantages of the present invention are to be apparent from the following drawing and detailed description of several embodiments, and also from the appended claims.

### BRIEF DESCRIPTION OF DRAWINGS

The following drawings form part of the present specification and are included to further demonstrate certain aspects of the present disclosure, which can be better understood by reference to the drawing in combination with the detailed description of specific embodiments presented herein.

FIG. 1 is a schematic depicting an exemplary study scheme. CRM: reassessment method; RP2D: recommended Phase 2 dose; PK: pharmacokinetics; PD: pharmacodynamics; PDAC: pancreatic ductal adenocarcinoma; CRC: colorectal cancer; CCA: cholangiocarcinoma; TBD: to be decided.

FIG. 2 is a graph showing a representative size exclusion chromatography (SEC) profile for the anti-Galectin-9 antibody. The high molecular weight peaks are labeled.

FIGS. 3A-3F include bar graphs showing levels of Galectin-9 expression as measured in T cells (CD3⁺), macrophages (CD11b+), and tumor cells (Epcam+) in S2 and S3 organoid fractions derived from a pancreatic adenocarcinoma biopsy using anti-Galectin-9 G9.2-17 Fab fragment and a commercially available anti-Galectin-9 antibody (9M1-3). S2 fraction: organoids. S3 fraction: single cells. Corresponding isotype for G9.2-17 Fab (“Fab isotype”) and “fluorescence minus one” (FMO) 9M1-3 (“Gal9 FMO”) were used as controls for specificity, background staining and fluorescence bleed through from other channels. FIG. 3A shows levels of Galectin-9 in CD3⁺ cells as measured in the S3 fraction. FIG. 3B shows levels of Galectin-9 in CD11b+ cells as measured in the S3 fraction. FIG. 3C shows levels of Galectin-9 in Epcam⁺ cells as measured in the S3 fraction. FIG. 3D shows levels of Galectin-9 in CD3⁺ cells as measured in the S2 fraction. FIG. 3E shows levels of Galectin-9 in CD11b⁺ cells as measured in the S2 fraction. FIG. 3F shows levels of Galectin-9 in Epcam⁺ cells as measured in the S2 fraction.

FIGS. 4A-4F include bar graphs showing levels of Galectin-9 expression as measured in T cells (CD3⁺), macrophages (CD11b+), and tumor cells (Epcam+) in S2 and S3 organoid fractions derived from a colorectal carcinoma biopsy using anti-Galectin-9 G9.2-17 Fab fragment and a commercially available anti-Galectin-9 antibody (9M1-3). S2 fraction: organoids. S3 fraction: single cells. Corresponding isotype for G9.2-17 Fab (“Fab isotype”) and FMO 9M1-3 (“Gal9 FMO”) were used controls for specificity, background staining and fluorescence bleed through from other channels. FIG. 4A shows levels of Galectin-9 in CD3⁺ cells as measured in the S3 fraction. FIG. 4B shows levels of Galectin-9 in CD11b⁺ cells as measured in the S3 fraction. FIG. 4C shows levels of Galectin-9 in Epcam⁺ cells as measured in the S3 fraction. FIG. 4D shows levels of Galectin-9 in CD3⁺ cells as measured in the S2 fraction. FIG. 4E shows levels of Galectin-9 in CD11b⁺ cells as measured in the S2 fraction. FIG. 4F shows levels of Galectin-9 in Epcam⁺ cells as measured in the S2 fraction.

FIGS. 5A-5F include bar graphs showing levels of Galectin-9 expression as measured in T cells (CD3+), macrophages (CD11b+), and tumor cells (Epcam+) in S2 and S3 organoid fractions derived from a second pancreatic adenocarcinoma biopsy using anti-Galectin-9 G9.2-17 Fab fragment and a commercially available Galectin-9 antibody (9M1-3). S2 fraction: organoids. S3 fraction: single cells. Corresponding isotype for G9.2-17 Fab (“Fab isotype”) and FMO 9M1-3 (“Gal9 FMO”) were used as controls for specificity, background staining and fluorescence bleed through from other channels. FIG. 5A shows levels of Galectin-9 in CD3+ cells as measured in the S3 fraction. FIG. 5B shows levels of Galectin-9 in CD11b+ cells as measured in the S3 fraction. FIG. 5C shows levels of Galectin-9 in Epcam+ cells as measured in the S3 fraction. FIG. 5D shows levels of Galectin-9 in CD3+ cells as measured in the S2 fraction. FIG. 5E shows levels of Galectin-9 in CD11b+ cells as measured in the S2 fraction. FIG. 5F shows levels of Galectin-9 in Epcam+ cells as measured in the S2 fraction.

FIGS. 6A-6C include photographs of immunohistochemical analysis of various tumors using anti-Galectin-9 antibody 1G3. All magnifications are 200×. FIG. 6A shows chemotherapy-treated colorectal cancer with heterogeneous intensity score 2 and 3 (moderate and high) Galectin-9 expression. Galectin-9 staining was observed at the cell membrane in particular; additionally, intraglandular macrophages are moderately positive and stromal reaction in tumor shows multinucleated macrophage giant cells with moderately strong Galectin-9 expression. FIG. 6B shows liver metastasis of colorectal carcinoma with high (intensity score 3) Galectin-9 expression. Staining is located on the membrane and in the cytoplasm. FIG. 6C shows Galectin-9 positive (intensity score 2) entrapped bile ducts and Galectin-9 negative cancer.

FIG. 7 includes a graph showing the fraction of annexin V- and propidium iodide (PI)-positive cells plotted as a function of antibody concentration used. MOLM-13 cells were co-incubated with varying concentrations of either G9.2-17 or human IgG4 isotype antibody and recombinant human Galectin-9 for 16 hours. Cells were stained with annexin V and propidium iodide prior to analysis by flow cytometry. Each condition was performed in triplicate. Analysis was performed on FlowJo software.

FIGS. 8A-8B depict graphs showing results of a study in which mice treated with G9.2-17 mIgG2a alone or in combination with aPD-1 mAb. Mice (n=10/group) with orthotopically implanted KPC tumors were treated with commercial αPD-1 (200 μg) mAb or G9.2-17 mIg2a (200 μg), or a combination of G9.2-17 and αPD-1, or matched isotype once weekly for three weeks. Tumors were removed and weighed (FIG. 8A) and subsequently processed and stained for flow cytometry (FIG. 7B). Each point represents one mouse; *p<0.05; **p<0.01; ***p<0.001; ****p<0.0001; by unpaired Student's t-test. FIG. 8B depicts bar graphs showing tumors were excised from control and treated animals at the end of experiment (Day 18) and processed for flow cytometry of intra-tumoral immune cells and related activation and immunosuppressive markers. Mouse tumors were digested before flow. Flow cytometry was carried out on the Attune NxT flow cytometer (ThermoFisher Scientific, Waltham, MA). Data were analyzed using FlowJo v.10.1 (Treestar, Ashland, OR)

FIGS. 9A-9B depict graphs showing the results of ADCC assays performed with the IgG1 form of G9.2-17 (FIG. 9A) and the IgG4 form of G9.2-17 (FIG. 9B). As expected for a human IgG4 mAb, G9.2-17 does not mediate ADCC (FIG. 9B). This was tested against the IgG1 human counterpart of G9.2-17 as a positive control, which mediates ADCC and ADCP, as expected (FIG. 9A).

FIGS. 10A-10B depict graphs showing the effect of 9.2-17 in a B16F10 subcutaneous syngeneic model. Tumors were engrafted subcutaneously and treated with G9.2-17 IgG1 mouse mAb, anti-PD-1 antibody or a combination of G9.2-17 IgG1 mouse mAb and anti-PD-1 antibody. FIG. 10A depicts a graph showing the effect on tumor volume. FIG. 10B depicts a graph showing intratumoral CD8 T cell infiltration. Results show that intra-tumoral presence effector T cells were enhanced in the combination arm.

FIGS. 11A-11B include charts showing cholangiocarcinoma patient-derived tumor cultures ex vivo (organoids) treated with G9.2-17. Patient derived tumor cultures ex vivo (organoids) were treated with G9.2-17 or isotype control for three days. Expression of CD44 (FIG. 11A), and TNFα (FIG. 11B) in CD3+ T cells from PDOTS was assessed.

FIG. 12 includes a graph showing the effect of G2.9-17 on TGF-beta1 secretion measurements in whole blood of an exemplary healthy human donor. TGF-beta1 release from donor cryopreserved macrophages incubated in the presence of M2 polarization cocktails. IgG4 isotype is a negative control antibody. Data represent mean+SEM of triplicate measures. Significance was determined by two-way ANOVA with Dunnett's multiple comparison test. *p<0.05

FIG. 13 includes a graph showing the effect of G2.9-17 on IL-10 secretion in whole blood of an exemplary healthy human donor. IL-10 release from donor cryopreserved macrophages incubated in the presence of M2 polarization cocktails (IL-4/IL-13 or Gal-9). IgG4 isotype is a negative control antibody. Data represent the mean (+ SEM) of triplicate. Significance was determined by two-way ANOVA with Tukey's multiple comparisons test, * P<0.05.

### DETAILED DESCRIPTION OF INVENTION

Provided herein are methods of using anti-Galectin-9 antibodies, e.g., G9.2-17 (IgG4), for treating solid tumors, for example, pancreatic ductal adenocarcinoma (PDAC), colorectal cancer (CRC), hepatocellular carcinoma (HCC), cholangiocarcinoma (CAA), renal cell carcinoma (RCC), urothelial, head and neck, breast cancer, lung cancer, or other GI solid tumors. In some embodiments, the cancers are metastatic. In some embodiments, the methods disclosed herein provide specific doses and/or dosing schedules, for example, 0.2 mg/kg to 16 mg/kg of the antibody once every week (e.g., 10 mg/kg or 16 mg/kg once every week). It was discovered that G9.2-17 (IgG4) has an unexpectedly quick clearance rate in human subjects as compared with conventional antibody therapeutics. Accordingly, a treatment regimen comprising a dosing schedule of once very week was developed to ensure a systemic exposure level of the anti-Galectin 9 antibody that achieves therapeutic effect. In some instances, the methods disclosed herein target specific patient populations, for example, patients who have undergone prior treatment and show disease progression through the prior treatment, or patients who are resistant (de novo or acquired) to the prior treatment.

Galectin-9, a tandem-repeat lectin, is a beta-galactoside-binding protein, which has been shown to have a role in modulating cell-cell and cell-matrix interactions. It is found to be strongly overexpressed in Hodgkin's disease tissue and in other pathologic states. It has in some instances also been found circulating in the tumor microenvironment (TME).

Galectin-9 is found to interact with Dectin-1, an innate immune receptor which is highly expressed on macrophages in PDAC, as well as on cancer cells (Daley, et al. Nat Med. 2017; 23(5):556-6). Regardless of the source of Galectin-9, disruption of its interaction with Dectin-1 has been shown to lead to the reprogramming of CD4+ and CD8+ cells into indispensable mediators of anti-tumor immunity. Thus, Galectin-9 serves as a valuable therapeutic target for blocking the signaling mediated by Dectin-1. Accordingly, in some embodiments, the anti-Galectin-9 antibodies describe herein disrupt the interaction between Galectin-9 and Dectin-1.

Galectin-9 is also found to interact with TIM-3, a type I cell surface glycoprotein expressed on the surface of leukemic stem cells in all varieties of acute myeloid leukemia (except for M3 (acute promyelocytic leukemia)), but not expressed in normal human hematopoietic stem cells (HSCs). TIM-3 signaling resulting from Galectin-9 ligation has been found to have a pleiotropic effect on immune cells, inducing apoptosis in Th1 cells (Zhu et al., Nat Immunol., 2005, 6:1245-1252) and stimulating the secretion of tumor necrosis factor-α (TNF-α), leading to the maturation of monocytes into dendritic cells, resulting in inflammation by innate immunity (Kuchroo et al., Nat Rev Immunol., 2008, 8:577-580). Further Galectin-9/TIM-3 signaling has been found to co-activate NF-κB and β-catenin signaling, two pathways that promote LSC self-renewal (Kikushige et al., Cell Stem Cell, 2015, 17(3):341-352). An anti-Galectin-9 antibody that interferes with Galectin-9/TIM-3 binding could have a therapeutic effect, especially with respect to leukemia and other hematological malignancies. Accordingly, in some embodiments, the anti-Galectin-9 antibodies described herein disrupt the interaction between Galectin-9 and TIM-3.

Further, Galectin-9 is found to interact with CD206, a mannose receptor highly expressed on M2 polarized macrophages, thereby promoting tumor survival (Enninga et al., J Pathol. 2018 August; 245(4):468-477). Tumor-associated macrophages expressing CD206 are mediators of tumor immunosuppression, angiogenesis, metastasis, and relapse (see, e.g., Scodeller et al., Sci Rep. 2017 Nov. 7; 7(1): 14655, and references therein). Specifically, M1 (also termed classically activated macrophages) are trigged by Th1-related cytokines and bacterial products, express high levels of IL-12, and are tumoricidal. By contrast, M2 (so-called alternatively activated macrophages) are activated by Th2-related factors, express high level of anti-inflammatory cytokines, such as IL-10, and facilitate tumor progression (Biswas and Mantovani; Nat Immunol. 2010 October; 11(10):889-96). The pro-tumoral effects of M2 include the promotion of angiogenesis, advancement of invasion and metastasis, and the protection of the tumor cells from chemotherapy-induced apoptosis (Hu et al., Tumour Biol. 2015 December; 36(12): 9119-9126, and references therein). Tumor-associated macrophages are thought be of M2-like phenotype and have a protumor role. Galectin-9 has been shown to mediate myeloid cell differentiation toward an M2 phenotype (Enninga et al., Melanoma Res. 2016 October; 26(5):429-41). It is possible that Galectin-9 binding CD206 may result in reprogramming TAMs towards the M2 phenotype, similar to what has been previously shown for Dectin-1. Without wishing to be bound by theory, blocking the interaction of Galectin-9 with CD206 may provide one mechanism by which an anti-Galectin-9 antibody, e.g., a G9.2-17 antibody, can be therapeutically beneficial. Accordingly, in some embodiments, the anti-Galectin-9 antibodies described herein disrupt the interaction between Galectin-9 and CD206.

Galectin-9 has also been shown to interact with protein disulfide isomerase (PDI) and 4-1BB (Bi S, et al. Proc Natl Acad Sci USA. 2011; 108(26):10650-5; Madireddi et al. J Exp Med. 2014; 211(7): 1433-48).

Anti-Galectin-9 antibodies can serve as therapeutic agents for treating diseases associated with Galectin-9 (e.g., those in which a Galectin-9 signaling plays a role). Without being bound by theory, an anti-Galectin-9 antibody may block a signaling pathway mediated by Galectin-9. For example, the antibody may interfere with the interaction between Galectin-9 and its binding partner (e.g., Dectin-1, TIM-3 or CD206), thereby blocking the signaling triggered by the Galectin-9/Ligand interaction. Alternatively, or in addition, an anti-Galectin-9 antibody may also exert its therapeutic effect by inducing blockade and/or cytotoxicity, for example, ADCC, CDC, or ADCP against pathologic cells that express Galectin-9. A pathologic cell refers to a cell that contributes to the initiation and/or development of a disease, either directly or indirectly. See, e.g., WO2019/084553, WO2020/198390, WO2020/0223702, and WO2021022256, the relevant disclosures of each of which are incorporated by reference for the subject matter and purpose referenced herein.

The anti-Galectin-9 antibodies disclosed herein are capable of suppressing the signaling mediated by Galectin-9 (e.g., the signaling pathway mediated by Galectin-9/Dectin-1 or Galectin-9/Tim-3) or eliminating pathologic cells expressing Galectin-9 via, e.g., ADCC. Accordingly, the anti-Galectin-9 antibodies described herein can be used for inhibiting any of the Galectin-9 signaling and/or eliminating Galectin-9 positive pathologic cells, thereby benefiting treatment of diseases associated with Galectin-9.

Anti-Galectin-9 antibodies such as G9.2-17 (e.g., G9.2-17 (IgG4)) were found to be effective in inducing apoptosis against cells expressing Galectin-9. Further, the anti-tumor effects of anti-Galectin-9 antibodies such as G9.2-17 were demonstrated in a mouse model, either by itself, or in combination with a checkpoint inhibitor (e.g., an anti-PD-1 antibody). As reported herein, the efficacy of G9.2-17 was tested in mouse models of PDAC and melanoma as well as in patient derived organoid tumor models (PDOTs). The orthotopic PDAC KPC mouse model (LSL-KrasG12D/+; LSL-Trp53R172H/+; Pdx-1-Cre) that was used recapitulates many features of human disease, including unresponsiveness to approved checkpoint inhibitors (Bisht and Feldmann G; Animal models for modeling pancreatic cancer and novel drug discovery; Expert Opin Drug Discov. 2019; 14(2): 127-142; Weidenhofer et al., Animal models of pancreatic cancer and their application in clinical research; Gastrointestinal Cancer: Targets and Therapy 2016; 6). The B16F10 melanoma mouse model has been a long-standing standard to test immunotherapies (Curran et al., PD-1 and CTLA-4 combination blockade expands infiltrating T cells and reduces regulatory T and myeloid cells within B16 melanoma tumors; Proc Natl Acad Sci USA. 2010; 107(9):4275-4280).

PDOTs isolated from fresh human tumor samples retain autologous lymphoid and myeloid cell populations, including antigen-experienced tumor infiltrating CD4 and CD8 T lymphocytes, and respond to immune therapies in short-term ex vivo culture (Jenkins et al. Ex Vivo Profiling of PD-1 Blockade Using Organotypic Tumor Spheroids. Cancer Discov. 2018; 8(2):196-215; Aref et al., 3D microfluidic ex vivo culture of organotypic tumor spheroids to model immune checkpoint blockade; Lab Chip. 2018; 18(20):3129-3143). As reported herein, expression of Galectin-9 on cancer cells was observed in patient-derived organoid assays.

In vivo studies were performed with G9.2-17 mouse IgG1 (G9.2-17 mIgG1 contains the exact same binding epitope as G9.2-17 human IgG4 and has the same effector function), which achieves significant reduction of tumor growth already as a single agent in the orthotopic KPC model, where approved checkpoint inhibitors do not work. In the B16F10 model G9.2-17 significantly exceeds the efficacy of anti-PD-1. In both models, modulation of the intra-tumoral immune microenvironment using G9.2-17 mIgG1 through the upregulation of effector T cell activity and inhibition of immunosuppressive signals, as well as the augmentation of intra-tumoral CD8 T cell infiltration was demonstrated.

These results demonstrate that the anti-tumor methods disclosed herein, involving an anti-Galectin-9 antibody, optionally in combination the checkpoint inhibitor, would achieve superior therapeutic efficacy against the target solid tumors.

Accordingly, described herein are therapeutic uses of anti-Galectin-9 antibodies for treating certain cancers as disclosed herein.

### Antibodies Binding to Galectin-9

The present disclosure provides anti-Galectin-9 antibody G9.2-17 and functional variants thereof for use in the treatment methods disclosed herein.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-Galectin-9 antibody, encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, the EU definition, the “Contact” numbering scheme, the IMGT″ numbering scheme, the “AHo” numbering scheme, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; Edelman et al., Proc Natl Acad Sci USA. 1969 May; 63(1):78-85; and Almagro, J. Mol. Recognit. 17:132-143 (2004); MacCallum et al., J. Mol. Biol. 262:732-745 (1996), Lefranc M P et al., Dev Comp Immunol, 2003 January; 27(1):55-77; and Honegger A and Pluckthun A, J Mol Biol, 2001 Jun. 8; 309(3):657-70. See also hgmp.mrc.ac.uk and bioinf.org.uk/abs).

In some embodiments, the anti-Galectin-9 antibody described herein is a full-length antibody, which contains two heavy chains and two light chains, each including a variable domain and a constant domain. Alternatively, the anti-Galectin-9 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

Any of the antibodies described herein, e.g., anti-Galectin-9 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

Reference antibody G9.2-17 refers to an antibody capable of binding to human Galectin-9 and comprises a heavy chain variable region of SEQ ID NO: 7 and a light chain variable domain of SEQ ID NO: 8, both of which are provided below. In some embodiments, the anti-Galectin-9 antibody for use in the methods disclosed herein is the G9.2-17 antibody. In some embodiments, the anti-Galectin-9 antibody for use in the methods disclosed herein is an antibody having the same heavy chain complementarity determining regions (CDRs) as reference antibody G9.2-17 and/or the same light chain complementarity determining regions as reference antibody G9.2-17. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/).

The heavy and light chain CDRs of reference antibody G9.2-17 is provided in Table 1 below (determined using the Kabat methodology):

TABLE 1
Heavy and Light Chain CDRs of G9.2-17

| | | | |
|---|---|---|---|
| G9.2-17 | V_{L} CDR1 | RASQSVSSAVA | SEQ ID NO: 1 |
| | V_{L} CDR2 | SASSLYS | SEQ ID NO: 2 |
| | V_{L} CDR3 | QQSSTDPIT | SEQ ID NO: 3 |
| | V_{H} CDR1 | FTVSSSSIH | SEQ ID NO: 4 |
| | V_{H} CDR2 | YISSSSGYTYYADSVKG | SEQ ID NO: 5 |
| | V_{H} CDR3 | YWSYPSWWPYRGMDY | SEQ ID NO: 6 |

In some examples, the anti-Galectin-9 antibody for use in the methods disclosed herein may comprise (following the Kabat scheme) a heavy chain complementarity determining region 1 (CDR1) set forth as SEQ ID NO: 4, a heavy chain complementarity determining region 2 (CDR2) set forth as SEQ ID NO: 5, and a heavy chain complementarity determining region 3 (CDR3) set forth as SEQ ID NO: 6 and/or may comprise a light chain complementarity determining region 1 (CDR1) set forth as SEQ ID NO: 1, a light chain complementarity determining region 2 (CDR2) set forth as SEQ ID NO: 2, and a light chain complementarity determining region 3 (CDR3) set forth as SEQ ID NO: 3. The anti-Galectin-9 antibody, including the reference antibody G9.2-17, can be in any format as disclosed herein, for example, a full-length antibody or a Fab. The term “G9.2-17(IgG4)” used herein refers to a G9.2-17 antibody which is an IgG4 molecule. Likewise, the term “G9.2-17 (Fab)” refers to a G9.2-17 antibody, which is a Fab molecule.

In some embodiments, the anti-Galectin-9 antibody or binding portion thereof comprises heavy and light chain variable regions, wherein the light chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the light chain variable region CDR1, CDR2, and CDR3 amino acid sequences set forth in SEQ ID NOs: 1, 2, and 3, respectively. In some embodiments, the anti-Galectin-9 antibody or binding portion thereof comprises heavy and light chain variable regions, wherein the heavy chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the heavy chain variable region CDR1, CDR2, and CDR3 amino acid sequences set forth in SEQ ID NO: 4, 5, and 6, respectively.

Additional Galectin-9 antibodies, e.g., which bind to the CRD1 and/or CRD2 region of Galectin-9 are described in co-owned, co-pending U.S. patent application Ser. No. 16/173,970 and in co-owned, co-pending International Patent Applications PCT/US18/58028 and PCT/US2020/024767, the contents of each of which are herein incorporated by reference in their entireties.

In some embodiments, the anti-Galectin-9 antibody disclosed herein comprises light chain CDRs that have at least 80% (e.g., 80%, 85%, 90%, 91%, 92%, 93%, 94%, 95%, 96%, 97%, 98%, or 99% and any increment therein) sequence identity, individually or collectively, as compared with the corresponding V_{L} CDRs of reference antibody G9.2-17. Alternatively or in addition, in some embodiments, the anti-Galectin-9 antibody comprises heavy chain CDRs that have at least 80% (e.g., 80%, 85%, 90%, 91%, 92%, 93%, 94%, 95%, 96%, 97%, 98%, or 99% and any increment therein) sequence identity, individually or collectively, as compared with the corresponding V_{H} CDRs of reference antibody G9.2-17.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In other embodiments, the anti-Galectin-9 antibody described herein comprises a V_{H} that comprises the HC CDR1, HC CDR2, and HC CDR3, which collectively contain up to 8 amino acid residue variations (8, 7, 6, 5, 4, 3, 2, or 1 variation(s), including additions, deletions, and/or substitutions) relative to the HC CDR1, HC CDR2, and HC CDR3 of reference antibody G9.2-17. Alternatively or in addition, in some embodiments, the anti-Galectin-9 antibody described herein comprises a V_{H} that comprises the LC CDR1, LC CDR2, and LC CDR3, which collectively contain up to 8 amino acid residue variations (8, 7, 6, 5, 4, 3, 2, or 1 variations(s) including additions, deletions, and/or substitutions) relative to the LC CDR1, LC CDR2, and LC CDR3 of reference antibody G9.2-17.

In one example, the amino acid residue variations are conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-Galectin-9 antibodies disclosed herein, having the heavy chain CDRs disclosed herein, contains framework regions derived from a subclass of germline V_{H} fragment. Such germline V_{H} regions are well known in the art. See, e.g., the IMGT database (www.imgt.org) or at www.vbase2.org/vbstat.php. Examples include the IGHV1 subfamily (e.g., IGHV1-2, IGHV1-3, IGHV1-8, IGHV1-18, IGHV1-24, IGHV1-45, IGHV1-46, IGHV1-58, and IGHV1-69), the IGHV2 subfamily (e.g., IGHV2-5, IGHV2-26, and IGHV2-70), the IGHV3 subfamily (e.g., IGHV3-7, IGHV3-9, IGHV3-11, IGHV3-13, IGHV3-15, IGHV3-20, IGHV3-21, IGHV3-23, IGHV3-30, IGHV3-33, IGHV3-43, IGHV3-48, IGHV3-49, IGHV3-53, IGHV3-64, IGHV3-66, IGHV3-72, and IGHV3-73, IGHV3-74), the IGHV4 subfamily (e.g., IGHV4-4, IGHV4-28, IGHV4-31, IGHV4-34, IGHV4-39, IGHV4-59, IGHV4-61, and IGHV4-B), the IGHV subfamily (e.g., IGHV5-51, or IGHV6-1), and the IGHV7 subfamily (e.g., IGHV7-4-1).

Alternatively, or in addition, in some embodiments, the anti-Galectin-9 antibody, having the light chain CDRs disclosed herein, contains framework regions derived from a germline Vκ fragment. Examples include an IGKV1 framework (e.g., IGKV1-05, IGKV1-12, IGKV1-27, IGKV1-33, or IGKV1-39), an IGKV2 framework (e.g., IGKV2-28), an IGKV3 framework (e.g., IGKV3-11, IGKV3-15, or IGKV3-20), and an IGKV4 framework (e.g., IGKV4-1). In other instances, the anti-Galectin-9 antibody comprises a light chain variable region that contains a framework derived from a germline Vλ fragment. Examples include an IGλ1 framework (e.g., IGλV1-36, IGλV1-40, IGλV1-44, IGλV1-47, IGλV1-51), an IG22 framework (e.g., IGλV2-8, IGλV2-11, IGλV2-14, IGλV2-18, IGλV2-23), an IGλ3 framework (e.g., IGλV3-1, IGλV3-9, IGλV3-10, IGλV3-12, IGλV3-16, IGλV3-19, IGλV3-21, IGλV3-25, IGλV3-27), an IGλ4 framework (e.g., IGλV4-3, IGλV4-60, IGλV4-69), an IGλ5 framework (e.g., IGλV5-39, IGλV5-45), an IGλ6 framework (e.g., IGλV6-57), an IGλ7 framework (e.g., IGλV7-43, IGλV7-46), an IGλ8 framework (e.g., IGλV8-61), an IGλ9 framework (e.g., IGλV9-49), or an IGλ10 framework (e.g., IGλV10-54).

In some embodiments, the anti-Galectin-9 antibody for use in the method disclosed herein can be an antibody having the same heavy chain variable region (V_{H}) and/or the same light chain variable region (V_{L}) as reference antibody G9.2-17, the V_{H} and V_{L} region amino acid sequences are provided below:

V_{H}:
(SEQ ID NO: 7)
EVQLVESGGGLVQPGGSLRLSCAASGFTVSSSSIHWVRQAPGKGLEWVAY
ISSSSGYTYYADSVKGRFTISADTSKNTAYLQMNSLRAEDTAVYYCARYW
SYPSWWPYRGMDYWGQGTLVTVSS
V_{L}:
(SEQ ID NO: 8)
DIQMTQSPSSLSASVGDRVTITCRASQSVSSAVAWYQQKPGKAPKLLIYS
ASSLYSGVPSRFSGSRSGTDFTLTISSLQPEDFATYYCQQSSTDPITFGQ
GTKVEIKR

In some embodiments, the anti-Galectin-9 antibody has at least 80% sequence identity (e.g., 80%, 85%, 90%, 91%, 92%, 93%, 94%, 95%, 96%, 97%, 98%, or 99% identity) to the heavy chain variable region of SEQ ID NO: 7. Alternatively or in addition, the anti-Galectin-9 antibody has at least 80% sequence identity (e.g., 80%, 85%, 90%, 91%, 92%, 93%, 94%, 95%, 96%, 97%, 98%, or 99% identity) to the light chain variable region of SEQ ID NO: 8.

In some instances, the anti-Galectin-9 antibody disclosed herein is a functional variant of reference antibody G9.2-17. A functional variant can be structurally similar as the reference antibody (e.g., comprising the limited number of amino acid residue variations in one or more of the heavy chain and/or light chain CDRs as G9.2-17 as disclosed herein, or the sequence identity relative to the heavy chain and/or light chain CDRs of G9.2-17, or the V_{H} and/or V_{L} of G9.2-17 as disclosed herein) with substantially similar binding affinity (e.g., having a KD value in the same order) to human Galectin-9.

In some embodiments, the anti-Galectin-9 antibody as described herein can bind and inhibit the activity of Galectin-9 by at least 20% (e.g., 31%, 35%, 40%, 45%, 50%, 60%, 70%, 80%, 90%, 95% or greater, including any increment therein). The apparent inhibition constant (Ki^{app} or K_{i,app}), which provides a measure of inhibitor potency, is related to the concentration of inhibitor required to reduce enzyme activity and is not dependent on enzyme concentrations. The inhibitory activity of an anti-Galectin-9 antibody described herein can be determined by routine methods known in the art.

The K_{i,}^{app} value of an antibody may be determined by measuring the inhibitory effect of different concentrations of the antibody on the extent of the reaction (e.g., enzyme activity); fitting the change in pseudo-first order rate constant (v) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of K_{i,}^{app} versus substrate concentration.

$$v = A \cdot \frac{( [ E ] ) - [ I ] - {K}_{i}^{app} ) + \sqrt{{( [ E ] - [ I ] - {K}_{i}^{app} )}^{2} + 4 [ E ] \cdot } {K}_{i}^{app}}{2} \qquad ( Equation 1 )$$

Where A is equivalent to vₒ/E, the initial velocity (vₒ) of the enzymatic reaction in the absence of inhibitor (I) divided by the total enzyme concentration (E). In some embodiments, the anti-Galectin-9 antibody described herein has a Ki^{app} value of 1000, 900, 800, 700, 600, 500, 400, 300, 200, 100, 50, 40, 30, 20, 19, 18, 17, 16, 15, 14, 13, 12, 11, 10, 9, 8, 7, 6, 5 pM or less for the target antigen or antigen epitope. In some embodiments, the anti-Galectin-9 antibody has a lower Ki^{app} for a first target (e.g., the CRD2 of Galectin-9) relative to a second target (e.g., CRD1 of the Galectin-9). Differences in Ki^{app} (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some examples, the anti-Galectin-9 antibody inhibits a first antigen (e.g., a first protein in a first conformation or mimic thereof) greater relative to a second antigen (e.g., the same first protein in a second conformation or mimic thereof; or a second protein). In some embodiments, any of the anti-Galectin-9 antibodies is further affinity matured to reduce the Ki^{app} of the antibody to the target antigen or antigenic epitope thereof.

In some embodiments, the anti-Galectin-9 antibody suppresses Dectin-1 signaling, e.g., in tumor infiltrating immune cells, such as macrophages. In some embodiments, the anti-Galectin-9 antibody suppresses Dectin-1 signaling triggered by Galectin-9 by at least 30% (e.g., 31%, 35%, 40%, 50%, 60%, 70%, 80%, 90%, 95% or greater, including any increment therein). Such inhibitory activity can be determined by conventional methods, such as routine assays. Alternatively or in addition, the anti-Galectin-9 antibody suppresses the T cell immunoglobulin mucin-3 (TIM-3) signaling initiated by Galectin-9. In some embodiments, the anti-Galectin-9 antibody suppresses the T cell immunoglobulin mucin-3 (TIM-3) signaling, e.g., in tumor infiltrating immune cells, e.g., in some embodiments, by at least 30% (e.g., 31%, 35%, 40%, 50%, 60%, 70%, 80%, 90%, 95% or greater, including any increment therein). Such inhibitory activity can be determined by conventional methods, such as routine assays.

In some embodiments, the anti-Galectin-9 antibody suppresses the CD206 signaling, e.g., in tumor infiltrating immune cells. In some embodiments, the anti-Galectin-9 antibody suppresses the CD206 signaling triggered by Galectin-9 by at least 30% (e.g., 31%, 35%, 40%, 50%, 60%, 70%, 80%, 90%, 95% or greater, including any increment therein). Such inhibitory activity can be determined by conventional methods, such as routine assays. In some embodiments, the anti-Galectin-9 antibody blocks or prevents binding of Galectin-9 to CD206 by at least 30% (e.g., 31%, 35%, 40%, 50%, 60%, 70%, 80%, 90%, 95% or greater, including any increment therein). Such inhibitory activity can be determined by conventional methods, such as routine assays.

In some embodiments, the anti-Galectin-9 antibody induces cell cytotoxicity, such as ADCC, in target cells expressing Galectin-9, e.g., wherein the target cells are cancer cells or immune suppressive immune cells. In some embodiments, the anti-Galectin-9 antibody induces apoptosis in immune cells, such as T cells, or cancer cells by at least 30% (e.g., 31%, 35%, 40%, 50%, 60%, 70%, 80%, 90%, 95% or greater, including any increment therein). Such inhibitory activity can be determined by conventional methods, such as routine assays. In some embodiments, any of the anti-Galectin-9 antibodies described herein induce cell cytotoxicity such as complement-dependent cytotoxicity (CDC) against target cells expressing Galectin-9.

Antibody-dependent cell-mediated phagocytosis (ADCP) is an important mechanism of action for antibodies that mediate part or all of their action though phagocytosis. In that case, antibodies mediate uptake of specific antigens by antigen presenting cells. ADCP can be mediated by monocytes, macrophages, neutrophils, and dendritic cells, through FcγRIIa, FcγRI, and FcγRIIIa, of which FcγRIIa (CD32a) on macrophages represent the predominant pathway.

In some embodiments, the anti-Galectin-9 antibody induces cell phagocytosis of target cells, e.g., cancer cells or immune suppressive immune cells expressing Galectin-9 (ADCP). In some embodiments, the anti-Galectin-9 antibody increases phagocytosis of target cells, e.g., cancer cells or immune suppressive immune cells, by at least 30% (e.g., 31%, 35%, 40%, 50%, 60%, 70%, 80%, 90%, 95% or greater, including any increment therein).

In some embodiments, the anti-Galectin-9 antibody described herein induces cell cytotoxicity such as complement-dependent cytotoxicity (CDC) against target cells, e.g., cancer cells or immune suppressive immune cells. In some embodiments, the anti-Galectin-9 antibody increases CDC against target cells by at least 30% (e.g., 31%, 35%, 40%, 50%, 60%, 70%, 80%, 90%, 95% or greater, including any increment therein).

In some embodiments, the anti-Galectin-9 antibody induces T cell activation, e.g., in tumor infiltrating T cells, i.e., suppress Galectin-9 mediated inhibition of T cell activation, either directly or indirectly. In some embodiments, the anti-Galectin-9 antibody promotes T cell activation by at least 30% (e.g., 31%, 35%, 40%, 50%, 60%, 70%, 80%, 90%, 95% or greater, including any increment therein). T cell activation can be determined by conventional methods, such as using well-known assays for measuring cytokines and checkpoint inhibitors (e.g., measurement of CD44, TNF alpha, IFNgamma, and/or PD-1). In some embodiments, the anti-Galectin-9 antibody promotes CD4+ cell activation by at least 30% (e.g., 31%, 35%, 40%, 50%, 60%, 70%, 80%, 90%, 95% or greater, including any increment therein). In a non-limiting example, the anti-Galectin antibody induces CD44 expression in CD4+ cells. In some embodiments, the anti-Galectin-9 antibody increases CD44 expression in CD4+ cells by at least 30% (e.g., 31%, 35%, 40%, 50%, 60%, 70%, 80%, 90%, 95% or greater, including any increment therein). In a non-limiting example, the anti-Galectin antibody induces IFNgamma expression in CD4+ cells. In some embodiments, the anti-Galectin-9 antibody increases IFNgamma expression in CD4+ cells by at least 30% (e.g., 31%, 35%, 40%, 50%, 60%, 70%, 80%, 90%, 95% or greater, including any increment therein). In a non-limiting example, the anti-Galectin antibody induces TNFalpha expression in CD4+ cells. In some embodiments, the anti-Galectin-9 antibody increases TNFalpha expression in CD4+ cells by at least 30% (e.g., 31%, 35%, 40%, 50%, 60%, 70%, 80%, 90%, 95% or greater, including any increment therein).

In some embodiments, the anti-Galectin-9 antibody promotes CD8+ cell activation by at least 30% (e.g., 31%, 35%, 40%, 50%, 60%, 70%, 80%, 90%, 95% or greater), including any increment therein). In a non-limiting example, the anti-Galectin antibody induces CD44 expression in CD8+ cells. In some embodiments, the anti-Galectin-9 antibody increases CD44 expression in CD8+ cells by at least 30% (e.g., 31%, 35%, 40%, 50%, 60%, 70%, 80%, 90%, 95% or greater, including any increment therein). In a non-limiting example, the anti-Galectin antibody induces IFNgamma expression in CD8+ cells. In some embodiments, the anti-Galectin-9 antibody increases IFNgamma expression in CD8+ cells by at least 30% (e.g., 31%, 35%, 40%, 50%, 60%, 70%, 80%, 90%, 95% or greater, including any increment therein). In a non-limiting example, the anti-Galectin antibody induces TNFalpha expression in CD8+ cells. In some embodiments, the anti-Galectin-9 antibody increases TNFalpha expression in CD8+ cells by at least 30% (e.g., 31%, 35%, 40%, 50%, 60%, 70%, 80%, 90%, 95% or greater, including any increment therein).

In some embodiments, an anti-Galectin-9 antibody as described herein has a suitable binding affinity for the target antigen (e.g., Galectin-9) or antigenic epitopes thereof. As used herein, “binding affinity” refers to the apparent association constant or K_{A}. The K_{A} is the reciprocal of the dissociation constant (K_{D}). The anti-Galectin-9 antibody described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased K_{D}. Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20).

These techniques can be used to measure the concentration of bound binding protein as a function of target protein concentration. Under certain conditions, the fractional concentration of bound binding protein ([Bound]/[Total]) is generally related to the concentration of total target protein ([Target]) by the following equation:

[Bound]/[Total]=[Target]/(Kd+[Target])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay. In some cases, the in vitro binding assay is indicative of in vivo activity. In other cases, the in vitro binding assay is not necessarily indicative of in vivo activity. In some cases, tight binding is beneficial, but in other cases tight binding is not as desirable in vivo, and an antibody with lower binding affinity is more desirable.

In some embodiments, the heavy chain of any of any of the anti-Galectin-9 antibodies as described herein further comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain) of any IgG subfamily as described herein.

In some embodiments, the heavy chain constant region of the antibodies described herein comprise a single domain (e.g., CH1, CH2, or CH3) or a combination of any of the single domains, of a constant region (e.g., SEQ ID NO: 4, 5, 6). In some embodiments, the light chain constant region of the antibodies described herein comprise a single domain (e.g., CL), of a constant region. Exemplary light and heavy chain sequences are listed below. Exemplary light and heavy chain sequences are listed below. The hIgG1 LALA sequence includes two mutations, L234A and L235A (EU numbering), which suppress FcgR binding as well as a P329G mutation (EU numbering) to abolish complement Clq binding, thus abolishing all immune effector functions. The hIgG4 Fab Arm Exchange Mutant sequence includes a mutation to suppress Fab Arm Exchange (S228P; EU numbering). An IL2 signal sequence (MYRMQLLSCIALSLALVTNS; SEQ ID NO: 9) can be located N-terminally of the variable region. It is used in expression vectors, which is cleaved during secretion and thus not in the mature antibody molecule. The mature protein (after secretion) starts with “EVQ” for the heavy chain and “DIM” for the light chain. Amino acid sequences of exemplary heavy chain constant regions are provided below:

hIgG1 Heavy Chain Constant Region
(SEQ ID NO: 10)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK*
hIgG1 LALA Heavy Chain Constant Region
(SEQ ID NO: 12)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVSNKA
LGAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK*
hIgG4 Heavy Chain Constant Region
(SEQ ID NO: 13)
ASTKGPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSPGK*
hIgG4 Heavy Chain Constant Region
(SEQ ID NO: 20)
ASTKGPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK*
hIgG4 mut Heavy Chain Constant Region
(SEQ ID NO: 14)
ASTKGPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSPGK*
hIgG4 mut Heavy Chain Constant Region
(SEQ ID NO: 21)
ASTKGPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRTPEVT
CVVVDVSQEDPEVQFNWYVDGVEVHNAKTKPREEQFNSTYRVVSVLIVLHQDWLNGKEYKCKVSNKGLPS
SIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK*

In some instances, the heavy chain constant region in an anti-Galectin-9 antibody disclosed herein (e.g., G9.2-17) may have the C-terminal Lysine (K) residue removed for, e.g., manufacturing purposes. The corresponding amino acid sequences of those having no terminal K residue are provided below:

hIgG1 Heavy Chain Constant Region with No C-Terminal Lysine
(SEQ ID NO: 24)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG*
hIgG1 LALA Heavy Chain Constant Region with No C-Terminal Lysine
(SEQ ID NO: 25)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVSNKA
LGAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG*
hIgG4 Heavy Chain Constant Region with No C-Terminal Lysine
(SEQ ID NO: 26)
ASTKGPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSPG*
hIgG4 Heavy Chain Constant Region with No C-Terminal Lysine
(SEQ ID NO: 27)
ASTKGPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPSCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLG*
hIgG4 mut Heavy Chain Constant Region with No C-Terminal Lysine
(SEQ ID NO: 28)
ASTKGPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSPG*
hIgG4 mut Heavy Chain Constant Region with No C-Terminal Lysine
(SEQ ID NO: 29)
ASTKGPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLG*

In some embodiments, anti-Galectin-9 antibodies having any of the above heavy chain constant regions are paired with a light chain having the following light chain constant region:

Light Chain Constant Region
(SEQ ID NO: 11)
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC

Exemplary full length anti-Galectin-9 antibodies are provided below:

G9.2-17 hIgG1 Heavy Chain
(SEQ ID NO: 16)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK*
G9.2-17 hIgG1 Heavy Chain with No C-terminal Lysine Residue
(SEQ ID NO: 30)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPG*
G9.2-17 hIgG1 LALA Heavy Chain
(SEQ ID NO: 17)
EVQLVESGGGLVQPGGSLRLSCAASGFTVSSSSIHWVRQAPGKGLEWVAYISSSSGYTYYADSVKGRETI
SADTSKNTAYLQMNSLRAEDTAVYYCARYWSYPSWWPYRGMDYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPEAAGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALGAPIEKTISKAKGQP
REPQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK*
G9.2-17 hIgG1 LALA Heavy Chain with No C-terminal Lysine Residue
(SEQ ID NO: 31)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPEAAGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALGAPIEKTISKAKGQP
REPQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPG*
G9.2-17 hIgG4 Heavy Chain
(SEQ ID NO: 18)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVFPLAPCSR
STSESTAALGCLVKDYFPEPVTVSWNSGALTSGVHTFPAVLQSSGLYSLSSVVTVPSSSLGTKTYTCNVD
HKPSNTKVDKRVESKYGPPCPSCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSPGK*
G9.2-17 hIgG4 Heavy Chain with No C-terminal Lysine Residue
(SEQ ID NO: 32)
EVQLVESGGGLVQPGGSLRLSCAASGFTVSSSSIHWVRQAPGKGLEWVAYISSSSGYTYYADSVKGRETI
SADTSKNTAYLQMNSLRAEDTAVYYCARYWSYPSWWPYRGMDYWGQGTLVTVSSASTKGPSVFPLAPCSR
STSESTAALGCLVKDYFPEPVTVSWNSGALTSGVHTFPAVLQSSGLYSLSSVVTVPSSSLGTKTYTCNVD
HKPSNTKVDKRVESKYGPPCPSCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSPG*
G9.2-17 hIgG4 Heavy Chain
(SEQ ID NO: 22)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVFPLAPCSR
STSESTAALGCLVKDYFPEPVTVSWNSGALTSGVHTFPAVLQSSGLYSLSSVVTVPSSSLGTKTYTCNVD
HKPSNTKVDKRVESKYGPPCPSCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSLGK*
G9.2-17 hIgG4 Heavy Chain with No C-terminal Lysine Residue
(SEQ ID NO: 33)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVFPLAPCSR
STSESTAALGCLVKDYFPEPVTVSWNSGALTSGVHTFPAVLQSSGLYSLSSVVTVPSSSLGTKTYTCNVD
HKPSNTKVDKRVESKYGPPCPSCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSLG*
G9.2-17 hIgG4 Fab Arm Exchange mut Heavy Chain
(SEQ ID NO: 19)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVFPLAPCSR
STSESTAALGCLVKDYFPEPVTVSWNSGALTSGVHTFPAVLQSSGLYSLSSVVTVPSSSLGTKTYTCNVD
HKPSNTKVDKRVESKYGPPCPPCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSPGK*
G9.2-17 hIgG4 Fab Arm Exchange mut Heavy Chain with No C-terminal Lysine Residue
(SEQ ID NO: 34)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVFPLAPCSR
STSESTAALGCLVKDYFPEPVTVSWNSGALTSGVHTFPAVLQSSGLYSLSSVVTVPSSSLGTKTYTCNVD
HKPSNTKVDKRVESKYGPPCPPCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSPG*
G9.2-17 hIgG4 Fab Arm Exchange mut Heavy Chain
(SEQ ID NO: 23)
EVQLVESGGGLVQPGGSLRLSCAASGFTVSSSSIHWVRQAPGKGLEWVAYISSSSGYTYYADSVKGRFTI
SADTSKNTAYLQMNSLRAEDTAVYYCARYWSYPSWWPYRGMDYWGQGTLVTVSSASTKGPSVFPLAPCSR
STSESTAALGCLVKDYFPEPVTVSWNSGALTSGVHTFPAVLQSSGLYSLSSVVTVPSSSLGTKTYTCNVD
HKPSNTKVDKRVESKYGPPCPPCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSLGK*
G9.2-17 hIgG4 Fab Arm Exchange mut Heavy Chain with No C-terminal Lysine Residue
(SEQ ID NO: 35)
EVQLVESGGGLVQPGGSLRLSCAASGFTVSSSSIHWVRQAPGKGLEWVAYISSSSGYTYYADSVKGRETI
SADTSKNTAYLQMNSLRAEDTAVYYCARYWSYPSWWPYRGMDYWGQGTLVTVSSASTKGPSVFPLAPCSR
STSESTAALGCLVKDYFPEPVTVSWNSGALTSGVHTFPAVLQSSGLYSLSSVVTVPSSSLGTKTYTCNVD
HKPSNTKVDKRVESKYGPPCPPCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSLG*
Any of the above heavy chain can be paired with a Light Chain of
shown below:
(SEQ ID NO: 15)
DIQMTQSPSSLSASVGDRVTITCRASQSVSSAVAWYQQKPGKAPKLLIYSASSLYSGVPSRFSGSRSGTD
FTLTISSLQPEDFATYYCQQSSTDPITFGQGTKVEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSEN
RGEC*

In some embodiments, the anti-Galectin-9 antibody comprises a heavy chain IgG1 constant region that has at least 80% (e.g., 80%, 85%, 90%, 91%, 92%, 93%, 94%, 95%, 96%, 97%, 98%, or 99% and any increment therein) sequence identity to SEQ ID NO: 10. In one embodiment, the constant region of the anti-Galectin-9 antibody comprises a heavy chain IgG4 constant region comprising SEQ ID NO: 10. In one embodiment, the constant region of the anti-Galectin-9 antibody comprises a heavy chain IgG1 constant region consisting of SEQ ID NO: 10.

In some embodiments, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 20. In one embodiment, the constant region of the anti-Galectin-9 antibody comprises a heavy chain IgG4 constant region comprising SEQ ID NO: 20. In one embodiment, the constant region of the anti-Galectin-9 antibody comprises a heavy chain IgG4 constant region consisting of SEQ ID NO: 20.

In some embodiments, the constant region is from human IgG4. In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 13. In one embodiment, the anti-Galectin-9 antibody comprises a heavy chain IgG4 constant region comprising SEQ ID NO: 13. In one embodiment, the anti-Galectin-9 antibody comprises a heavy chain IgG4 constant region consisting of SEQ ID NO: 13.

In some embodiments, the constant region is from human IgG4. In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 20. In one embodiment, the anti-Galectin-9 antibody comprises a heavy chain IgG4 constant region comprising SEQ ID NO: 20. In one embodiment, the anti-Galectin-9 antibody comprises a heavy chain IgG4 constant region consisting of SEQ ID NO: 20.

In any of these embodiments, the anti-Galectin-9 antibody comprises a light chain constant region that has at least 80% (e.g., 80%, 85%, 90%, 91%, 92%, 93%, 94%, 95%, 96%, 97%, 98%, or 99% and any increment therein) sequence identity to SEQ ID NO: 11. In some embodiments, the anti-Galectin-9 antibody comprises a light chain constant region comprising SEQ ID NO: 11. In some embodiments, the anti-Galectin-9 antibody comprises a light chain constant region consisting of SEQ ID NO: 11.

In some embodiments, the IgG is a mutant with minimal Fc receptor engagement. In one example, the constant region is from a human IgG1 LALA. In one embodiment, the anti-Galectin-9 antibody comprises a heavy chain IgG1 constant region that has at least 80% (e.g., 80%, 85%, 90%, 91%, 92%, 93%, 94%, 95%, 96%, 97%, 98%, or 99% and any increment therein) sequence identity to SEQ ID NO: 12. In one embodiment, the anti-Galectin-9 antibody comprises a heavy chain IgG1 constant region comprising SEQ ID NO: 12. In one embodiment, the anti-Galectin-9 antibody comprises a heavy chain IgG1 constant region consisting of SEQ ID NO: 12.

In some embodiments, the anti-Galectin-9 antibody comprises a modified constant region. In some embodiments, the anti-Galectin-9 antibody comprise a modified constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT Application No. PCT/GB99/01441; and/or UK Patent Application No. 9809951.8. In some embodiments, the IgG4 constant region is a mutant with reduced heavy chain exchange. In some embodiments, the constant region is from a human IgG4 Fab Arm Exchange mutant S228P.

In one embodiment, the constant region of the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 14. In one embodiment, the constant region of the anti-Galectin-9 antibody comprises a heavy chain IgG4 constant region comprising SEQ ID NO: 14. In one embodiment, the constant region of the anti-Galectin-9 antibody comprises a heavy chain IgG4 constant region consisting of SEQ ID NO: 14.

In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 21. In one embodiment, the anti-Galectin-9 antibody comprises a heavy chain IgG4 constant region comprising SEQ ID NO: 21. In one embodiment, the anti-Galectin-9 antibody comprises a heavy chain IgG4 constant region consisting of SEQ ID NO: 21.

In some embodiments, the anti-Galectin-9 antibody has chains corresponding to SEQ ID NO: 15 for the light chains; and the amino acid sequences of exemplary heavy chains correspond to SEQ ID NO: 10 (hIgG1); 12 (hIgG1 LALA); 13 (hIgG4); 20 (hIgG4); 14 (hIgG4 mut); and 21 (hIgG4 mut).

In some embodiments, the anti-Galectin-9 antibody has a light chain comprising, consisting essentially of, or consisting of SEQ ID NO: 15. In some embodiments, the anti-Galectin-9 antibody has a heavy chain comprising, consisting essentially of, or consisting of any one of the sequences selected from the group consisting of SEQ ID NO: 16-19, 22 and 23. In some embodiments, the anti-Galectin-9 antibody has a light chain comprising, consisting essentially of, or consisting of SEQ ID NO: 15 and a heavy chain comprising, consisting essentially of, or consisting of any one of the sequences selected from the group consisting of SEQ ID NO: 16-19. In some embodiments, the anti-Galectin-9 antibody has a light chain comprising SEQ ID NO: 15 and a heavy chain comprising any one of the sequences selected from the group consisting of SEQ ID NO: 16-19, 22 and 23. In some embodiments, the anti-Galectin-9 antibody has a light chain consisting essentially of SEQ ID NO: 15 and a heavy chain consisting essentially of any one of the sequences selected from the group consisting of SEQ ID NO: 16-19, 22 and 23. In some embodiments, the anti-Galectin-9 antibody has a light chain consisting of SEQ ID NO: 15 and a heavy chain consisting of any one of the sequences selected from the group consisting of SEQ ID NO: 16-19, 22 and 23. In one specific embodiment, the anti-Galectin-9 antibody has a light chain consisting essentially of SEQ ID NO: 15 and a heavy chain consisting essentially of SEQ ID NO: 19. In another specific embodiment, the anti-Galectin-9 antibody has a light chain consisting essentially of SEQ ID NO: 15 and a heavy chain consisting essentially of SEQ ID NO: 20.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 16. In one embodiment, the anti-Galectin-9 antibody comprises a heavy chain sequence comprising SEQ ID NO: 16. In one embodiment, the anti-Galectin-9 antibody comprises a heavy chain sequence consisting of SEQ ID NO: 16.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 17. In one embodiment, the anti-Galectin-9 antibody comprises a heavy chain sequence comprising SEQ ID NO: 17. In one embodiment, the anti-Galectin-9 antibody comprises a heavy chain sequence consisting of SEQ ID NO: 17.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 18. In one embodiment, the anti-Galectin-9 antibody comprises a heavy chain sequence comprising SEQ ID NO: 18. In one embodiment, the anti-Galectin-9 antibody comprises a heavy chain sequence consisting of SEQ ID NO: 18.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 22. In one embodiment, the anti-Galectin-9 antibody comprises a heavy chain sequence comprising SEQ ID NO: 22. In one embodiment, the anti-Galectin-9 antibody comprises a heavy chain sequence consisting of SEQ ID NO: 22.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 19. In one embodiment, the anti-Galectin-9 antibody comprises a heavy chain sequence comprising SEQ ID NO: 19. In one embodiment, the anti-Galectin-9 antibody comprises a heavy chain sequence consisting of SEQ ID NO: 19.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 23. In one embodiment, the anti-Galectin-9 antibody comprises a heavy chain sequence comprising SEQ ID NO: 23. In one embodiment, the anti-Galectin-9 antibody comprises a heavy chain sequence consisting of SEQ ID NO: 23.

In any of these embodiments, the anti-Galectin-9 antibody comprises a light chain sequence having at least 80% (e.g., 80%, 85%, 90%, 91%, 92%, 93%, 94%, 95%, 96%, 97%, 98%, or 99% and any increment therein) sequence identity to SEQ ID NO: 15. In some embodiments, the anti-Galectin-9 antibody comprises a light chain sequence comprising SEQ ID NO: 15. In some embodiments, the anti-Galectin-9 antibody comprises a light chain sequence consisting of SEQ ID NO: 15.

In specific examples, the anti-Galectin-9 antibody used in the treatment methods disclosed herein has a heavy chain of SEQ ID NO:19 and a light chain of SEQ ID NO:15. In some embodiments, the anti-Galectin-9 antibody used in the treatment methods disclosed herein is G9.2-17 IgG4. In some examples, such an anti-Galectin-9 antibody does not have the C-terminal lysine residue in its heavy chain.

### Preparation of Anti-Galectin-9 Antibodies

Antibodies capable of binding Galectin-9 as described herein can be made by any method known in the art, including but not limited to, recombinant technology. One example is provided below.

Nucleic acids encoding the heavy and light chain of an anti-Galectin-9 antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementarity ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987); Gossen and Bujard (1992); M. Gossen et al., Natl. Acad. Sci. USA, 89:5547-5551 (1992)) combined the tetracycline repressor (tetR) with the transcription activator (VP 16) to create a tetR-mammalian cell transcription activator fusion protein, Ta (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-Galectin-9 antibody, as also described herein. The recombinant expression vector can be introduced into a suitable host cell (e.g., a dhfr− CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-Galectin-9 antibody and the other encoding the light chain of the anti-Galectin-9 antibody. Both of the two recombinant expression vectors can be introduced into a suitable host cell (e.g., dhfr− CHO cell) by a conventional method, e.g., calcium phosphate-mediated transfection. Alternatively, each of the expression vectors can be introduced into a suitable host cell. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-Galectin-9 antibody as described herein, vectors (e.g., expression vectors) containing such; and host cells comprising the vectors are within the scope of the present disclosure.

Anti-Galectin-9 antibodies thus prepared can be characterized using methods known in the art, whereby reduction, amelioration, or neutralization of Galectin-9 biological activity is detected and/or measured. For example, in some embodiments, an ELISA-type assay is suitable for qualitative or quantitative measurement of Galectin-9 inhibition of Dectin-1 or TIM-3 signaling.

The bioactivity of an anti-Galectin-9 antibody can verified by incubating a candidate antibody with Dectin-1 and Galectin-9, and monitoring any one or more of the following characteristics: (a) binding between Dectin-1 and Galectin-9 and inhibition of the signaling transduction mediated by the binding; (b) preventing, ameliorating, or treating any aspect of a solid tumor; (c) blocking or decreasing Dectin-1 activation; (d) inhibiting (reducing) synthesis, production or release of Galectin-9. Alternatively, TIM-3 can be used to verify the bioactivity of an anti-Galectin-9 antibody using the protocol described above. Alternatively, CD206 can be used to verify the bioactivity of an anti-Galectin-9 antibody using the protocol described above.

In some embodiments, bioactivity or efficacy is assessed in a subject, e.g., by measuring peripheral and intra-tumoral T cell ratios, T cell activation, or by macrophage phenotyping.

Additional assays to determine bioactivity of an anti-Galectin-9 antibody include measurement of CD8+ and CD4+ (conventional) T-cell activation (in an in vitro or in vivo assay, e.g., by measuring inflammatory cytokine levels, e.g., IFNgamma, TNFalpha, CD44, ICOS granzymeB, Perforin, IL2 (upregulation); CD26L and IL-10 (downregulation)); measurement of reprogramming of macrophages (in vitro or in vivo), e.g., from the M2 to the M1 phenotype (e.g., increased MHCII, reduced CD206, increased TNF-alpha and iNOS), Alternatively, levels of ADCC can be assessed, e.g., in an in vitro assay, as described herein.

### Methods of Treatment

The present disclosure provides methods for treating solid tumors, including, but not limited to, PDAC, CRC, HCC, and cholangiocarcinoma, renal cell carcinoma, urothelial cancer, head and neck cancer, breast cancer, or other GI solid tumors, using any of the anti-Galectin antibodies, for example G9.2-17, e.g., G9.2-17 IgG4, either alone or in combination with a checkpoint inhibitor such as an anti-PD-1 antibody. Any of the anti-Galectin-9 antibodies described herein can be used in any of the methods described herein. In some embodiments, the anti-Galectin-9 antibody is G9.2-17 (e.g., G9.2-17 (IgG4)). Such antibodies can be used for treating diseases associated with Galectin-9. In some aspects, the present disclosure provides methods of treating cancer. In some embodiments, the present disclosure methods for reducing, ameliorating, or eliminating one or more symptom(s) associated with cancer.

### (A) Exemplary Target Solid Tumors

In some embodiments, the disclosure provides a method for treating a solid tumor in a subject, the method comprising administering to a subject in need thereof an effective amount of an anti-Galectin-9 antibody described herein, including but not limited to, G9.2-17 IgG4. In some examples, the method disclosed herein is applied to a human patient having pancreatic cancer, for example, ductal adenocarcinoma (PDAC). In some instances, the PDAC patient may have a metastatic cancer. In some examples, the method disclosed herein is applied to a human patient having colorectal cancer (CRC). In some embodiments, the colorectal cancer is metastatic. In some examples, the method disclosed herein is applied to a human patient having hepatocellular carcinoma. In some embodiments, the hepatocellular carcinoma is metastatic. In other examples, the method disclosed herein is applied to a human patient having cholangiocarcinoma. In some embodiments, the cholangiocarcinoma is metastatic.

Pancreatic ductal adenocarcinoma (PDAC) is a devastating disease with few long-term survivors (Yadav et al., Gastroenterology, 2013, 144, 1252-1261). Inflammation is paramount in PDAC progression as oncogenic mutations alone, in the absence of concomitant inflammation, are insufficient for tumorigenesis (Guerra et al., Cancer Cell, 2007, 11, 291-302). Innate and adaptive immunity cooperate to promote tumor progression in PDAC. In particular, specific innate immune subsets within the tumor microenvironment (TME) are apt at educating adaptive immune effector cells towards a tumor-permissive phenotype. Antigen presenting cell (APC) populations, including M2-polarized tumor-associated macrophages (TAMs) and myeloid dendritic cells (DC), induce the generation of immune suppressive Th2 cells in favor of tumor-protective Th1 cells (Ochi et al., J of Exp Med., 2012, 209, 1671-1687; Zhu et al., Cancer Res., 2014, 74, 5057-5069). Similarly, it has been shown that myeloid derived suppressor cells (MDSC) negate anti-tumor CD8⁺ cytotoxic T-Lymphocyte (CTL) responses in PDAC and promote metastatic progression (Connolly et al., J Leuk Biol., 2010, 87, 713-725; Pylayeva-Gupta et al., Cancer Cell, 2012, 21, 836-847; Bayne et al., Cancer Cell, 2012, 21, 822-835).

Pancreatic cancer remains a disease that is difficult to treat due to a typically late presentation, relatively high resistance to chemotherapy, and lack of effective immune and targeted therapies. Globally, approximately 455,000 new cases of pancreatic cancer have been reported in 2018, and an estimated 355,000 new cases are estimated to occur until 2040 annually, and almost as many deaths are reported as new cases on a yearly basis. It is projected to be the second leading cause of cancer-related deaths in the United States by the year 2030. Despite intervention, the median life expectancy for patients with metastatic pancreatic cancer is less than 1 year with current treatment, while most patients (as many as 80%) present at an advanced/metastatic stage, when the disease is beyond curative resection. Despite advancements in the detection and management of pancreatic cancer, the five-year survival rate of metastatic disease remains at ten percent. The current standard of care for metastatic pancreatic cancer is predominantly chemotherapy, while a distinct minority of patients (under ten percent) with BRCA1/2 mutations and mismatch repair deficient tumors may benefit from PARP inhibitors and potentially anti-PD-1 therapy. However, for the vast majority of patients with this disease, currently approved immunotherapies have been generally unsuccessful due to a highly immunosuppressive environment.

Colorectal cancer (CRC), also known as bowel cancer, colon cancer, or rectal cancer, is any cancer affecting the colon and the rectum. CRC is known to be driven by genetic alterations of tumor cells and is also influenced by tumor-host interactions. Recent reports have demonstrated a direct correlation between the densities of certain T lymphocyte subpopulations and a favorable clinical outcome in CRC, supporting a major role of T-cell-mediated immunity in repressing tumor progression of CRC.

CRC presents one of the largest cancer burdens in the world. Nowadays, it is the world's fourth most deadly cancer with almost 900,000 deaths annually globally. In the United States, 147,950 cases are predicted in 2020 with 53,200 estimated deaths (Colorectal Cancer Stats). Despite significant advances in standard of care therapies, the five-year survival rate for metastatic CRC remains around <20%. Death from CRC is expected to nearly double within the next 20 years. The current standard of care for CRC are chemotherapy regimens, combined and/or sequenced with anti-angiogenic therapy and anti-epidermal growth factor receptor modalities in selected patients. In addition, current immunotherapies are only efficacious (albeit producing profound and durable responses) in the small subset of patients whose tumors are mismatch-repair-deficient and microsatellite-instability-high (dMMR/MSI-H) (Dekker et al., 2019). Outcomes on immunotherapy in microsatellite stable CRC, which are the majority of patients with CRC are suboptimal and this represent a significant unmet medical need for active immunotherapy. CRC, the small subset that is dMMR/MSI-H derive benefit from immunotherapy (Huyghe et al., 2019), but the vast majority of patients with proficient mismatch repair or with microsatellite stable CRC, do not.

Hepatocellular carcinoma (HCC) is the most common type of primary liver cancer. Hepatocellular carcinoma occurs most often in people with chronic liver diseases, such as cirrhosis caused by hepatitis B or hepatitis C infection. HCC is usually accompanied by cirrhotic liver with extensive lymphocyte infiltration due to chronic viral infection. Many studies have demonstrated that tumor-infiltrating effector CD8+ T cells and T helper 17 (Th17) cells correlate with improved survival after surgical resection of tumors. However, tumor-infiltrating effector T cells fail to control tumor growth and metastasis (Pang et al., Cancer Immunol Immunother 2009; 58:877-886).

Cholangiocarcinoma (CCA) is a group of cancers that begin in the bile ducts. Cholangiocarcinoma is commonly classified by its location in relation to the liver. For example, intrahepatic cholangiocarcinoma, accounting for less than 10% of all cholangiocarcinoma cases, begins in the small bile ducts within the liver. In another example, perihilar cholangiocarcinoma (also known as a Klatskin tumor), accounting for more than half of the cholangiocarcinoma cases, begins in hilum, where two major bile ducts join and leave the liver. Others are classified as distal cholangiocarcinomas, which begin in bile ducts outside the liver.

CCAs are aggressive tumors, and most patients have advanced-stage disease at presentation. The incidence of CCA is rising, and effective therapies are urgently needed. Gemcitabine plus cisplatin remains the standard first-line systemic therapy for advanced CCA, although it leaves much to be desired, as median survival is less than 1 year. Beyond failure of first line therapy, available evidence to guide therapeutic decisions is scarce. Food and Drug Administration (FDA) only recently approved the first targeted therapy in this indication for patients harboring fibroblast growth factor receptor 2 gene fusions and other rearrangements in their tumors. Suboptimal response rates to immunotherapy in human clinical trials imply that the preponderance of CCAs are immune ‘cold’ tumors with a non-T-cell infiltrated microenvironment. In fact, immunotherapy to date has produced response rates not exceeding 17% and as of the date of this prospectus, no immune oncology agents have been approved (Zayac and Almhanna, 2020).

The silent presentation of these tumors combined with their highly aggressive nature and refractoriness to chemotherapy contribute to their alarming mortality where the five-year survival for patients with distant disease remains at a dismal 2% (Banales et al., 2020; Bile Duct Cancer Survival, 2020).

In some embodiments, methods are provided to increase anti-tumor activity (e.g., reduce cell proliferation, tumor growth, tumor volume, and/or tumor burden or load or reduce the number of metastatic lesions over time) by at least about 10%, 20%, 25%, 30%, 40%, 50%, 60%, 70%, 75%, 80%, 85%, 90%, 95%, or more as compared to levels prior to treatment or in a control subject. In some embodiments, reduction is measured by comparing cell proliferation, tumor growth, and/or tumor volume in a subject before and after administration of the pharmaceutical composition. In some embodiments, the method is provided to improve one or more symptoms of the cancer by at least about 10%, 20%, 30%, 40%, 50%, 60%, 70%, 80%, 90%, 95%, or more. In some embodiments, before, during, and after the administration of the pharmaceutical composition, cancerous cells and/or biomarkers in a subject are measured in a biological sample, such as blood, serum, plasma, urine, peritoneal fluid, and/or a biopsy from a tissue or organ. In some embodiments, the methods include administration of the compositions of the invention to reduce tumor volume, size, load or burden in a subject to an undetectable size, or to less than about 1%, 2%, 5%, 10%, 20%, 25%, 30%, 40%, 50%, 60%, 70%, 75%, 80%, or 90% of the 20 subject's tumor volume, size, load or burden prior to treatment. In other embodiments, methods are provided for reducing the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment. In other embodiments, methods include administration of the compositions of the invention to reduce the development of or the number or size of metastatic lesions in a subject to an undetectable rate, or to less than about 1%, 2%, 5%, 10%, 20%, 25%, 30%, 40%, 50%, 60%, 70%, 75%, 80%, or 90% of the rate prior to treatment.

The term “about” or “approximately” means within an acceptable error range for the particular value as determined by one of ordinary skill in the art, which are dependent in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to +5%, and more preferably still up to +1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, a symptom of the disease or disorder, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

### (B) Exemplary Patient Population for Treatment

A subject having any of the above noted cancers can be identified by routine medical examination, e.g., laboratory tests, organ functional tests, genetic tests, interventional procedure (biopsy, surgery) any and all relevant imaging modalities.

In some embodiments, the subject to be treated by the method described herein is a human cancer patient who has undergone or is subjected to an anti-cancer therapy regimen delivered systemically and/or locally, for example, chemotherapy, radiotherapy, tumor-treating fields (TTFields), immunotherapy, biological therapy, small molecule inhibitors, anti-hormonal therapy, cell-based therapy, and/or surgery, in any combination or sequence of the outlined therapeutic modalities. In some embodiments, subjects have received prior immune-modulatory or any other anti-tumor agents or treatment modalities listed above. Non-limiting examples of such immune-modulatory agents include, but are not limited to as anti-PD-1, anti-PD-L1, anti-CTLA-4, anti-TIGIT, anti-PVRIG, anti-LAG-3, anti-CD47, anti-CD40, anti-CSFR1, anti-CD73, anti-SIRP, anti-A2AR, anti-OX40, anti-CD137, platinum-based agent, etc. Non-limiting examples of platinum-based agents include cisplatin, carboplatin, oxaliplatin, nedaplatin, and lobaplatin. In some embodiments, the subject shows disease progression through the treatment. In other embodiments, the subject is resistant to the treatment (either de novo or acquired). In some embodiments, such a subject is demonstrated as having advanced malignancies (e.g., inoperable or metastatic). Alternatively, or in addition, in some embodiments, the subject has no standard therapeutic options available or ineligible for standard treatment options, which refer to therapies commonly used in clinical settings for treating the corresponding solid tumor.

Tumor-treating fields (TTFields) are a cancer treatment modality that uses alternating electric fields of intermediate frequency (˜100-500 kHz) and low intensity (1-3 V/cm) to disrupt cell division. In any of the embodiments described herein, the anti-Galectin-9 antibody, alone or in combination with a checkpoint inhibitor, such as an anti-PD-1 antibody, may be administered prior to, concurrent with, or after a tumor-treating fields (TTFields) regimen.

In some instances, the subject may be a human patient having a refractory disease, for example, a refractory PDAC, a refractory CRC, a refractory HCC, or a refractory cholangiocarcinoma. As used herein, “refractory” refers to the tumor that does not respond to or becomes resistant to a treatment. In some instances, the subject may be a human patient having a relapsed disease, for example, a relapsed PDAC, a relapsed CRC, a relapsed HCC, or a relapsed cholangiocarcinoma. As used herein, “relapsed” or “relapses” refers to the tumor that returns or progresses following a period of improvement (e.g., a partial or complete response) with treatment.

In some embodiments, the human patient to be treated by the methods disclosed herein meets one or more of the inclusion and exclusion criteria disclosed in Example 1 below. For example, the human patient may be 18 or older; having histologically confirmed unresectable metastatic or inoperable cancer (e.g., without standard therapeutic options), having a life expectancy >3 months, having recent archival tumor sample available for biomarker analysis (e.g., an archival species for Galectin-9 tumor tissue expression levels assessed by IHC); having a measurable disease, according to RECIST v1.1, having Eastern Cooperative Oncology Group (ECOG) performance status 0-1 or Karnofsky score >70; having no available standard of care options, having MSI-H (Microsatellite instability high and MSS (Microsatellite Stable); received at least one line of systemic therapy in the advanced/metastatic setting; having adequate hematologic and end organ function (defined in Example 1 below; e.g., e.g., neutrophil count ≥1×10⁹/L, platelet count ≥100×10⁹/L, for HCC in Part 1≥50×10⁹/L; hemoglobin ≥9.0 g/dL without transfusion in the previous week, Creatinine≤1.5×ULN, AST (SGOT)≤3×ULN (≤5×ULN when HCC or hepatic metastases are present), ALT (SGPT)≤3×ULN (≤5×ULN when HCC or hepatic metastases present), Bilirubin≤1.5×ULN (patients with known Gilbert's disease may have a bilirubin≤3.0×ULN), Albumin≥3.0 g/dL, INR and PTT≤1.5×ULN; and/or amylase and lipase≤1.5×ULN)); having completed treatment for brain metastases if any (see Example 1 below); having no evidence of active infection and no serious infection within the past month; having at least four (4) weeks s or 5 half lives (whichever is shorter) since the last dose of anti-cancer therapy before the first anti-Gal-9 antibody administration; having continued bisphosphonate treatment (zolendronic acid) or denosumab for bone metastases if applicable. CCR or CCA patients subject to the instant treatment may have at least one prior line of therapy in the metastatic setting is required. In some embodiments, CCR or CCA patients subject to the instant treatment have had at least one prior line of therapy in the metastatic setting.

Alternatively or in addition, the subject suitable for the treatment disclosed herein may not have one or more of the following: diagnosed with metastatic cancer of an unknown primary; any active uncontrolled bleeding, and any patients with a bleeding diathesis (e.g., active peptic ulcer disease); receiving any other investigational agents within 4 weeks or 5 half-lives of anti-galectin-9 antibody administration; receiving radiation therapy within 4 weeks of the first dose of the anti-Galectin-9 antibody, except for palliative radiotherapy to a limited field, such as for the treatment of bone pain or a focally painful tumor mass; having fungating tumor masses; for PDAC patients, having prior gemcitabine containing regimen less than 6 months from the begin of the treatment, patients having locally advanced PDAC; having active clinically serious infection >grade 2 NCI-CTCAE version 5.0; having symptomatic or active brain metastases; having ≥CTCAE grade 3 toxicity (see details and exceptions in Example 1); having history of second malignancy (see exceptions in Example 1); having evidence of severe or uncontrolled systemic diseases, congestive cardiac failure; having serious non-healing wound, active ulcer or untreated bone fracture; having uncontrolled pleural effusion, pericardial effusion, or ascites requiring recurrent drainage procedures; having spinal cord compression not definitively treated with surgery and/or radiation. Leptomeningeal disease, active or previously treated; having significant vascular disease; having active auto-immune disorder (see exceptions in Example 1); require systemic immunosuppressive treatment; having tumor-related pain (>grade 3) unresponsive to broad analgesic interventions (oral and/or patches); having uncontrolled hypercalcemia, despite use of bisphosphonates; having any history of an immune-related Grade 4 adverse event attributed to prior checkpoint inhibitor therapy (CIT); received an organ transplant(s); and/or on undergoing dialysis; for HCC patients and/or CCA patients, having any ablative therapy prior to the treatment; hepatic encephalopathy or severe liver adenoma; and/or having Child-Pugh score ≥7. In some instances, the human patient may not have metastatic hepatocellular carcinoma that progressed while receiving at least one previous line of systemic therapy; have refuse or not tolerated sorafenib; or have had standard therapy considered ineffective, intolerable, or inappropriate or for which no effective standard therapy is available.

In some embodiments, the human patient to be treated by the methods disclosed herein may meet one or more of the inclusion and exclusion criteria disclosed in Example 1 below. For example, the human patient may be older than 18 and have histologically confirmed unresectable metastatic cancer (e.g., adenocarcinomas and squamous cell carcinomas). The patient may have measurable disease, according to RECIST v. 1.1. In some instances, the human patient may have recent archival tumor sample (e.g., obtained within 5 years) available for biomarker analyses (e.g., galectin-9 tumor tissue expression, which may be assessed by IHC). In some instances, the human patient is a PDAC patient who has received at least one line of systemic therapy in the metastatic cancer setting. In some instances, the human patient is a metastatic PDAC patient who has or has not received systemic therapy before receiving an anti-Galectin-9 containing regimen. Such a patient may either be gemcitabine-containing regimen naïve or at least 6 months out of having been treated using a gemcitabine-containing regimen in a previous disease stage setting. The patient may have Eastern Cooperative Oncology Group (ECOG) performance status 0-1 and/or Karnofsky score >70. The patient may also have adequate hematologic and end organ function, e.g., neutrophil count ≥1×10⁹/L, platelet count ≥100×10⁹/L, for HCC in Part 1≥50×10⁹/L; hemoglobin ≥9.0 g/dL without transfusion in the previous week, Creatinine≤1.5×ULN, AST (SGOT)≤3×ULN (≤5×ULN when HCC or hepatic metastases are present), ALT (SGPT)≤3×ULN (≤5×ULN when HCC or hepatic metastases present), Bilirubin≤1.5×ULN (patients with known Gilbert's disease may have a bilirubin≤3.0×ULN), Albumin ≥3.0 g/dL, INR and PTT≤1.5×ULN; and/or amylase and lipase≤1.5×ULN. In some instances, the human patient shows no evidence of active infection or infections requiring parenteral antibiotics, and no serious infection within 4 weeks before the treatment starts. Pancreatic, biliary, or enteric fistulae allowed, provided they are controlled with an appropriate non-infected and patent drain.

Alternatively or in addition, the human patient subject to any treatment disclosed herein may be free of: (i) metastatic cancer of an unknown primary, (ii) clinically significant, active uncontrolled bleeding, any bleeding diathesis (e.g., active peptic ulcer disease); (iii) radiation therapy within 4 weeks of the first dose of the treatment, (iv) with fungating tumor masses; (v) ≥CTCAE grade 3 toxicity (except alopecia and vitiligo) due to prior cancer therapy; (v) history of second malignancy, (vi) evidence of severe or uncontrolled systemic diseases, congestive cardiac failure >New York Heart Association (NYHA) class 2, or myocardial infarction (MI) within 6 months, (vii) serious non-healing wound, active ulcer, or untreated bone fracture; (viii) uncontrolled pleural effusion, pericardial effusion, or ascites requiring recurrent drainage procedures; (ix) history of severe allergic, anaphylactic, or other hypersensitivity reactions to chimeric or humanized antibodies or fusion proteins; (x) significant vascular disease (e.g., aortic aneurysm requiring surgical repair or recent arterial thrombosis) within 6 months of the treatment, history of pulmonary embolism, stroke or transient ischemic attack within 3 months prior to the treatment, and/or history of abdominal fistula or gastrointestinal perforation within 6 months prior to the treatment; (xi) active auto-immune disorder (except type I diabetes, hypothyroidism requiring only hormone replacement, vitiligo, psoriasis, or alopecia); (xii) requires systemic immunosuppressive treatment; (xii) tumor-related pain (>grade 3) unresponsive to broad analgesic interventions (oral and/or patches); (xiii) uncontrolled hypercalcemia, despite use of bisphosphonates; (xiv) received organ transplant(s).

In some instances, the subject is a human patient having an elevated level of Galectin-9 as relative to a control level. The level of Galectin-9 can be a plasma or serum level of Galectin-9 in the human patient. In other examples, the level of Galectin-9 is the level of Galectin-9 of cancer cells within the tumor. In other examples, the level of Galectin-9 is the level of Galectin-9 of immune cells within the tumor. In other examples, the level of Galectin-9 can be the level of cell-surface Galectin-9, for example the level of Galectin-9 on cancer cells. In one example, the level of Galectin-9 can be the level of Galectin-9 expressed cancer cells, e.g., on the surface of cancer cells, or Galectin-9 expressed in immune cells, measured in patient-derived organotypic tumor spheroids (PDOT), which can be prepared by, e.g., the method disclosed in Examples below. A control level may refer to the level of Galectin-9 in a matched sample of a subject of the same species (e.g., human) who is free of the solid tumor. In some examples, the control level represents the level of Galectin-9 in healthy subjects. In some embodiments, the control level may be a baseline level prior to treatment.

To identify such a subject, a suitable biological sample can be obtained from a subject who is suspected of having the solid tumor and the biological sample can be analyzed to determine the level of Galectin-9 contained therein (e.g., free, cell-surface expressed, or total) using conventional methods, e.g., ELISA or FACS. In some embodiments, organoid cultures are prepared, e.g., as described herein, and used to assess Galectin-9 levels in a subject. Single cells derived from certain fractions obtained as part of the organoid preparation process are also suitable for assessment of Galectin-9 levels in a subject. In some instances, an assay for measuring the level of Galectin-9, either in free form or expressed on cell surface, involves the use of an antibody that specifically binds the Galectin-9 (e.g., specifically binds human Galectin-9). Any of the anti-Galectin-9 antibodies known in the art can be tested for suitability in any of the assays described above and then used in such assays in a routine manner. In some embodiments, an antibody described herein (e.g., a G9.2-17 antibody) can be used in such as assay. In some embodiments, an antibody described in U.S. Pat. No. 10,344,091 and WO2019/084553, the relevant disclosures of each of which are incorporated by reference for the purpose and subject matter referenced herein. In some examples, the anti-Galectin-9 antibody is a Fab molecule. Assay methods for determining Galectin-9 levels as disclosed herein are also within the scope of the present disclosure.

### (C) Exemplary Treatment Conditions

In some embodiments, the antibodies described herein, e.g., G9.2-17, are administered to a subject in need of the treatment at an amount sufficient to inhibit the activity of Galectin-9 (and/or Dectin-1 or TIM-3 or CD206) in immune suppressive immune cells in a tumor by at least 20% (e.g., 30%, 40%, 50%, 60%, 70%, 80%, 90% or greater) in vivo. In other embodiments, the antibodies described herein, e.g., G9.2-17, are administered in an amount effective in reducing the activity level of Galectin-9 (and/or Dectin-1 or TIM-3 or CD206) in immune suppressive immune cells in a tumor by at least 20% (e.g., 30%, 40%, 50%, 60%, 70%, 80%, 90% or greater) (as compared to levels prior to treatment or in a control subject). In some embodiments, the antibodies described herein, e.g., G9.2-17, are administered to a subject in need of the treatment at an amount sufficient to promote M1-like programming in TAMs by at least 20% (e.g., 30%, 40%, 50%, 60%, 70%, 80%, 90% or greater) in vivo (as compared to levels prior to treatment or in a control subject).

Conventional methods, known to those of ordinary skill in the art of medicine, can be used to administer the pharmaceutical composition to the subject, depending upon the type of disease to be treated or the site of the disease. In some embodiments, the anti-Galectin-9 antibody can be administered to a subject by intravenous infusion.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous infusion, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

An effective amount of the pharmaceutical composition described herein can be administered to a subject (e.g., a human) in need of the treatment via a suitable route, systemically or locally. In some embodiments, the anti-Galectin-9 antibodies are administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, sub-urothelial, oral, inhalation or topical routes. In one embodiment, the anti-Galectin-9 antibody is administered to the subject by intravenous infusion. In one embodiment, the anti-galectin-9 antibody is administered to the subject intraperitoneally.

As used herein, “an effective amount” refers to the amount of each active agent required to confer therapeutic effect on the subject, either alone or in combination with one or more other active agents. In some embodiments, the therapeutic effect is reduced Galectin-9 activity and/or amount/expression, reduced Dectin-1 signaling, reduced TIM-3 signaling, reduced CD206 signaling, or increased anti-tumor immune responses in the tumor microenvironment. Non-limiting examples of increased anti-tumor responses include increased activation levels of effector T cells or switching of the TAMs from the M2 to the M1 phenotype. In some cases, the anti-tumor response includes increased ADCC responses. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally contribute to the determination of the dosage. For example, antibodies that are compatible with the human immune system, such as humanized antibodies or fully human antibodies, are in some instances used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein are determined empirically in individuals who have been given one or more administration(s) of the antibody. Individuals are given incremental dosages of the antagonist. To assess efficacy of the antagonist, an indicator of the disease/disorder can be followed.

### (D) Anti-Galectin-9 Antibody Treatment

In some embodiments, the anti-Galectin-9 antibodies described herein are be used for treating the target cancer disclosed herein, i.e., free of other anti-cancer therapy concurrently with the therapy using the anti-Galectin-9 antibody. In some instances, the anti-Galectin-9 antibody such as G9.2-17(IgG4) disclosed herein may be used in a monotherapy (i.e., with the anti-Galectin-9 antibody as the sole active agent). In other instances, the anti-Galectin-9 antibody such as G9.2-17(IgG4) disclosed herein may be used in a combined therapy, e.g., in combination with a PD-1 inhibitor such as those disclosed herein.

In some embodiments, the disclosure provides a method for treating a solid tumor in a subject, the method comprising administering to a subject in need thereof effective amount of an anti-Galectin-9 antibody or an effective amount of a pharmaceutical composition comprising an anti-Galectin-9 antibody described herein or antigen binding fragment thereof. Any of the anti-Galectin-9 antibodies disclosed herein can be used in the methods disclosed herein, e.g., antibody G9.2-17 (IgG4) (e.g., having a heavy chain of SEQ ID NO: 19 and a light chain of SEQ ID NO:15).

In some embodiments, the antibody is administered once every two to six weeks, e.g., via intravenous infusion. In some examples, the antibody may be administered once every 2-4 weeks, for example, every 2 weeks. In other examples, the antibody may be administered once every week. In some embodiments, the anti-Galectin 9 antibody disclosed herein (e.g., G9.2-17 IgG4) is administered via a 30-minute to 6-hour infusion period intravenously. In some examples the intravenous infusion of the anti-Galectin 9 antibody may be performed for 30 minutes to 2 hours. In other examples, the anti-Galectin 9 antibody may be administered via a long infusion period, for example, about 2-6 hours, e.g., about 2-4 hours or about 4-6 hours. In specific examples, examples anti-Galectin 9 antibody may be infused intravenous in a period of about 3 hours, about 4 hours, about 5 hours, or about 6 hours.

In some embodiments, the anti-Galectin-9 antibody disclosed herein (e.g., G9.2-17(IgG4)) for use in treating a solid tumor (e.g., those disclosed herein) can be administered to the subject at a dose of about 0.2 mg/kg to about 32 mg/kg, e.g., the dose may be selected from 0.2 mg/kg, 0.63 mg/kg, 2 mg/kg, 4 mg/kg, 6 mg/kg, 6.3 mg/kg, 8 mg/kg, 10 mg/kg, 12 mg/kg, and 16 mg/kg or a higher dose level. In some embodiments, the anti-Galectin-9 antibody may be administered to the subject at a dose of about 1 mg/kg to about 32 mg/kg, e.g., the dose may be selected from 2 mg/kg, 4 mg/kg, 8 mg/kg, 12 mg/kg, and 16 mg/kg or a higher dose level. In some examples, the anti-Galectin-9 antibody may be administered to the subject at a dose of about 0.2 mg/kg to about 32 mg/kg, e.g., the dose may be selected from 0.2 mg/kg, 0.63 mg/kg, 2 mg/kg, 4 mg/kg, 6 mg/kg, 6.3 mg/kg, 10 mg/kg, or 16 mg/kg or a higher dose level.

In some embodiments, the anti-Galectin-9 antibody is administered once every 2 weeks. In some embodiments, the anti-Galectin-9 antibody is administered once every 2 weeks for one cycle, once every 2 weeks for two cycles, once every 2 weeks for 3 cycles, once every 2 weeks for 4 cycles, or once every 2 weeks for more than 4 cycles. In some embodiments, the anti-Galectin-9 antibody is administered once every 2 weeks for 4 cycles. In some embodiments, the anti-Galectin-9 antibody is administered once every 4 or 6 weeks. In some embodiments, the duration of treatment is 12-24 months or longer. In some embodiments, the cycles extend for a duration of 3 months to 6 months, or 6 months to 12 months or 12 months to 24 months or longer. In some embodiments, the cycle length is modified, e.g., temporarily or permanently to a longer duration, e.g., 3 weeks or 4 weeks. In some embodiments, the use further comprises administering to the subject an immune checkpoint inhibitor, e.g., an anti-PD-1 antibody, as described herein, e.g., administered according to a regimen described herein. In some embodiments, the interval or cycle is one week. In some embodiments, the interval or cycle is 2 weeks. In specific embodiments, the interval or cycle is 2 weeks. In specific embodiments, the interval or cycle is 3 weeks. In specific embodiments, the interval or cycle is 4 weeks.

In solid tumor is selected from pancreatic ductal adenocarcinoma (PDAC), colorectal cancer (CRC), hepatocellular carcinoma (HCC), cholangiocarcinoma (CAA), renal cell carcinoma (RCC), urothelial, head and neck, breast cancer, lung cancer, and other GI solid tumors, and in some embodiments, the regimen or dosing schedule is once every 2 weeks for one cycle, once every 2 weeks for two cycles, once every 2 weeks for three cycles, once every 2 weeks for four cycles, or once every 2 weeks for more than four cycles. In some embodiments, the treatment is once every 2 weeks for 1 to 3 months, once every 2 weeks for 3 to 6 months, once every 2 weeks for 6 to 12 months, or once every 2 weeks for 12 to 24 months, or longer. In some embodiments, the antibody is administered via intravenous infusion.

In some embodiments, the regimen or dosing schedule is once every 3 weeks or 4 weeks for one cycle, once every 3 weeks or 4 weeks for two cycles, once every once every 3 weeks or 4 weeks for three cycles, once every 3 weeks or 4 weeks for four cycles, or once every 3 weeks or 4 weeks for more than four cycles. In some embodiments, the treatment is once every 3 weeks or 4 weeks for 1 to 3 months, once every 3 weeks or 4 weeks for 3 to 6 months, once every 3 weeks or 4 weeks for 6 to 12 months, or once every 3 weeks or 4 weeks for 12 to 24 months, or longer. In some embodiments, the treatment is once every 3 weeks or 4 weeks for 1 to 3 months, once every 6 weeks for 3 to 6 months, once every 3 weeks or 4 weeks for 6 to 12 months, or once every 3 weeks or 4 weeks for 12 to 24 months, or longer. In some embodiments, the treatment is longer than 24 months when clinically indicated. In some embodiments, the antibody is administered via intravenous infusion.

In other embodiments, the anti-Galectin-9 antibody such as G9.2-17 (IgG4) may be administered to a human patient at a suitable dose (e.g., the doses disclosed herein) once every week. For example, 2.0 mg/kg of G9.2-17(IgG4) may be administered to the human patient once every week. For example, 6.3 mg/kg of G9.2-17(IgG4) may be administered to the human patient once every week. In another example, 10 mg/kg of G9.2-17(IgG4) may be administered to the human patient once every week. Alternatively, 12 mg/kg of G9.2-17(IgG4) may be administered to the human patient once every week. In yet another example, 16 mg/kg of G9.2-17(IgG4) may be administered to the human patient once every week.

In some instances, the anti-Galectin-9 antibody may be given to the human patient for at least 2 cycles, at least 3 cycles, at least 4 cycles, at least 5 cycles, at least 6 cycles, or more. In some instances, the treatment period may be 6 months to 12 months. In other instances, the treatment period may be 12 months to 24 months. In other instances, the treatment period may be longer than 24 months.

In some instances, the anti-Gal-9 antibody such as G9.2-17(IgG4) disclosed herein may be administered to a subject at a flat dose, e.g., about 650 mg to about 1120 mg, once every week to once every 4 weeks. In some examples, the anti-Gal-9 antibody is administered to a subject at a about 650 mg to about 700 mg once every week. In some examples, the anti-Gal-9 antibody is administered to a subject at a about 650 mg to about 700 mg once every two weeks. In some examples, the anti-Gal-9 antibody is administered to a subject at a about 1040 mg to about 1120 mg once every week. In some examples, the anti-Gal-9 antibody is administered to a subject at a about 1040 mg to about 1120 mg once every two weeks.

In some embodiments, the dosage(s) is adjusted in accordance with the patient's response to treatment. In some embodiments, the dosages are altered between treatment intervals. In some embodiments, the treatment may be temporarily stopped. In some embodiments, the treatment may be temporarily stopped. In some embodiments, anti-Galectin-9 therapy is temporarily stopped. In some embodiments, a checkpoint inhibitor therapy employed in combination with the anti-Galectin-9 antibody is temporarily stopped. In some embodiments, both are temporarily stopped.

Alternatively, a human patient may start with a low dose of the anti-Galectin-9 antibody such as G9.2-17 (IgG4) disclosed herein, for example, 0.2 mg/kg, 0.63 mg/kg, or 2 mg/kg. When no adverse effects are observed, the dose of the antibody may be elevated, for example, to 6.3 mg/kg, 10 mg/kg, or 16 mg/kg.

Given that pro-tumor action of Galectin-9 is mediated through interaction with immune cells (e.g., interactions with lymphoid cells via TIM-3, CD44, and 41BB, and with macrophages via dectin-1 and CD206) and given that Galectin-9 is expressed in a large number of tumors, targeting Galectin-9, e.g., using a Galectin-9 binding antibody to inhibit interaction with its receptors provides a therapeutic approach that can be applied across a variety of different tumor types.

### (E) Combined Therapy

In some embodiments, any of the anti-Galectin-9 antibodies described herein (e.g., a G9.2-17 antibody such as G9.2-17(IgG4) as disclosed herein) can be used in any of the methods described herein, administered in combination with a second therapeutic, e.g., a checkpoint inhibitor, such as an anti-PD-1 antibody or an anti-PD-L1 antibody. Non-limiting examples of checkpoint inhibitors and administration regimen are provided elsewhere.

Accordingly, the treatment method disclosed herein may further comprise administering to the subject an inhibitor of a checkpoint molecule, for example, PD-1. Examples of PD-1 inhibitors include anti-PD-1 antibodies, such as pembrolizumab, nivolumab, tislelizumab, dostarlimab, and cemiplimab. Such checkpoint inhibitors can be administered simultaneously or sequentially (in any order) with the anti-Galectin-9 antibody according to the present disclosure. In some embodiments, the checkpoint molecule is PD-L1. Examples of PD-L1 inhibitors include anti-PD-L1 antibodies, such as durvalumab, avelumab, and atezolizumab. In some embodiments, the checkpoint molecule is CTLA-4. An example of a CTLA-4 inhibitor is the anti-CTLA-4 antibody ipilimumab. In some embodiments, the inhibitor targets a checkpoint molecule selected from CD40, GITR, LAG-3, OX40, TIGIT and TIM-3.

In some embodiments, the anti-Galectin-9 antibody improves the overall response, e.g., at 3 months, relative to a regimen comprising the inhibitor of the checkpoint molecule (e.g., anti-PD-1, for example, nivolumab) alone.

In some embodiments, the anti-PD-1 antibody is PD-1 is nivolumab, and the method described herein comprises administration of nivolumab to the subject at a dose of 240 mg intravenously once every two weeks.

In some embodiments, the antibody that binds PD-1 is co-used with the anti-Galectin-9 antibody disclosed herein (e.g., G9.2-17(IgG4)). In some instances, the anti-PD-1 antibody can be is administered using a flat dose. In some embodiments, the antibody that binds PD-1 is nivolumab, which can be administered to the subject at a dose of about 240 mg every two weeks or about 480 mg once every 4 weeks. In some embodiments, the antibody that binds PD-1 is prembrolizumab, which can be administered at a dose of about 200 mg once every 3 weeks. In some embodiments, the antibody that binds PD-1 is cemiplimab, which can be administered at a dose of about 350 mg intravenously once every 3 weeks. In some embodiments, the antibody that binds PD-1 is tislelizumab, which can be administered at a dose of about 200 mg intravenously once every 3 weeks or at a dose of about 400 mg intravenously once every 6 weeks. In some embodiments, the antibody that binds PD-1 is dostarlimab, which can be administered at a dose of about 500 mg intravenously every three weeks or about 1000 mg intravenously every six weeks.

In some embodiments, the antibody that binds PD-L1 (anti-PD-L1 antibody) is co-used with the anti-Galectin-9 antibody disclosed herein (e.g., G9.2-17(IgG4)). In some instances, the antibody that binds PD-L1 is administered using a flat dose. In some examples, the anti-PD-L1 antibody is atezolizumab, which may be administered at a dose of 1200 mg intravenously once every 3 weeks. In some examples, the anti-PD-L1 antibody antibody is Avelumab, which may be administered at a dose of 10 mg/kg intravenously every 2 weeks. In some embodiments, the anti-PD-L1 antibody is durvalumab, which may be administered at a dose of 1500 mg intravenously every 4 weeks.

In specific examples, any of the methods disclosed herein comprise (i) administering to a human patient having a target solid tumor as disclosed herein (e.g., pancreatic ductal adenocarcinoma (PDAC or PDAC), CRC, HCC, CCA, RCC, urothelial cancer, head and neck cancer, breast cancer, lung cancer, or other GI solid tumors) any of the anti-Galectin-9 antibodies disclosed herein (e.g., G9.2-17 such as the antibody having the heavy chain of SEQ ID NO:19 and the light chain of SEQ ID NO:5) at a dose of about 0.2 to about 32 mg/kg (e.g., about 3 mg/kg or about 15 mg/kg) once every two weeks; and (ii) administering to the human patient an effective amount of an anti-PD-1 antibody (e.g., nivolumab, prembrolizumab, tislelizumab, or cemiplimab, dostarlimab, durvalumab, avelumab, and atezolizumab).

In other specific examples, any of the methods disclosed herein comprise (i) administering to a human patient having a target solid tumor as disclosed herein (e.g., pancreatic ductal adenocarcinoma (PDAC or PDAC), CRC, HCC, CCA, RCC, urothelial cancer, head and neck cancer, breast cancer, lung cancer, or other GI solid tumors) any of the anti-Galectin-9 antibodies disclosed herein (e.g., G9.2-17 such as the antibody having the heavy chain of SEQ ID NO:19 and the light chain of SEQ ID NO:5) at a dose of about 0.2 to about 32 mg/kg (e.g., about 10 mg/kg or about 16 mg/kg) once every week; and (ii) administering to the human patient an effective amount of an anti-PD-1 or anti-PD-L1 antibody (e.g., nivolumab, prembrolizumab, tislelizumab, or cemiplimab, dostarlimab, durvalumab, avelumab, and atezolizumab).

Without being bound by theory, it is thought that anti-Galectin-9 antibodies, through their inhibition of Dectin-1, can reprogram immune responses against tumor cells via, e.g., inhibiting the activity of γδ T cells infiltrated into tumor microenvironment, and/or enhancing immune surveillance against tumor cells by, e.g., activating CD4+ and/or CD8+ T cells. Thus, combined use of an anti-Galectin-9 antibody and an immunomodulatory agent such as those described herein would be expected to significantly enhance anti-tumor efficacy.

In some embodiments, the methods are provided, wherein the anti-Galectin-9 antibody is administered concurrently with a checkpoint inhibitor. In some embodiments, the anti-Galectin-9 antibody is administered before or after a checkpoint inhibitor. In some embodiments, the checkpoint inhibitor is administered systemically. In some embodiments, the checkpoint inhibitor is administered locally. In some embodiments, the checkpoint inhibitor is administered by intravenous administration, e.g., as a bolus or by continuous infusion over a period of time, by intramuscular, intraperitoneal, intracerebrospinal, subcutaneous, intra-arterial, intra-articular, intravesical, intrasynovial, intrathecal, intratumoral, or sub-urothelial route. In one embodiment, the checkpoint inhibitor is administered to the subject by intravenous infusion.

In some instances, the checkpoint inhibitor such as any of the anti-PD-1 antibodies disclosed herein and any of the anti-Galectin 9 antibodies disclosed herein such as G9.2-17 (e.g., G9.2-17(IgG4)) may have same day administration. In some examples, the checkpoint inhibitor can be administered to a subject prior to administration of the anti-Galectin 9 antibody. In other instances, the administration of the checkpoint inhibitor, e.g., anti-PD-1 antibody, and the administration of the anti-Galectin 9 antibody are performed on two consecutive days. The checkpoint inhibitor, e.g., anti-PD-1 antibody, may be administered to the subject on the first day of dosing and the anti-Galectin-9 antibody can be administered to the subject on the subsequent day.

In other instances, the checkpoint inhibitor such as any of the anti-PD-1 antibodies disclosed herein may be administered about 1-7 days (e.g., 1 day, 2 days, 3 days, 4 days, 5 days, 6 days, or 7 days) prior to administration of the anti-Galectin 9 antibodies disclosed herein such as G9.2-17.

In some examples, the anti-Galectin 9 antibody can be administered to a subject prior to administration of the checkpoint inhibitor, e.g., an anti-PD-1 antibody. In other instances, the administration of the anti-Galectin 9 antibody and the administration of the checkpoint inhibitor, e.g., anti-PD-1 antibody, are performed on two consecutive days. The anti-Galectin-9 antibody may be administered to the subject on the first day of dosing and checkpoint inhibitor, e.g., anti-PD-1 antibody, can be administered to the subject on the subsequent day.

In other instances, the anti-Galectin-9 antibodies disclosed herein, such as G9.2-17, may be administered about 1-7 days (e.g., 1 day, 2 days, 3 days, 4 days, 5 days, 6 days, or 7 days) prior to administration of the checkpoint inhibitor, such as any of the anti-PD-1 antibodies disclosed herein.

In any of the method embodiments described herein, the anti-galectin-9 antibody can be administered (alone or in combination with an anti-PD-1 antibody) once every 2 weeks for one cycle, once every 2 weeks for two cycles, once every 2 weeks for three cycles, once every 2 weeks for four cycles, or once every 2 weeks for more than four cycles. In some embodiments, the treatment is 1 to 3 months, 3 to 6 months, 6 to 12 months, 12 to 24 months, or longer. In some embodiments, the treatment is once every 2 weeks for 1 to 3 months, once every 2 weeks for 3 to 6 months, once every 2 weeks for 6 to 12 months, or once every 2 weeks for 12 to 24 months, or longer.

Alternatively or in addition, the anti-Galectin-9 antibody may be used in combination with a regimen comprising UGN-102, UGN-201, or UGN-302. In one embodiment, UGN-102, UGN-201, or UGN-302 are formulated in a hydrogel e.g., a reverse-thermal hydrogel technology-based hydrogel. In some examples, the anti-Galectin-9 antibody can be administered prior to UGN-102, UGN-201, or UGN-302. In some examples, the anti-Galectin-9 antibody can be administered concurrently with UGN-102, UGN-201, or UGN-302. In some examples, the anti-Galectin-9 antibody may be administered after UGN-102, UGN-201, or UGN-302.

### (F) Monitoring Treatment Responses

A response to treatment, e.g., a treatment of a solid tumor as described herein, can be assessed according to RECIST or the RECIST 1.1 criteria and/or irRC, irRECIST, iRECIST, imRECISTPDAC, as described in Example 1 below and Eisenhower et al., New response evaluation criteria in solid tumours: Revised RECIST guideline (version 1.1); European Journal Of Cancer 45 (2009) 228-247; or Borcoman et al., Annals of Oncology 30: 385-396, 2019; Nishino et al., Clin Cancer Res 2013; 19(14): 3936-3943, the contents of each of which is herein incorporated by reference in its entirety.

In some embodiments, methods are provided for improving and or controlling the overall response/tumor burden/tumor size (e.g., at approximately 2, 3, 6 or 12 months, or a later time) comprising administering an anti-Galectin-9 antibody described herein, e.g., as compared to a baseline level obtained prior to initiation of G9.2-17 IgG4 treatment regimen. In some embodiments, the methods are for improving and or controlling the overall response/tumor burden/tumor size at approximately 2 months. In some embodiments, where the anti-Galectin-9 antibody is administered in a combination regimen with a checkpoint inhibitor, e.g., an anti-PD-1 antibody or an anti-PD-L1 antibody, treating can improve or control the overall response/tumor burden/tumor size (e.g., at approximately 2, 3, 6 or 12 months, or a later time), e.g., as compared to a baseline level obtained prior to initiation of treatment. In some embodiments, methods are provided, which result in a complete response, a partial response or stable disease (e.g., as measured at approximately 2 months, 3 months, 6 months or 12 months, or at a later time or at any other clinically indicated time point), comprising administering an anti-Galectin-9 antibody described herein. Such a response can be temporary over a certain time period or permanent.

In some embodiments, the methods improve the likelihood of a complete response, a partial response or stable disease (e.g., as measured at approximately 2 months, 3 months, 6 months or 12 months, or at a later time or at any other clinically indicated time point), e.g., as compared to a baseline level obtained prior to initiation of G9.2-17 IgG4 treatment regimen. Such a response can be temporary over a certain time period or permanent. In some embodiments, treating can result in reduced or attenuated progressive disease (e.g., as measured at approximately 2 months, 3 months, 6 months or 12 months, or at a later time or at any other clinically indicated time point), e.g., as compared to a baseline level obtained prior to initiation of G9.2-17 IgG4 treatment regimen. Such an attenuation may be temporary or permanent. In any of these embodiments, anti-Galectin-9 antibody may be administered in combination with a checkpoint inhibitor, e.g., an anti-PD-1 antibody.

In some embodiments, the disclosure provides methods for attenuating disease progression or reducing progressive disease (e.g., as measured at approximately 3 months, 6 months or 12 months, or at a later time or at any other clinically indicated time point). The method comprising administering to the subject a therapeutically effective amount of an anti-Galectin-9 antibody as disclosed herein. In any of these embodiments, the anti-Galectin-9 antibody may be administered in combination with a checkpoint inhibitor, e.g., an anti-PD-1 antibody.

In any of the methods described herein, Partial response, stable disease, complete response, a partial response, stable disease, progressive disease, disease progressing (e.g., as measured at approximately 2 months, 3 months, 6 months or 12 months, or at a later time or at any other clinically indicated time point), can be assessed according to irC criteria, RECIST criteria, RECIST1.1., irRECIST or iRECIST, or imRECIST criteria, or other criteria known in the art (see, e.g., Borcoman et al., Annals of Oncology 30: 385-396, 2019′ iRC: Hoos et al., J. Immunother. 30 (1): 1-15).

A partial response is a decrease in the size of a tumor, or in the extent of cancer in the body, i.e., the tumor burden, in response to treatment as compared to a baseline level before the initiation of the treatment. For example, according to the RECIST response criteria, a partial response is defined as at least a 30% decrease in the sum of diameters of target lesions, taking as reference the baseline sum diameters. Progressive disease is a disease that is growing, spreading, or getting worse. For example, according to the RECIST response criteria, progressive disease includes disease in which at least a 20% increase in the sum of diameters of target lesions is observed, and the sum must also demonstrate an absolute increase of at least 5 mm. Additionally, the appearance of one or more new lesions is also considered progression. A tumor that is neither decreasing nor increasing in extent or severity as compared to a baseline level before initiation of the treatment is considered stable disease. For example, according to the RECIST response criteria, stable disease occurs when there is neither sufficient shrinkage to qualify for partial response nor sufficient increase to qualify for progressive disease, taking as reference the smallest sum diameters while on study.

In some embodiments, the disclosure provides methods for reducing or maintaining tumor size in a subject, including a human subject (e.g., as measured at approximately 2 months, 3 months, 6 months or 12 months, or at a later time or at any other clinically indicated time point), either permanently or over a minimum time period, relative to a baseline tumor size prior to initiation of the treatment in the subject, the method comprising administering to the subject a therapeutically effective amount of an anti-Galectin-9 antibody alone or in combination with a checkpoint inhibitor, e.g., an anti-PD-1 antibody. Tumor size, e.g., the diameters of tumors, can be measured according to methods known in the art, which include measurements from CT and MRI images in combination with various software tools, according to specific measurement protocols, e.g., as described in Eisenhower et al., referenced above. Accordingly, in some embodiments, tumor size is measured in regularly scheduled restaging scans (e.g., CT with/without contrast, MRI with/without contrast, PET-CT (diagnostic CT) and/or X-ray, ultrasound and/or other relevant imaging modality). In some embodiments, tumor size reduction, maintenance of tumor size refers to the size of target lesions. In some embodiments, tumor size reduction, maintenance of tumor size refers to the size of non-target lesions. According to RECIST 1.1, when more than one measurable lesion is present at baseline, all lesions up to a maximum of five lesions total (and a maximum of two lesions per organ) representative of all involved organs should be identified as target lesions. All other lesions (or sites of disease) including pathological lymph nodes should be identified as non-target lesions.

In some embodiments, the disclosure provides methods for increasing the likelihood of reducing or maintaining a tumor burden (e.g., as measured at approximately 2 months, 3 months, 6 months or 12 months, or at a later time or at any other clinically indicated time point), the methods comprising administering to the subject a therapeutically effective amount of an anti-Galectin-9 antibody as disclosed herein, alone or in combination with a checkpoint inhibitor, e.g., an anti-PD-1 antibody. In some embodiments, treating can result in in a greater likelihood of a reduction of tumor burden, or maintenance of tumor burden, (e.g., as measured at approximately 2 months, 3 months, 6 months or 12 months, or at a later time or at any other clinically indicated time point). As used herein, tumor burden refers to amount of cancer, the size or the volume of the tumor in the body of a subject, accounting for all sites of disease. Tumor burden can be measured using methods known in the art, including but not limited to, FDG positron emission tomography (FDG-PET), magnetic resonance imaging (MRI), and optical imaging, comprising bioluminescence imaging (BLI) and fluorescence imaging (FLI).

In some embodiments, the methods described herein increase in the time to disease progression or in progression free survival (e.g., as measured at approximately 2 months, 3 months, 6 months or 12 months, or at a later time or at any other clinically indicated time point post initiation of treatment). Progression free survival can be either permanent or progression free survival over a certain amount of time. In some embodiments, the methods provide a greater likelihood of progression free survival (either permanent progression free survival or progression free survival over a certain amount of time, e.g., 3, 6 or 12 months or e.g., as measured at approximately 2 months, 3 months, 6 months, or 12 months, at a later time, or at any other clinically indicated time point post initiation of treatment). Progression-free survival (PFS) is defined as the time from random assignment in a clinical trial, e.g., from initiation of a treatment to disease progression or death from any cause. In some embodiments, the methods achieve longer survival or greater likelihood of survival, e.g., at a certain time, e.g., at 6 or 12 months.

A response to treatment, e.g., a treatment of a solid tumor as described herein, can be assessed according to iRECIST criteria, as described in Seymour et al, iRECIST: guidelines for response criteria for use in trials; The Lancet, Vol 18, March 2017, the contents of which is herein incorporated by reference in its entirety. iRECIST was developed for the use of modified RECIST1.1 criteria specifically in cancer immunotherapy trials, to ensure consistent design and data collection and can be used as guidelines to a standard approach to solid tumor measurements and definitions for objective change in tumor size for use in trials in which an immunotherapy is used. iRECIST is based on RECIST 1.1. Responses assigned using iRECIST have a prefix of “i” (i.e., immune)—e.g., “immune” complete response (iCR) or partial response (iPR), and unconfirmed progressive disease (iUPD) or confirmed progressive disease (iCPD) or stable disease (iSD) to differentiate them from responses assigned using RECIST 1.1, and all of which are defined in Seymour et al. RECIST 1.1. In some embodiments criteria can be compared to baseline levels prior to initiation of treatment. In any of these embodiments, the anti-Galectin-9 antibody may be administered alone or in combination with a checkpoint inhibitor, e.g., an anti-PD-1 antibody such as those disclosed herein.

Accordingly, in some embodiments, the disclosure provides methods for improving overall response (iOR) or achieving “immune” complete response (iCR), a partial response (iPR) or stable disease (iSD) (e.g., as measured at approximately 2 months, 3 months, 6 months or 12 months, or at a later time or at any other clinically indicated time point), as compared to the baseline level of disease prior to initiation of the treatment. The reduction in the “immune” response, e.g., iCR, iPR, or iSD can be temporary over a certain time period or permanent. In some embodiments, treating can improve the likelihood of a complete response (iCR), a partial response (iPR) or stable disease (iSD) (e.g., as measured at approximately 2 months, 3 months, 6 months or 12 months, or at a later time or at any other clinically indicated time point), e.g., In some embodiments, the disclosure provides methods for attenuating disease progression or reducing progressive disease, e.g., reducing unconfirmed progressive disease (iUPD) or reducing confirmed progressive disease (iCPD)) (e.g., as measured at approximately 2 months, 3 months, 6 months or 12 months, or at a later time or at any other clinically indicated time point), the method comprising administering to the subject a therapeutically effective amount of an anti-Galectin-9 antibody as disclosed herein. Any of these above mentioned iRECIST criteria can be compared to baseline levels prior to initiation of treatment. In any of these methods the anti-Galectin-9 antibody may be administered alone or in combination with a checkpoint inhibitor, e.g., an anti-PD-1 antibody.

The reduction in iUPD or iCPD can be temporary over a certain time period or permanent. In some embodiments, treating can result in greater likelihood of overall reduction in unconfirmed progressive disease (iUPD) or confirmed progressive disease (iCPD) (e.g., as measured at approximately 2 months, 3 months, 6 months or 12 months, or at a later time or at any other clinically indicated time point In some embodiments, the disclosure provides methods for reducing the number of new lesions in a subject, including a human subject, according to iRECIST criteria (e.g., as measured at approximately 2 months, 3 months, 6 months or 12 months, or at a later time or at any other clinically indicated time point), the methods comprising administering to the subject a therapeutically effective amount of an anti-Galectin-9 antibody as disclosed herein. Reduced number of lesions can be relative to baseline levels prior to initiation of treatment, and the reduction can be temporary over a certain time period or permanent. In any of these embodiments, the anti-Galectin-9 antibody may be administered in combination with a checkpoint inhibitor, e.g., an anti-PD-1 antibody or anti-PD-L1 antibody.

Additional criteria can be used to measure a treatment response. For example, tumor burden can be measured according to the irRC criteria (Hoos et al., 2007). In the irRC, tumor burden is measured by combining “index” lesions with new lesions, i.e., new lesions are considered a change in tumor burden. In the irRC, an immune-related Complete Response (irCR) is the disappearance of all lesions, measured or unmeasured, and no new lesions; an immune-related Partial Response (irPR) is a 50% drop in tumor burden from baseline as defined by the irRC; and immune-related Progressive Disease (irPD) is a 25% increase in tumor burden from the lowest level recorded. Everything else is considered immune-related Stable Disease (irSD).

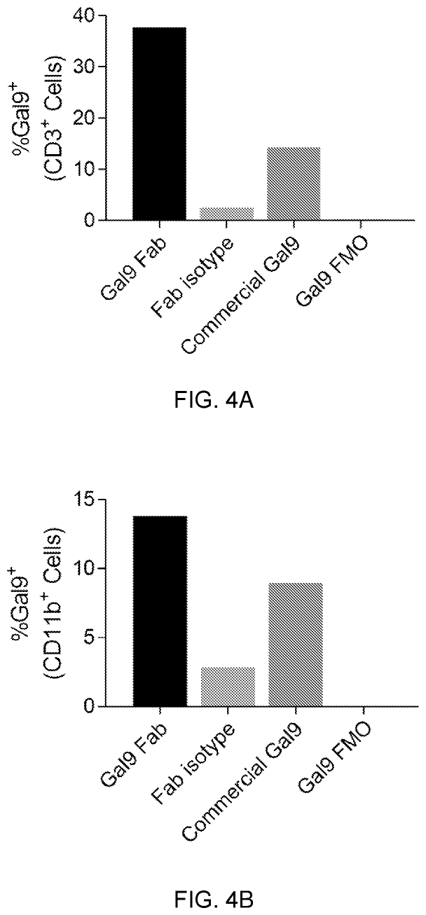

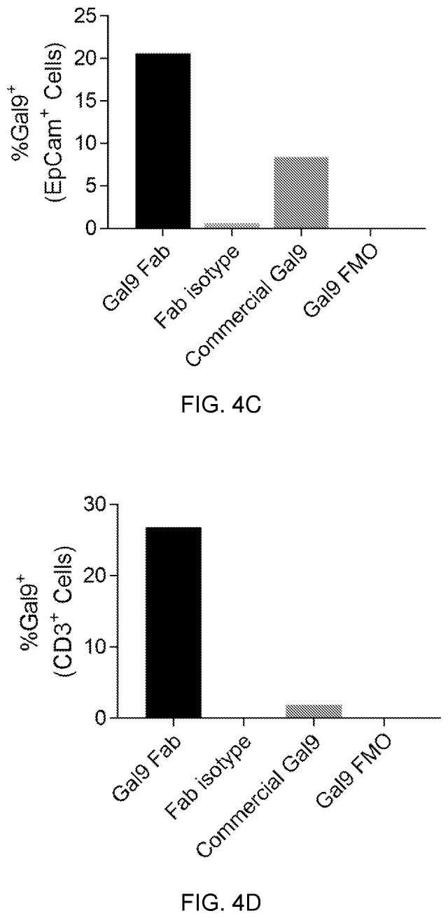

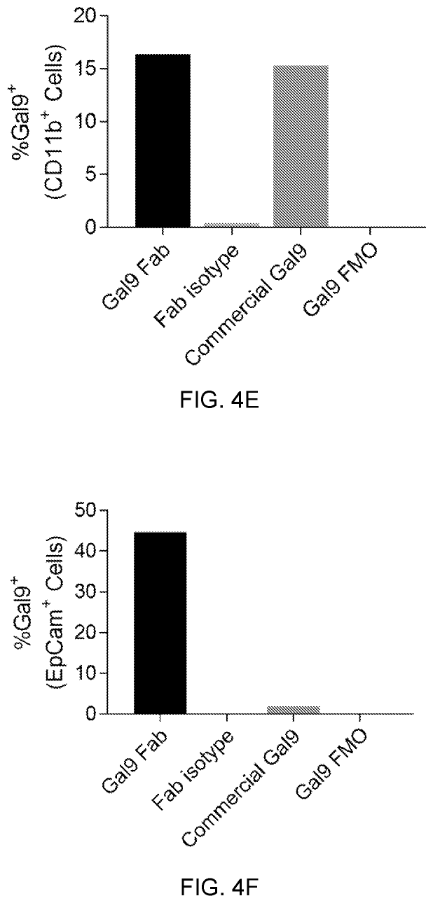

Immune-related RECIST (irRECIST) is based on unidimensional measurements of RECIST, and Specific immune-related criteria were further redefined in the irRECIST. Recently, new criteria were evaluated based on atezolizumab data in NSCLC, the immune-modified RECIST (imRECIST), requiring a confirmation of disease progression at least 4 weeks after initial assessment (Hodi et al, JCO 2018; 36(9): 850-858). For a comparison of RECIST 1.1., irRC, irRECIST, iRECIST and imRECIST, see, e.g., FIG. 4 in Borcoman et al., Annals of Oncology 30: 385-396, 2019; Nishino et al., Clin Cancer Res 2013; 19(14): 3936-3943, the contents of which is herein incorporated by reference in its entirety. Any of these criteria are suitable in determining response rate in any of the methods described herein.

A subject being treated by any of the anti-galectin-9 antibodies disclosed herein (e.g., G9.2-17), either alone or in combination with a checkpoint inhibitor (e.g., an anti-PD-1 or anti-PD-L1 antibody) as disclosed herein may be monitored for occurrence of adverse effects (for example, severe adverse effects). Exemplary adverse effects to monitor are provided in Example 1 below. If occurrence of adverse effects is observed, treatment conditions may be changed for that subject. For example, the dose of the anti-galectin-9 antibody may be reduced and/or the dosing interval may be extended. Suitability and extent of reduction may be assessed by a qualified clinician. In one embodiment, a reduction level of 30 or 50% of the previous dose level is implemented. In one specific example, a reduction level as per clinician's assessment or at least by 30% is implemented (to dose level 1, the level at first dose reduction). If required, one more dose reduction by 30% of dose level −1 is implemented (dose level −2, the level at second dose reduction). In another example, one more dose reduction by 50% of dose level −1 is implemented (dose level −2). In some embodiments, one or more dose reductions by about 10% to about 80% of a previous dose level are implemented. In some embodiments, one or more dose reductions by about 10% to about 20%, about 20% to about 30%, about 30% to about 40%, about 40% to about 50%, about 50% to about 60%, or about 70% to about 80% of a previous dose level are implemented. In some embodiments, one or more dose reductions by 10% to 20%, 20% to 30%, 30% to 40%, 40% to 50%, 50% to 60%, or 70% to 80% of a previous dose level are implemented. In some embodiments, one or more dose reductions by about 10%, by about 20%, by about 30%, by about 40%, by about 50%, by about 60%, by about 70%, or by about 80% of a previous dose level are implemented. In some embodiments, one or more dose reductions by 10%, by 20%, by 30%, by 40%, by 50%, by 60%, by 70%, or by 80% of a previous dose level are implemented. Alternatively, or in addition, the dose of the checkpoint inhibitor can be reduced and/or the dosing interval of the checkpoint inhibitor may be extended. In some instances (e.g., occurring of life-threatening adverse effects), the treatment may be terminated.

### (G) Modulating Immune Responses

Response to treatment can also be characterized by one or more of immunophenotype in blood and tumors, cytokine profile (serum), soluble galectin-9 levels in blood (serum or plasma), galectin-9 tumor tissue expression levels and pattern of expression by immunohistochemistry (tumor, stroma, immune cells), tumor mutational burden (TMB), PD-L1 expression (e.g., by immunohistochemistry), mismatch repair status, or tumor markers relevant for the disease (e.g., as measured at approximately 3 months, 6 months or 12 months, or at a later time or at any other clinically indicated time point). Examples of such tumor markers include, but are not limited to, CA15-3, CA-125, CEA, CA19-9, alpha fetoprotein. These parameters can be compared to baseline levels prior to initiation of treatment. In any of these embodiments, the anti-Galectin-9 antibody may be administered alone or in combination with a checkpoint inhibitor, e.g., an anti-PD-1 antibody or an anti-PD-L1 antibody.

In any of the methods disclosed herein, the subject may examined for one or more of the following features before, during, and/or after the treatment: (a) one or more tumor markers in blood samples from the subject, optionally wherein the one or more tumor markers comprise CA15-3, CA-125, CEA, CA19-9, and/or alpha fetoprotein, and any other tumor-type specific tumor markers; (b) cytokine profile; and (c) galectin 9 serum/plasma levels, d) peripheral blood mononuclear cell immunophenotyping, e) tumor tissue biopsy/excisional specimen multiplex immunophenotyping, f) tumor tissue biopsy/excisional specimen galectin-9 expression levels and pattern, g) any other immune score test such as: PD-L1 immunohistochemistry, tumor mutational burden (TMB), tumor microsatellite instability status, as well as panels such as: Immunoscore®-HalioDx, ImmunoSeq-Adaptive Biotechnologies, TIS, developed on the NanoString nCounter® gene expression system, 18-gene signature, PanCancer IO 360™ assay (NanoString Technologies) etc. Other suitable biomarkers specific to the target tumor such as PDAC may also be used. In one non-limiting example, PD-L1 (SP263) (Roche, Ventana) can be used for detection of PD-L1 in cancer tissues using immunohistochemistry.

In some embodiments, the methods are described herein for changing levels of immune cells and immune cell markers in the blood or in tumors, e.g., immune activation, comprising an anti-Gal-9 antibody is administered alone or in combination with a checkpoint inhibitor, e.g., an anti-PD-1 antibody. Such changes can be measured in patient blood and tissue samples using methods known in the art, such as multiplex flow cytometry and multiplex immunohistochemistry. For example, a panel of phenotypic and functional PBMC immune markers can be assessed at baseline prior to commencement of the treatment and at various time point during treatment. Table 2 lists non-limiting examples of markers useful for these assessment methods. Flow cytometry (FC) is a fast and highly informative method of choice technology to analyze cellular phenotype and function and has gained prominence in immune phenotype monitoring. It allows for the characterization of many subsets of cells, including rare subsets, in a complex mixture such as blood, and represents a rapid method to obtain large amounts of data. Advantages of FC are high speed, sensitivity, and specificity. Standardized antibody panels and procedures can be used to analyze and classify immune cell subtypes. Multiplex IHC is a powerful investigative tool, which provides objective quantitative data describing the tumor immune context in both immune subset number and location and allows for multiple markers to be assessed on a single tissue section. Computer algorithms can be used to quantify IHC-based biomarker content from whole slide images of patient biopsies, combining chromogenic IHC methods and stains with digital pathology approaches.

Accordingly, in some embodiments, methods are described herein, for modulating an immune response, e.g., modulation of immune activation markers such as those in Table 2 comprising administering an anti-gal9 antibody alone or in combination with a checkpoint inhibitor therapy. In some embodiments, modulation comprises in one or more of (1) an increase in more CD8 cells in plasma or tumor tissue, (2) a reduction in T regulatory cells (Tregs) in plasma or tumor tissue, (3) an increase in M1 macrophages in plasma or tumor tissue and (4) a decrease in MDSCs in plasma or tumor tissue, and (5) a decrease in M2 macrophages in plasma or tumor tissue (e.g., as measured at approximately 2 months, 3 months, 6 months or 12 months, or at a later time or at any other clinically indicated time point). In some embodiments, the markers that are assessed using the techniques described above or known in the art are selected from CD4, CD8 CD14, CD11b/c, and CD25. These parameters can be compared to baseline levels prior to initiation of treatment.

TABLE 2
PBMC phenotyping markers

| | PBMC phenotyping markers | | PBMC phenotyping markers |
|---|---|---|---|
| CD3 | Total T cells | CD16 | NK cells |
| CD4 | CD4+ T cells | CD11b | Monocytes/macrophages |
| CD8 | CD8+ T cells | CD11c | Monocytes/macrophages, DCs |
| CD25 | Treg activation | CD14 | Monocyte subsets, |
| | | | macrophages |
| CD27 | T cell maturation; B cell | CD33 | Total myeloid cells |
| | naïve/memory | | |
| CD38 | T cell maturation; B cell | FceR1 a | Antigen presenting DC cells |
| | naïve/memory | | |
| CD45RA | Naïve/memory cells | CD19 | Total B cells |
| CD45RO | Naïve/memory cells | T-bet | T cells subsets |
| CD56 | NKT/NK cells (T cell subset) | gdTCR | Gamma delta T cells |
| CD127 | T cell subsets | CD274 | Checkpoint |
| | | (PDL-1) | |
| CD152 | Checkpoint | Tim-3 | Checkpoint |
| (CLTA-4) | | | |
| CD279 | Checkpoint | TCR | iNKT cells |
| (PD-1) | | Va24-Ja18 | |
| FoxP3 | Treg cells | Live/dead | General |
| HLA-DR | Activation/Antigen presentation | CD45 | General |

In some embodiments, methods are described herein, comprising administering an anti-gal9 alone or in combination with a checkpoint inhibitor therapy, for modulating proinflammatory and anti-inflammatory cytokines. In some embodiments, methods are provided for one or more of (1) increasing levels of IFNgamma in plasma or tumor tissue; (2) increasing levels of TNFalpha in plasma or tumor tissue; (3) decreasing levels of IL-10 in plasma or tumor tissue (e.g., as measured at approximately 3 months, 6 months or 12 months, or at a later time or at any other clinically indicated time point). These parameters can be compared to baseline levels prior to initiation of treatment.

In some embodiments, cytokine levels or immune cell levels may be assessed between a pre dose 1 tumor biopsy and repeat biopsy conducted at a feasible time. In some embodiments, cytokine levels or immune cell levels may be assessed between 2 repeat biopsies. In some embodiments, methods are provided for modulating one or more of soluble galectin-9 levels in blood (serum or plasma), or galectin-9 tumor tissue expression levels and pattern of expression by immunohistochemistry (tumor, stroma, immune cells), (e.g., as measured at approximately 3 months, 6 months or 12 months, or at a later time or at any other clinically indicated time point). In some embodiments, the methods decrease soluble galectin-9 levels in blood (serum or plasma), or galectin-9 tumor tissue expression levels or pattern of expression by immunohistochemistry (tumor, stroma, immune cells) (e.g., as measured at approximately 3 months, 6 months or 12 months, or at a later time or at any other clinically indicated time point). Galectin-9 levels can be compared to baseline levels prior to initiation of treatment. In some embodiments, Galectin-9 levels may be compared to a control group not receiving the treatment or healthy subjects. In any of these embodiments, the anti-Galectin-9 antibody may be administered alone or in combination with a checkpoint inhibitor, e.g., an anti-PD-1 antibody. In some embodiments, methods for modulating PD-L1 expression are provided, e.g., as assessed by immunohistochemistry, comprising administering an anti-Galectin-9 antibody, alone or in combination with a checkpoint inhibitor, e.g., an ant-Galectin-9 antibody. In some embodiments, the methods modulate in one or more tumor markers (increase or decrease) relevant for the disease (e.g., as measured at approximately 2 months, 3 months, 6 months or 12 months, or at a later time or at any other clinically indicated time point). Examples of such tumor markers include, but are not limited to, CA15-3, CA-125, CEA, CA19-9, alpha fetoprotein. These parameters can be compared to baseline levels prior to initiation of treatment. In any of these embodiments, the anti-Galectin-9 antibody may be administered alone or in combination with a checkpoint inhibitor, e.g., an anti-PD-1 antibody.

In some embodiments, the disclosure provides methods of modulating an immune response in a subject. As used herein, the term “immune response” includes T cell-mediated and/or B cell-mediated immune responses that are influenced by modulation of immune cell activity, for example, T cell activation. In one embodiment of the disclosure, an immune response is T cell mediated. As used herein, the term “modulating” means changing or altering, and embraces both upmodulating and downmodulating. For example, “modulating an immune response” means changing or altering the status of one or more immune response parameter(s). Exemplary parameters of a T cell mediated immune response include levels of T cells (e.g., an increase or decrease in effector T cells) and levels of T cell activation (e.g., an increase or decrease in the production of certain cytokines). Exemplary parameters of a B cell mediated immune response include an increase in levels of B cells, B cell activation and B cell mediated antibody production.

When an immune response is modulated, some immune response parameters may decrease and others may increase. For example, in some instances, modulating the immune response causes an increase (or upregulation) in one or more immune response parameters and a decrease (or downregulation) in one or more other immune response parameters, and the result is an overall increase in the immune response, e.g., an overall increase in an inflammatory immune response. In another example, modulating the immune response causes an increase (or upregulation) in one or more immune response parameters and a decrease (or downregulation) in one or more other immune response parameters, and the result is an overall decrease in the immune response, e.g., an overall decrease in an inflammatory response. In some embodiments an increase in an overall immune response, i.e., an increase in an overall inflammatory immune response, is determined by a reduction in tumor weight, tumor size or tumor burden or any RECIST or iRECIST criteria described herein. In some embodiments an increase in an overall immune response is determined by increased level(s) of one or more proinflammatory cytokine(s), e.g., including two or more, three or more, etc. or a majority of proinflammatory cytokines (one or more, two or more, etc. or a majority of anti-inflammatory and/or immune suppressive cytokines and/or one or more of the most potent anti-inflammatory or immune suppressive cytokines either decrease or remain constant). In some embodiments an increase in an overall immune response is determined by increased levels of one or more of the most potent proinflammatory cytokines (one or more anti-inflammatory and/or immune suppressive cytokines including one or more of the most potent cytokines either decrease or remain constant). In some embodiments an increase in an overall immune response is determined by decreased levels of one or more, including a majority of, immune suppressive and/or anti-inflammatory cytokines (the levels of one or more, or a majority of, proinflammatory cytokines, including e.g., the most potent proinflammatory cytokines, either increase or remain constant). In some embodiments, an increase in an overall immune response is determined by increased levels of one or more of the most potent anti-inflammatory and/or immune suppressive cytokines (one or more, or a majority of, proinflammatory cytokines, including, e.g., the most potent proinflammatory cytokines either increase or remain constant). In some embodiments an increase in an overall immune response is determined by a combination of any of the above. Also, an increase (or upregulation) of one type of immune response parameter can lead to a corresponding decrease (or downregulation) in another type of immune response parameter. For example, an increase in the production of certain proinflammatory cytokines can lead to the downregulation of certain anti-inflammatory and/or immune suppressive cytokines and vice versa.

In some embodiments, the disclosure provides methods for modulating an immune response (e.g., as measured at approximately 2 months, 3 months, 6 months or 12 months, or at a later time or at any other clinically indicated time point) in a subject, including a human subject, comprising administering to the subject a therapeutically effective amount of an anti-Galectin-9 antibody as disclosed herein. In some embodiments, the disclosure provides methods for modulating levels of immune cells and immune cell markers, including but not limited to those described herein in Table 2, e.g., as compared to baseline levels prior to initiation of treatment, e.g., as compared to a baseline level obtained prior to initiation of the anti-Gal9 antibody treatment regimen, in the blood or in tumors of a subject, including a human subject, comprising administering to the subject a therapeutically effective amount of an anti-Galectin-9 antibody as disclosed herein. In some embodiments, the overall result of modulation is upregulation of proinflammatory immune cells and/or down regulation of immune-suppressive immune cells. In some embodiments, the disclosure provides methods for modulating levels of immune cells, wherein the modulating encompasses one or more of (1) increasing CD8 cells in plasma or tumor tissue, (2) reducing Tregs in plasma or tumor tissue, (3) increasing M1 macrophages in plasma or tumor tissue and (4) decreasing MDSC in plasma or tumor tissue, and (5) decreasing in M2 macrophages in plasma or tumor tissue, and wherein the methods comprise administering to the subject a therapeutically effective amount of an anti-Galectin-9 antibody as disclosed herein. In some embodiments, the markers to assess levels of such immune cells include but are not limited to CD4, CD8 CD14, CD11b/c, and CD25. In some embodiments, the disclosure provides methods for modulating levels of proinflammatory and immune suppressive cytokines (e.g., as measured at approximately 2 months, 3 months, 6 months or 12 months, or at a later time or at any other clinically indicated time point), e.g., as compared to baseline levels prior to initiation of treatment, in the blood or in tumors of a subject, including a human subject, comprising administering to the subject a therapeutically effective amount of an anti-Galectin-9 antibody as disclosed herein. In some embodiments, the overall result of modulation is upregulation of proinflammatory cytokines and/or down regulation of immune-suppressive cytokines. In some embodiments, the disclosure provides methods for modulating levels of cytokines cells, wherein the modulating encompasses one or more of (1) increasing levels of IFNgamma in plasma or tumor tissue; (2) increasing levels of TNFalpha in plasma or tumor tissue; (3) decreasing levels of IL-10 in plasma or tumor tissue.

In some embodiments, the disclosure provides methods for changing one or more of soluble galectin-9 levels in blood (serum or plasma), or in galectin-9 tumor tissue expression levels and pattern of expression by immunohistochemistry (tumor, stroma, immune cells) (e.g., as measured at 2 weeks, 4 weeks, 1 month, 2 month, 3 months, 6 months or 12 months, or at a later time or at any other clinically indicated time point), comprising administering to the subject a therapeutically effective amount of an anti-Galectin-9 antibody as disclosed herein. In some embodiments of the methods, one or more of soluble galectin-9 levels in blood (serum or plasma), or in galectin-9 tumor tissue expression levels and pattern of expression by immunohistochemistry (tumor, stroma, immune cells) remain unchanged. In some embodiments, the methods provided herein decrease one or more of soluble galectin-9 levels in blood (serum or plasma), or in galectin-9 tumor tissue expression levels and pattern of expression by immunohistochemistry (tumor, stroma, immune cells) (e.g., e.g., as measured at 2 weeks, 4 weeks, 1 month, 2 month, 3 months, 6 months or 12 months, or at a later time or at any other clinically indicated time point). Galectin-9 levels can be compared to baseline levels prior to initiation of treatment. In some embodiments, the Galectin-9 levels may be compared to healthy subjects. In some embodiments, treating results in a change in PD-L1 expression, e.g., by immunohistochemistry. 16 mg/kg or higher dose level 16 mg/kg or higher dose level 16 mg/kg or a higher dose level.

In some embodiments, the disclosure provides methods for changing PD-L1 expression, e.g., as assessed by immunohistochemistry (e.g., as measured at 2 weeks, 4 weeks, 1 month, 2 month, 3 months, 6 months or 12 months, or at a later time or at any other clinically indicated time point), comprising administering to the subject a therapeutically effective amount of an anti-Galectin-9 antibody as disclosed herein. In some embodiments of the methods, PD-L1 expression, e.g., as assessed by immunohistochemistry, remains unchanged. PD-L1 levels can be compared to baseline levels prior to initiation of treatment. In some embodiments, the methods provided herein decrease PD-L1 expression, e.g., as assessed by immunohistochemistry. PD-L1 levels may be measured using routine methods known in the art. In one non-limiting example, PD-L1 (SP263) (Roche, Ventana) can be used for detection of PD-L1 in cancer tissues using immunohistochemistry.

In some embodiments, the disclosure provides methods for changing one or more tumor markers (increasing or decreasing) relevant for the disease (e.g., as measured at 2 weeks, 4 weeks, 1 month, 3 months, 6 months or 12 months, or at a later time or at any other clinically indicated time point), comprising administering to the subject a therapeutically effective amount of an anti-Galectin-9 antibody as disclosed herein. In some embodiments of the methods, one or more tumor markers (increasing or decreasing) relevant for the disease, remain unchanged. Examples of such tumor markers include, but not limited to CA15-3, CA-125, CEA, CA19-9, alpha fetoprotein. Levels of tumor markers can be compared to baseline levels prior to initiation of treatment. In some embodiments, the methods provided herein decrease the occurrence of one or more tumor markers relevant for the disease.

In some embodiments, the disclosure provides methods for changing one or more biomarkers (increasing or decreasing) relevant for the disease (e.g., as measured at 2 weeks, 4 weeks, 1 month, 2 months, 3 months, 6 months or 12 months, or at a later time or at any other clinically indicated time point), comprising administering to the subject a therapeutically effective amount of an anti-Galectin-9 antibody as disclosed herein. Levels of biomarkers in clinical tissues from patients can be measured using routine methods, such as multiplex Immunofluorescence (mIF) technology, as described herein in the examples. An exemplary panel of biomarkers may include CD3, CD4, CD8, CD45RO, FoxP3, CD11b, CD14, CD15, CD16, CD33, CD68, CD163, HLA-DR, Arginase1, Granzyme B, Ki67, PD-1, PD-L1, F4/80, Ly6G/C and PanCK.

In any of these methods described herein for modulating an immune response, cytokines, biomarkers, such as Galectin-9 or PD-L1 levels, or tumor markers, any of the anti-Galectin-9 antibodies described herein may be used. In some embodiments, the antibody comprises a light chain complementarity determining region 1 (CDR1) set forth as SEQ ID NO: 1, a light chain complementarity determining region 2 (CDR2) set forth as SEQ ID NO: 2, and a light chain complementarity determining region 3 (CDR3) set forth as SEQ ID NO: 3 and/or comprises a heavy chain complementarity determining region 1 (CDR1) set forth as SEQ ID NO: 4, a heavy chain complementarity determining region 2 (CDR2) set forth as SEQ ID NO: 5, and a heavy chain complementarity determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15.

In some embodiments, the antibody is G9.2-17 IgG4. In some embodiments, the anti-Galectin-9 antibody is administered to the subject at a dose of about 0.2 mg/kg to about 32 mg/kg, e.g., the dose may be selected from 0.2 mg/kg, 0.63 mg/kg, 2 mg/kg, 4 mg/kg, 6 mg/kg, 6.3 mg/kg, 8 mg/kg, 10 mg/kg, 12 mg/kg, and 16 mg/kg or a higher dose level. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg or a higher dose level. In some embodiments, the anti-Galectin-9 antibody is administered to the subject at a dose of about 0.2 mg/kg to about 32 mg/kg, e.g., the dose may be selected from 0.2 mg/kg, 0.63 mg/kg, 2 mg/kg, 4 mg/kg, 6 mg/kg, 6.3 mg/kg, 10 mg/kg, or 16 mg/kg or a higher dose level. In some embodiments, the antibody is administered once every two weeks, e.g., via intravenous infusion.

In some embodiments, the method further comprises administering to the subject an immune checkpoint inhibitor, e.g., an anti-PD-1 antibody or an anti-PD-L1 antibody. In some embodiments, the solid tumor is selected from pancreatic ductal adenocarcinoma (PDAC), colorectal cancer (CRC), hepatocellular carcinoma (HCC), cholangiocarcinoma (CAA), renal cell carcinoma (RCC), urothelial, head and neck, breast cancer, lung cancer, and other GI solid tumors, and in some embodiments, the solid tumor is a metastatic tumor.

In some embodiments, the disclosure provides methods for improving quality of life and/or improving symptom control (e.g., as measured at 1 month, 3 months, 6 months or 12 months, or at a later time or at any other clinically indicated time point) in a subject, including a human subject, comprising administering to the subject a therapeutically effective amount of an anti-Galectin-9 antibody as disclosed herein. Improved quality of life and symptom control may be compared to baseline prior to initiation of treatment. In some embodiments, improvements can be measured on the ECOG scale.

Kits for Use in Treatment of Diseases Associated with Galectin-9

The present disclosure also provides kits for use in treating or alleviating a disease associated with Galectin-9, for example associated with Galectin-9 binding to a cell surface glycoprotein (e.g., Dectin-1, TIM3, CD206, etc.), or pathologic cells (e.g., cancer cells) expressing Galectin-9. Examples include solid tumors such as PDAC, CRC, HCC, cholangiocarcinoma and other GI solid tumors, and others described herein. Such kits can include one or more containers comprising an anti-Galectin-9 antibody, e.g., any of those described herein, and optionally a second therapeutic agent (e.g., a checkpoint inhibitor such as an anti-PD-1 antibody as disclosed herein) to be co-used with the anti-Galectin-9 antibody, which is also described herein.

In some embodiments, the kit can comprise instructions for use in accordance with any of the methods described herein. The included instructions can comprise a description of administration of the anti-Galectin-9 antibody, and optionally the second therapeutic agent, to treat, delay the onset, or alleviate a target disease as those described herein. In some embodiments, the kit further comprises a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an anti-Galectin-9 antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease associated with Galectin-9 (e.g., Dectin-1, TIM-3, or CD206 signaling). In some embodiments, instructions are provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. In some embodiments, a kit has a sterile access port (for example the container may be an intravenous solution bag or a vial having a stopper pierceable by a hypodermic injection needle). In some embodiments, the container also has a sterile access port (for example the container is an intravenous solution bag or a vial having a stopper pierceable by a hypodermic injection needle). At least one active agent in the composition is an anti-Galectin-9 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: A Phase 1/2 Open-Label, Multi-Center Study of the Safety, Pharmacokinetics, and Anti-Tumor Activity of Anti-Galectin-9 Monoclonal Antibody Alone and in Combination with an Anti-PD-1 Antibody in Patients with Metastatic Solid Tumors

Galectin-9 is a molecule overexpressed by many solid tumors, including those in pancreatic cancer, colorectal cancer, and hepatocellular carcinoma. Moreover, Galectin-9 is expressed on tumor-associated macrophages, as well as intra-tumoral immunosuppressive gamma delta T cells, thereby acting as a potent mediator of cancer-associated immunosuppression. As described herein, monoclonal antibodies targeting Galectin-9 (e.g., G9.2-17, IgG4) have been developed. Data have demonstrated that the G9.2-17 halts pancreatic tumor growth by 50% in orthotopic KPC models and extended the survival of KPC animals by more than double. Without wishing to be bound by theory, it is thought that the anti-Galectin-9 antibodies reverse the M2 to M1 phenotype, facilitating intra-tumoral CD8⁺ T cell activation. In additional, antibody G9.2-17 (IgG4) (having a heavy chain of SEQ ID NO: 19 and a light chain of SEQ ID NO:15) has been found to synergize with anti-PD-1.

G9.2-17 (IgG4) is a fully human IgG4 monoclonal antibody (mAb) targeting galectin-9 (-gal-9) protein. Gal-9 functions as an immuno-suppressor, conferring immune privilege to tumor cells and disabling immune mediated cancer attack by regulating macrophages, T-cells, myeloid derived suppressor cells as well as cancer cell susceptibility to cytotoxic T-cell-induced death. Based on the available data, G9.2-17 (IgG4) blockade of gal-9 interferes with the immunosuppressive functions of gal-9 resulting in effective immune activation and tumor growth inhibition across multiple preclinical models.

Gal-9 can be overexpressed and/or secreted in many solid tumor types including pancreatic adenocarcinoma, cholangiocarcinoma (CCA), colorectal cancer (CRC), breast cancer, bladder cancer, ovarian cancer, non-small cell and small cell lung cancer, nasopharyngeal cancer, malignant melanoma, ovarian cancer etc., and high levels of tissue and/or circulating gal-9 correlate with aggressive tumor features and adverse survival outcome.

Therefore, the target indications of G9.2-17 (IgG4) are relapsed or refractory, metastatic solid tumors, where G9.2-17 (IgG4) is investigated both as a single agent, or and in combination with a checkpoint inhibitor (e.g., a programmed cell death 1 [PD 1] antibody such as nivolumab, pembrolizumab, cemiplimab, dostarlimab, or tislelizumab).

Dose escalation (Part 1) is conducted in all solid tumor types in order to establish the safety and tolerability profile of G9.2-17 (IgG4), assess its immunogenicity potential, establish the pharmacokinetic (PK) and pharmacodynamic (PD) profile, and arrive at the recommended Phase 2 dose (RP2D). This may be the maximal tolerated dose (MTD). The expansion cohorts (Part 2) are planned in: first line metastatic pancreatic ductal adenocarcinoma (PDAC), as well as CRC and CCA, both as a single agent and in combination with an anti-PD1 antibody.

No other therapies targeting gal-9 are currently known to be approved or in clinical trials for any indication.

In nonclinical studies conducted to-date, no significant toxicities have been observed at doses that are ˜500-fold above those intended for human administration. Furthermore, G9.2-17 (IgG4) has been shown to be highly specific for gal-9 and has been demonstrated to be efficacious in multiple animal models of cancer. The patient populations targeted for enrollment are at late stages in their disease and have failed at standard of care treatments prior to enrollment in this study. G9.2-17 (IgG4), either taken alone or in combination with a checkpoint inhibitor such as an anti-PD-1 antibody (e.g., nivolumab, pembrolizumab, tislelizumab, dostarlimab, or cemiplimab) would be expected to benefit treatment of malignant tumors such as malignant solid tumors.

### Objectives and Endpoints

### Part 1: Dose Escalation

| OBJECTIVES | ENDPOINTS |
|---|---|
| Primary | |
| To establish the safety and tolerability, | Evaluation of safety parameters including adverse |
| and to determine the recommended | events, vital sign measurements, clinical safety |
| Phase 2 dose (RP2D) for G9.2-17 (IGG4) | laboratory tests, 12-lead ECG, |
| | echocardiography/cardiac ultrasound (ECHO), |
| | physical examinations |
| | Evaluation of dose-limiting toxicities (DLTs) |
| | Determination of RP2D |
| Secondary | |
| To characterize the pharmacokinetic (PK) | Evaluation of PK parameters of G9.2-17 (IGG4) |
| profile of G9.2-17 (IgG4) | (including but not limited to area under the curve from |
| | time zero until 336 h [AUC_{0-336 h}], maximum observed |
| | serum concentration [Cₘₐₓ], time to reach Cₘₐₓ [Tₘₐₓ], |
| | estimated half-life [_{t1/2}]) |
| To assess the pharmacodynamics (PD) of | Peripheral blood mononucleocyte (PBMC) |
| G9.2-17 (IGG4) | immunophenotyping by flow cytometry |
| | Pre-specified cytokine profile (serum) by ELISA |
| | Soluble gal-9 levels in blood (serum) by ELISA |
| | Gal-9 tumor tissue expression levels, and pattern of |
| | expression by IHC (expression on tumor cells and |
| | immune cells as well as a combined score) |
| | Tumor tissue multiplex immunophenotyping |
| | Programmed death ligand-1 (PD-L1) expression by |
| | IHC |
| | Mismatch repair (MMR) status by IHC |
| To assess the immunogenicity of G9.2-17 | Evaluation of ADA |
| (IgG4) | |
| Exploratory | |
| To evaluate preliminary efficacy of G9.2- | Evaluation of confirmed objective response rate |
| 17 (IgG4) | (ORR) |
| | Evaluation of disease control rate (DCR) |
| | Evaluation of duration of response (DoR) |
| | Evaluation of progression-free survival (PFS) |
| | Evaluation of overall survival (OS) |

### Part 2: Cohort Expansion

| Objectives | Endpoints |
|---|---|
| Primary | |
| To establish the safety and tolerability of | Evaluation of safety parameters including adverse |
| G9.2-17 (IgG4) as single treatment and in | events, vital sign measurements, clinical safety |
| combination with an anti-PD-1 antibody. | laboratory tests, 12-lead ECG, ECHO, physical |
| | examinations |
| | Evaluation of DLTs |
| To assess the ORR in patients with CRC | Evaluation of the confirmed ORR-3 in patients with |
| or CCA treated with G9.2-17 (IGG4) | CRC or CCA |
| alone or in combination with an anti-PD-1 | |
| antibody. | |
| To assess PFS in patients with PDAC | Evaluation of PFS-6 in patients with PDAC |
| treated with G9.2-17 (IgG4) | |
| Secondary | |
| To characterize the pharmacokinetic (PK) | Evaluation of PK parameters of G9.2-17 (IgG4) |
| profile of G9.2-17 (IgG4) | |
| To assess the pharmacodynamics (PD) of | PBMC immunophenotyping by flow cytometry |
| G9.2-17 (IgG4) | Cytokine profile (serum) by ELISA |
| | Soluble gal-9 levels in blood (serum) by ELISA |
| | Gal-9 tumor tissue expression levels, and pattern of |
| | expression by IHC (expression on tumor cells and |
| | immune cells as well as a combined score) |
| | Tumor tissue multiplex immunophenotyping |
| | PD-L1 expression by IHC |
| | MMR status by IHC |
| | TMB in tissue |
| To assess the immunogenicity of G9.2-17 | Evaluation of ADA |
| (IGG4) | |
| To evaluate efficacy of G9.2-17 (IgG4) | Confirmed ORR |
| | PFS |
| | DCR |
| | DoR |
| | OS |

### Study Design

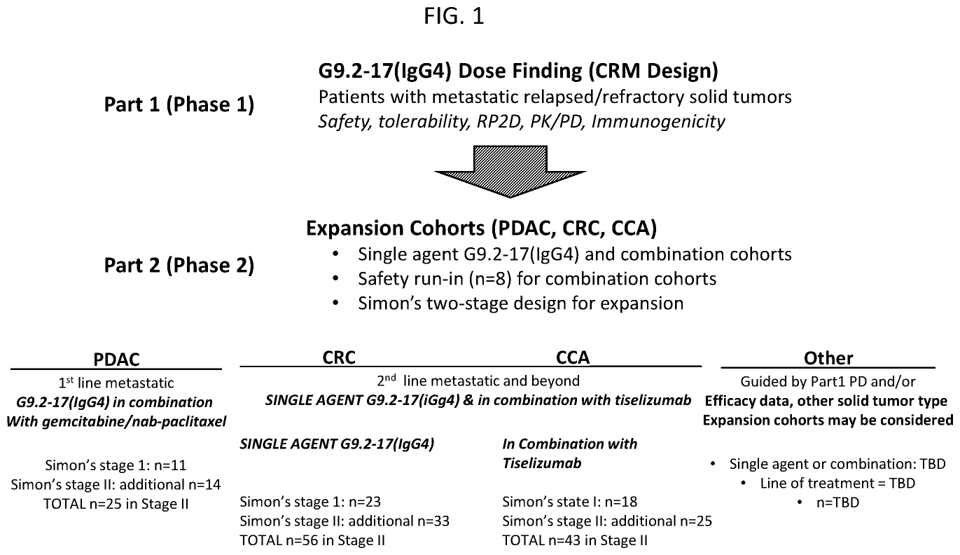

This is an open-label, uncontrolled, multicenter Phase 1/2 study with a dose escalation phase (Part 1) and a cohort expansion phase (Part 2) in patients with relapsed/refractory metastatic solid tumors. This study is conducted at up to 20 sites in the United States. The study duration is estimated to be 12-24 months. Follow-up for survival continues for up to 2 years. A study schema is presented in FIG. 1.

This study includes both monotherapy of G9.2-17 (IgG4) and combination of G9.2-17 and an anti-PD-1 antibody such as nivolumab. Doses of G9.2-17 may range from about 3 mg/kg to 15 mg/kg once every two weeks. In an alternative embodiment, doses of G9.2-17 may range from about 0.2 mg/kg to 16 mg/kg or higher dose level once every two weeks. The antibody is administered by intravenous infusion.

### Treatment Duration and Treatment Periods

### Treatment Duration

Study drug administration continues until progression of disease, unacceptable toxicity, or withdrawal from the study. Patients who discontinue the study drug prior to disease progression and are not being treated with other systemic anti-cancer therapy(ies), are followed on the study until the time of disease progression.

### Treatment Periods

The study consists of the following periods in both Part 1 and Part 2:

Screening period: up to 4 weeks prior to first dose (Day −28 to Day −1)

Treatment period: 28-day treatment cycles as presented in the Schedule of Assessments (SoA; Tables 5-6 below)

Post-treatment period: 30 days after last treatment (End of Treatment Visit/Early Termination Visit)

IMAR follow-up period: 90-days after last treatment (G9.2-17 (IgG4)+an anti-PD-1 antibody arm)

Follow-up period: Long-term follow-up for up to 2 years (visits every 3 months) for patients discontinuing treatment due to reasons other than progression of disease, and not receiving additional systemic anticancer treatments

### Part 1: Dose Escalation Phase

A dose-finding study is conducted using a continuous reassessment method (CRM) (O'Quigley et al., 1990) to establish DLTs and the RP2D. Two to six patients per treatment cohort 1-6 are assigned to receive sequentially higher IV injections of G9.2-17 (IGG4) every two weeks (Q2W) on Day 1 and Day 15 of each 28-day cycle, starting at a dose of 0.2 mg/kg. Patients assigned to a specific dose escalation cohort receive the corresponding study dose for that cohort. They receive study drug at one of 8 dose levels until progression of disease, unacceptable toxicity, or withdrawal from the study for other reasons. Patients who withdraw for reasons other than toxicity or tolerability issues during the first treatment cycle only are replaced.

For cohorts 1-6, two patients at a time are dosed under the CRM design. Dose escalations are based on analysis of patient safety data focusing on occurrences of DLTs at previous dose levels and other relevant safety and dosing data from previous cohorts. Dose escalations may occur after a minimum of 28 days (1 cycle). No dose level skipping is allowed.

Following the completion of cohort 6 under the CRM design, a once weekly (QW) G9.2-17 (IGG4) dosing schema is evaluated, provided the RP2D has not been reached within the CRM design. Cohorts 7 and 8 are not evaluated with the CRM design. Patients are only allowed to enter Cohort 7 once no DLT has been identified.

For cohorts 7 and 8, four patients at a time are dosed per cohort. Four patients per dose level in cohorts 7 and 8 are assigned to receive sequentially higher IV injections of G9.2-17 (IGG4) every week (QW) on Days 1, 8, 15, and 22 of each 28-day cycle. Starting with the first four patients in cohort 7, dose escalations to the next cohort only occur if no DLTs are identified. If a single DLT is documented in cohort 7, no further patients are dosed within that cohort and cohort 8 is not activated.

Part 1, cohorts 1-8 enroll approximately 36 patients. A total of 6 dosage levels are evaluated within the CRM design:
Dose Escalation Cohort 1=0.2 mg/kg Q2W
Dose Escalation Cohort 2=0.63 mg/kg Q2W
Dose Escalation Cohort 3=2 mg/kg Q2W
Dose Escalation Cohort 4=6.3 mg/kg Q2W
Dose Escalation Cohort 5=10 mg/kg Q2W
Dose Escalation Cohort 6=16 mg/kg Q2W
   Additional 2 dosage levels are included for the consideration of RP2D:
Dose Escalation Cohort 7=10 mg/kg QW
Dose Escalation Cohort 8=16 mg/kg QW

Patients treated in early cohorts prior to identification of the RP2D are allowed to dose escalate up to the highest dose level cleared. After a complete cycle, dose escalations may occur after a minimum of 28 days (1 cycle). Dose escalations may not occur in the middle of a cycle. Patients can continue to dose escalate to the highest approved dose level until they are discontinued for toxicity or disease progression, or for other reasons (e.g., a patient elects to discontinue from the study).

Dose escalations are based on the development of DLTs in patients treated at previous dose levels. For each dose cohort, prior DLT probabilities are specified from GLP-compliant toxicity studies as well as from preclinical models. For the specified target DLT rate and total number of dose levels, the skeleton for a power model d\hat{ }exp(a) is generated according to the approach of Lee and Cheung, using a prior MTD adjusted by PK/PD data, located at the median dose level and a spacing measure of delta=0.05 (Lee and Cheung, 2011). The prior distribution on the parameter “a” has a mean zero normal distribution with the least informative prior variance. The trial is stopped for safety if the lower limit of an Agresti and Coull binomial confidence interval (CI) for the lowest study dose level exceeds the target DLT rate (Agresti and Coull, 1998). The RP2D is the MTD dose derived from Part 1.

If a DLT occurs in any patient during the first 28 days of treatment, that patient is permanently discontinued from study drug administration.

For patients who experience toxicities (including IMARs) outside of the DLT window, dose reduction is allowed only if clinical benefit is expected and may continue to be derived with lower doses of G9.2-17 (IgG4). The dose of G9.2-17 (IgG4) is initially reduced by 50%, and potentially by a further reduction of 50%, as defined by the dose modification guidance provided in Table 3. No further dose reductions are allowed.

TABLE 3
Recommended Dose Modifications for G9.2-17 (IgG4)
(AEs outside the DLT window and other than IMARs)

| Adverse Reaction | Severity | G9.2-17 (IgG4) Dose Modification |
|---|---|---|
| Non-hematologic and | ≥Grade 4 | Permanently discontinue treatment with |
| hematologic toxicities | | G9.2-17 (IgG4). |
| Hematologic toxicities | Grade 3 | Withhold treatment until toxicity resolves |
| | | to ≤Grade 2. |
| | | Resume G9.2-17 (IgG4) at 50% reduction |
| | | from previous dose. |
| | | If recurs again at ≥Grade 3 severity, hold |
| | | until resolved to ≤Grade 2 and |
| | | implement a further 50% dose reduction. |
| | | No further dose reductions are allowed. |
| Non-hematologic | Grade 3 | Withhold treatment until toxicity resolves |
| toxicities | | to ≤Grade 2. |
| Management of | | Resume G9.2-17 (IgG4) at 50% reduction |
| Immune-Mediated | | from previous dose. |
| Adverse Reactions | | If recurs again at ≥Grade 3 severity, hold |
| (IMARs) Caused by | | until resolved to ≤Grade 2 and |
| G9.2-17 (IgG4)) | | implement a further 50% dose reduction. |
| | | No further dose reductions are allowed. |
| Gastrointestinal | Grade 3 | Withhold G9.2-17 (IgG4) until toxicity |
| (Nausea, Vomiting) | Not requiring hospitalization or | reverses to ≤Grade 2 |
| | parenteral nutrition support; | Resume G9.2-17 (IgG4) with no dose |
| | managed by supportive care | reduction |
| Electrolyte | Grade 3 corrected to ≤Grade 2 | If recurrent, withhold G9.2-17 (IgG4) |
| Abnormalities | within 24 hours | until toxicity reverses to ≤Grade 2 |
| Electrolyte | ≥Grade 3 lasting <24-72 hours | Then resume G9.2-17 (IgG4) at a 50% |
| Abnormalities | Not clinically complicated; | reduction from the previous dose |
| | resolves spontaneously or | If recurrent, withhold G9.2-17 (IgG4) |
| | responds to conventional | until toxicity reverses to ≤Grade 2 |
| | medical interventions | Resume G9.2-17 (IgG4) at an additional |
| Amylase or Lipase | ≥Grade 3 | 50% dose reduction |
| Elevation | Not associated with symptoms | No further dose reductions are allowed |
| | or clinical manifestations of | |
| | pancreatitis | |

### Part 1 Completion

Part 1 is completed when up to six patients have received the dose that has been identified as RP2D. The RP2D is based, in part, on the continual reassessment method (CRM) study design, PK and PD data parameters, additional safety and efficacy data and any other factors to be considered.

### Backfill Cohorts

The purpose of backfill cohorts is to assess the safety, tolerability, and the biological effect of G9.2-17 (IGG4) in patients whose tumors are gal-9 positive. The gal-9 status of the RP2D cohort is retrospectively determined. If fewer than 6 patients with gal-9 positive tumors are treated at the RP2D, patients designated for the backfill cohort require prospective assessment of gal-9 tumor status by IHC. Up to 6 additional patients, whose tumors are gal-9 positive, may be enrolled to backfill cohorts at the RP2D dose level.

### Part 2: Cohort Expansion Phase

The second part of the protocol adopts a Simon's two-stage optimal design and includes approximately 223 patients. It is planned to expand cohorts for PDAC, CRC and CCA and/or potentially other solid tumor types which are based on implementing tumor-specific consideration for expansion cohorts and clinical trial endpoints. The rationale behind this approach is to ensure recruitment feasibility, as well as to capture the clinical need for specific indications.

CRC and CCA patients receive one of two treatments (4 treatment arms total):
LYT 200 as a single agent
G9.2-17 (IGG4)+an anti-PD-1 antibody as combination treatment.

The anti-PD-1 antibody should be administered prior to G9.2-17 (IgG4). If for any reason same-day administration cannot be accomplished, an anti-PD-1 antibody should be administered on the first day, and G9.2-17 (IgG4) on the subsequent day.

In some instances, this study may investigate the use of the anti-Galectin-9 antibody G9.2-17 (IgG4) alone (single agent arms of the study) or in conjunction with nivolumab (e.g., at a 240 mg flat dose administered once every two weeks).

Patients with CRC and CCA

Treatment of single agent cohorts or combination agent cohorts for CRC and CCA patients may be executed in parallel.

### G9.2-17 (IgG4) Single Treatment

The starting dose of G9.2-17 (IgG4) in the single treatment is the RP2D identified in Part 1. For the CRC and CCA single treatment arms, the optimal two-stage design (Stages I and II) are used to test the null hypothesis that the ORR3 is ≤5% versus the alternative hypothesis that the ORR3 is ≥15% within the single-agent arms.

After testing the investigational drug on 23 patients in Stage I, this trial arm is terminated if ≤1 patient responds. If the trial goes on to the Stage II of Simon's optimal design, approximately 33 patients are treated additionally in each of the single-agent arms. If the total number of responding patients is ≤5, the investigational drug within that arm is rejected. If ≥6 patients have a confirmed ORR 3, the Part 3 expansion cohort for that arm is activated and described in an amendment to the protocol.

Dose reduction is allowed only if the clinical benefit is expected and may continue to be expected to derive with lower doses of G9.2-17 (IgG4). The dose of G9.2-17 (IgG4) is initially reduced by 50%, and potentially by a further reduction of 50%, as defined by the dose modification guidance provided in the protocol. No further dose reductions are allowed.

### G9.2-17 (IgG4)+an Anti-PD-1 Antibody Combination Treatment

The dose of G9.2-17 (IGG4) in the combination treatment with an anti-PD-1 antibody (e.g., nivolumab or pembrolizumab) is the RP2D-1, which is the dose immediately preceding the RP2D dose identified in Part 1. The optimal two-stage design is also used to test the null hypothesis that the ORR 3 is ≤10% versus the alternative hypothesis that the ORR 3 is ≥25%.

To ensure patient safety, a safety run-in is performed in which the first 8 patients are dosed. This arm continues to enroll only if ≤2 patients develop a DLT, which is below the target toxicity level (TTL) of 25%. If 3 or more patients develop a DLT this combination arm is terminated for the cancer type being treated. In this combination treatment run-in cohort, patients who withdraw for reasons other than toxicity or tolerability issues during the first treatment cycle only are replaced. If a DLT occurs, in any of the 8 safety run in patients, during the first 28 days of treatment, that patient is permanently discontinued from study drug administration.

For patients who experience toxicities outside of the DLT window, dose reduction is allowed when clinical benefit is expected and may continue to be derived with lower doses of G9.2-17 (IgG4). The dose of G9.2-17 (IgG4) is initially reduced by 50%, and potentially by a further reduction of 50%, as defined by the dose modification guidance provided in the protocol. No further dose reductions are allowed. Dose modifications for an anti-PD-1 antibody are allowed.

If an IMAR occurs/recurs that is not managed by dose reduction of either agent, both study medications must be discontinued.

After testing the combination on 18 patients in the first stage, the respective trial arm is terminated if ≤2 patients respond. If the trial goes on to Stage II of Simon's optimal design, approximately 25 patients are treated additionally in each of the combination arms. If the total number of responding patients is ≤7, the combination within that arm is rejected. If ≥8 patients have a confirmed ORR 3, the expansion cohort for that arm is activated and described in an amendment to the protocol.

Patients with PDAC

The Part 2 cohort for patients with metastatic PDAC entails combination treatment of G9.2-17 (IgG4) in the first line metastatic setting.

The dose of G9.2-17 (IGG4) is the RP2D-1 dose, which is the dose level in the cohort immediately preceding the RP2D dose identified in Part 1. To ensure patient safety, a safety run-in is performed in which the first 8 patients are dosed and that arm is continued only if ≤2 patients develop a DLT, which is below the target toxicity level (TTL) of 25%. If 3 or more patients develop a DLT, this combination treatment arm is terminated. In this combination treatment run-in cohort, patients who withdraw for reasons other than toxicity or tolerability issues during the first treatment cycle only are replaced. If a DLT occurs, in any of the 8 safety run in patients, during the first 28 days of treatment, that patient is permanently discontinued from study drug administration.

For patients who experience toxicities outside of the DLT window, dose reduction is allowed when clinical benefit is expected and may continue to be derived with lower doses of G9.2-17 (IgG4). The dose of G9.2-17 (IgG4) is initially reduced by 50%, and potentially by a further 50%. No further dose reductions are allowed.

If an IMAR occurs/recurs that is not managed by dose reduction of either agent, both study medications must be discontinued.

The primary efficacy endpoint is patient PFS6.

### Part 2 Completion

Completion of Part 2 is dependent upon patient ORR 3 for CRC and CCA patients, and PFS 6 for PDAC.

### Part 3: Expansion

If a promising efficacy signal is identified within one or more of the trial arms that is attributable to the tumor type, an expansion cohort is launched to confirm the finding as described above. The sample size for each of the expansion arms is determined based on the point estimates determined in Part 2, in combination with a predetermined level of precision for the 95% CI around the ORR/OS and PFS. A protocol amendment is submitted with details around the expansion population, treatment regimen, and statistical analysis plan prior to initiating Part 3.

### Dose-Limiting Toxicity Criteria

Dose-limiting toxicities assessed in this trial are defined as a clinically significant hematologic and/or non-hematologic AE or abnormal laboratory value assessed as unrelated to metastatic tumor disease progression, intercurrent illness, or concomitant medications and is possibly related or related to the study drug and occurring during the first cycle (28 days) on study. Any patient that experiences a DLT in Part 1 or Part 2 during the first 28 days of treatment is permanently discontinued from study drug administration.

A DLT is a toxicity that meets any of the following criteria:
Any death not clearly due to the underlying disease or extraneous causes
Indications of potential drug induced liver injury (Hy's Law cases) as follows:
   ALT or AST>3×the upper limit of normal (ULN) with confirmation by repeat testing 24 hours later, AND
   Serum total bilirubin (TBL)>2×ULN with confirmation by repeat testing 24 hours later
   No other explanation can be found for the elevated TBL and/or ATs, such as viral hepatitis (A, B or C), alcoholic or autoimmune hepatitis, pre-existing or acute liver disease, gall bladder obstruction or bile duct disease, Gilbert syndrome, disease progression, or another medication capable of causing the observed effect.
All Grade 4 non-hematologic and hematological toxicities of any duration
All Grade 3 non-hematologic and hematological toxicities. Exceptions are as follow:
   Grade 3 nausea, vomiting and diarrhea that does not require hospitalization or total parenteral nutrition support and can be managed with supportive care to ≤ Grade 2 within 48 h.
   Grade 3 electrolyte abnormalities that are corrected to ≤ Grade 2 within 24 h.
   Grade 3 electrolyte abnormality that lasts <24-72 hours, is not clinically complicated, and resolves spontaneously or responds to conventional medical interventions.
   ≥Grade 3 amylase or lipase that is not associated with symptoms or clinical manifestations of pancreatitis.

### End of Study Definition

End of study for Part 1 of the study is defined at the point when the RP2D has been identified and all patients have been treated with G9.2-17 (IGG4) until confirmed disease progression.

End of study for Part 2 of the study is defined for each of the three tumor types following the completion of Simon's two-stage optimal design and all enrolled patients have been treated with G9.2-17 (IGG4) (alone or in combination) until confirmed disease progression.

In both Part 1 and Part 2 patients are followed for OS for up to 2 years following the last dose of G9.2-17 (IgG4) if they discontinue treatment due to reasons other than progression of disease and they not receiving additional systemic anticancer treatments.

The end of the study is defined as the date of the last patient's last visit.

### Trial Stopping Rules

### Part 1

The trial is stopped for safety if the lower limit of an Agresti and Coull binomial CI for the lowest study dose level exceeds the target DLT rate (Agresti and Coull, 1998).

### Part 2

After testing the investigational drug on 23 patients in Stage I of Simon's optimal design for CRC and CCA single treatment arms, the respective trial arm is stopped if ≤1 patient responds. If the trial goes on to the Stage II of Simon's optimal design, a trial arm is stopped if the total number of responding patients is ≤5 within that arm.

Similarly, for the G9.2-17 (IgG4)+an anti-PD-1 antibody combination in CRC and CCA, Simon's optimal design also guides trial stopping. After testing the combination on 18 patients in Stage I, the respective trial arm is stopped if ≤2 patients respond. If the trial goes on to Stage II, a trial arm is stopped if the total number of responding patients is ≤7 within that arm.

To ensure patient safety in both combination treatment arms, a safety run-in is performed in which the first 8 patients are dosed. For each cancer type (e.g., CCA, CRC, and/or PDAC) enrollment continues only if ≤2 patients develop a DLT, which is below the target toxicity level (TTL) of 25%. If 3 or more patients with a given cancer type develop a DLT in a combination treatment arm, enrollment for that cancer type in that arm is terminated.

### Study Population

### Inclusion Criteria

Participants are eligible to be included in the study only if all the following criteria apply:

### Part 1 and Part 2

1. Written Informed Consent (mentally competent patient, able to understand and willing to sign the informed consent form)
2. Age ≥18 years, male or non-pregnant female
3. Histologically confirmed unresectable metastatic cancer (adenocarcinomas and squamous cell carcinomas allowed). Patients with resectable disease are excluded.
4. Able to comply with the study protocol
5. Life expectancy >3 months
6. Eastern Cooperative Oncology Group (ECOG) performance status 0-1
7. Coronavirus SARS-COV-2 (COVID-19) negative patients
8. Patient able and willing to undergo pre- and on/post-treatment biopsies. The planned biopsies should not expose the patient to substantially increased risk of complications. Every effort is made that the same lesion is biopsied on repeat biopsies.
9. Measurable disease, according to Response Evaluation Criteria in Solid Tumors (RECIST) v1.1. Note that lesions intended to be biopsied should not be target lesions.
10. Adequate hematologic and end organ function, defined by the following laboratory results obtained prior to first dose of study drug treatment:
   a. neutrophil count ≥1×10⁹/L
   b. platelet count ≥100×10⁹/L; for hepatocellular carcinoma (HCC) in Part 1≥50×10⁹/L
   c. hemoglobin ≥9.0 g/dL without transfusion in the previous week
   d. creatinine ≤1.5× upper limit of normal (ULN)
   e. aspartate aminotransferase AST (SGOT)≤3×ULN (≤5×ULN when HCC or hepatic metastases are present)
   f. alanine aminotransferase (ALT [SGPT])≤3×ULN (≤5×ULN when HCC or hepatic metastases present)
   g. bilirubin≤1.5×ULN (patients with known Gilbert's disease may have a bilirubin ≤3.0×ULN)
   h. albumin ≥3.0 g/dL
   i. international normalized ratio (INR) and partial thromboplastin time (PTT)≤1.5×ULN
   j. amylase and lipase≤1.5×ULN
11. No evidence of active infection or infections requiring parenteral antibiotics, and no serious infection within 4 weeks before study start
12. Women of child-bearing potential must have a negative pregnancy test within 72 h prior to start of treatment. For women of childbearing potential: agreement to remain abstinent (refrain from heterosexual intercourse) or to use contraceptive methods that result in a failure rate of <1% per year during the treatment period and for at least 180 days after the last study treatment.
   A woman is of childbearing potential if she is post-menarche, has not reached a postmenopausal state (≥12 continuous months of amenorrhea with no identified cause other than menopause), and has not undergone surgical sterilization (removal of ovaries and/or uterus).
   Examples of contraceptive methods with a failure rate of <1% per year include bilateral tubal ligation, male sterilization, hormonal contraceptives that inhibit ovulation, hormone-releasing intrauterine devices and copper intrauterine devices. The reliability of sexual abstinence should be evaluated in relation to the duration of the clinical trial and the preferred and usual lifestyle of the patient. Periodic abstinence (e.g., calendar, ovulation, symptom-thermal, or post ovulation methods) and withdrawal are not acceptable methods of contraception. Fertile men must practice effective contraceptive methods during the study, unless documentation of infertility exists.
13. Four (4) weeks or 5 half-lives (whichever is shorter) since the last dose of anti-cancer therapy before the first G9.2-17 (IgG4) administration
14. Continuation of bisphosphonate treatment (e.g., zoledronic acid) or denosumab for bone metastases, which have been stable for at least 6 months before C1D1, is allowed
15. Biliary or gastric outlet obstruction allowed, provided it is effectively drained by endoscopic, operative, or interventional means
16. Pancreatic, biliary, or enteric fistulae allowed, provided they are controlled with an appropriate non-infected and patent drain (if any drains or stents are in situ, patency needs to be confirmed before study start)

Additionally, for Part 1 only:
17. Patients:
   a. who has already received at least one prior line of systemic therapy for metastatic disease, or
   b. who has a tumor type for which there are no available standard of care options.

Additionally, for Part 2 only:
18. PDAC expansion cohort: 1st line metastatic patients who are either gemcitabine-containing regimen naïve or at least 3 months out of having been treated using a gemcitabine-containing regimen previously in a neoadjuvant or adjuvant/locally advanced setting
19. CRC and CCA expansion cohorts—patients who have received at least one prior line of therapy in the metastatic setting

### Exclusion Criteria

Participants are excluded from the study if any of the following criteria apply:
1. Patient unwilling or unable to follow protocol requirements
2. Patient diagnosed with metastatic cancer of an unknown primary
3. Prior or current illicit drug addiction (medical and recreational marijuana/cannabidiol (CBD)/Tetrahydrocannabinol (THC) would not be considered “illicit”)
4. Clinically significant, active uncontrolled bleeding, and any patients with a bleeding diathesis (e.g., active peptic ulcer disease). Prophylactic or therapeutic use of anticoagulants is allowed.
5. Pregnant and/or lactating females
6. Receiving any other investigational agents or participating in any other clinical trial involving another investigational agent for treatment of solid tumors within 4 weeks or 5 half-lives of the administered drug (whichever is shorter) prior to Cycle 1, Day 1 of the study, or other investigational therapy or major surgery within 4 weeks of the date of consent, or planned surgery within 4 weeks of envisaged study start (this includes dental surgery).
7. Radiation therapy within 4 weeks of the first dose of study drug, except for palliative radiotherapy to a limited field, such as for the treatment of bone pain or a focally painful tumor mass, and which does not jeopardize required measurable lesions for response assessment (RECIST v1.1).
8. Patients with fungating tumor masses
9. Patients with locally advanced PDAC without distant organ metastatic deposits
10. Grade 4 immune-mediated toxicities with a prior checkpoint inhibitor. Grade 2 or Grade 3 pneumonitis or any other Grade 3 checkpoint inhibitor-related toxicity that led to immunotherapy treatment discontinuation. Low-grade (<Grade 3) toxicities, such as neuropathy from prior treatments, manageable electrolyte abnormalities and lymphopenia, alopecia and vitiligo are allowed.
11. History of second malignancy, except those treated with curative intent more than five years previously without relapse or low likelihood of recurrence (for example, non-melanotic skin cancer, cervical carcinoma in situ, early (or localized) prostate cancer, or superficial bladder cancer)
12. Active brain or leptomeningeal metastases. Patients with brain metastases are eligible provided they have shown clinically and radiographically stable disease (SD) for at least 4 weeks after definitive therapy and have not used steroids (>10 mg/day of prednisone or equivalent) for at least 4 weeks prior to the first dose of study drug
13. Evidence of severe or uncontrolled systemic diseases, congestive heart failure >New York Heart Association (NYHA) class 2, myocardial infarction (MI) within 6 months, or laboratory finding that makes it undesirable for the patient to participate in the trial
14. Any medical condition that is considered significant to compromise the safety of the patient or that impairs the interpretation of G9.2-17 (IgG4) toxicity assessment
15. Serious non-healing wound, active ulcer, or untreated bone fracture
16. Uncontrolled pleural effusion, pericardial effusion, or ascites requiring recurrent drainage procedures. For the purposes of this study, “recurrent” is defined as ≥3 drains in the previous 30 days.
17. History of severe allergic, anaphylactic, or other hypersensitivity reactions to chimeric or humanized antibodies or fusion proteins
18. Significant vascular disease (e.g., aortic aneurysm requiring surgical repair or recent arterial thrombosis) within 6 months of Cycle 1, Day 1
19. History of pulmonary embolism, stroke or transient ischemic attack within 3 months prior to Cycle 1, Day 1
20. History of abdominal fistula or gastrointestinal perforation within 6 months prior to Cycle 1, Day 1
21. Active auto-immune disorder (except type I/II diabetes, hypothyroidism requiring only hormone replacement, vitiligo, psoriasis, or alopecia areata)
22. Requires systemic immunosuppressive treatment, including, but not limited to cyclophosphamide, azathioprine, methotrexate, thalidomide, and anti-TNF agents. Patients who have received or are receiving acute, low dose systemic immunosuppressant medications (e.g., ≤ 10 mg/day of prednisone or equivalent) may be enrolled. Replacement therapy (e.g., thyroxine, insulin, physiologic corticosteroid replacement therapy [eg, ≤10 mg/day of prednisone equivalent] for adrenal or pituitary insufficiency) is not considered a form of systemic treatment. The use of inhaled corticosteroids and mineralocorticoids (eg, fludrocortisone), topical steroids, intranasal steroids, intra-articular, and ophthalmic steroids is allowed.
23. Severe tumor-related pain (≥Grade 3, per the Common Terminology Criteria for Adverse Events, (CTCAE) v.5.0) unresponsive to broad analgesic interventions (oral and/or patches)
24. Hypercalcemia (Grade 3 per CTCAE v 5.0), despite use of bisphosphonates
25. Any other diseases, metabolic dysfunction, physical examination finding, or clinical laboratory finding giving reasonable suspicion of a disease or condition that contraindicates the use of an investigational drug or that may affect the interpretation of the results or render the patient at high risk of treatment complications
26. Received organ transplant(s)
27. Patients undergoing dialysis
28. For patients enrolled into an anti-PD-1 antibody combination cohorts, no prior exposure to any anti-PD-1 or anti-PD-L1 agent in any prior lines of therapy. Additionally, patients diagnosed as dMMR/MSI-H are excluded.
29. For Part 1, hormonal androgen deprivation therapy is allowed to continue for patients with metastatic castration-resistant prostate cancer.
30. Any ablative therapy (Radio Frequency Ablation or Percutaneous Ethanol Injection) for HCC<6 weeks prior trial entry
31. Hepatic encephalopathy or severe liver adenoma
32. Child-Pugh score ≥7

Additionally, for Part 2 only:
33. Hypersensitivity to the active substance or to any of the excipients listed in the ingredients of an anti-PD-1 antibody

Additionally, for Part 2 combination (G9.2-17 (IGG4)+an anti-PD-1 antibody) arms only:
34. Received live vaccine within 28 days of treatment start. Inactivated vaccines (i.e. influenza and Covid-19) are allowed.

### Study Drug and Other Interventions

Study intervention(s) is/are defined as any investigational agent(s), marketed product(s), placebo, or medical device(s) intended to be administered/used to/in a study participant according to the study protocol.

Agents Administered in Combination with G9.2-17 IgG4

### Nivolumab

Nivolumab (OPDIVO®) is a programmed death receptor-1 (PD-1) blocking antibody indicated for the treatment of multiple tumor types. Nivolumab can be used as an exemplary anti-PD-1 antibody in combination with the anti-Galectin-9 antibody disclosed herein such as G9.2-17 IgG4.

Nivolumab can be administered as an intravenous infusion over 30 minutes (unless guided otherwise) at 240 mg every 2 weeks, in a 28-day cycle. As per the FDA label, there are no contraindications for administrations of nivolumab.

Nivolumab AEs are presented in the Tables below according to their frequency of occurrence.

Adverse Events (non-IMAR-Related) Reported for Nivolumab According to Frequency

Common: AEs occurring in >30% of patients

| | | | |
|---|---|---|---|
| Fatigue | Lymphocytopenia | Hyponatremia | Decreased Appetite |
| Myalgia | Decreased Appetite | C | |

Less Common: AE's occurring in 10-29% of patients

| | | | |
|---|---|---|---|
| Nausea | Vomiting | Constipation | Weakness |
| Hyperkalemia | Hypokalemia | Hypercalcemia | Hypocalcemia |
| Hypomagnesemia | Swelling | Fever | Abdominal pain |
| Weight loss | Pain | Thrombocytopenia | Pneumonia |

Rare (but severe) AEs occurring in <10% of patients

| | |
|---|---|
| Stomatitis | Peripheral neuropathy |
| Bronchitis | Upper respiratory tract infection |

Source: Section 6.1, OPDIVO Package Insert

### Adverse Events Reported for Nivolumab to be Treated as IMARs*

Common: AEs occurring in >30% of patients

| | |
|---|---|
| Cough | Dyspnea |

Less Common: AE's occurring in 10-29% of patients

| | | | |
|---|---|---|---|
| Increased serum AST | Increased ALT | Increased ALP | Increased serum creatinine |
| Chest pain | | Rash | Itching |
| Diarrhea | Colitis | Joint pain | |

Rare (but severe) AEs occurring in <10% of patients

| | |
|---|---|
| Immune-mediated pneumonitis | Immune-mediated hypo- and hyperthyroidism |
| Immune-mediated hepatitis | Immune mediated nephritis and renal dysfunction |
| Immune-mediated colitis | |

### Study Intervention Administration

All patients receive G9.2-17 (IgG4). G9.2-17 (IgG4) is administered via IV infusion, weekly, or every 2 weeks, until progression of disease, unacceptable toxicity, or withdrawal of consent.

In Part 1, patients receive G9.2-17 (IgG4) alone at sequentially increasing doses starting at 0.2 mg/kg.

In Part 2, patients receive the RP2D of G9.2-17 (IgG4) (as determined in Part 1) as a single agent or the G9.2-17 (IgG4) RP2D-1 in combination with an anti-PD-1 antibody as follows:
Patients with CRC or CCA
   G9.2-17 (IgG4) in CRC
   G9.2-17 (IgG4) in CCA
   G9.2-17 (IgG4)+an anti-PD-1 antibody in CRC
   G9.2-17 (IgG4)+an anti-PD-1 antibody in CCA
Other solid tumor types (based on data from Part 1)
   G9.2-17 (IgG4) as a single agent and/or in combination with a checkpoint inhibitor or chemotherapy that is determined based on each tumor type

See Table 4 for a summary description of each study intervention.

Patients who experience a DLT in Part 1 do not resume treatment. Patients who experience a DLT in Part 2 have their treatment interrupted. Their treatment may resume at the same or reduced dose of G9.2-17 (IgG4) if they are experiencing a clinical benefit.

TABLE 4
Summary Characteristics of Study Interventions

| | | G9.2-17 (IgG4) + an anti-PD1 |
|---|---|---|
| Arm Name | G9.2-17 (IgG4) | antibody |
| Intervention | G9.2-17 (IgG4) | an anti-PD1 antibody |
| Name | | |
| Intervention | Biologic | Biologic |
| Type | | |
| Dose | Vial | Vial |
| Formulation | | |
| Unit Dose | 30 mg/mL | e.g., for nivolumab, 40 mg/ml vial |
| Strength(s) | | and 100 mg/10 mL vial |
| Dosage Level(s) | Part 1: increasing doses starting at Q2W | Part 2 only: |
| | 0.2 mg/kg with a maximum dose of QW | an anti-PD1 antibody: (based on |
| | 16 mg/kg | approved or investigational dose of |
| | Part 2: RP2D for single agent G9.2-17 | the antibody, e.g., for nivolumab, |
| | (IGG4) or RP2D-1 for G9.2-17 (IGG4) in | 240 mg) |
| | combination with an anti-PD1 antibody or | |
| | chemotherapy | |
| Frequency of | Q2W: Days 1 and 15 of each 28-day cycle | Days 1 and 15 of each 28-day cycle |
| Administration | (Part 1, Cohorts 1-6) | |
| | QW: Days 1, 8, 15, and 22 of each 28-day | |
| | cycle (Part 1, Cohorts 7-8 and Part 2) | |
| Route of | IV infusion | IV infusion |
| Administration | | |
| Use | experimental | experimental |
| IMP and non- | IMP | IMP |
| IMP | | |
| Sourcing | Provided centrally | Provided centrally |
| Packaging and | Study Intervention is provided in | Study Intervention is provided in |
| Labeling | appropriate configurations; each is | appropriate configurations; each is |
| | labeled as required per country | labeled as required per country |
| | requirement | requirement |

IMP: investigational medicinal product,
IV: intravenous,
mAb: monoclonal antibody,
RP2D: recommended Phase 2 dose;
RP2D-1: dose level in cohort immediately preceding the RP2D

### Preparation of G9.2-17 (IgG4)

Manufacture and packaging of the investigational medicinal product (IMP) G9.2-17 (IgG4) is in accordance with applicable current Good Manufacturing Practice (cGMP) and the product meets applicable criteria for use in humans.

G9.2-17 (IgG4) drug product is diluted to the target dose prior to administration. All dilutions should be performed in a controlled and sterile environment (patient dose is prepared for and delivered via an approximately 60 minutes IV infusion).

G9.2-17 (IgG4) is a sterile liquid and is stored at 2° C. to 8° C. and protected from light.

### Dose De-Escalation

If a patient is experiencing clinical benefit from G9.2-17 (IgG4), and protocol efficacy assessment criteria, and the patient is experiencing adverse reactions that are not attributed to G9.2-17 (IgG4), then treatment with G9.2-17 (IgG4) alone may continue.

G9.2-17 (IgG4) may be continued if:
the patient's clinical status is not deteriorating rapidly; AND
the combination agent is discontinued due to AEs that are attributed to the combination agent only.

If an IMAR occurs/recurs that is not managed by dose reduction of either agent, both study medications must be discontinued.

Nab-paclitaxel is not recommended in patients who have total bilirubin >5×ULN or AST>10×ULN. In addition, Nab-paclitaxel is not recommended in patients with metastatic adenocarcinoma of the pancreas who have moderate to severe hepatic impairment (total bilirubin >1.5×ULN and AST≤10×ULN). The starting dose should be reduced for patients with moderate or severe hepatic impairment.

### Dose Modifications for Specific AEs Related to Administration of Nivolumab

Recommendations for nivolumab modifications based on specific AEs are provided below. There are no recommended dose modifications of nivolumab for hypothyroidism or hyperthyroidism.

Recommended dose modification for Nivolumab for AEs other than IMAR is provided below:

| Adverse | | Dose |
|---|---|---|
| Reaction | Severity* | Modification |
| OAEs | Grade 3 adverse events | Withhold dose^{a} |
| | First occurrence | Permanently |
| | Recurrence of same Grade 3 adverse reaction | discontinue |
| | Life-threatening or Grade 4 adverse reaction | Permanently |
| | Grade 3 myocarditis | discontinue |
| | Requirement for ≥10 mg/day prednisone or | |
| | equivalent for >12 weeks | |
| | Persistent Grade 2 or 3 adverse reactions | |
| | lasting ≥12 weeks | |

*Toxicity was graded per NCI CTCAE V4.
^{a}Resume treatment when adverse reaction improves to Grade 0 or 1Source: OPDIVO Highlights of Prescribing Information, Revised April 2019.

### Dose Modification for IMARs

If an IMAR occurs, see guidance provided herein on dose management of G9.2-17 IgG4 and/or nivolumab.

### Discontinuation of Study Intervention

In rare instances, it may be necessary for a patient to permanently discontinue study intervention. If study intervention is permanently discontinued due to reasons other than disease progression, and the patient is not being treated with other anti-cancer therapy(ies), the patient continues to be evaluated for disease progression for up to 2 years. See the SoA for data to be collected at the time of discontinuation of study intervention and follow-up and for any further evaluations that need to be completed.

Every effort must be made by study personnel to keep patients on study treatment until one of the reasons for study treatment termination are met (disease progression, toxicity related to the study drug, withdrawal of consent). If the patient has radiographic progression but no unequivocal clinical progression and alternate treatment is not initiated, the patient may continue on study treatment. However, if patients have unequivocal clinical progression without radiographic progression, study treatment should be stopped, and patients advised regarding available treatment options.

A patient may be discontinued prior to disease progression for any of the following reasons:
A DLT per definition in Section 3.4.4.
An AE occurs/recurs outside of the DLT window that requires discontinuation of study treatment(s)
An IMAR occurs/recurs that requires discontinuation of study treatment(s)
Termination of the study by PureTech Health, LLC
Intercurrent illness or medical condition that prevents further administration of treatment or may jeopardize the patient's safety if they continue on study treatment
Pregnancy
Use of a non-protocol anti-cancer therapy

Patients may also be discontinued prior to disease progression for any of the following reasons:
Significant deviation from protocol on the part of the patient (includes lack of compliance)

The explanation of why the patient is discontinuing study treatment should be documented in the case report form (CRF). If the patient discontinues study treatment due to toxicity, “Dose-Limiting Toxicity” or “Adverse Event” is recorded as the primary reason for withdrawal. If a patient is prematurely discontinued from the study at any time due to an AE or SAE, the patient must be followed until resolution to Grade 2 or less, unless it is unlikely to improve because of the underlying disease.

### Concomitant Therapy

Any medication or vaccine (including over-the-counter or prescription medicines, recreational drugs, vitamins, and/or herbal supplements) that the participant is receiving at the time of enrollment or receives during the study must be recorded along with:
Reason for use
Dates of administration including start and end dates
Dosage information including dose and frequency

### Permitted Medications

The following concomitant medications are allowed:
Any standard of care pre-medication for patients on a combination treatment regimen.
Continuation of bisphosphonate treatment (e.g., zoledronic acid) or denosumab for bone metastases, which have been stable for at least 6 months before treatment (C1D1),
The use of inhaled corticosteroids and mineralocorticoids (e.g., fludrocortisone), topical steroids, intranasal steroids, intra-articular, and ophthalmic steroids
Prophylactic or therapeutic use of anticoagulants
Vaccination for COVID-19, common flu and/or other common clinically required indications (e.g., tetanus, pneumococcus, HBV, etc.) is allowed before or during the study period. The timing and type of vaccine must be recorded.

### Prohibited Medications

Following medications are not allowed while on this study:
Concomitant administration of other investigational agents, other than G9.2-17 (IGG4), for any indication.
Systemic immunosuppressive treatment, including, but not limited to cyclophosphamide, azathioprine, methotrexate, thalidomide, and anti-TNF agents. However, patients are allowed to take acute, low dose systemic immunosuppressant medications (e.g., ≤10 mg/day of prednisone or equivalent).
Replacement therapy (e.g., thyroxine, insulin, physiologic corticosteroid replacement therapy [eg, ≤10 mg/day of prednisone equivalent] for adrenal or pituitary insufficiency) is not considered a form of systemic treatment.

### Supportive Care

Patients should receive full supportive care during the study, including transfusions of blood and blood products, and treatment with antibiotics, antiemetics, antidiarrheals, and analgesics, and other care as deemed appropriate, and in accordance with institutional guidelines

### Schedule of Assessments

TABLE 5
Schedule of Assessments for Cohorts 1-6

| | Pre-Dose | Treatment Phase | | | | | | | | | | |
|---|---|---|---|---|---|---|---|---|---|---|---|---|
| | Screening | Cycle 1 (28 days) | | | | Cycle 2 (28 days) | | | Cycle 3 (28 days) | | | |
| Study Day | −28 to −1 | 1 | 2 | 8 | 15 | 1 | 8 | 15 | 1 | 2 | 8 | 15 |
| Cycle Day | | C1 | C1 | C1 | C1 | C2 | C2 | C2 | C3 | C3 | C3 | C3 |
| | | D 1 | D 2 | D 8 | D 15 | D 1 | D 8 | D 15 | D 1 | D 2 | D 8 | D 15 |
| +/− days | | | 1 | 1 | 1 | 2 | 1 | 2 | 2 | 1 | 1 | 2 |
| allowable | | | | | | | | | | | | |
| Study drug | | X | | X | X | X | X | X | X | | X | X |
| administration ^{A} | | | | | | | | | | | | |

Study Procedures & Examinations

| | | | | | | | | | | | |
|---|---|---|---|---|---|---|---|---|---|---|---|
| Eligibility | X | | | | | | | | | | |
| assessment and | | | | | | | | | | | |
| written informed | | | | | | | | | | | |
| consent | | | | | | | | | | | |
| Demographics ^{B} | X | | | | | | | | | | |
| Medical history ^{C} | X | | | | | | | | | | |
| Previous and | X | X | X | X | X | X | X | X | X | X | X |
| concomitant | | | | | | | | | | | |
| medications ^{D} | | | | | | | | | | | |
| Adverse events ^{E} | | X | X | X | X | X | X | X | X | X | X |
| ECHO/MUGA ^{F} | X | | | | | | | | | | |
| 12-lead ECG | X | X | | | | X | | X | | | |
| (QTcF) | | | | | | | | | | | |
| Physical exam ^{G} | X | X | | | X | | X | | | | X |
| ECOG | X | X | | | X | | X | X | | | X |
| Vital Signs^{H} | X | X | | | X | | X | | | | |
| Tumor imaging | X | | | | | | | X | | | |
| assessment (CT | | | | | | | | | | | |
| with contrast | | | | | | | | | | | |
| preferred) N | | | | | | | | | | | |

Clinical Labs

| | | | | | | | |
|---|---|---|---|---|---|---|---|
| Pregnancy test ^{I} | X | | | X | | X | |
| Hematology ^{J} | X | X | X | X | X | X | X |
| Serum Chemistry ^{K} | X | X | X | X | X | X | X |
| TSH, Free T4, | X | X | | X | | X | |
| serum lipase, | | | | | | | |
| amylase, PTH, | | | | | | | |
| FSH, LH, free | | | | | | | |
| cortisol | | | | | | | |
| Blood | X | X | X | X | X | X | X |
| Coagulation ^{L} | | | | | | | |
| Urinalysis ^{M} | X | X | X | X | X | X | X |

Pharmacodynamics and Pharmacokinetics

| | | | | | | | | | | | |
|---|---|---|---|---|---|---|---|---|---|---|---|
| Tumor biopsy ^{O} | X | | | | | | | | | | X ± |
| | | | | | | | | | | | 7 d |
| Tumor type- | X | X | | | X | X | X | X | | | X |
| relevant | | | | | | | | | | | |
| biomarkers ^{P} | | | | | | | | | | | |
| Blood sampling | | X | X | X | X | X | | X | X | X | X |
| for biomarker PD | | | | | | | | | | | |
| analysis ^{Q} | | | | | | | | | | | |
| PK blood | | X | X | X | X | X | | X | X | X | X |
| sampling ^{R} | | | | | | | | | | | |
| Immunogenicity | | X | | | | X | | X | | | |
| (ADA) ^{S} | | | | | | | | | | | |
| Survival | | | | | | | | | | | |
| Follow-up | | | | | | | | | | | |

| | | | | Post Treatment | | |
|---|---|---|---|---|---|---|
| | Treatment Phase | | | End of Treatment | | |
| | Cycle 4 (28 days) | | | or Early Termination | IMAR Follow-Up^{T} | Long Term |
| | and beyond | | | 30 days after | 90 days after | Follow-Up ^{U} |
| Study Day | 1 | 8 | 15 | last dose | last dose | Every 3 mons |
| Cycle Day | CX | CX | CX | — | — | — |
| | D 1 | D 8 | D 15 | | | |
| +/− days | 2 | 1 | 2 | 3 | 7 | n/a |
| allowable | | | | | | |
| Study drug | X | X | X | | | |
| administration ^{A} | | | | | | |
| Study Procedures & Examinations | | | | | | |
| Eligibility | | | | | | |
| assessment and | | | | | | |
| written informed | | | | | | |
| consent | | | | | | |
| Demographics ^{B} | | | | | | |
| Medical history ^{C} | | | | | | |
| Previous and | X | | X | X | | |
| concomitant | | | | | | |
| medications ^{D} | | | | | | |
| Adverse events ^{E} | X | | X | X | X | |
| ECHO/MUGA ^{F} | X | | | | | |
| 12-lead ECG | X | | | | | |
| (QTcF) | | | | | | |
| Physical exam ^{G} | X | | X | X | X | |
| ECOG | X | | X | X | | |
| Vital Signs^{H} | X | | X | X | | |
| Tumor imaging | | | | X | | X U |
| assessment (CT | | | | repeat if end | | |
| with contrast | | | | of study is >8 | | |
| preferred) N | | | | weeks after | | |
| | | | | last scan | | |
| Clinical Labs | | | | | | |
| Pregnancy test ^{I} | X | | | X | | |
| Hematology ^{J} | X | | X | X | X | |
| Serum Chemistry ^{K} | X | | X | X | X | |
| TSH, Free T4, | X | | | X | X | |
| serum lipase, | | | | | | |
| amylase, PTH, | | | | | | |
| FSH, LH, free | | | | | | |
| cortisol | | | | | | |
| Blood | X | | X | X | X | |
| Coagulation ^{L} | | | | | | |
| Urinalysis ^{M} | X | | X | X | X | |
| Pharmacodynamics and Pharmacokinetics | | | | | | |
| Tumor biopsy ^{O} | | | | | | |
| Tumor type- | X | | X | X | | |
| relevant | | | | | | |
| biomarkers ^{P} | | | | | | |
| Blood sampling | X | | | X | | |
| for biomarker PD | | | | | | |
| analysis ^{Q} | | | | | | |
| PK blood | X | | | | | |
| sampling ^{R} | | | | | | |
| Immunogenicity | X | | | X | | |
| (ADA) ^{S} | | | | | | |
| Survival | | | | | | X ^{U} |
| Follow-up | | | | | | |

ADA: anti-drug antibodies; AE: adverse event; ALT: alanine aminotransferase; APTT: activated partial thromboplastin time; AST: aspartate aminotransferase; C: cycle; CPK: creatine phosphokinase; COVID19: Coronavirus SARS-COV-2; CRP: C-reactive protein; CT: computed tomography; D or d: day(s); ECG: electrocardiogram; ECOG: Eastern Cooperative Oncology Group; ECHO: echocardiography/cardiac ultrasound; FSH: follicle-stimulating hormone; IMAR: immune-mediated adverse reaction; INR: international normalized ratio; LDH: lactate dehydrogenase; LH: luteinizing hormone; min: minute(s); MUGA: multigated acquisition scan; PD: pharmacodynamics; PK: pharmacokinetics; PT: prothrombin time; PTH: parathyroid hormone; PTT: partial thromboplastin time; QTcF: QT interval, Fridericia's Correction Formula; RBC: red blood cell count; SGOT: serum glutamic-oxaloacetic transaminase; SGPT: serum glutamic pyruvic transaminase; TSH: thyroid stimulating hormone; WBC: white blood cell count.
^{A} Study drug administration: G9.2-17 (IGG4) treatment is administered, on C1D1 and C1D15 on every cycle. an anti-PD1 antibody is administered on Day 1 of every cycle on the G9.2-17 (IGG4) combination regimen. Study drug may be administered on Days 1, 8 and 15 +/- 3 days from C2 onwards.
^{B} Demographics: Data include age, gender, race, and ethnicity.
^{C} Medical history: In addition to general medical history, data collection also includes oncology history, surgical/transplant and radiation therapy history and COVID-19 history and testing.
^{D} Previous and concomitant medications (including vaccines and complementary treatments/supplements): Data to include name, indication, dose, route, start and end dates for each. Allergies and intolerances, dose modifications while on study, schedule of dosing changes and reasons for them should also be obtained.
^{E} Adverse events: Any AEs starting or worsening after study drug administration is recorded. AEs should be followed until resolved to one of the following: baseline, stabilized or deemed irreversible. All SAEs are to be collected until 30 days after last dose of study medication. All study-procedure-related SAEs must be collected from the date of patient's written consent.
^{F} ECHO/MUGA: This assessment of heart function is conducted at Screening and repeated on Day 1 of Cycle 4; the assessment window is +/- 5 days. It should be conducted more frequently when clinically indicated and once every 3 months.
^{G} Physical exam: Include height at screening for determination of body surface area. Include weight at all scheduled exam times. A Neurological exam is conducted only on patients who have stable and/or pre-treated brain metastases.
^{H}Vital Signs: temperature, heart rate, blood pressure, respiratory rate.
^{I} Pregnancy test (blood or urine): Only for women of childbearing potential with uterus in situ. Test results must be available before scheduled dosing.
^{J} Hematology: Analysis includes complete blood count, differential, platelets, hemoglobin. Collect blood samples pre-dose.
^{K} Serum chemistry: Analysis includes albumin, alkaline phosphatase, bilirubin (total, direct), blood urea nitrogen, calcium, CPK, creatinine, electrolytes (sodium, potassium, chloride, magnesium, phosphorus), gamma glutamyl transferase (gamma GT), glucose, hemoglobin Alc (HgbAlc) (only if history of Type 1 or Type 2 diabetes mellitus), LDH, SGPT (ALT) or SGOT (AST), total protein. Fasting glucose to be assessed only if clinically indicated. Collect blood samples pre-dose.
^{L} Blood Coagulation: Collect blood samples pre-dose. Analysis includes APTT, PT, PTT, and INR (if on allowable anti-coagulants), CRP, and troponin.
^{M} Urinalysis: Analysis includes color, appearance, dipstick for specific gravity, protein, white blood cell-esterase, glucose, ketones, urobilinogen, nitrite, WBC, RBC, pH. (Urine culture and sensitivity to be run only if patient is clinically symptomatic.)
^{N} Tumor imaging assessment: For screening, the assessment must be performed within the 28-day screening period. On study, assessments are done every 8 weeks ± 7 days (ie, C3D 1, C5D 1, C7D 1, C9D 1, etc.) and at the End of Treatment if not assessed within the previous 4-6 weeks. Assessments may be performed more frequently if clinically indicated. If an objective response is seen on a scan, a confirmation scan is done 4 weeks (+7 d) later. After this confirmatory scan, the scheduled scans are to be resumed at a frequency of every 8 weeks + 7 days from the date of the confirmatory scan.
^{O} Tumor biopsies: If patient MMR/MSI status is unknown at screening, the test should be run at the local laboratory. In Part 2, TMB tissue analysis is performed. The on-study biopsy is scheduled for C3D 15 ± 7 days and should occur only after the tumor imaging scan in Cycle 3. It is recognized that a variety of clinical factors may make it difficult to obtain adequate specimens. Decisions not to perform biopsy on-treatment should be discussed with the Medical Monitor.
^{P} Tumor type-relevant biomarkers: Blood samples are to be collected at screening and every cycle pre-dose administration as appropriate for the tumor type. Blood sampling may be decreased to every 3rd cycle after 6 months of treatment.
^{Q} PD blood sampling: Blood samples are collected pre-dose administration on dosing days. May be decreased to every 3rd cycle after 6 months of treatment.
^{R} PK blood sampling: Cycle 1 and Cycle 3 Day 1: blood samples are collected pre-dose and at end of study drug infusion (EOI), 2 and 4 h (±30 min) post-study drug administration. Cycle 1 and Cycle 3 Day 15, blood samples are collected pre-dose and at EOI only. Cycle 1 and Cycle 3 Day 2 and 8 (non-dosing days), PK blood samples are collected at only one time point. Cycle 2 and Cycle 4: blood samples are collected Day 1 only and should occur pre-dose and at EOI. Blood samples for PK are collected every 2 cycles thereafter (i.e., C6D 1, C8D 1, etc.) pre-dose and at EOI.
^{S} ADA blood sampling: Blood samples are collected Day 1 of Cycles 1-4, blood samples are collected Day 1, pre-dose. Thereafter, it is collected every 2 cycles, Day 1, pre-dos (ie, C6D 1, C8D 1, etc.).
^{T}All patients treated with G9.2-17 (IGG4) + an anti-PD1 antibody must return 90-days +/− 7 days after last dose of study drug for an assessment of potential immune-mediated adverse reactions (IMARs).
^{U} Long-Term Follow-up: Tumor imaging should continue, where possible, for patients discontinuing treatment due to reasons other than progression of disease and not receiving additional systemic anticancer treatments. Survival data is collected at a minimum every 3 months. It can be collected more frequently to support data cleaning or regulatory submission efforts. Follow-up can be conducted by telephone, electronic messaging or chart review and will continue for up to 2 years after the patient has the End of Treatment/Early Termination visit.

TABLE 6
Schedule of Assessments for Cohorts 7 and 8

| | | Treatment Phase | | | | | | | | | | | |
|---|---|---|---|---|---|---|---|---|---|---|---|---|---|
| | Pre-Dose | Cycle 1 | | | | | Cycle 2 | | | | Cycle 3 | | |
| | Screening | (28 days) | | | | | (28 days) | | | | (28 days) | | |
| Study Day | −28 to −1 | 1 | 3 | 8 | 15 | 22 | 1 | 8 | 15 | 22 | 1 | 3 | 8 |
| Cycle Day | | C1 | C1 | C1 | C1 | C1 | C2 | C2 | C2 | C2 | C3 | C3 | C3 |
| | | D 1 | D 3 | D 8 | D 15 | D 22 | D 1 | D 8 | D 15 | D 22 | D 1 | D 3 | D 8 |
| +/− days | | | 1 | 1 | 1 | 1 | 2 | 1 | 2 | 2 | 2 | 1 | 1 |
| allowable | | | | | | | | | | | | | |
| Study drug | | X | | X | X | X | X | X | X | X | X | | X |
| administration ^{A} | | | | | | | | | | | | | |

Study Procedures & Examinations

| | | | | | | | | | | | | | |
|---|---|---|---|---|---|---|---|---|---|---|---|---|---|
| Eligibility | X | | | | | | | | | | | | |
| assessment and | | | | | | | | | | | | | |
| written informed | | | | | | | | | | | | | |
| consent | | | | | | | | | | | | | |
| Demographics ^{B} | | | | | | | | | | | | | |
| Medical history ^{C} | | | | | | | | | | | | | |
| Previous and | X | X | X | X | X | X | X | X | X | X | X | X | X |
| concomitant | | | | | | | | | | | | | |
| medications ^{D} | | | | | | | | | | | | | |
| Adverse events ^{E} | | X | | X | X | X | X | X | X | X | X | X | X |
| ECHO/MUGA ^{F} | X | | | | | | | | | | | | |
| 12-lead ECG | X | X | | | | | X | | | | X | | |
| (QTcF) | | | | | | | | | | | | | |
| Physical exam ^{G} | X | X | | X | X | X | X | X | X | X | X | | X |
| ECOG | X | X | | X | X | X | X | X | X | X | X | | X |
| Vital Signs^{H} | X | X | | X | X | X | X | X | X | X | X | | X |
| Tumor imaging | X | | | | | | | | | | X | | |
| assessment (CT | | | | | | | | | | | | | |
| with contrast | | | | | | | | | | | | | |
| preferred) N | | | | | | | | | | | | | |

Clinical Labs

| | | | | | | | | | | | |
|---|---|---|---|---|---|---|---|---|---|---|---|
| Pregnancy test ^{I} | X | | | | | X | | | | X | |
| Hematology ^{J} | X | X | X | X | X | X | X | X | X | X | X |
| Serum Chemistry ^{K} | X | X | X | X | X | X | X | X | X | X | X |
| TSH, Free T4, | X | X | | | | X | | | | X | |
| serum lipase, | | | | | | | | | | | |
| amylase, PTH, | | | | | | | | | | | |
| FSH, LH, free | | | | | | | | | | | |
| cortisol | | | | | | | | | | | |
| Blood | | | | | | | | | | | |
| Coagulation ^{L} | | | | | | | | | | | |
| Urinalysis M | X | X | X | X | X | X | X | X | X | X | X |

Pharmacodynamics and Pharmacokinetics

| | | | | | | | | | | | |
|---|---|---|---|---|---|---|---|---|---|---|---|
| Tumor biopsy ^{O} | X | | | | | | | | | | |
| Tumor type- | X | X | | | X | | X | X | X | | |
| relevant | | | | | | | | | | | |
| biomarkers ^{P} | | | | | | | | | | | |
| Blood sampling | | X | X | X | X | | X | | X | X | X |
| for biomarker | | | | | | | | | | | |
| PD analysis ^{Q} | | | | | | | | | | | |
| PK blood | | X | X | X | X | X | X | | X | X | X |
| sampling ^{R} | | | | | | | | | | | |
| Immunogenicity | | X | | | X | | X | X | X | | |
| (ADA) ^{S} | | | | | | | | | | | |
| Survival Follow- | | | | | | | | | | | |
| up | | | | | | | | | | | |

| | | | | | | | Post Treatment | | |
|---|---|---|---|---|---|---|---|---|---|
| | | | | | | | End of | | |
| | | | | | | | Treatment | | Lo [symbol] |
| | Treatment Phase | | | | | | or Early | IMAR | Te [symbol] |
| | | | Cycle 4 | | | | Termination | Follow-Up^{T} | Foll [symbol] - |
| | Cycle 3 | | (28 days) | | | | 30 days | 90 days | U [symbol] |
| | (28 days) | | and beyond | | | | after | after | Eve [symbol] |
| Study Day | 15 | 22 | 1 | 8 | 15 | 22 | last dose | last dose | 3 m [symbol] |
| Cycle Day | C3 | C3 | CX | CX | CX | CC | — | — | — |
| | D 15 | D 22 | D 1 | D 8 | D 15 | D 22 | | | |
| +/− days | 2 | 2 | 2 | 1 | 2 | 2 | 3 | 7 | n/ [symbol] |
| allowable | | | | | | | | | |
| Study drug | X | X | X | X | X | X | | | |
| administration ^{A} | | | | | | | | | |
| Study Procedures & Examinations | | | | | | | | | |
| Eligibility | | | | | | | | | |
| assessment and | | | | | | | | | |
| written informed | | | | | | | | | |
| consent | | | | | | | | | |
| Demographics ^{B} | | | | | | | | | |
| Medical history ^{C} | | | | | | | | | |
| Previous and | X | X | X | X | X | X | X | | |
| concomitant | | | | | | | | | |
| medications ^{D} | | | | | | | | | |
| Adverse events ^{E} | X | X | X | X | X | X | X | X | |
| ECHO/MUGA ^{F} | | | X | | | | | | |
| 12-lead ECG | | | X | | | | | | |
| (QTcF) | | | | | | | | | |
| Physical exam ^{G} | X | X | X | X | X | X | X | X | |
| ECOG | X | X | X | X | X | X | X | | |
| Vital Signs^{H} | X | X | X | X | X | X | X | | |
| Tumor imaging | | | | | | | X | | X |
| assessment (CT | | | | | | | repeat if | | |
| with contrast | | | | | | | end of study | | |
| preferred) N | | | | | | | is >8 weeks | | |
| | | | | | | | after last scan | | |
| Clinical Labs | | | | | | | | | |
| Pregnancy test ^{I} | | | X | | | | X | | |
| Hematology ^{J} | X | X | X | X | X | X | X | X | |
| Serum Chemistry ^{K} | X | X | X | X | X | X | X | X | |
| TSH, Free T4, | | | X | | | | X | X | |
| serum lipase, | | | | | | | | | |
| amylase, PTH, | | | | | | | | | |
| FSH, LH, free | | | | | | | | | |
| cortisol | | | | | | | | | |
| Blood | | | | | | | | | |
| Coagulation ^{L} | | | | | | | | | |
| Urinalysis M | X | X | X | X | X | X | X | X | |
| Pharmacodynamics and Pharmacokinetics | | | | | | | | | |
| Tumor biopsy ^{O} | X ± | X ± | | | | | | | |
| | 7 d | 7 d | | | | | | | |
| Tumor type- | X | | X | | X | | X | | |
| relevant | | | | | | | | | |
| biomarkers ^{P} | | | | | | | | | |
| Blood sampling | X | | X | | | | X | | |
| for biomarker | | | | | | | | | |
| PD analysis ^{Q} | | | | | | | | | |
| PK blood | X | X | X | | | | | | |
| sampling ^{R} | | | | | | | | | |
| Immunogenicity | | | X | | | | X | | |
| (ADA) ^{S} | | | | | | | | | |
| Survival Follow- | | | | | | | | | [symbol] |
| up | | | | | | | | | |

ADA: anti-drug antibodies; AE: adverse event; ALT: alanine aminotransferase; APTT: activated partial thromboplastin time; AST: aspartate aminotransferase; C: cycle; CPK: creatine phosphokinase; COVID19: Coronavirus SARS-COV-2; CRP: C-reactive protein; CT: computed tomography; D or d: day(s); ECG: electrocardiogram; ECOG: Eastern Cooperative Oncology Group; ECHO: echocardiography/cardiac ultrasound; FSH: follicle-stimulating hormone; IMAR: immune-mediated adverse reaction; INR: international normalized ratio; LDH: lactate dehydrogenase; LH: luteinizing hormone; min: minute(s); MUGA: multigated acquisition scan; PD: pharmacodynamics; PK: pharmacokinetics; [symbol] prothrombin time; PTH: parathyroid hormone; PTT: partial thromboplastin time; QTcF: QT interval, Fridericia's Correction Formula; RBC: red blood cell count; SGOT: serum glutamic-oxaloacetic transaminase; SGPT: serum glutamic pyruvic transaminase; TSH: thyroid stimulating hormone; WBC: white blood cell count.
^{A} Study drug administration: G9.2-17 (IGG4) treatment is administered, on CXD1, CXD8, CXD15, and CXD22 on every weekly cycle (Cohorts 7-8). an anti-PD1 antibody [symbol] administered on Day 1 of every cycle on the G9.2-17 (IGG4) combination regimen. Study drug may be administered on Days 1, 8 and 15 +/− 3 days from C2 onwards.
^{B} Demographics: Data include age, gender, race, and ethnicity.
^{C} Medical history: In addition to general medical history, data collection also includes oncology history, surgical/transplant and radiation therapy history and COVID-19 history testing.
^{D} Previous and concomitant medications (including vaccines and complementary treatments/supplements): Data to include name, indication, dose, route, start and end dates for each. Allergies and intolerances, dose modifications while on study, schedule of dosing changes and reasons for them should also be obtained.
^{E} Adverse events: Any AEs starting or worsening after study drug administration is recorded. AEs should be followed until resolved to one of the following: baseline, stabilized deemed irreversible. All SAEs are to be collected until 30 days after last dose of study medication. All study-procedure-related SAEs must be collected from the date of patient's written consent.
^{F} ECHO/MUGA: This assessment of heart function is conducted at Screening and repeated on Day 1 of Cycle 4; the assessment window is +/−5 days. It should be conducted [symbol] frequently when clinically indicated and once every 3 months.
^{G} Physical exam: Include height at screening for determination of body surface area. Include weight at all scheduled exam times. A Neurological exam is conducted only on pa [symbol] who have stable and/or pre-treated brain metastases.
^{H}Vital Signs: temperature, heart rate, blood pressure, respiratory rate.
^{I} Pregnancy test (blood or urine): Only for women of childbearing potential with uterus in situ. Test results must be available before scheduled dosing.
^{J} Hematology: Analysis includes complete blood count, differential, platelets, hemoglobin. Collect blood samples pre-dose.
^{K} Serum chemistry: Analysis includes albumin, alkaline phosphatase, bilirubin (total, direct), blood urea nitrogen, calcium, CPK, creatinine, electrolytes (sodium, potassium, chloride, magnesium, phosphorus), gamma glutamyl transferase (gamma GT), glucose, hemoglobin Alc (HgbAlc) (only if history of Type 1 or Type 2 diabetes mellitus), LDH, SGPT (ALT) or SGOT (AST), total protein. Fasting glucose to be assessed only if clinically indicated. Collect blood samples pre-dose.
^{L} Blood Coagulation: Collect blood samples pre-dose. Analysis includes APTT, PT, PTT, and INR (if on allowable anti-coagulants), CRP, and troponin.
^{M} Urinalysis: Analysis includes color, appearance, dipstick for specific gravity, protein, white blood cell-esterase, glucose, ketones, urobilinogen, nitrite, WBC, RBC, pH. (Uri [symbol] culture and sensitivity to be run only if patient is clinically symptomatic.)
^{N} Tumor imaging assessment: For screening, the assessment must be performed within the 28-day screening period. On study, assessments are done every 8 weeks ± 7 days (ie, C3D1, C5D1, C7D1, C9D1, etc.) and at the End of Treatment if not assessed within the previous 4-6 weeks. Assessments may be performed more frequently if clinically indicat [symbol] an objective response is seen on a scan, a confirmation scan is done 4 weeks (+7 d) later. After this confirmatory scan, the scheduled scans are to be resumed at a frequency of e [symbol] 8 weeks ± 7 days from the date of the confirmatory scan.
^{O} Tumor biopsies: If patient MMR/MSI status is unknown at screening, the test should be run at the local laboratory. In Part 2, TMB tissue analysis is performed. The on-study biopsy is scheduled for C3D15 + 7 days and should occur only after the tumor imaging scan in Cycle 3. It is recognized that a variety of clinical factors may make it difficult to obtain adequate specimens. Decisions not to perform biopsy on-treatment should be discussed with the Medical Monitor.
^{P} Tumor type-relevant biomarkers: Blood samples are to be collected at screening and every cycle pre-dose administration as appropriate for the tumor type. Blood sampling m [symbol] decreased to every 3rd cycle after 6 months of treatment.
^{Q} PD blood sampling: Blood samples are collected pre-dose administration on dosing days. May be decreased to every 3rd cycle after 6 months of treatment.
^{R} PK blood sampling: Cycle 1 and Cycle 3 Day 1: blood samples are collected pre-dose, end of study drug infusion (EOI) and 1 h (±15 min) post-study drug administration. C [symbol] 1 and Cycle 3 Day 3, blood samples are collected at one time point, any time. Cycle 1 and Cycle 3 Day 8, Day 15, and Day 22, blood samples are collected pre-dose and at EOI [symbol] Cycle 2 and every even cycle there after: blood samples are collected Day 1 only and should occur pre-dose and at EOI. Blood samples for PK will not be collected on every odd cycle after Cycle 3.
^{S} ADA blood sampling: Blood samples are collected Day 1 and Day 15 of Cycles 1 and 2, pre-dose. Thereafter, it is collected every cycle, Day 1, pre-dose (ie, C3D 1, C4D 1, et [symbol]
^{T}All patients treated with G9.2-17 (IGG4) + an anti-PD1 antibody must return 90-days +/− 7 days after last dose of study drug for an assessment of potential immune-mediate [symbol] adverse reactions (IMARs).
^{U} Long-Term Follow-up: Tumor imaging should continue, where possible, for patients discontinuing treatment due to reasons other than progression of disease and not receivi [symbol] additional systemic anticancer treatments. Survival data is collected at a minimum every 3 months. It can be collected more frequently to support data cleaning or regulatory submission efforts. Follow-up can be conducted by telephone, electronic messaging or chart review and will continue for up to 2 years after the patient has the End of Treatment/Early Termination visit. [symbol]
indicates data missing or illegible when filed

### Study Assessments and Procedures

A signed, written ICF approved by an Institutional Review Board (IRB) must be obtained from the potential patient before he/she can participate in any study-specific procedures, including study-specific screening procedures.

Patients are entered in the study once all screening procedures have been completed and it is determined that they meet all eligibility criteria.
Study procedures and their respective timing for Part 1, Cohorts 1-6 are summarized in the SoA (Table 8). Study procedures and their respective timing for Part 1, Cohorts 7 and 8 are summarized in the SoA (Table 9) Protocol waivers or exemptions are not allowed.
Adherence to all study requirements, including those specified in the SoA, is essential and required for study conduct.
Immediate safety concerns should be discussed immediately upon occurrence or awareness to determine the need for intervention or study discontinuation.
All screening evaluations must be completed and reviewed to confirm that potential participants meet all eligibility criteria. A screening log is maintained to record details of all participants screened and to confirm eligibility or record reasons for screening failure, as applicable.
Procedures conducted as part of the participant's routine clinical management (e.g., blood count) and obtained before signing of the ICF may be utilized for screening or baseline purposes provided the procedures met the protocol-specified criteria and were performed within the time frame defined in the SoA.

### Assessments by Visit

The SoA (Tables 5-6) provides a list of assessments to be performed during the screening period (up to 28 days), the treatment period (presented as 28-day cycles), the End of Treatment/Early Termination period, IMAR follow-up and the long-term follow-up period. Optional visits are allowed during each treatment cycle if medically indicated, during which any of the study assessments may be performed.

During the COVID-19 pandemic many governments require citizens to practice social distancing, and more vulnerable populations are advised to self-isolate. These types of constraints may affect the ability to run this clinical study as originally intended. Planned site visits can be adapted so that the study can safely continue during the pandemic. Possible modifications may include:
visit and/or study procedure postponement
replacement with telephone/video call(s)
replacement with home visits
visits performed at an alternative clinical location
visits performed by a health-care provider outside the study team
visit and/or study procedure completely cancelled.
   Screening Period (between Day −28 and Day −1)

The following procedures must be conducted within 4 weeks of initiating treatment: Study Procedures & Examinations
Written informed consent
Verify inclusion and exclusion criteria for patient eligibility
Patient demographics
Medical history
Previous and concomitant medications
ECHO/multigated acquisition scan (MUGA)
12-lead ECG (QT interval corrected using Fridericia's formula [QTcF])
Physical examination—in patients with stable and pre-treated brain metastases, perform a neurological exam
ECOG performance status
Vital signs
Tumor imaging assessment (computed tomography [CT] or magnetic resonance imaging (MRI), with or without contrast; or positron emission tomography (PET)-CT; CT with contrast is preferred)

### Clinical Labs

Pregnancy test for women of childbearing potential (WOCBP)
Hematology
Serum chemistry
Thyroid stimulating hormone (TSH), free T4 or thyroxine (fT4), serum lipase, amylase, parathyroid hormone (PTH), follicle-stimulating hormone (FSH), luteinizing hormone (LH), free cortisol
Blood coagulation
Urinalysis

### Pharmacodynamics and Pharmacokinetics

Tumor biopsy
   Biopsy can be omitted if it is deemed that the procedure is a risk to the patient.
   If a biopsy is unavailable, site will make every effort to obtain an archival tumor tissue sample available as a formalin-fixed paraffin-embedded (FFPE) block. Acceptable archival samples include those obtained via a core needle biopsy or excisional surgery within the last five years.
dMMR-MSI-H status (if the MMR and MSI status of the patients has not been previously determined, the test must be run at the local laboratory)
TMB in tissue for Part 2 G9.2-17 (IGG4)+an anti-PD1 antibody combo arm only
Tumor type-relevant biomarkers

### Treatment Period

Each treatment cycle has a duration of 28 days. Refer to Table 5 for Cohorts 1-6 of Part 1, and Table 6 for Cohorts 7 and 8 of Part 1 for timing of visits.

### Treatment Procedures for Day 1 of Each Cycle (CXD1; ±2 Days Beginning Cycle 2)

The following procedures are performed on Day 1 of each treatment cycle.

### Study Procedures and Examinations

Concomitant medications
AEs
12-lead ECG (QTcF)
Physical examination
ECOG performance status
Vital signs

### Clinical Labs

Pregnancy test for WOCBP
Hematology
Serum chemistry
TSH, fT4, lipase, amylase, PTH, FSH, LH, free cortisol
Blood coagulation
Urinalysis

### PK/PD Assessments

PD blood sampling
PK blood sampling
ADA blood sampling
Tumor type-relevant biomarkers

### Study Drug Administration

Administered only after all pre-dose assessments and procedures are completed.

Additionally, beginning on Day 1 of Cycle 3, the following assessment is performed every 8 weeks:
Tumor imaging assessment (CT or MRI, with or without contrast; or PET-CT; CT with contrast is preferred)

Furthermore, beginning on Day 1 of Cycle 4, the following assessment is performed every 3 months:
ECHO/MUGA

### Cohorts 1-6: Treatment Procedures for Days 2 and 8 of Cycle 1 and Cycle 3 (CXD2±1 Day and CXD8±1 Day)

### Study Procedures and Examinations

Concomitant medications
AEs

### PK/PD Assessments

PD blood sampling
PK blood sampling

### Cohorts 1-6: Treatment Procedures for Day 15 of Each Cycle (CXD15±1 Day for Cycle 1 and +2 Days for Beginning Cycle 2)

The following procedures are performed on Day 15 of each treatment cycle.

### Study Procedures and Examinations

Concomitant medications
AEs
Physical examination
ECOG performance status
Vital signs

### Clinical Labs

Hematology
Serum chemistry
Blood coagulation
Urinalysis

### PK/PD Assessments

PD blood sampling on C1D15 and C3D15 only
PK blood sampling on C1D15 and C3D15 only
Tumor type-relevant biomarkers
Tumor biopsy on C3D15±7 days (Cycle 3 ONLY; can be eliminated if it is deemed too risky for the patient)

### Study Drug Administration

Administered only after all pre-dose assessments and procedures are completed.

### Cohorts 7 and 8: Treatment Procedures for Day 3 of Cycle 1 and Cycle 3 (C1D3±1 Day and C3D3±1 Day)

### Study Procedures and Examinations

Concomitant medications
AEs

### PK/PD Assessments

PD blood sampling
PK blood sampling

### Cohorts 7 and 8: Treatment Procedures for Day 8 of Each Cycle (CXD8±1 Day)

### Study Procedures and Examinations

Concomitant medications
AEs
Physical examination
ECOG performance status
Vital signs

### Clinical Labs

Hematology
Serum chemistry
Blood coagulation
Urinalysis

### PK/PD Assessments

PD blood sampling
PK blood sampling for odd numbered Cycles only

### Study Drug Administration

Administered only after all pre-dose assessments and procedures are completed.

### Cohorts 7 and 8: Treatment Procedures for Days 15 and 22 of Each Cycle (CXD15±1 Day for Cycle 1 and ±2 Days for Beginning Cycle 2)

The following procedures are performed on Days 15 and 22 of each treatment cycle.

### Study Procedures and Examinations

Concomitant medications
AEs
Physical examination
ECOG performance status
Vital signs

### Clinical Labs

Hematology
Serum chemistry
Blood coagulation
Urinalysis

### PK/PD Assessments

PD blood sampling on C1D15 and C3D15 only
PK blood sampling on odd numbered Cycles only
Tumor type-relevant biomarkers
Tumor biopsy on C3D15±7 days (Cycle 3 ONLY; can be eliminated if it is deemed too risky for the patient)
ADA blood sampling on C1D15 and C2D15 only

### Study Drug Administration

Administered only after all pre-dose assessments and procedures are completed.

### Additional Treatment Beyond Cycle 4

Treatment cycles beyond Cycle 4 can be repeated as indicated in the SoA (Tables 5-6). If the patient is experiencing clinical benefit, even in the event of radiological progression, the patient can continue on treatment.

### End of Treatment or Early Termination Procedures

The following procedures are done 30 days (+3 days) after the last dose, including patients who have discontinued treatment early.

### Study Procedures and Examinations

Concomitant medications
AEs
Physical examination
ECOG
Vital signs
Tumor imaging assessment: Confirmatory scan if end of study is >8 weeks after previous scan.

### Clinical Labs

Pregnancy test for WOCBP
Hematology
Serum chemistry
TSH, fT4, lipase, amylase, PTH, FSH, LH, free cortisol
Blood coagulation
Urinalysis

### PD Assessments

PD blood sampling
ADA blood sampling
Tumor type-relevant biomarkers

### IMAR 90-Day Follow-Up

All patients on the combination treatment with an anti-PD1 antibody in Part 2 must return on Day 90±7 for a safety follow-up in order to evaluate any possible delayed IMARs. The visit includes the following:

### Study Procedures and Examinations

AEs
Physical examination
Vital signs

### Clinical Labs

Hematology
Serum chemistry
TSH, fT4, lipase, amylase, PTH, FSH, LH, free cortisol
Blood coagulation
Urinalysis

### Long-Term Follow-Up

OS is assessed every 3 months for up to 2 years after the patient has the End of Treatment/Early Termination. Tumor imaging assessment continues, where possible, for patients discontinuing treatment due to reasons other than progression of disease and not receiving additional systemic anticancer treatments.

Survival data as well as information on any new anticancer therapy initiated after disease progression is collected at a minimum every 3 months. It can be collected more frequently to support data cleaning or regulatory submission efforts. Follow-up may be performed by telephone interview, electronic messaging or chart review and is reported on the CRF. During the Follow-Up Period, deaths, regardless of causality are collected and reported within 24 h of discovery or notification of the event.

### RECIST v1.1 Criteria for Tumor Assessment

At the screening tumor assessment, tumor lesions/lymph nodes are categorized as measurable or non-measurable with measurable tumor lesions recorded according to the longest diameter in the plane of measurement (except for pathological lymph nodes, which are measured in the shortest axis). When more than one measurable lesion is present at screening all lesions up to a maximum of five lesions total (and a maximum of two lesions per organ) representative of all involved organs should be identified as target lesions. Target lesions should be selected on the basis of their size (lesions with the longest diameter). A sum of the diameters for all target lesions is calculated and reported as the baseline sum diameters.

All other lesions (or sites of disease) including pathological lymph nodes should be identified as non-target lesions and should also be recorded at screening. Measurements are not required, and these lesions should be followed as ‘present’, ‘absent’, or ‘unequivocal progression’.

Tumor target lesions are assessed according to the RECIST v1.1 Guidelines (Eisenhauer et al., 2009) using the following disease response measures:

Evaluation of target lesions:
Complete Response (CR): Disappearance of all target lesions. Any pathological lymph nodes (whether target or non-target) must have reduction in short axis to <10 mm.
Partial Response (PR): At least a 30% decrease in the sum of diameters of target lesions, taking as reference the baseline sum diameters.
Progressive Disease: At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).
Stable Disease (SD): Neither sufficient shrinkage to qualify for PR nor sufficient increase to qualify for PD, taking as reference the smallest sum diameters while on study.

### Evaluation of Non-Target Lesions:

CR: Disappearance of all non-target lesions and normalization of tumor marker level. All lymph nodes must be non-pathological in size (<10 mm short axis).
Non-CR/Non-progressive disease (non-PD): Persistence of one or more non-target lesion(s) and/or maintenance of tumor marker level above the normal limits.
Progressive Disease: Unequivocal progression of existing non-target lesions. (Note: the appearance of one or more new lesions is also considered progression).

A summary is provided in Table 7 below.

TABLE 7
Evaluation of Overall Timepoint Response for
Patients with Measurable Disease at Baseline

| | Non-target | New | |
|---|---|---|---|
| Target Lesions | Lesions | Lesions | Overall Response |
| CR | CR | Nc | CR |
| CR | Non-CR/Non-PD | No | PR |
| CR | NE | No | PR |
| PR | Non-PD or NE | No | PR |
| SD | Non-PD or NE | No | SD |
| Not all evaluated | Non-PD | No | NE |
| Progressive disease | Any | Yes | Progressive disease |
| | | or No | |
| Any | Progressive disease | Yes | Progressive disease |
| | | or No | |
| Any | Any | Yes | Progressive disease |

CR: Complete Response,
Non-PD: Non-progressive Disease,
PR: Partial Response,
SD: Stable Disease,
NE: Non-evaluable
*When target lesions show SD/PR and some subset of non-target lesions is non-evaluable, a careful decision must be made whether to call the overall response at this timepoint SD/PR or NE. This is based on whether the non-evaluable lesions, if they showed growth, could cause an overall response of progressive disease in the context of the other lesion responses seen. If the non-evaluable non-target lesions comprise a significant proportion of the overall disease burden, the appropriate timepoint response is NE.

The disease response measures at different timepoints allow for the calculation of the following:
Disease control rate (DCR), defined as percentage of patients who have achieved CR, PR and SD.
Objective response rate (ORR), defined as the proportion of patients with tumor size reduction of a predefined amount (tumor shrinkage of ≥30%).
Progression-free survival (PFS), defined as the time from study drug treatment initiation to disease progression (tumor growth by ≥30%).
Duration of response (DoR), defined as the length of time that a tumor continues to respond to treatment without the cancer growing or spreading.
Overall survival (OS) defined as the time from study drug treatment initiation to death from any cause.

### Safety Assessment

### Physical Examinations

Medical and physical examinations must be performed by a qualified physician, nurse practitioner, or physician assistant, and should include a thorough review of all body systems. Additionally, height (at screening only) and weight are measured.

### Vital Signs

Vital signs are measured in a post-supine position after 5 minutes rest and include temperature, blood pressure (systolic and diastolic), heart rate, and respiratory rate.

### Electrocardiograms

12-lead ECG is obtained as outlined in the SoA (see Tables 5-6) using an ECG machine that automatically calculates the heart rate and measures heart rate, PR interval, QRS duration, distance in time on the ECG tracing from the start of the QRS complex to the end of T-wave (QT) interval, and QTcF intervals.

### Clinical Safety Laboratory Assessments

Patients have blood samples collected for routine clinical laboratory testing (approximately 5 mL at each timepoint), according to the SoA (Tables 5-6); additional tests may be performed at any time during the study as determined necessary.

The clinical laboratory parameters are analyzed at the site's local laboratory. Laboratory assessments completed include hematology and serum chemistry and is defined as following:
Serum Chemistry: Includes glucose, total protein, albumin, electrolytes [sodium, potassium, chloride, magnesium, phosphorus], calcium, bilirubin (total, direct), SGPT (ALT) or SGOT (AST), alkaline phosphatase, gamma glutamyl transferase (gamma GT), lactate dehydrogenase (LDH), creatinine, hemoglobin Alc (HgbAlc) (only if history of Type 1 or Type 2 diabetes mellitus), blood urea nitrogen, creatine phosphokinase (CPK)
   TSH, fT4, lipase, amylase, PTH, FSH, LH, free cortisol additionally at specified visits
   Fasting glucose is assessed only if clinically indicated
Hematology: Includes complete blood count, differential, platelets, hemoglobin
Coagulation: Includes prothrombin time (PT) and PTT, activated partial thromboplastin time (APTT) and INR (if on allowable anti-coagulants) C-reactive protein (CRP), and troponin
Urinalysis: Patients have urine samples collected for routine urinalysis. The urinalysis includes color, appearance, and dipstick for specific gravity, protein, white blood cell-esterase, glucose, ketones, urobilinogen, nitrite, white blood cell count (WBC), red blood cell count (RBC), and pH, and urine culture (if patient is clinically symptomatic).
If clinically significant values do not return to normal/baseline or Grade 1 within a period of time judged reasonable, the etiology should be identified.
All protocol-required laboratory tests must be conducted in accordance with the laboratory manual and the SoA (Tables 5-6).
If laboratory values from non-protocol specified laboratory tests performed at the institution's local laboratory require a change in participant management or are considered clinically significant (e.g., SAE or AE or dose modification), then the results must be recorded.

### Pregnancy Testing

WOCBP should only be included after a confirmed menstrual period and a negative highly sensitive urine or serum pregnancy test.

Additional pregnancy testing should be performed during the treatment period and at the End of Treatment/Early Termination visit according to the SoA (Tables 5-6), and as required locally.

Pregnancy testing is performed whenever a menstrual cycle is missed or when pregnancy is otherwise suspected.

If the patient has prior history of bilateral salpingo-oophorectomy and/or hysterectomy, record these surgical procedures; a pregnancy test is not required for these patients.

### Pharmacokinetics Assessments

The following serum PK parameters are calculated for G9.2-17 (IGG4), if possible:
AUC₀₋₃₃₆ₕ
Cₘₐₓ
Tₘₐₓ
t_{1/2}
serum concentration vs. time profiles

Blood samples of approximately 5 mL are collected and processed to serum at each timepoint as specified in the SoA (Tables 5-6).

### PK Schedule for Cohorts 1-6:

Cycle 1 and Cycle 3 Day 1
Pre-dose
At the End of Infusion (EOI)
2 h (±30 min) after EOI
4 h (±30 min) after EOI

Cycle 1 and Cycle 3 Day 15
Pre-dose
At EOI

cycle 1 and cycle 3 day 2 and 8 (non-dosing days)
Any point during the visit

Cycle 2 and Cycle 4 Day 1
Pre-dose
At EOI

Every 2 Cycles beyond Cycle 4 on Day 1 (i.e., C6D1, C8D1 etc.)
Pre-dose
At EOI

### PK Schedule for Cohorts 7 and 8:

Every odd numbered Cycle Day 1 (i.e., C1D1, C3D1, etc.)
Pre-dose
At the End of Infusion (EOI)
1 h (±15 min) after EOI

Every odd numbered Cycle Day 3 (i.e., C1D3, C3D3, etc.)
Any point during the visit

Odd numbered Cycle Days 8, 15, and 22 (i.e. C1D8, C3D8, etc.)
Pre-dose
At EOI

Even numbered Cycle Day 1 (i.e., C2D1, C4D1, etc.)
Pre-dose
At EOI

If the dose of study drug is determined to be interrupted, additional PK and safety assessments are collected upon resumption of dosing; additional PK assessments may be performed during the interruption. If the dose of study drug is reduced, additional PK assessments are collected pre-administration of the reduced dose (within 2 h pre-dosing), and 2 to 4 h after starting the reduced study drug dose. Additional PK, and other blood assessments may be taken if clinically indicated. Centers that are not able to hold patients more than 2 h post-dose due to COVID-19 restrictions, contribute samples at EOI and 2 h post-dose only.

Instructions for the collection and handling of biological samples are provided. The actual date and time (24-h clock time) of each sample are recorded.

Samples are used to evaluate the serum concentration levels of total G9.2-17 (IGG4) and free/partially free G9.2-17 (IGG4) by a designated laboratory. Concentrations are determined using validated assays. A minimum of two 50 μL aliquots of serum are needed to determine total G9.2-17 (IGG4) concentrations. A minimum of two 100 μL aliquots of serum are needed to determine free and partially free G9.2-17 (IGG4) concentrations and residual serum in a third aliquot. Samples collected for analyses of G9.2-17 (IGG4) plasma concentration may also be used to evaluate safety or efficacy aspects related to concerns arising during or after the study.

Genetic analyses is not performed on these blood samples. Participant confidentiality is maintained. At visits during which blood samples for the determination of PD, ADA, safety lab of G9.2-17 (IGG4) is taken, one sample of sufficient volume can be used.

### Genetics

Genetics are not evaluated in this study.

### Pharmacodynamic Biomarkers

Planned time points for biomarker assessments are provided in the SoA (Tables 5-6); sampling may be decreased to every 3rd cycle after 6 months of treatment.

Collection of biological samples for other biomarker research is also part of this study. The following samples for biomarker research are required and are collected from all participants in this study as specified in the SoA:
Blood samples, to be collected prior to study drug administration (approximately 15 mL pre-dose)
Tumor biopsy (tissue sample)

Samples are tested for PD biomarkers (by flow cytometry, ELISA, IHC, or multiplex phenotyping) to evaluate their association with the observed clinical responses to G9.2-17 (IGG4) using validated assays.

The following biomarkers are assessed for this study:
Tumor markers (blood): CA15-3, CA-125, carcinoembryonic antigen (CEA), CA19-9, alpha fetoprotein, neuron-specific enolase (NSE), cytokeratin-fragment-21 (CYFRA-21) to be assessed every cycle pre-dose administration as needed per tumor type. This may be decreased to every 3 cycles after 6 months of treatment, following the same schedule as tumor imaging assessments, as appropriate.
PBMC phenotype (blood): e.g., CD3, CD4, CD8, CD45RO, forkhead-box-protein P3 (FOXP3), CD11B, CD14, CD15, CD16, CD33, CD68, human leukocyte antigen (HLA) DR, CD163, arginase 1, granzyme B, KI67, PD-1, PD L1, pan cytokeratin (PAN CK)
Cytokines (blood): eg, interferon gamma (IFN γ), IL 10, IL 12p70, IL 13, IL 1β, IL 2, IL 4, IL 6, IL 8, TNF α, MIP-1b, monocyte chemoattractant protein 1 (MCP-1), MIP-1a, IL 17a, IL 5, TGF β
Gal-9 in blood and tumor tissue
PD-L1 (tissue)
Mismatch repair status (tissue)
Tumor Mutational Burden (TMB)

Exploratory biomarker changes, if any, are correlated with safety and response outcomes.

Samples may be stored for a maximum of 2 years (or according to local regulations) following the last patient's last visit for the study at a facility selected to enable further analysis of the effect of G9.2-17 (IGG4) on pharmacodynamic biomarkers.

### Immunogenicity Assessments

Blood samples (approximately 3 mL) are collected from all participants according to the SoA (Tables 5-6) and processed to serum. Additionally, serum samples should also be collected at the end of treatment/early termination visit from patients who discontinued study intervention or were withdrawn from the study.

### Cohorts 1-6: Cycle 1 to Cycle 4 Day 1

Pre-dose

### Cohorts 1-6: Every 2 Cycles Beyond Cycle 4 on Day 1 (i.e., C6D1, C8D1 Etc.):

Pre-dose

### Cohorts 7 and 8: Every Cycle Day 1

Pre-dose

### Cohorts 7 and 8: Cycle 1 Day 15 and Cycle 2 Day 15 Only

Pre-dose

A minimum of two aliquots of 500 μL serum each, with residual serum in a third tube are obtained. Samples are shipped to a lab designated for analysis using a validated assay. These samples are tested.

Serum samples are screened for antibodies binding to G9.2-17 (IgG4) (ADA) and the titer of confirmed positive samples is reported. Other analyses may be performed to verify the stability of antibodies to G9.2-17 (IgG4) and/or further characterize the immunogenicity of G9.2-17 (IgG4).

The detection and characterization of antibodies to G9.2-17 (IgG4) is performed using a validated assay method. All samples collected for detection of antibodies to study intervention are evaluated for G9.2-17 (IgG4) serum concentration to enable interpretation of the antibody data. Antibodies may be further characterized and/or evaluated for their ability to neutralize the activity of the study intervention. Samples may be stored for a maximum of 2 years (or according to local regulations) following the last patient's last visit for the study at a suitable facility to enable further analysis of immune responses to G9.2-17 (IgG4).

### Other Assessments

### Demographics

At Screening, patient demographic data is collected. These include age, gender, race, and ethnicity.

### Medical History

The medical history includes oncology history, surgical/transplant history radiation therapy history, and COVID 19 history and testing.
Personal medical history, including prior treatments/surgeries, record of any implants in situ or past implants, prior and/or current use of medical devices, concomitant medications (name, indication, dose, route, start and end dates dose modifications if any and reason), pre-existing symptoms, and AEs), hereditary diseases at risk of based on family history and complete family history to the best knowledge of the patient
Record of any dental work performed in the past 12 months
For patients with previously resected pancreatic adenocarcinoma, record whether the primary tumor was localized to the head of pancreas, pancreatic body or the pancreatic tail.
Bowel habits/typical frequency and consistency
Record any dietary requirements or preferences (for example, practice of a particular diet regimen: intermittent fasting, keto diet etc.)
Record and allergies past and present (allergen, severity)

### Prior and Concomitant Medications

Prior and concomitant medications, including vaccines and complementary treatments/supplements, are documented for each patient at each scheduled visit (Tables 5-6).

### Tumor Imaging Assessments

Tumor assessments are performed using CT or MRI with or without contrast; a PET-CT is performed.

CT with contrast is the preferred modality (MRI, PET-CT, or other imaging modalities instead of, or in addition to, the CT scan, if CT is not feasible or appropriate, given location of the disease). Assessment should include the chest/abdomen/pelvis at a minimum and should include other anatomic regions as indicated, based on the patient's tumor type and/or disease history. Imaging scans must be de-identified and archived in their native format as part of the patient study file. While the type of scan obtained, as appropriate for the disease, the same method should be used for the duration of the study.

On study, assessments are done every 8 weeks±7 days according to the SoA (i.e., C3D1, C5D1, C7D1, C9D1, etc.) and at the End of Treatment if not assessed within the last 4-6 weeks. Assessments may be performed more frequently if clinically indicated. For Part 2 only, if an objective response is seen on a scan, a confirmation scan is done 4 weeks (+7 days) later. After a confirmatory scan, the scheduled scans are to be resumed at a frequency of every 8 weeks (±7 days) from the date of the confirmatory scan.

### Tumor Biopsies

Pre- and on-treatment biopsies are collected. A pre-treatment biopsy is collected during screening. If a pre-treatment biopsy is unobtainable as per the reasons outlined in the inclusion criteria, and the patient is enrolled in the study, an archival tumor tissue specimen from that patient is collected from a primary tumor and/or a metastatic deposit. Excisional or core biopsy (FFPE tissue block(s) OR fresh tissue in formalin) obtained currently or within 5 years before study start from the primary tumor lesion or a metastatic deposit. If both primary and metastatic tissues are available, use of metastatic deposit tissue is prioritized. If information of treatment(s) received before and after tissue acquisition are available, this is collected as well.

The on-treatment biopsy is scheduled for C3D15±7 days and should occur only after the tumor imaging scan in Cycle 3. In instances where the procedure cannot be performed within the protocol-specified timeframe, alternatives may be permitted but must be discussed with the Study Director/Medical Monitor. It is recognized that a variety of clinical factors may make it difficult to obtain adequate specimens. Decisions not to complete biopsy on-treatment should be discussed with the Medical Monitor.

### ECHO/MUGA

ECHO and/or MUGA are obtained at the timepoints indicated in the SoA (Tables 5-6). If clinically indicated, the assessment is to be repeated once every 3 months.

### ECOG

ECOG performance status is assessed at the timepoints indicated in the SoA (Tables 5-6) using the following grading (Oken et al., 1982).
Grade 0: Fully active, able to carry on all pre-disease performance without restriction
Grade 1: Restricted in physically strenuous activity but ambulatory and able to carry out work of a light or sedentary nature, e.g., light housework, office work
Grade 2: Ambulatory and capable of all self-care but unable to carry out any work activities. Up and about more than 50% of waking hours
Grade 3: Capable of only limited self-care, confined to bed or chair more than 50% of waking hours
Grade 4: Completely disabled. Cannot carry on any self-care. Totally confined to bed or chair
Grade 5: Dead

### Adverse Events (AEs), Serious Adverse Events (SAEs), and Other Safety Reporting

An AE is defined in the ICH Guideline for GCP as “any untoward medical occurrence in a patient or clinical investigation patient administered a pharmaceutical product and that does not necessarily have a causal relationship with this treatment.”

This definition of AEs is broadened in this study to include any such occurrence (e.g., sign, symptom, or diagnosis) or worsening of a pre-existing medical condition from the time that a patient has signed informed consent to the time of initiation of the investigational drug. Worsening indicates that the pre-existing medical condition (e.g., diabetes, migraine headaches, gout, hypertension, etc.) has increased in severity, frequency, or duration of the condition or an association with significantly worse outcomes.

### Serious Adverse Events

A SAE is defined as an AE that:
Results in death;
Is life threatening (places the patient at immediate risk of death);
Requires in-patient hospitalization or prolongation of existing hospitalization;

A hospitalization meeting the definition for “serious” is any inpatient hospital admission that includes a minimum of an overnight stay in a health care facility. Inpatient admission does not include rehabilitation facilities, hospice facilities, skilled nursing facilities, nursing homes, routine emergency room admissions, same day surgeries (as outpatient/same day/ambulatory procedures), or social admission (eg, patient has no place to sleep).
Results in persistent or significant disability/incapacity; or
Is a congenital anomaly/birth defect
Important medical events that may not result in death, be life threatening, or require hospitalization may be considered an SAE when, based upon appropriate medical judgment, they may jeopardize the patient and may require medical or surgical intervention to prevent one of the outcomes listed in this definition. Examples of such medical events include anaphylaxis and allergic bronchospasm requiring intensive treatment in an emergency room or at home, blood dyscrasias or convulsions that do not result in inpatient hospitalization.

### Relatedness

For all AEs, enough information should be obtained to determine the causality of the AE (e.g., study drug or other illness). The relationship of the AE to the study treatment is assessed following the definitions below:
Unrelated: any event that does not follow a reasonable temporal sequence from administration of study drug AND that is likely to have been produced by the patient's clinical state or other modes of therapy administered to the patient.
Unlikely Related: any event that does not follow a reasonable temporal sequence from administration of study drug OR that is likely to have been produced by the patient's clinical state or other modes of therapy administered to the patient.
Possibly Related: any reaction that follows a reasonable temporal sequence from administration of study drug OR that follows a known response pattern to the suspected drug AND that could not be reasonably explained by the known characteristics of the patient's clinical state or other modes of therapy administered to the patient.
Related: any reaction that follows a reasonable temporal sequence from administration of study drug AND that follows a known response pattern to the suspected drug AND that recurs with re-challenge, AND/OR is improved by stopping the drug or reducing the dose.

### Adverse Event Management

AEs are not recorded prior to the administration of the first dose of study medication. AEs that start, or symptoms related to medical history that worsen after study drug administration are recorded. AEs should be followed until they are either resolved, have returned to baseline, or are determined to be a stable or chronic condition. All SAEs are collected until 30 days after the last dose of study medication. All study-procedure-related SAEs must be collected from the date of patient's written consent.

### Immune-Mediated Adverse Reactions

Immune-mediated adverse reactions (IMARs) are identified for an anti-PD1 antibody.

The specific IMARs noted are:
Immune-Mediated Hepatitis
Immune-Mediated Nephritis
Immune-Mediated Pneumonitis
Immune-Mediated Pneumonitis
Immune-Mediated Colitis and Diarrhea Immune-Mediated Endocrinopathies
Immune-Mediated Skin Reactions
Other Immune-Mediated Adverse Reactions: arthritis, encephalitis, rhabdomyolysis, myositis, myocarditis, pancreatitis, and uveitis.

The monitoring plan is intended to limit the severity and duration of IMARs that occur during combination drug development, and encompass scheduled visits for a physical exam, vital signs, safety laboratory assessments including blood hematology, biochemistry, assessing endocrine functions each Day 1 of a new dosing cycle (pre-dose), assessing coagulation status and urine analyses. The Schedule of Assessments (Tables 5-6) also encompasses assessing the ejection fraction once every three months and conducting regular ECGs.

A summary of management of IMARs caused by G9.2-17 (IgG4), either alone or in combination with other therapeutic agents, is provided in Tables 8-9 below.

TABLE 8
Management of Immune-Mediated Adverse Reactions (IMARs) Caused by G9.2-17 (IgG4)

| IMAR | Guidance for Management |
|---|---|
| Immune-mediated | Monitor patients for signs with radiographic imaging and for |
| pneumonitis | clinical symptoms of pneumonitis. |
| | Withhold G9.2-17 (IGG4) for moderate and permanently |
| | discontinue for severe or life-threatening pneumonitis. |
| | Administer corticosteroids at a dose of 1 to 2 mg/kg/day prednisone |
| | equivalents for moderate (Grade 2) or more severe (Grade 3-4) |
| | pneumonitis, followed by corticosteroid taper. |
| | Withhold G9.2-17 (IGG4) until resolution to < Grade 2. For |
| | moderate (Grade 2) pneumonitis. Resume treatment at a 50% dose |
| | reduction. |
| | Upon recurrence of Grade 2 pneumonitis at the reduced dose of |
| | G9.2-17 (IGG4), permanently discontinue G9.2-17 (IGG4). |
| Immune-mediated colitis | Monitor patients for signs and symptoms of colitis. |
| | Administer corticosteroids at a dose of 0.5 to 1 mg/kg/day |
| | prednisone equivalents followed by corticosteroid taper for |
| | moderate (Grade 2) colitis of more than 5 days duration. |
| | If worsening or no improvement occurs despite initiation of |
| | corticosteroids, increase dose to 1 to 2 mg/kg/day prednisone |
| | equivalents. |
| | When initial Grade 2 colitis resolves to < Grade 2, resume G9.2-17 |
| | (IGG4) at a 50% dose reduction. |
| | If ≥ Grade 2 colitis recurs, withhold G9.2-17 (IGG4) until colitis |
| | resolves to < Grade 2. Resume G9.2-17 (IGG4) at an additional |
| | 50% dose reduction. No further dose reductions for G9.2-17 (IGG4) |
| | are allowed. |
| | Administer corticosteroids at a dose of 1 to 2 mg/kg/day prednisone |
| | equivalents followed by corticosteroid taper for severe (Grade 3) or |
| | life threatening (Grade 4) colitis. |
| | When Grade 3 colitis resolves to ≤ Grade 2, resume G9.2-17 |
| | (IGG4) at a 50% dose reduction. |
| | Permanently discontinue G9.2-17 (IGG4) for life-threatening |
| | (Grade 4) or for recurrent colitis of ≥ Grade 2 upon re-initiation of |
| | G9.2-17 (IGG4). |
| Immune-mediated hepatitis | Monitor for changes in liver function. |
| | Administer corticosteroids at a dose of 0.5 to 1 mg/kg/day |
| | prednisone equivalents for moderate (Grade 2) transaminase |
| | elevations. |
| | Withhold G9.2-17 (IGG4) for moderate (Grade 2) immune- |
| | mediated hepatitis. When resolved to <Grade 2 resume G9.2-17 |
| | (IGG4) at a 50% dose reduction. |
| | If ≥ Grade 2 hepatitis recurs, permanently discontinue G9.2-17 |
| | (IGG4). |
| | Administer corticosteroids at a dose of 1 to 2 mg/kg/day prednisone |
| | equivalents followed by corticosteroid taper for severe (Grade 3) or |
| | life-threatening (Grade 4) transaminase elevations, with or without |
| | concomitant elevation in total bilirubin. |
| | Permanently discontinue G9.2-17 (IGG4) for severe (Grade 3) or |
| | life-threatening (Grade 4) immune-mediated hepatitis. |
| Immune-mediated | Administer hormone replacement as clinically indicated and |
| endocrinopathies: | corticosteroids at a dose of 1 mg/kg/day prednisone equivalents |
| Hypophysitis | followed by corticosteroid taper for moderate (Grade 2) or greater |
| | hypophysitis. |
| | Withhold G9.2-17 (IGG4) for moderate (Grade 2) or severe (Grade |
| | 3) and when resolved to < Grade 2, resume G9.2-17 (IGG4) at a |
| | 50% dose reduction. |
| | If ≥ Grade 2 toxicity recurs, permanently discontinue G9.2-17 |
| | (IGG4). |
| | Permanently discontinue G9.2-17 (IGG4) for life-threatening |
| | (Grade 4) hypophysitis. |
| Adrenal insufficiency | Monitor patients for signs and symptoms of adrenal insufficiency. |
| | Withhold G9.2-17 (IGG4) for moderate (Grade 2) and once |
| | resolved to < Grade 2, resume G9.2-17 (IGG4) at a 50% dose |
| | reduction. |
| | If ≥ Grade 2 toxicity recurs, permanently discontinue G9.2-17 |
| | (IGG4). |
| | Administer corticosteroids at a dose of 1 to 2 mg/kg/day |
| | prednisone equivalents followed by a corticosteroid taper for |
| | severe (Grade 3) or life-threatening (Grade 4) adrenal |
| | insufficiency. |
| | Permanently discontinue G9.2-17 (IGG4) for severe (Grade 3) |
| | or life-threatening (Grade 4) adrenal insufficiency, or for |
| | recurrent colitis of ≥ Grade 2 upon re-initiation of G9.2-17 |
| | (IGG4). |
| Hypothyroidism and | Monitor thyroid function prior to and periodically during an anti- |
| Hyperthyroidism | PD1 antibody treatment. |
| | Administer hormone-replacement therapy for hypothyroidism. |
| | Initiate medical management for control of hyperthyroidism. |
| | There are no recommended dose adjustments of G9.2-17 (IGG4) |
| | for < Grade 3 hypothyroidism or hyperthyroidism |
| | Discontinue G9.2-17 (IGG4) for ≥ Grade 3 hypothyroidism or |
| | hyperthyroidism |
| Type 1 Diabetes Mellitus | Withhold G9.2-17 (IGG4) in cases of severe (Grade 3) |
| | hyperglycemia until metabolic control is achieved. |
| | Upon resolution to ≤ Grade 2, resume G9.2-17 (IGG4) at a 50% |
| | dose reduction. |
| | If ≥ Grade 2 toxicity recurs, permanently discontinue G9.2-17 |
| | (IGG4). |
| | Permanently discontinue G9.2-17 (IGG4) for life-threatening |
| | (Grade 4) hyperglycemia. |
| Immune-mediated nephritis | Monitor patients for elevated serum creatinine prior to and |
| and renal dysfunction | periodically during treatment. |
| (Defined as renal | Administer corticosteroids at a dose of 0.5 to 1 mg/kg/day |
| dysfunction or ≥ Grade 2 | prednisone equivalents for moderate (Grade 2) or severe (Grade 3) |
| increased creatinine, | increased serum creatinine, if worsening or no improvement occurs, |
| requirement for | increase dose of corticosteroids to 1 to 2 mg/kg/day prednisone |
| corticosteroids, and no | equivalents. |
| clear alternate etiology) | Withhold G9.2-17 (IGG4) for moderate (Grade 2) or severe (Grade |
| | 3) increased serum creatinine. When resolved to < Grade 2, resume |
| | G9.2-17 (IGG4) at a 50% dose reduction. |
| | If ≥ Grade 2 toxicity recurs, permanently discontinue G9.2-17 |
| | (IGG4). |
| | Administer corticosteroids at a dose of 1 to 2 mg/kg/day prednisone |
| | equivalents followed by corticosteroid taper for life-threatening |
| | (Grade 4) increased serum creatinine. |
| | Permanently discontinue G9.2-17 (IGG4) for life-threatening |
| | (Grade 4) increased serum creatinine. |
| Immune-mediated skin | Withhold for severe and permanently discontinue for life- |
| adverse reactions | threatening rash. |
| (Immune-mediated rash, | For symptoms or signs of SJS or TEN, withhold G9.2-17 (IGG4) |
| including Stevens-Johnson | and refer the patient for specialized care for assessment and |
| syndrome (SJS) and toxic | treatment. |
| epidermal necrolysis | If SJS or TEN is confirmed, permanently discontinue G9.2-17 |
| (TEN)) | (IGG4). |
| | For immune-mediated rash ≥ Grade 2, administer corticosteroids at |
| | a dose of 1 to 2 mg/kg/day prednisone equivalents followed by a |
| | corticosteroid taper for severe (Grade 3) or life-threatening (Grade |
| | 4) rash. |
| | Withhold G9.2-17 (IGG4) for moderate (Grade 2) and severe |
| | (Grade 3) rash and when resolved to < Grade 2, resume G9.2-17 |
| | (IGG4) at a 50% dose reduction. |
| | If ≥ Grade 2 rash recurs, withhold G9.2-17 (IGG4) until resolved to < |
| | Grade 2 and resume G9.2-17 (IGG4) at an additional 50% dose |
| | reduction. No further dose reductions are allowed. |
| | Permanently discontinue G9.2-17 (IGG4) for life threatening |
| | (Grade 4) rash. |
| Immune-mediated | Monitor for changes in neurologic function. |
| encephalitis | Evaluation of patients with neurologic symptoms may include, but |
| | not be limited to, consultation with a neurologist, brain MRI, and |
| | lumbar puncture. |
| | Withhold G9.2-17 (IGG4) in patients with new-onset moderate to |
| | severe neurologic signs or symptoms and evaluate to rule out |
| | infectious or other causes of moderate to severe neurologic |
| | deterioration. |
| | If other etiologies are ruled out, administer corticosteroids at a dose |
| | of 1 to 2 mg/kg/day prednisone equivalents for patients with |
| | immune-mediated encephalitis, followed by corticosteroid taper. |
| | Permanently discontinue G9.2-17 (IGG4) for immune-mediated |
| | encephalitis. |
| Other Immune-Mediated | If uveitis occurs in combination with other immune-mediated |
| Adverse Reactions | adverse reactions, consider a Vogt-Koyanagi-Harada-like |
| (May include: myocarditis, | syndrome, and may require treatment with systemic steroids to |
| rhabdomyolysis, myositis, | reduce the risk of permanent vision loss. |
| uveitis, iritis, pancreatitis, | For any suspected immune-mediated adverse reactions, exclude |
| facial and abducens nerve | other causes. |
| paresis, demyelination, | Based on the severity (Grade) of the adverse reaction, permanently |
| polymyalgia rheumatica, | discontinue or withhold G9.2-17 (IGG4), administer high-dose |
| autoimmune neuropathy, | corticosteroids, and if appropriate, initiate hormone-replacement |
| Guillain-Barré syndrome, | therapy. |
| hypopituitarism, systemic | Upon improvement to Grade 1 or less, initiate corticosteroid taper |
| inflammatory response | and continue to taper over at least 1 month. |
| syndrome, gastritis, | Consider restarting G9.2-17 (IGG4) after completion of |
| duodenitis, sarcoidosis, | corticosteroid taper based on the severity of the event, resuming at a |
| histiocytic necrotizing | 50% dose reduction. |
| lymphadenitis (Kikuchi | |
| lymphadenitis), motor | |
| dysfunction, vasculitis, | |
| aplastic anemia, | |
| pericarditis, and | |
| myasthenic syndrome.) | |

TABLE 9
Management of Immune-Mediated Adverse Reactions (IMARs) Caused
by G9.2-17 (IgG4) + Anti-PD1 Antibody Combination Treatment

| IMAR | Guidance for Management |
|---|---|
| Immune-mediated | Monitor patients for signs with radiographic imaging and for |
| pneumonitis | symptoms of pneumonitis. |
| | Grade 1, consider holding an anti-PD1 antibody until there is |
| | significant improvement in patient's signs and symptoms |
| | Withhold an anti-PD1 antibody and G9.2-17 (IGG4) for moderate |
| | (Grade 2) and permanently discontinue for severe (Grade 3) or life- |
| | threatening (Grade 4) pneumonitis. Administer corticosteroids at a |
| | dose of 1 to 2 mg/kg/day prednisone equivalents for moderate (Grade |
| | 2). For more severe (Grade 3-4) pneumonitis, permanently |
| | discontinue G9.2-17 (IGG4) and an anti-PD1 antibody and treatment |
| | in a hospital setting with IV methylprednisolone 2 to 4 mg/kg/day is |
| | recommended, followed by corticosteroid taper. |
| | Withhold an anti-PD1 antibody and G9.2-17 (IGG4) until resolution |
| | to < Grade 2 for moderate (Grade 2) pneumonitis. Resume G9.2-17 |
| | (IGG4) at a 50% dose reduction. |
| | An anti-PD1 antibody may be restarted only if symptoms resolve |
| | completely or are controlled on prednisolone ≤10 mg/day. Withhold |
| | an anti-PD1 antibody for Grade 2 pneumonitis, and only restart if |
| | symptoms resolve completely or are controlled on prednisolone ≤10 |
| | mg/day. Permanently discontinue treatment with an anti-PD1 |
| | antibody if symptoms persist with the use of corticosteroids. |
| | Upon recurrence of ≥ Grade 2 pneumonitis, permanently discontinue |
| | treatment with both the study medication and an anti-PD1 antibody |
| Immune-mediated colitis | Monitor patients for signs and symptoms of colitis. |
| and diarrhea | Withhold treatment with an anti-PD1 antibody and G9.2-17 (IGG4) |
| | for Grade 2 diarrhea or colitis and start oral prednisolone 0.5 |
| | mg/kg/day (nonenteric coated). If worsening or no improvement |
| | occurs despite initiation of corticosteroids, increase dose to 1 to 2 |
| | mg/kg/day prednisone equivalents. |
| | Taper steroids over 2 to 4 weeks with resolving signs and symptoms. |
| | Resume treatment with an anti-PD1 antibody when signs and |
| | symptoms improve or resolve to baseline. Administer prednisolone 1 |
| | to 2 mg/kg/day for colitis Grade 3 or Grade 4, converting to oral |
| | prednisolone and tapering this dose over at least 4 weeks when |
| | improvement is evident. Permanently discontinue an anti-PD1 |
| | antibody and G9.2-17 (IGG4) for recurrent severe (Grade 3) or life- |
| | threatening Grade 4 diarrhea or colitis. |
| | Resume G9.2-17 (IGG4) at a 50% dose reduction when signs and |
| | symptoms improve or resolve to baseline. |
| | If ≥ Grade 3 recurs, then permanently discontinue treatment with both |
| | the study medication and an anti-PD1 antibody. |
| | If ≥ Grade 2 colitis recurs, withhold G9.2-17 (IGG4) and an anti-PD1 |
| | antibody until colitis resolves to baseline. Apply treatment regimen |
| | outlined above. Resume G9.2-17 (IGG4) at an additional 50% dose |
| | reduction. No further dose reductions for G9.2-17 (IGG4) are |
| | allowed. |
| | If ≥ Grade 2 colitis recurs, permanently discontinue treatment with |
| | both study medications. |
| | Permanently discontinue G9.2-17 (IGG4) and an anti-PD1 antibody |
| | for life-threatening (Grade 4). |
| Immune-mediated | Monitor for changes in liver function. |
| hepatitis | Administer corticosteroids at a dose of 0.5 to 1 mg/kg/day prednisone |
| | equivalents for moderate (Grade 2) transaminase elevations. |
| | Withhold an anti-PD1 antibody and G9.2-17 (IGG4) for moderate |
| | (Grade 2) immune-mediated hepatitis. When resolved to < Grade 2, |
| | and prednisolone tapered to ≤10 mg, resume an anti-PD1 antibody |
| | and G9.2-17 (IGG4). G9.2-17 (IGG4) is to be resumed at a 50% dose |
| | reduction. |
| | If ≥ Grade 2 hepatitis recurs, permanently discontinue treatment with |
| | both G9.2-17 (IGG4) and an anti-PD1 antibody. |
| | Administer corticosteroids at a dose of 0.5 to 2 mg/kg/day prednisone |
| | equivalents followed by corticosteroid taper for moderate (grade 2), |
| | severe (Grade 3) or life-threatening (Grade 4) transaminase |
| | elevations, with or without concomitant elevation in total bilirubin. |
| | Permanently discontinue treatment with both G9.2-17 (IGG4) and an |
| | anti-PD1 antibody for severe (Grade 3) or life-threatening (grade 4) |
| | immune-mediated hepatitis. |
| Immune-mediated | Administer hormone replacement as clinically indicated and |
| endocrinopathies: | corticosteroids. Withhold G9.2-17 (IGG4) and an anti-PD1 antibody |
| Hypophysitis | for moderate (Grade 2) hypophysitis. First administer oral |
| | prednisolone 0.5 to 1 mg/kg/day and add hormone replacement as |
| | clinically indicated until resolved/improved to ≤ Grade 2. Taper |
| | corticosteroids over at least 1 month. Resume G9.2-17 (IGG4) at a |
| | 50% dose reduction when resuming. Discontinuation is usually not |
| | necessary for asymptomatic and manageable patients who are treated |
| | by an endocrinologist. |
| | If ≥ Grade 2 toxicity recurs, permanently discontinue treatment with |
| | both G9.2-17 (IGG4) and an anti-PD1 antibody. |
| | Permanently discontinue treatment G9.2-17 (IGG4) and an anti-PD1 |
| | antibody for severe (grade 3) and life-threatening (Grade 4) |
| | hypophysitis. |
| Adrenal insufficiency | Monitor patients for signs and symptoms of adrenal insufficiency. |
| | Withhold G9.2-17 (IGG4) and an anti-PD1 antibody for moderate |
| | (Grade 2) adrenal insufficiency. First administer oral prednisolone 0.5 |
| | to 1 mg/kg/day as clinically indicated until resolved/improved to ≤ |
| | Grade 2. Taper corticosteroids over at least 1 month. Resume G9.2-17 |
| | (IGG4) at a 50% dose reduction when resuming. Discontinuation is |
| | usually not necessary for asymptomatic and manageable patients who |
| | are treated by an endocrinologist. |
| | If ≥ Grade 2 toxicity recurs, permanently discontinue treatment |
| | with both G9.2-17 (IGG4) and an anti-PD1 antibody. |
| | Permanently discontinue treatment G9.2-17 (IGG4) and an anti- |
| | PD1 antibody for severe (grade 3) and life-threatening (Grade 4) |
| | adrenal insufficiency. |
| Hypothyroidism and | Monitor thyroid function prior to and periodically during an anti-PD1 |
| Hyperthyroidism | antibody treatment. |
| | Administer hormone-replacement therapy for hypothyroidism. |
| | Initiate medical management for control of hyperthyroidism. |
| | Administer oral prednisolone 0.5 mg/kg/day in patients with thyroid |
| | pain. |
| | Thyrotoxic patients should be treated with a beta blocker and may |
| | require additional treatment with a carbimazole. |
| | Hold an anti-PD1 antibody for thyroiditis Grade 3 or 4 until this |
| | condition resolves to a Grade 0-1, then consider resuming treatment. |
| | Discontinue G9.2-17 (IGG4) and an anti-PD1 antibody for ≥Grade 3 |
| | and 4 hypothyroidism or hyperthyroidism |
| Type 1 Diabetes Mellitus | Monitor patients for hyperglycemia or other signs and symptoms of |
| | diabetes. |
| | Withhold G9.2-17 (IGG4) and an anti-PD1 antibody in cases of |
| | severe (Grade 3) hyperglycemia until metabolic control is achieved, |
| | until blood glucose has been stabilized to baseline or Grade 0 to 1 and |
| | the patient is hyperglycemia symptom-free. Administer insulin as |
| | clinically indicated for type 1 diabetes. |
| | Follow G9.2-17 (IGG4) IMAR guidance for Type 1 Diabetes Mellitus |
| | when resolved to ≤ Grade 2. Permanently discontinue treatment with |
| | both study medications for life-threatening (Grade 4) hyperglycemia. |
| Immune-mediated | Monitor patients for renal function prior to and periodically during |
| nephritis and renal | treatment. In the event of immune-mediated nephritis, the frequency |
| dysfunction | of renal function tests should be increased. |
| (Defined as renal | Withhold G9.2-17 (IGG4) and an anti-PD1 antibody for moderate |
| dysfunction or ≥ Grade 2 | (Grade 2) or severe (Grade 3) increased nephritis. When resolved to < |
| increased creatinine, | Grade 2, resume G9.2-17 (IGG4) at a 50% dose reduction. If ≥ Grade |
| requirement for | 2 toxicity recurs, permanently discontinue treatment with both G9.2- |
| corticosteroids, and no | 17 (IGG4) and an anti-PD1 antibody. |
| clear alternate etiology) | Ensure hydration and administer corticosteroids at a dose of 0.5 to 1 |
| | mg/kg/day prednisone equivalents for moderate (Grade 2) or 1 to 2 |
| | mg/kg/day prednisone equivalents for severe (Grade 3) nephritis. |
| | Permanently discontinue treatment with both G9.2-17 (IGG4) and an |
| | anti-PD1 antibody for life-threatening (Grade 4) nephritis and |
| | recurrent Grade 3 nephritis as well. |
| Immune-mediated skin | For ≤ Grade 2 rashes, treatment that can be controlled or resolved |
| adverse reactions | with moderate strength topical or oral steroids while continuing an |
| | anti-PD1 antibody and G9.2-17 (IGG4). |
| (An anti-PD1 antibody | Monitor patients for suspected severe skin reactions and exclude other |
| can cause immune- | causes. |
| mediated rash, including | Withhold both medications for severe (Grade 3) until signs and |
| Stevens-Johnson | symptoms become mild, and permanently discontinued for moderate |
| syndrome (SJS) and toxic | (Grade 2) to severe (Grade 3) rashes that do not improve or worsen |
| epidermal necrolysis | while treated with oral or IV prednisolone, and permanently |
| (TEN), some cases with | discontinue for life-threatening (Grade 4) rash. |
| fatal outcome.) | For symptoms or signs of SJS or TEN, permanently discontinue an |
| | anti-PD1 antibody and G9.2-17 (IGG4) and refer the patient for |
| | specialized care for assessment and treatment. |
| | The recommended dose of oral prednisolone is 0.5 to 1 mg/kg/day for |
| | 3 days with tapering over 2 to 4 weeks (moderate rashes) and using |
| | methyl prednisolone 0.5 to 1 mg/kg/day with converting to oral |
| | prednisolone and tapering over at least 4 weeks (severe rashes). |
| | When resolved to < Grade 2, resume G9.2-17 (IGG4) at a 50% dose |
| | reduction. |
| Immune-mediated | Monitor for changes in neurologic function. |
| encephalitis | Evaluation of patients with neurologic symptoms may include, but not |
| | be limited to, consultation with a neurologist, brain MRI, and lumbar |
| | puncture. |
| | Withhold G9.2-17 (IGG4) and an anti-PD1 antibody in patients with |
| | new-onset moderate to severe neurologic signs or symptoms and |
| | evaluate to rule out infectious or other causes of moderate to severe |
| | neurologic deterioration. |
| | If other etiologies are ruled out, administer corticosteroids at a dose of |
| | 1 to 2 mg/kg/day prednisone equivalents for patients with immune- |
| | mediated encephalitis, followed by corticosteroid taper. |
| | Permanently discontinue treatment with G9.2-17 (IGG4) and an anti- |
| | PD1 antibody for immune-mediated encephalitis. |
| Other Immune-Mediated | For any suspected immune-mediated adverse reactions, exclude other |
| Adverse Reactions | causes. |
| The following immune- | Based on the severity of the adverse reaction, permanently |
| mediated adverse | discontinue or withhold G9.2-17 (IGG4) and an anti-PD1 antibody, |
| reactions occurred in less | administer high-dose corticosteroids, and if appropriate, initiate |
| than 1% of patients | hormone-replacement therapy. |
| treated with an anti-PD1 | Upon improvement to Grade 1 or less, initiate corticosteroid taper and |
| antibody: arthritis, | continue to taper over at least 1 month. |
| encephalitis, | Consider restarting G9.2-17 (IGG4) and an anti-PD1 antibody after |
| rhabdomyolysis, | completion of corticosteroid taper based on the severity of the event. |
| myositis, myocarditis, | Resume G9.2-17 (IGG4) at a 50% dose reduction. Immune-mediated |
| pancreatitis, and uveitis. | adverse reactions may occur after discontinuation of an anti-PD1 |
| | antibody therapy. |
| Infusion reactions | The symptoms of infusion-related reactions that may be observed |
| (Reported in less than | with an anti-PD1 antibody include fever, chills/rigor, nausea, pruritus, |
| 1.0% of patients in | angioedema, hypotension, headache, bronchospasm, urticaria, rash, |
| clinical trials.) | vomiting, myalgia, dizziness, or hypertension. |
| | Patients should be closely monitored for these signs and symptoms |
| | during the infusion. |
| | For Grade 1 reactions, decrease infusion rate by 50%; for Grade 2 |
| | reactions, stop infusion and only resume with caution at 50% of |
| | previous rate if infusion-related reaction has resolved or decreased to |
| | Grade 1 in severity. For Grade 3 or Grade 4 infusion-related |
| | reactions, stop infusion and permanently discontinue an anti-PD1 |
| | antibody. Subsequent infusions should be given after premedication |
| | and at the reduced infusion rate. |
| Complications of | Transplant-related complications include hyperacute graft-versus- |
| Allogeneic | host-disease (GVHD), acute GVHD, chronic GVHD, hepatic veno- |
| Hematopoietic Stem Cell | occlusive disease (VOD) after reduced intensity conditioning, and |
| Transplantation (HSCT) | steroid-requiring febrile syndrome (without an identified infectious |
| (Fatal and other serious | cause). |
| complications can occur | These complications may occur despite intervening therapy between |
| in patients who receive | PD-1 blockade and allogeneic HSCT. |
| allogeneic HSCT before | Follow patients closely for evidence of transplant-related |
| or after being treated with | complications and intervene promptly. |
| a PD-1 receptor blocking | Consider the benefit versus risks of treatment with a PD-1 receptor |
| antibody.) | blocking antibody prior to or after an allogeneic HSCT. |
| Embryo-Fetal Toxicity | Advise pregnant women of the potential risk to a fetus. |
| (Based on its mechanism | Advise females of reproductive potential to use effective |
| of action and data from | contraception during treatment with a an anti-PD1 antibody- |
| animal studies, an anti- | containing regimen and for at least 5 months after the last dose of an |
| PD1 antibody can cause | anti-PD1 antibody. |
| fetal harm when | |
| administered to a | |
| pregnant woman.) | |

### Dose-Reduction Procedure for Adverse Event Management

In the event where dose-reduction is used for AE management in Part 2 of the study, two dose reductions of 50% each are allowed. Dose reductions are pursued when clinical benefit is expected and may continue to be derived.

### Clinical Laboratory Abnormalities and Other Abnormal Assessments as AEs and SAEs

Abnormal laboratory findings (eg, clinical chemistry, hematology, and urinalysis) or other abnormal assessments (eg, ECGs or vital signs) that are judged as clinically significant are recorded as AEs and SAEs if they meet the definition of an AE or SAE. Clinically significant abnormal laboratory findings or other abnormal assessments that are detected during the study or are present at screening and significantly worsen following the start of the study are reported as AEs or SAEs. However, clinically significant abnormal laboratory findings or other abnormal assessments that are associated with the disease being studied, unless judged as more severe than expected for the patient's condition, or that are present or detected at the start of the study and do not worsen, will not be reported as AEs or SAEs.

Laboratory measurements that deviate clinically significantly from previous measurements may be repeated. If warranted, additional or more frequent testing than is specified in the protocol should be done to provide adequate documentation of AEs and the resolution of AEs.

### Time Period and Frequency for Collecting AE and SAE Information

All AEs and SAEs are collected from the start of intervention until the follow-up visit at the time points specified in the SoA (Tables 5-6).

Medical occurrences that begin before the start of study intervention but after obtaining informed consent are recorded as Medical History/Current Medical Conditions, not as AEs.

All SAEs are recorded and reported immediately and under no circumstance should this exceed 24 h.

### Follow-Up of AEs and SAEs

After the initial AE/SAE report, it is required to proactively follow each participant at subsequent visits/contacts. All SAEs are followed until resolution, stabilization, the event is otherwise explained, or the participant is lost to follow-up.

### Statistical Considerations

The study is completed when the last patient has had their last visit. The database is locked for the primary analysis after the last patient has had their primary endpoint event. A final study analysis is performed after study completion.

### Statistical Hypotheses

The current study is designed to identify the MTD of G9.2-17 (IgG4) (Part 1) by assessing DLTs, followed by an assessment of drug activity (alone or in combination) in the three disease types using Simon's two-stage optimal design. Study hypotheses for Part 2 are detailed below.

CRC and CCA G9.2-17 (IgG4) single agent treatment arms
Null hypothesis: ORR 3 is ≤5%
Alternative hypothesis: ORR 3 is ≥15%.

CRC and CCA G9.2-17 (IgG4)+an anti-PD1 antibody combination treatment arms
Null hypothesis: ORR 3 is ≤10%
Alternative hypothesis: ORR 3 is ≥25%.

### Analysis Sets

The intent-to-treat (ITT) population is defined as those patients who received at least one dose of the study drug, unless otherwise specified. The primary efficacy analyses are performed for the ITT. Patient disposition is performed for the ITT.

The Efficacy Population is defined as all patients in the ITT and having at least one measurable ORR 3 or PFS 6 assessment. This population is used for a sensitivity analysis.

The per-protocol (PP) Population is defined as any patient who received at least one full cycle of G9.2-17 (IGG4) and without major protocol deviations.

The safety population (SAF) is defined as all patients who receive at least one dose of the study drug. The safety analyses are performed for the SAF.

The PK/PD population is defined as those patients who have received at least one full cycle of G9.2-17 (IGG4).

### Primary Endpoint(s)

### Safety Analysis—Part 1 and Part 2

All safety analyses are made on the SAF unless otherwise specified.

### Adverse Events

Treatment-emergent adverse events (TEAEs) are defined as events that occur on or after the first dose of study medication. The MedDRA coding dictionary is used for the coding of AEs. TEAEs, serious or CTCAE Grade 3 or Grade 4 TEAEs, and TEAEs related to treatment are summarized overall and by system organ class and preferred term by treatment group. These summarize the number of events and the number and percent of patients with a given event. In addition, the number and percent of patients with TEAEs are provided by maximum severity. A summary of all TEAEs by system organ class and preferred term occurring in ≥5% of patients in either treatment group is provided.

DLTs, the MTD and the RP2D are summarized.

### Laboratory Assessments

All laboratory-based data is presented as listings of all values as well as of abnormal results judged to be clinically significant, which is reported as AEs. Numeric summaries of all observed findings and changes from baseline screening laboratory evaluations are provided by visit and treatment group, including chemistry, hematology, and urinalysis results. No inferential comparisons are planned.

### Vital Signs

Numeric summaries of all observed findings and changes from baseline screening vital signs are provided by time point and treatment group, including blood pressure, heart rate, respiratory rate, and temperature. No inferential analyses are planned for vital signs.

### ECGs, ECHO/MUGA, and Physical Examination

Physical examination data and changes are presented as listings. ECG results are presented as listings and summarized by treatment group and visit, based on incidence of clinically significant abnormalities. No inferential comparisons across treatment groups are planned.

### Primary Efficacy Analysis—Part 2

Disease response is assessed according to RECIST v1.1 and is summarized descriptively for the ITT, PP, and Efficacy Populations.

The primary efficacy endpoints are:
ORR 3 for CRC and CCA
PFS 6 for PDAC

### Secondary Endpoint(s)

### Pharmacokinetics, Pharmacodynamics, and Immunogenicity

PK, PD, and immunogenicity are summarized descriptively for the PK/PD population in both Part 1 and Part 2.

### Secondary Efficacy Analysis—Part 2

Disease response (ORR, PFS, DCR, DoR, and OS) is assessed according to RECIST v1.1 and is summarized descriptively for the ITT, PP, and Efficacy Populations.

### Exploratory Endpoints

Analysis of exploratory endpoints is detailed in the SAP.

### Other Analysis

Other collected data not specifically mentioned is presented in patient listings.

### Disposition, Demographics, Baseline Characteristics, and Medical History

Disposition information is summarized including the number of enrolled patients, screening failures, treated patients, and the number of patients withdrawn by reason.

Demographics, baseline characteristics, and medical history is summarized by treatment group and overall using descriptive statistics for the ITT and PP.

### Prior and Concomitant Medications

Number and percentage of patients taking prior and concomitant medications is summarized by treatment group and overall for the ITT and PP.

### Example 2: Anti-Galectin-9 Antibody Stability Study

The candidate IgG4 antibody underwent stability analysis after storage under several different conditions and at different concentrations. Stability analysis was performed via size exclusion chromatography (SEC) using a TOSOH TSKgel Super SW mAb column. SEC profiles before and after storage were compared to identify any issues with protein stability (e.g., aggregation or degradation).

### Materials and Methods

### Sample Preparation

The anti-Galectin-9 antibody was stored at −80° C. until use. Prior to analysis, samples were thawed in a room temperature water bath and stored on ice until analysis. Prior to handling, absorbance at 280 nm was measured using Nanodrop. The instrument was blanked using TBS (20 mM Tris pH 8.0, 150 mM NaCl). The sample was then transferred to polypropylene microcentrifuge tubes (USA Scientific, 1615-5500) and centrifuged at 4° C., 16.1k×g for 30 minutes. Samples were filtered through a 0.22 μm filter (Millipore; SLGV004SL). Post-filtration absorbance was measured.

### HPLC Analysis

Sample conditions tested included the following: ambient stability (0 hours at room temperature, 8 hours at room temperature), refrigerated stability (0 hours at 4° C., 8 hours at 4° C., 24 hours at 4° C.), and freeze/thaw stability (1× freeze/thaw, 3× freeze/thaw, 5× freeze/thaw). Each condition was run in duplicate at three different concentrations: stock, 10× dilution, and 100× dilution. One hundred μL samples were prepared for each condition and stored in a polypropylene microcentrifuge tube. Dilutions were prepared in TBS when necessary. Absorbance at 280 nm was read prior to analysis. Room temperature samples were stored on the benchtop for the durations indicated. 4° C. samples were either stored on ice or in 4° C. refrigerator for the periods indicated in Table 10. Freeze-thaw samples were snap-frozen in liquid nitrogen and then thawed in a room temperature water bath. The freeze and thaw process were performed either once, three or five times, and then the samples were stored at 4° C. until analysis.

SEC analysis was performed using a TOSOH TSKgel SuperSW mAb HR column on a Shimadzu HPLC with a UV detector at 280 nm. The columns were loaded with 25 μL of sample and run at 0.5 mL/min for 40 minutes. The KBI buffer formulation was used as the mobile phase.

### Results

The concentrations of the antibody were determined using UV absorbance measurements before and after filtration, as shown in Table 10. Two 2 mL samples supplied by KBI were thawed, one vial for use in room temperature and freeze/thaw conditions, and the other vial for use in the 4° C. conditions. Absorbance readings showed nearly complete recovery after filtration.

TABLE 10
Protein Recovery after Sample Preparation

| | | Pre-Filtration | Post-Filtration | Recovery |
|---|---|---|---|---|
| Vial | Read | (mg/mL) | (mg/mL) | (%) |
| 1 | 1 | 9.574 | 9.416 | 98.4 |
| (Used for RT | 2 | 9.435 | 9.553 | 101.3 |
| and | 3 | 9.504 | 9.541 | 100.4 |
| Freeze/Thaw) | Average | 9.50 ± 0.07 | 9.50 ± 0.07 | 100.0 ± 1.5 |
| 2 | 1 | 9.618 | 9.401 | 98.6 |
| (Used for 4° C.) | 2 | 9.814 | 9.704 | 98.9 |
| | 3 | 9.451 | 9.394 | 99.4 |
| | Average | 9.63 ± 0.18 | 9.53 ± 0.16 | 98.9 ± 0.4 |

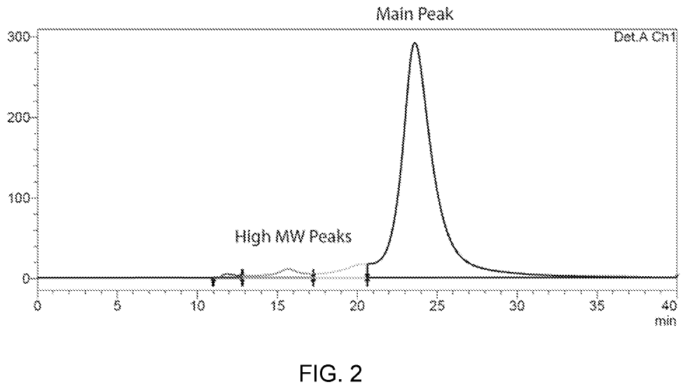

Two or three high molecular weight peaks that eluted earlier than the main peak were observed (FIG. 2). These peaks comprised approximately 5% of the total sample under each condition assayed (Table 11). No significant differences in protein concentration were observed under all assayed conditions.

TABLE 11
Stability Results

| | | Dilution | | Concentration | High Molecular Weight Peaks | | | | |
|---|---|---|---|---|---|---|---|---|---|
| Condition | Time | Sample | | (mg/mL) | 1 | 2 | 3 | Total | Main |
| Room | 0 hr | 1 | 1 | 9.3 ± 0.3 | 0.06 | 3.024 | 4.307 | 7.39 | 92.61 |
| Temperature | | | 2 | 9.36 ± 0.03 | 0.615 | 0.273 | 3.822 | 4.71 | 95.29 |
| | | 10 | 1 | 0.96 ± 0.012 | 0.34 | 1.18 | 3.183 | 4.70 | 95.30 |
| | | | 2 | 1.00 ± 0.02 | 0.418 | 1.225 | 2.541 | 4.18 | 95.82 |
| | | 100 | 1 | 0.147 ± 0.003 | 0.25 | 2.1278 | 2.472 | 4.85 | 95.15 |
| | | | 2 | 0.14 ± 0.05 | 0.17 | 1.507 | 2.684 | 4.36 | 95.64 |
| | 8 hr | 1 | 1 | 9.5 ± 0.19 | 0.597 | 1.41 | 1.997 | 4.00 | 96.00 |
| | | | 2 | 9.46 ± 0.04 | 0.501 | 1.219 | 2.147 | 3.87 | 96.13 |
| | | 10 | 1 | 1.03 ± 0.02 | 0.413 | 1.173 | 2.51 | 4.10 | 95.90 |
| | | | 2 | 1.026 ± 0.002 | 0.367 | 1.22 | 2.592 | 4.18 | 95.82 |
| | | 100 | 1 | 0.14 ± 0.012 | 0.839 | 1.584 | 2.342 | 4.77 | 95.24 |
| | | | 2 | 0.104 ± 0.008 | 0.723 | 1.578 | 2.719 | 5.02 | 94.98 |
| 4° C. | 1 hr | 1 | 1 | 9.68 ± 0.05 | 0.623 | 1.489 | 2.066 | 4.18 | 95.82 |
| | | | 2 | 9.6 ± 0.15 | 0.463 | 1.617 | 2.999 | 5.08 | 94.92 |
| | | 10 | 1 | 0.96 ± 0.03 | 0.436 | 1.122 | 2.438 | 4.00 | 96.00 |
| | | | 2 | 0.96 ± 0.02 | 0.432 | 1.173 | 2.799 | 4.40 | 95.60 |
| | | 100 | 1 | 0.106 ± 0.003 | 0.503 | 1.834 | 2.73 | 5.07 | 94.93 |
| | | | 2 | 0.103 ± 0.004 | 0.538 | 1.603 | 2.789 | 4.93 | 95.07 |
| | 8 hr | 1 | 1 | 9.59 ± 0.07 | 0.285 | 1.135 | 2.699 | 4.12 | 95.88 |
| | | | 2 | 9.87 ± 0.010 | 0.382 | 0.85 | 2.74 | 3.97 | 96.03 |
| | | 10 | 1 | 0.99 ± 0.015 | 1.342 | 1.168 | 2.647 | 5.16 | 94.84 |
| | | | 2 | 0.98 ± 0.03 | 0.901 | 1.79 | 2.547 | 5.24 | 94.76 |
| | | 100 | 1 | 0.100 ± 0.002 | 0 | 1.768 | 4.856 | 6.62 | 93.38 |
| | | | 2 | 0.097 ± 0.003 | 0 | 0.98 | 3.653 | 4.63 | 95.37 |
| | 24 hr | 1 | 1 | 9.60 ± 0.04 | 0.466 | 1.563 | 2.988 | 5.02 | 94.98 |
| | | | 2 | 9.68 ± 0.08 | 0.491 | 1.166 | 2.521 | 4.18 | 95.82 |
| | | 10 | 1 | 0.973 ± 0.005 | 0.579 | 1.095 | 2.888 | 4.56 | 95.44 |
| | | | 2 | 0.98 ± 0.04 | 0.36 | 1.106 | 2.488 | 3.95 | 96.05 |
| | | 100 | 1 | 0.097 ± 0.001 | 0.588 | 1.413 | 2.95 | 4.95 | 95.05 |
| | | | 2 | 0.099 ± 0.002 | 0.587 | 1.463 | 2.886 | 4.94 | 95.06 |
| Freeze | 1x | 11 | 1 | 9.5 ± 0.10 | 0.439 | 1.143 | 2.292 | 3.87 | 96.13 |
| Thaw | | | 2 | 9.04 ± 0.08 | 0.489 | 1.597 | 2.58 | 4.67 | 95.33 |
| | | 10 | 1 | 1.09 ± 0.03 | 0.388 | 1.228 | 2.741 | 4.36 | 95.64 |
| | | | 2 | 1.08 ± 0.05 | 0.387 | 1.243 | 2.932 | 4.56 | 95.44 |
| | | 100 | 1 | 0.12 ± 0.010 | 0.467 | 1.207 | 2.355 | 4.03 | 95.97 |
| | | | 2 | 0.11 ± 0.011 | 0.627 | 1.65 | 3.09 | 5.37 | 94.63 |
| | 3x | 1 | 1 | 8.1 ± 0.8 | 0.478 | 1.152 | 1.791 | 3.42 | 96.58 |
| | | | 2 | 9.0 ± 0.7 | 0.5 | 1.18 | 1.99 | 3.67 | 96.33 |
| | 5x | 1 | 1 | 8.9 ± 0.6 | 0.505 | 1.578 | 2.612 | 4.70 | 95.31 |
| | | | 2 | 8.6 ± 0.4 | 0.464 | 1.662 | 3.008 | 5.13 | 94.87 |

In summary, the anti-Galectin-9 antibody showed consistent stability after storage under all conditions analyzed, as indicated by no significant change in the SEC profile. There was no significant loss of protein after filtration, and two to three high molecular weight peaks were identified, comprising approximately 5% of the total sample. The results suggest that the antibody is stable under all conditions tested, with no aggregate formation or degradation observed.

### Example 3. Assessment of Galectin-9 Expression in Tumor Biopsy-Derived Organoid Fractions

Tumor organoids can be applied for the prediction of patient outcome, since the use of tumor models with similar characteristics to the original tumors may result in more accurate predictions of drug responses in patients. (See, e.g., Trends in Biotechnology; 36(4): 358-371, Apr. 1, 2018).

Galectin-9 levels in a tumor may function as an indicator to predict a drug response. Biopsy derived organoids can be used as a proxy to assess levels of Galectin-9 in the original tumor. Accordingly, the ability to assess Galectin-9 levels in single cell or organoid fractions was tested.

Biopsies were received from representative pancreatic adenocarcinoma and colorectal cancers and processed as follows. Human surgically resected tumor specimens were received fresh in DMEM media on ice and minced in 10 cm dishes. Minced tumors were resuspended in DMEM+10% FBS with 100 U/mL collagenase type IV to obtain spheroids. Partially digested samples were pelleted and then re-suspended in fresh DMEM+10% FBS and strained over both 100 mm and 40 mm filters to generate S1 (>100 mm), S2 (40-100 mm), and S3 (<40 mm) spheroid fractions, which were subsequently maintained in ultra-low-attachment tissue culture plates.

S2 fractions were digested by trypsin for 15 minutes to generate into single cells. For flow cytometry preparation, cell pellets from S2 and S3 fractions were re-suspended and cell labeling was performed after Fc receptor blocking (#422301; BioLegend, San Diego, CA) by incubating cells with fluorescently conjugated mAbs directed against human CD45 (HI30), CD3 (UCHT1), CD11b (M1/70), Epcam (9C4) and Gal9 (9M1-3; all Biolegend) or Gal9 Fab of G9.2-17 or Fab isotype. Dead cells were excluded from analysis using zombie yellow (BioLegend). Flow cytometry was carried out on the Attune NXT flow cytometer (Thermo Scientific). Data were analyzed using FlowJo v.10.1 (Treestar, Ashland, OR).

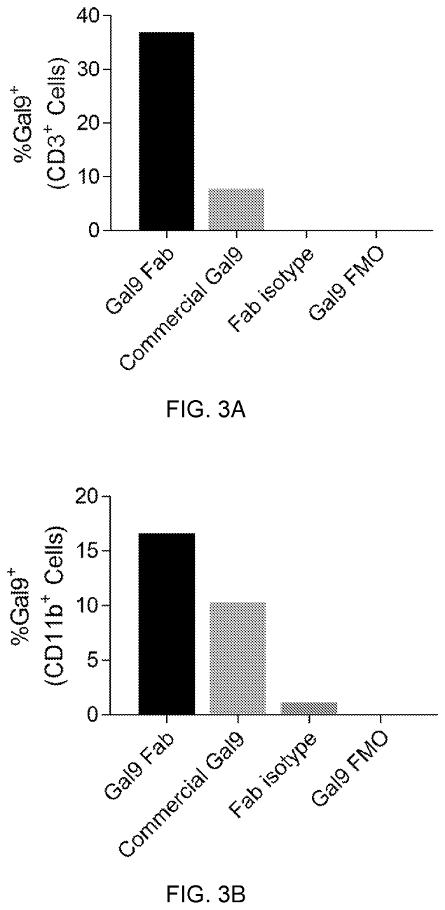

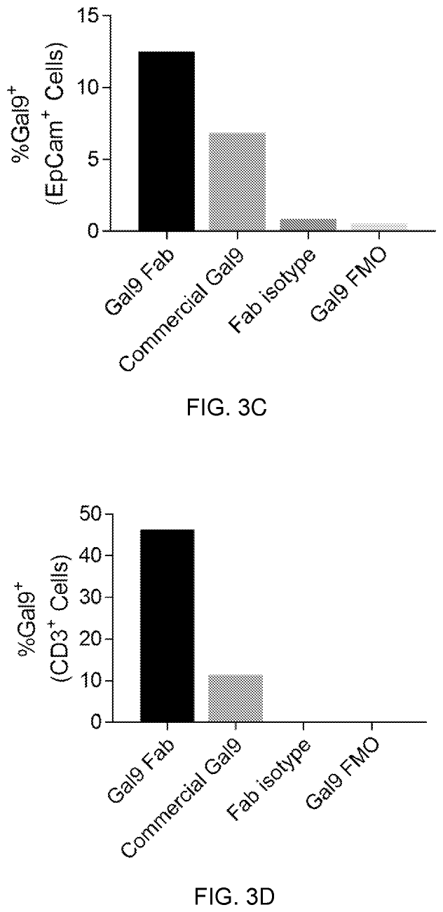

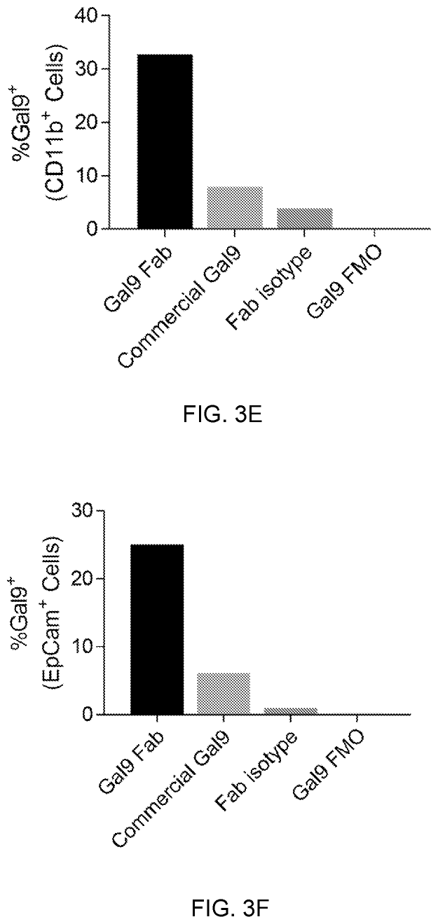

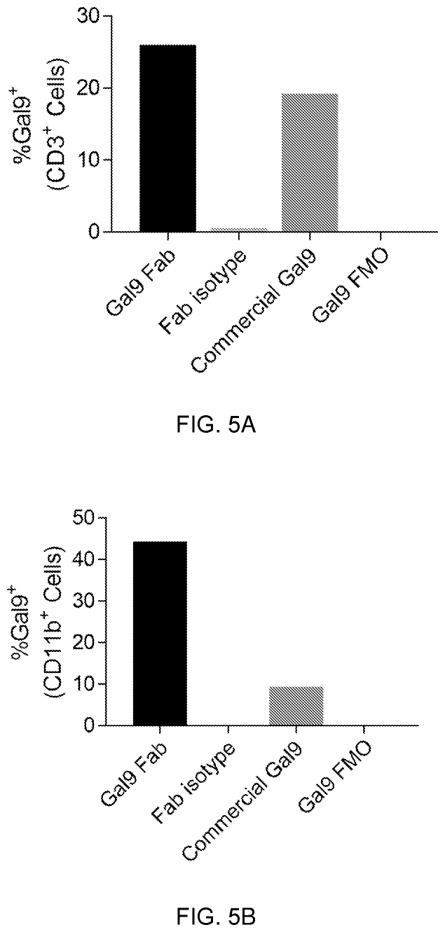

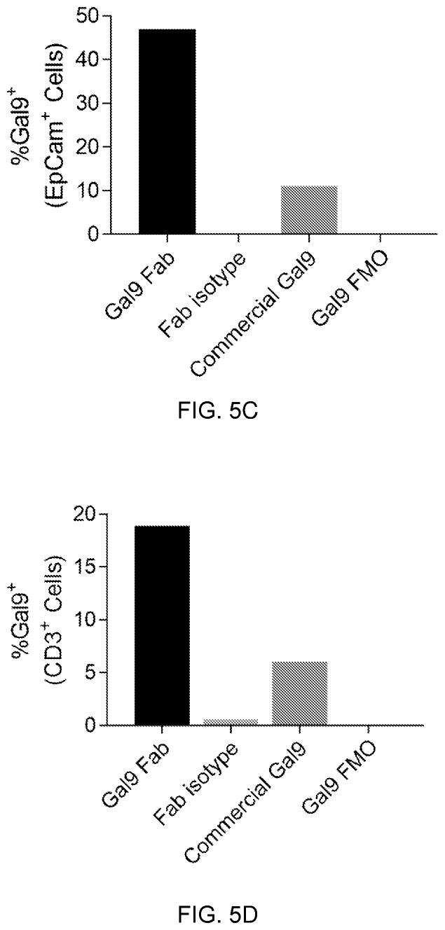

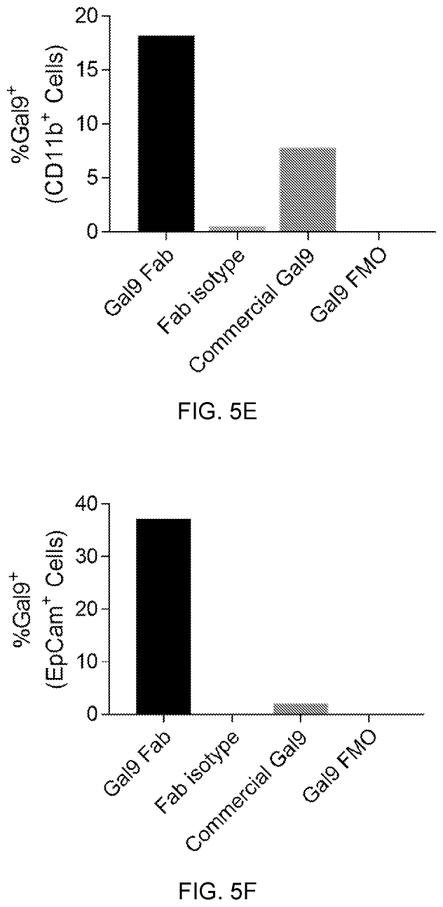

Results are shown in FIGS. 3A-3F, 4A-4F and 5A-5F and indicate that levels of Galectin-9 detected by the Gal9 G9.2-17 Fab in S2 single cell and S3 organoid fractions correlate. Accordingly, both S2 single cells and S3 organoids can be used for assessment of Galectin-9 levels in organoids derived from tumor biopsies.

### Example 4. Preparation of Patient-Derived Organotypic Tumor Spheroids (PDOTs) for Cellular Analysis

Biopsy-derived organoids can be a useful measure to assess the ability of a therapeutic to stimulate an immune response. Accordingly, S2 fractions described in the previous Example 3 above used for ex vivo culture were treated with anti-Galectin-9 antibody G9.2-17 and prepared for immune profiling.

An aliquot of the S2 fraction was pelleted and resuspended in type I rat tail collagen (Corning) at a concentration of 2.5 mg/mL following the addition of 10×PBS with phenol red with pH adjusted using NaOH. pH 7.0-7.5 was confirmed using PANPEHA Whatman paper (Sigma-Aldrich). The spheroid-collagen mixture was then injected into the center gel region of a 3-D microfluidic culture device as described in Jenkins et al., Cancer Discov. 2018 February; 8(2):196-215; Ex Vivo Profiling of PD-1 Blockade Using Organotypic Tumor Spheroids, the contents of which is herein incorporated by reference in its entirety. Collagen hydrogels containing patient-derived organotypic tumor spheroids (PDOTS) were hydrated with media with or without anti-Galectin-9 monoclonal antibody G9.2-17 after 30 minutes at 37° C. The PDOTS were then incubated at 37° C. for 3 days.

Cell pellets were re-suspended in the FACS buffer and 1×10⁶ cells were first stained with zombie yellow (BioLegend) to exclude dead cells. After viability staining, cells were incubated with an anti-CD16/CD32 mAb (eBiosciences, San Diego, CA) for blocking FcγRIII/II followed by antibody staining with 1 μg of fluorescently conjugated extracellular mAbs. Intracellular staining for cytokines and transcription factors was performed using the Fixation/Permeabilization Solution Kit (eBiosciences). Useful human flow cytometry antibodies included CD45 (HI30), CD3 (UCHT1), CD4 (A161A1), CD8 (HIT8a), CD44 (BJ18), TNFα (MAb11), IFNγ (4S.B3), and Epcam (9C4); all Biolegend. Flow cytometry was carried out on the LSR-II flow cytometer (BD Biosciences). Data were analyzed using FlowJo v.10.1 (Treestar, Ashland, OR).

### Example 5. Assessment of Galectin-9 Levels in Plasma and Serum of Cancer Patients

Plasma and serum Galectin-9 levels were assessed in patient samples and compared to healthy volunteers. Blood (10 ml) was drawn from peripheral venous access from 10 healthy controls and 10 inoperable cancer patients. Serum and plasma were extracted from each sample of blood. Blood was collected in standard EDTA tubes PicoKine™ ELISA; Catalog number: EK1113 was used essentially according to manufacturer's instructions. Results of individual values are tabulated in Table 12 and Table 13.

TABLE 12
Patient Samples

| | | Serum | Plasma |
|---|---|---|---|
| Cancer Type | Patient No. | (pg/ml) | (pg/ml) |
| Breast cancer with metastases in liver and bones | Patient 1 | 11362.29 | 12107.56 |
| Melanoma brain and lung metastases braf + | Patient 2 | 978.97 | 1106.79 |
| Melanoma lung metastases braf − | Patient 3 | 838.83 | 695.08 |
| Rectal cancer with liver metastases | Patient 4 | 579.42 | 725.62 |
| Locally advanced gastric cancer | Patient 5 | 666.67 | 645.2 |
| Gastric cancer with liver, spleen and adrenal | Patient 6 | 674.3 | 877.69 |
| metastases | | | |
| Stage III ovarian cancer | Patient 7 | 1439.61 | 1341.6 |
| Metaststic canvcer of the unknown primary | Patient 8 | 1432.39 | 1671.8 |
| Testicular cancer | Patient 9 | 1352.56 | 1696.11 |
| Sarcoma | Patient 10 | 968.18 | 1073.57 |
| | Average | 2029.322 | 2194.102 |

TABLE 13
Healthy Volunteer Samples

| Sample Number | Serum | Plasma |
|---|---|---|
| Control 1 | 536.4 | 611.97 |
| Control 2 | 476.43 | 592.58 |
| Control 3 | 612.66 | 651.43 |
| Control 4 | 269.75 | 414.41 |
| Control 5 | 460.26 | 602.28 |
| Control 6 | 206.66 | 405.8 |
| Control 7 | 385.88 | 439.85 |
| Control 8 | 525.283 | 654.2 |
| Control 9 | 711.047 | 718.68 |
| Control 10 | 296.85 | 349.09 |
| Average | 448.122 | 544.029 |

### Example 6. Assessment of Galectin-9 Expression and Localization Using Immunohistochemical Analysis

The ability to use immunohistochemical analysis to determine Galectin-9 expression levels in tumors was assessed using paraffin-embedded biopsy-derived tumor samples.

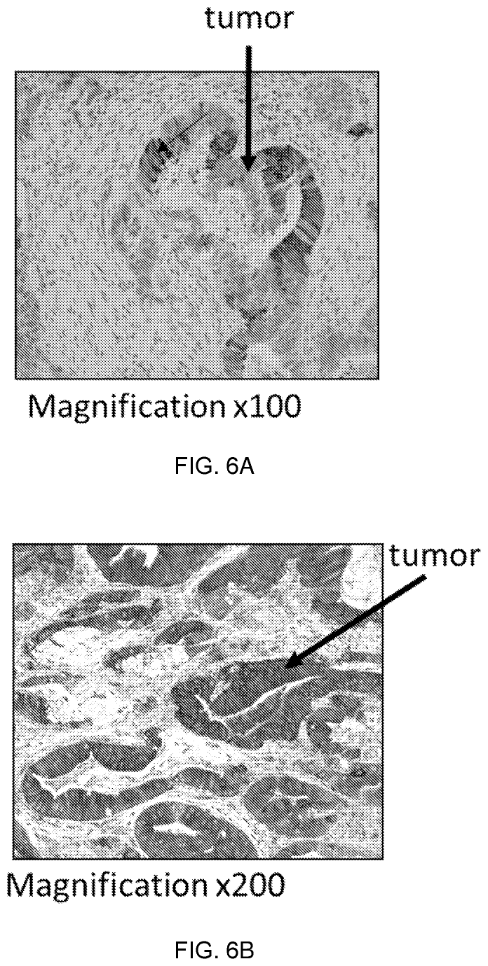

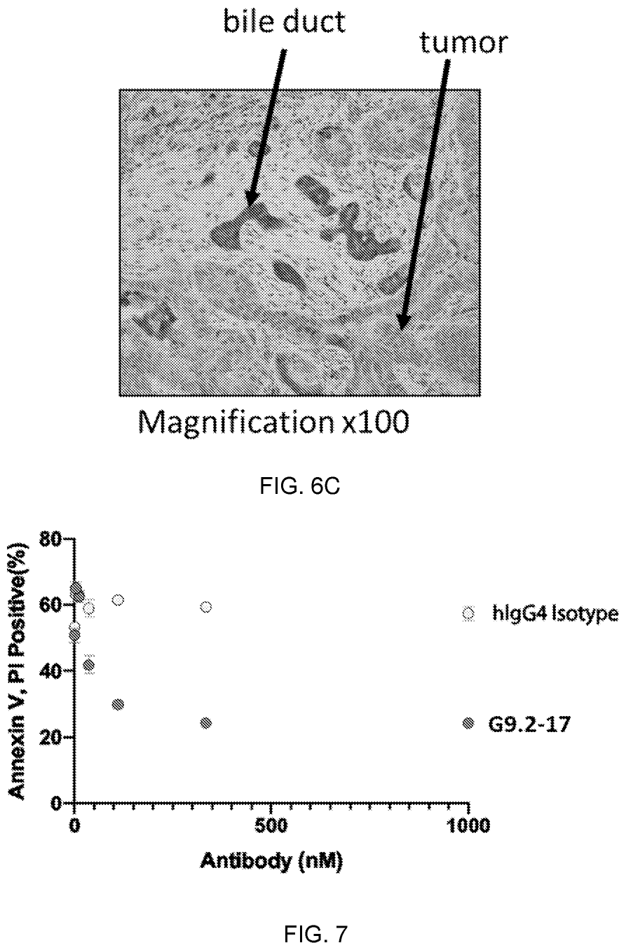

In brief, slides were deparaffinized (xylene: 2×3 min; absolute alcohol: 2×3 min., methanol: 1×3 min) and rinsed in cold tap water. For antigen retrieval, citrate buffer (pH 6) was preheated to 100° ° C. in a water bath and slides were incubated in citrate buffer for 5 minutes. Slides were left to cool for about 10 min at room temperature and put in running water. Slides were washed in PBS, a pap pen circle was drawn around the section, and sections were incubated in blocking buffer (DAKO-Peroxidase blocking solution-S2023) for 5 minutes. Serum free blocker was added (Novocastra serum free Protein Blocker), and then rinsed off with PBS. Primary antibody (Sigma, anti-Galectin-9 clone 1G3) was used at 1:2000 dilution in DAKO-S2022 diluent and sections were incubated over night at 4 C. Slides were washed with PBS and then incubated with the secondary antibody (anti-mouse) for 45 minutes at room temperature. Slides were washed and stained with ABC VECTOR STAIN (45 mins), washed with PBS, stained with DAB (1 ml stable DAB buffer+1 drop DAB)) for 5 minutes and washed in running water. Haematoxylin was added for 1 minute and 70% ETOH+1% HCL was applied to avoid over staining. Slides were left in running water for 2-3 min, then dipped in water, then absolute alcohol, and then xylene, 2 times for 30 seconds each. Cover slip and images were captured. Galectin-9 staining in a chemotherapy treated colorectal cancer and a liver metastasis of colorectal carcinoma are shown in FIGS. 6A-6B. Results from Galectin-9 negative cholangiocarcinoma is shown in FIG. 6C.

### Example 7. Cross-Reactivity of Anti-Galectin-9 Antibody G9.2-17 with Other Galectins

In order to assess antibody specificity and cross-reactivity with other Galectins, anti-Galectin-9 antibody G9.2-17 was tested for binding against a human proteomic array consisting of all members of the Galectin family—and at two working concentrations. Antibody specificity was evaluated using CDI's HuProt Human Proteome Microarray (˜75% of the human proteome). The microarray was incubated with the primary antibody, rinsed, incubated with a fluorescently labelled secondary antibody and subsequently analyzed for the amount of fluorescence detected for each target protein. Results were compiled as microarray images. The results indicated that anti-Galectin-9 antibody G9.2-17 is highly specific to Galectin-9 and does not cross-react with any other Galectin family members.

### Example 8. Anti-Galectin-9 Antibody Protects T Cells from Galectin-9 Mediated Apoptosis

To investigate actions of anti-Galectin-9 antibody G9.2-17, an apoptosis assay was performed to determine if T cells are dying by the process of apoptosis or by other mechanisms.

In brief, MOLM-13 (human leukemia) cells were cultured in RPMI media supplemented with 10% FBS, 2 mM L-glutamine, 10 mM HEPES, 1 mM sodium pyruvate, 4.5 g/L glucose and 1.5 g/L sodium bicarbonate at 37° C. in 5% CO₂. Cells were then transferred into serum-free RPMI media and suspended at a concentration of 2.5e6 cells/mL in serum-free media. Cells were seeded into the wells of a tissue culture grade 96-well plate at a density of 2e5 cells/well (80 μL of cell suspension per well). Monoclonal anti-Galectin-9 antibody or matched isotype was added to each well and incubated at 37° C., 5% CO₂ for 30 min. Following this incubation, recombinant, full length human Galectin-9 (R&D Systems 2045-GA, diluted in PBS) was added to a final concentration of 200 nM. Cells were incubated at 37° C., 5% CO₂ for 16 hours. Cells were then stained with Annexin V-488 and propidium iodide (PI) prior to analysis by flow cytometry. Each condition was performed in triplicate. PI is impermeant to live cells and apoptotic cells, but stains dead cells with red fluorescence, binding tightly to the nucleic acids in the cell. After staining a cell population with Alexa Fluor® 488 annexin V and PI in buffer, apoptotic cells showed green fluorescence, dead cells showed red and green fluorescence, and live cells showed little or no fluorescence. The cells were distinguished using a flow cytometer with the 488 nm line of an argon-ion laser for excitation. Analysis was then performed on FlowJo software. The fraction of annexin V- and propidium iodide (PI)-positive cells is plotted as a function of antibody concentration used in FIG. 7. As shown in FIG. 7, the level of apoptotic T cells treated with the anti-Gal9 antibody was much lower than T cells treated with a human IgG4 isotype control antibody, indicating that the anti-Galectin-9 antibody G9.2-17 protects T cells against galectin-9 mediated cell apoptosis.

### Example 9: Evaluation of Anti-Gal-9 Antibodies Alone or in Combination with Checkpoint Inhibition in a Mouse Model of Pancreatic Cancer and Tumor Mass and Immune Profile of Mice Treated with G9.2-17 mIgG1

The effect of G9.2-17 mIgG1 on tumor weight and on immune profile was assessed in a mouse model of pancreatic cancer. 8-week old C57BL/6 male (Jackson Laboratory, Bar Harbor, ME) mice were administered intra-pancreatic injections of FC1242 PDAC cells derived from Pdx1Cre; KrasG12D; Trp53R172H (KPC) mice (Zambirinis C P, et al., TLR9 ligation in pancreatic stellate cells promotes tumorigenesis. J Exp Med. 2015; 212:2077-94). Tumor cells were suspended in PBS with 50% Matrigel (BD Biosciences, Franklin Lakes, NJ) and 1×10⁵ tumor cells were injected into the body of the pancreas via laparotomy. Mice (n=10/group) received one pre-treatment dose i.p. followed by 3 doses (q.w.) of commercial αGalectin 9 mAb (RG9-1, 200 μg, BioXcell, Lebanon, NH) or G9.2-17 mIgG1 (200 μg), or paired isotype, either G9.2-Iso or rat IgG2a (LTF-2, BioXcell, Lebanon, NH) (200 μg) (one dose per week for three weeks). Mice were sacrificed 3 weeks later, and tumors were harvested for analyses by flow cytometry. Tissue was processed and prepared and flow cytometric analysis was performed following routine practice. See, e.g., U.S. Pat. No. 10,450,374.

Tumor Mass and Immune Profile of Mice Treated with G9.2-17 mIgG2a Alone or in Combination with αPD-1 mAb

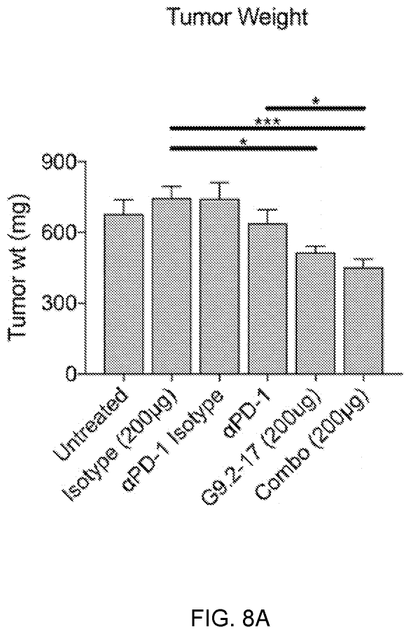

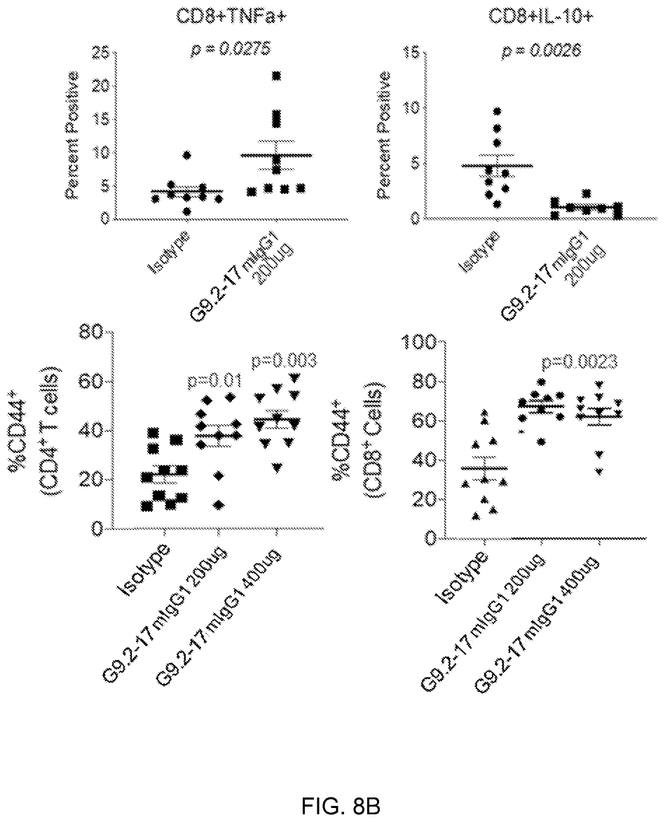

The effect of G9.2-17 mIgG2a on tumor weight and on immune profile was assessed in a mouse model of pancreatic cancer, alone or in combination with immunotherapy. 8-week old C57BL/6 male mice (Jackson Laboratory, Bar Harbor, ME) were administered intra-pancreatic injections of FC1242 PDAC cells derived from Pdx1Cre; KrasG12D; Trp53R172H (KPC) mice. Tumor cells were suspended in PBS with 50% Matrigel (BD Biosciences, Franklin Lakes, NJ) and 1×105 tumor cells were injected into the body of the pancreas via laparotomy. Mice received one pre-treatment dose i.p. followed by 3 doses (q.w.) of G9.2-17 mIgG2a (200 μg) or a neutralizing αPD-1 mAb (29F.1A12, 200 μg, BioXcell, Lebanon, NH), separately or in combination, or paired isotype (LTF-2 and C1.18.4, BioXcell, Lebanon, NH) as indicated. Mice were sacrificed on day 26 and tumors were harvested for analyses. Tissue was processed and prepared and flow cytometric analysis was performed following routine practice. See, e.g., U.S. Pat. No. 10,450,374. Each point represents one mouse; *p<0.05; **p<0.01; ***p<0.001; ****p<0.0001; by unpaired Student's t-test. These results show single-agent treatment with G9.2-17 mIgG2a reduces tumor growth at both of the dose levels, whereas anti-PD-1 alone had no effect on tumor size (FIGS. 8A-8B).

### Example 10: Evaluation of Anti-Gal-9 Antibodies in Two Syngeneic Models of Colorectal and Melanoma Cancer in Immunocompetent Mice

Gal-9 antibodies G9.2-17 and G9.1-8m13 are evaluated in syngeneic models of colorectal and melanoma cancer in immunocompetent mice. Structures of these two antibodies are either provided herein or disclosed in PCT/US2020/024767, the relevant disclosures of which are incorporated by reference for the subject matter and purpose referenced herein. Test articles are formulated and prepared on a weekly basis for the duration of the study.

### Experimental Design

Pre-study animals (female C57BL/6, 6-8 weeks of age (Charles River Labs) are acclimatized for 3 days and then are unilaterally implanted subcutaneously on the left flank with 5e5 B16.F10 (melanoma cell line) or MC38 cells (colorectal cancer cell line) resuspended in 100 μl PBS. Pre-study tumor volumes are recorded for each experiment beginning 2-3 days after implantation. When tumors reach an average tumor volume of 50-100 mm³ (preferably 50-75 mm³) animals are matched by tumor volume into treatment or control groups to be used for dosing and dosing initiated on Day 0. The study design for testing of Anti-Gal9 IgG1 and Anti-Gal9 IgG2 is summarized in Table 14 and Table 15.

TABLE 14
Anti-Gal9 IgG1 (B16F10 and MC38)

| | | | | | Route of | | |
|---|---|---|---|---|---|---|---|
| | | | | | Adminis- | | Total |
| | | | Dose | Dose | tration | | Number |
| Group | -n- | Test Agent | (μg/mouse) | Volume | (ROA) | Schedule | of Doses |
| 1 | 8 | Control Untreated | — | — | — | — | — |
| 2 | 8 | Control mIgG1 | 200 μg | 200 μl | IV | Q4Dx6 | 6 |
| 3 | 8 | Control mIgG1 | 400 μg | 200 μl | IV | Q4Dx6 | 6 |
| 4 | 8 | Control mIgG2 | 200 μg | 200 μl | IP | BIWx4 | 8 |
| 5 | 8 | Anti-Gal9 mIgG1 | 200 μg | 200 μl | IV | Q4Dx6 | 6 |
| 6 | 8 | Anti-Gal9 mIgG1 | 400 μg | 200 μl | IV | Q4Dx6 | 6 |
| 7 | 8 | Anti-Gal9 mIgG1 | 200 μg | 200 μl | IV | Q4Dx6 | 6 |
| | | (G9.1-8m13) | | | | | |
| 8 | 8 | Anti-Gal9 mIgG1 | 400 μg | 200 μl | IV | Q4Dx6 | 6 |
| | | (G9.1-8m13) | | | | | |
| 9 | 8 | Anti-Gal9 mIgG1 | 200 μg | 200 μl | IV IP | Q4Dx6 | 6 8 |
| | | + mAnti-PD-1 | 200 μg | 200 μl | | BIWx4 | |
| 10 | 8 | Anti-Gal9 mIgG1 | 400 μg | 200 μl | IV IP | Q4Dx6 | 6 8 |
| | | + mAnti-PD-1 | 200 μg | 200 μl | | BIWx4 | |
| 11 | 8 | Anti-Gal9 mlgG1 | 200 μg | 200 μl | IV IP | Q4Dx6 | 6 8 |
| | | (G9.1-8m13) + | 200 μg | 200 μl | | BIWx4 | |
| | | mAnti-PD-1 | | | | | |
| 12 | 8 | Anti-Gal9 mIgG1 | 400 μg | 200 μl | IV IP | Q4Dx6 | 6 8 |
| | | (G9.1-8m13) + | 200 μg | 200 μl | | BIWx4 | |
| | | mAnti-PD-1 | | | | | |
| 13 | 8 | mAnti-PD-1 | 200 μg | 200 μl | IP | BIWx4 | 8 |

TABLE 15
Anti-Gal9 IgG2 (B16F10 and MC38)

| | | | | | Route of | | |
|---|---|---|---|---|---|---|---|
| | | | | | Adminis- | | Total |
| | | | Dose | Dose | tration | | Number |
| Group | -n- | Test Agent | (μg/mouse) | Volume | (ROA) | Schedule | of Doses |
| 1 | 10 | Control Untreated | — | — | — | — | — |
| 2 | 10 | Control mIgG2 | 200 μg | 200 μl | IV | Q4Dx6 | 6 |
| 3 | 10 | Control mIgG2 | 400 μg | 200 μl | IV | Q4Dx6 | 6 |
| 4 | 10 | Control mlgG2 | 200 μg | 200 μl | IP | BIWx4 | 8 |
| 5 | 10 | Anti-Gal9 mlgG2 | 200 μg | 200 μl | IV | Q4Dx6 | 6 |
| 6 | 10 | Anti-Gal9 mIgG2 | 400 μg | 200 μl | IV | Q4Dx6 | 6 |
| 5 | 10 | Anti-Gal9 mlgG2 | 200 μg | 200 μl | IV | Q4Dx6 | 6 |
| | | (G9.1-8m13) | | | | | |
| 6 | 10 | Anti-Gal9 mIgG2 | 400 μg | 200 μl | IV | Q4Dx6 | 6 |
| | | (G9.1-8m13) | | | | | |
| 7 | 10 | Anti-Gal9 mlgG2 | 200 μg | 200 μl | IV | Q4Dx6 | 6 8 |
| | | + mAnti-PD-1 | 200 μg | 200 μl | IP | BIWx4 | |
| 8 | 10 | Anti-Gal9 mIgG2 | 400 μg | 200 μl | IV IP | Q4Dx6 | 6 8 |
| | | + mAnti-PD-1 | 200 μg | 200 μl | | BIWx4 | |
| 7 | 10 | Anti-Gal9 mlgG2 | 200 μg | 200 μl | IV | Q4Dx6 | 6 8 |
| | | (G9.1-8m13) + | 200 μg | 200 μl | IP | BIWx4 | |
| | | mAnti-PD-1 | | | | | |
| 8 | 10 | Anti-Gal9 mIgG2 | 400 μg | 200 μl | IV IP | Q4Dx6 | 6 8 |
| | | (G9.1-8m13) + | 200 μg | 200 μl | | BIWx4 | |
| | | mAnti-PD-1 | | | | | |
| 9 | 10 | mAnti-PD-1 | 200 μg | 200 μl | IP | BIWx4 | 8 |

Tumor volumes are taken three times weekly. A final tumor volume is taken on the day the study reaches endpoint. A final tumor volume is taken if an animal is found moribund. Animals are weighed three times weekly. A final weight is taken on the day the study reaches end point or if animal is found moribund. Animals exhibiting ≥10% weight loss when compared to Day 0 are provided DietGel® ad libitum. Any animal exhibiting >20% net weight loss for a period lasting 7 days or if mice display >30% net weight loss when compared to Day 0 is considered moribund and is euthanized. The study endpoint is set when the mean tumor volume of the control group (uncensored) reaches 1500 mm3. If this occurs before Day 28, treatment groups and individual mice are dosed and measured up to Day 28. If the mean tumor volume of the control group (uncensored) does not reach 1500 mm3 by Day 28, then the endpoint for all animals is the day when the mean tumor volume of the control group (uncensored) reaches 1500 mm3 up to a maximum of Day 60. Blood is collected from all animals from each group. For blood collection, as much blood as possible is collected via a cardiac puncture into K₂EDTA tubes (400 μl) and serum separator tubes (remaining) under deep anesthesia induced by isoflurane inhalation. The blood collected into K₂EDTA tubes is placed on wet ice until used for performing immune panel flow.

Blood collected into serum separator tubes is allowed to clot at room temperature for at least 15 minutes. Samples are centrifuged at 3500 for 10 minutes at room temperature. The resultant serum is separated, transferred to uniquely labeled clear polypropylene tubes, and frozen immediately over dry ice or in a freezer set to maintain −80° C. until shipment for the bridging ADA assay (shipped within one week).

Tumors from all animals are collected as follows. Tumors less than 400 mm³ in size are snap frozen, placed on dry ice, and stored at −80° C. until used for RT-qPCR analysis. For tumors of 400-500 mm³ in size, whole tumors are collected into MACS media for use in the Flow Panel. For tumors greater than 500 mm³ in size, a small piece (about 50 mm³) is snap frozen placed on dry ice and stored at −80ºC for RT-qPCR, and the remaining tumor is collected in MACS media for flow cytometry. For flow cytometry, tumors are placed in MACS media and stored on wet ice until processed.

Spleen, liver, colon, lungs, heart, and kidneys from all animals are retained in 10% neutral buffered formalin (NBF) for 18-24 hours, transferred to 70% ethanol and stored at room temperature. Formalin fixed samples are paraffin embedded.

### Example 11: Evaluation of Gal-9 Antibody in a Models of Cholangiocarcinoma

The efficacy of anti-Gal-9 antibody is assessed in a mouse model of cholangiocarcinoma as described in S. Rizvi, et al. (YAP-associated chromosomal instability and cholangiocarcinoma in mice, Oncotarget, 9 (2018) 5892-5905), the contents of which is herein incorporated by reference in its entirety. In this transduction model, in which oncogenes (AKT/YAP) are instilled directly into the biliary tree, tumors arise from the biliary tract in immunocompetent hosts with species-matched tumor microenvironment. Dosing is described in Table 16.

TABLE 16
Dosing

| | | | | | Route of | | |
|---|---|---|---|---|---|---|---|
| | | | | | Adminis | | Total |
| | | | Dose | Dose | tration | | Number |
| Group | -n- | Test Agent | (μg/mouse) | Volume | (ROA) | Schedule | of Doses |
| 1 | 10 | Control Untreated | — | — | — | — | — |
| 2 | 10 | Control mIgG2 | 200 μg | 200 μl | IV | Q4Dx6 | 6 |
| 3 | 10 | Control mIgG2 | 400 μg | 200 μl | IV | Q4Dx6 | 6 |
| 4 | 10 | Control mIgG2 | 200 μg | 200 μl | IP | BIWx4 | 8 |
| 5 | 10 | Anti-Gal9 mIgG2 | 200 μg | 200 μl | IV | Q4Dx6 | 6 |
| | | (G9.2-17) | | | | | |
| 6 | 10 | Anti-Gal9 mIgG2 | 400 μg | 200 μl | IV | Q4Dx6 | 6 |
| | | (G9.2-17) | | | | | |
| 7 | 10 | Anti-Gal9 mIgG2 | 200 μg | 200 μl | IV | Q4Dx6 | 6 |
| | | (G9.1.8-m13) | | | | | |
| 8 | 10 | Anti-Gal9 mIgG2 | 400 μg | 200 μl | IV | Q4Dx6 | 6 |
| | | (G9.1.8-m13) | | | | | |

In brief, murine CCA cells (described in S. Rizvi, et al) are harvested and washed in DMEM. Male C57BL/6 mice from Jackson Labs are anesthetized using 1.5-3% isoflurane. Under deep anesthesia, the abdominal cavity is opened by a 1 cm incision below the xiphoid process. A sterile cotton tipped applicator is used to expose the superolateral aspect of the medial lobe of the liver. Using a 27-gauge needle, 40 μL of standard media containing 1×10\hat{ }6 cells is injected into the lateral aspect of the medial lobe. Cotton tipped applicator is held over the injection site to prevent cell leakage and blood loss. Subsequently, the abdominal wall and skin are closed in separate layers with absorbable chromic 3-0 gut suture material.

Two weeks post implantation, animals are matched by tumor volume into treatment or control groups to be used for dosing and dosing initiated on Day 0. Tumor volumes are measured, and animals weighed three times weekly. A final tumor volume and weight is taken on the day the study reaches endpoint (4 weeks or when tumor burden of control becomes 1500 mm3). Blood is collected from all animals from each group.

### Example 12: In Vitro and In Vivo Characterization of Anti-Gal9 Antibody G9.2-17

In vivo and in vitro pharmacodynamics and pharmacology studies and safety pharmacology were conducted as disclosed below. In vivo studies were conducted with an IgG1 version of anti-galectin-9 mAb G9.2-17 for mouse studies based on the fact that this antibody was developed to have the exact same V_{H} and V_{L} chains and thus the exact same binding epitope as G9.2-17 and the same cross reactivity profile as well as binding affinities across species and same functional profile like G9.2-17.

### In Vitro Studies

G9.2-17 has multi-species cross-reactivity (human, mouse, rat, cynomolgus monkey), with equivalent <1 nmol binding affinities, as assessed in vitro. See, e.g., PCT/US2020/024767, the relevant disclosures of which are incorporated by reference for the subject matter and purpose as referenced herein. G9.2-17 does not cross react with the CRD1 domain of galectin-9 protein. It has excellent stability and purification characteristics, and no cross-reactivity to any of the other galectin proteins that exist in primates.

Table 17 below summarizes results from in vitro pharmacology studies.

TABLE 17
In Vitro Primary Pharmacodynamics

| Objective | Assays | Key Results |
|---|---|---|
| Bead based | Binding of G9.2-17 to | Bead based measurements of G9.2-17 binding to the |
| binding - human | CRD1 and CRD2 | human galectin-9 CRD1 and CRD2 domains show |
| | domain of human | that G9.2-17 is specific to only the human CRD2 |
| | galectin-9 | domain of galectin-9. The mouse IgG1 version of |
| | | G9.2-17 show similar specificity to only the CRD2 |
| | | domain of galectin-9. KD Values (nM): G9.2-17 = |
| | | 0.15 ± 0.02, G9.2-17 mIgG1 = 0.18 ± 0.02. |
| Bead based | Binding of G9.2-17 to | Bead based measurements of G9.2-17 binding to the |
| binding - mouse | CRD2 domain of mouse | mouse galectin-9 CRD2 domain show that G9.2-17 |
| | galectin-9 | binds with <1 nMol to the mouse CRD2 domain. |
| | | The mouse IgG1 version of G9.2-17 show similar |
| | | affinity to the CRD2 domain of mouse galectin-9. |
| | | KD Values (nM): G9.2-17 = 0.30 ± 0.03; G9.2-17 |
| | | mIgG1 = 0.30 ± 0.1. |
| Bead based | Binding of G9.2-17 to | Bead based measurements of G9.2-17 binding to the |
| binding - rat | CRD2 domain of rat | rat galectin-9 CRD2 domain show that G9.2-17 binds |
| | galectin-9 | with <1 nMol to the rat CRD2 domain. The mouse |
| | | IgG1 version of G9.2-17 show similar affinity to the |
| | | CRD2 domain of rat galectin-9. KD Values (nM): |
| | | KD Values (nM): G9.2-17 = 0.31 ± 0.06; G9.2-17 |
| | | mIgG1 = 0.35 ± 0.06. |
| Bead based | Binding of G9.2-17 to | Bead based measurements of G9.2-17 binding to the |
| binding - | CRD2 domain of | cynomolgus galectin-9 CRD2 domain show that |
| cynomolgus | cynomolgus monkey | G9.2-17 binds with <1 nMol to the cynomolgus |
| monkey | galectin-9 | CRD2 domain. The mouse IgG1 version of G9.2-17 |
| | | show similar affinity to the CRD2 domain of |
| | | cynomolgus galectin-9. KD Values (nM): G9.2-17 = |
| | | 0.31 ± 0.03; G9.2-17 mIgG1 = 0.30 ± 0.10. |
| Binding - ELISA | ELISA based binding | G9.2-17 binding to human Galectin-9 CRD2 was |
| | assessment of G9.2-17 | assessed in ELISA format over a concentration |
| | to human CRD2 domain | range. G9.2-17 was titrated over immobilized |
| | of galectin-9 | Galectin-9 CRD2 and the resultant saturation curve |
| | | indicates that G9.2-17 has < 1 nMol to the CRD2 |
| | | domain of galectin-9. |
| | | The mouse IgG1 version of G9.2-17 show similar |
| | | affinity to the CRD2 domain of galectin-9 when |
| | | assayed in this format. |
| | | KD Values (nM): G9.2-17 = 0.42 ± 0.07; G9.2-17 |
| | | mIgG1 = 0.45 ± 0.04. |
| Binding -SPR | SPR based binding | SPR measurements using the OneStep method on a |
| human | assessment of G9.2-17 | Pioneer SPR showed high binding of G9.2-17 to |
| | to human CRD2 domain | human galectin-9 CRD2. The resultant binding |
| | of galectin-9 | between the antibody and immobilized human |
| | | galectin-9 CRD2 had no measurable off rate even |
| | | after continued dissociation for over 30 minutes. |
| | | This suggests that G9.2-17 has a KD below the |
| | | measurable limit of assay. The mouse IgG1 version |
| | | of G9.2-17 showed similar behavior, with no |
| | | measurable off rate even over an extended |
| | | dissociation time. KD Values (nM): G9.2-17 = below |
| | | limit of detection; G9.2-17 mIgG1 = below limit of |
| | | detection. |
| Binding - SPR | SPR based binding | SPR measurements using the OneStep method on a |
| mouse | assessment of G9.2-17 | Pioneer SPR showed high binding of G9.2-17 to |
| | to mouse CRD2 domain | mouse galectin-9 CRD2. Binding of G9.2-17 to |
| | of galectin-9 | mouse galectin-9 CRD2 had a KD value of 1.8 ± 0.4 |
| | | nM. The mouse IgG1 version of G9.2-17 showed |
| | | similar behavior, with a KD- value of 3.05 ± 0.03 nM. |
| | | KD Values (nM): G9.2-17 = 1.8 ± 0.4; G9.2-17 |
| | | mIgG1 = 3.05 ± 0.03. |
| Binding - Cell- | Assessment of cell | An assessment of G9.2-17 binding to galectin-9 on |
| based | surface based (CRL- | the cell surface was performed using the galectin-9 |
| | 2134 cell line) binding | positive CRL-2134 cell line. First, staining of |
| | of G9.2-17 | CRL2134 with G9.2-17 showed increased signal |
| | | compared to staining of the galectin-9 negative HEK- |
| | | 293 cell line. A saturation curve was then generated |
| | | by titrating G9.2-17 for surface staining of CRL- |
| | | 2134 cells. The curve was generated based on the |
| | | fraction of the cell population that were positive for |
| | | galectin-9 as compared unstained cells. Using the |
| | | generated saturation curve, a cell based KD of |
| | | 0.41 ± 0.07 nM was calculated. This assay was also |
| | | performed with the mouse IgG1 variant of G9.2-17 |
| | | with a resulting cell-based KD of 2.9 ± 0.7 nM. |
| Cell-based | G9.2-17 potency | MOLM-13 cells are sensitive to high concentrations |
| potency | assessment using | of human galectin-9. Incubation of MOLM-13 cells |
| T-cell apoptosis | MOLM-13 T cell-based | for 16 h in the presence of 200 nM galectin-9 results |
| | apoptosis assay | in significant cell death. The addition of G9.2-17 |
| | | protects MOLM-13 from galectin-9 mediated cell |
| | | death in a dose dependent manner, significantly |
| | | reducing the population of necrotic cells. This effect |
| | | is specific for G9.2-17 as well as the mouse IgG1 |
| | | variant of G9.2-17 while the matched human IgG4 |
| | | and mouse IgG1 isotypes show no protection against |
| | | galectin-9 mediated cell death. |
| Non-cell based | G9.2-17 potency | The receptor-ligand interaction between CD206 and |
| potency | assessment using non- | galectin-9 was assayed in ELISA format. Full length |
| T-cell apoptosis | cell based, competition | galectin-9 was immobilized and recombinant, His- |
| | ELISA CD206 binding | tagged CD206 was titrated to confirm CD206 does |
| | assay | bind to galectin-9. In order to determine whether or |
| | | not G9.2-17 blocked the binding between galectin-9 |
| | | and its native receptor CD206, a competitive ELISA |
| | | assay was utilized. Blockade of the galectin-9- |
| | | CD206 interaction resulted in reduced ELISA signal |
| | | compared to the unblocked condition in a dose |
| | | dependent manner. |
| Functional assay: | Bead based | G9.2-17 does not mediate ADCC or ADCP activity. |
| non-cell based | ADCC/ADCP assay | |
| ADCC/ADCP | | |
| assay | | |
| Protein array - | Protein Array - Cross | HuProt ™ array was used for the High-Spec ® |
| cross reactivity | reactivity | antibody cross-reactivity assay. Arrays contained |
| | | native and not denatured proteins. G9.2-17 |
| | | recognized galectin-9 (CDI clone or the positive |
| | | control antigen) as the top hit with high affinity. |
| Expression | Assessing cell surface | Dose dependent effect was observed in detection of |
| | and intra-cellular | cell surface galectin-9 on KPC cells, peaking at 20% |
| | galectin-9 levels by flow | using 60 nM G9.2-17 Fab. Intracellular galectin-9 |
| | cytometry on | expression was uniformly detected in 10% of the |
| | permeabilized and non | cells at 15 nM, 30 nM and 60 nM of G9.2-17 Fab. |
| | permeabilized mouse | |
| | pancreatic cancer (KPC) | |
| | cells | |
| Expression | Assessing cell surface | 27.6% of B16F10 express galectin-9 on their surface |
| | and intra-cellular | and 98.8% intracellularly. 6.9% of MC38 express |
| | galectin-9 levels by flow | galectin-9 on their surface and 41.5% intracellularly. |
| | cytometry on | |
| | permeabilized and non | |
| | permeabilized mouse | |
| | melanoma (B16F10) and | |
| | colorectal cancer | |
| | (MC38) cells | |
| Mechanism of | Patient derived tumor | Activation of T cells measured through IFNg, TNFα |
| Action | cultures ex vivo | and CD44. n = 20 tumors processed. T cell |
| | (organoids) treated with | reactivation from baseline observed in n = 12 out of |
| | G9.2-17 | 20 (60%) of tumors processed. |
| Expression | Patient derived tumor | T cells galectin-9 expression (12.5-63.7% |
| | cultures ex vivo | CD3+CD45+ intra PTOD T cells). Myeloid cell |
| | (organoids) profiling for | galectin-9 expression (15-45.9% CD45+CD11b+ |
| | galectin-9 expression on | intra PDOT myelod cells). Tumor cell galectin-9 |
| | T cells, tumor cells and | expression (9.15-33.5% CD45-EpCAM+ intra PDOT |
| | macrophages | tumor cells) n = 6 PDOTs |
| Expression | Measuring galectin-9 | Sera from healthy controls (n = 16) and cancer |
| | levels in serum of | patients (n = 22; n = 10 primary and n = 12 |
| | healthy controls and | metastatic) with gastrointestinal malignancies. |
| | cancer patients | Galectin-9 serum levels are significantly increased in |
| | | cancer patients vs controls (p = 0.001) |
| Expression | Measuring galectin-9 | Sera and plasma from healthy controls (n = 10) and |
| | levels in serum and | cancer patients (n = 10) with metastatic tumors of |
| | plasma of healthy | diverse site of origin was tested for galectin-9 |
| | controls and cancer | expression. |
| | patients | |

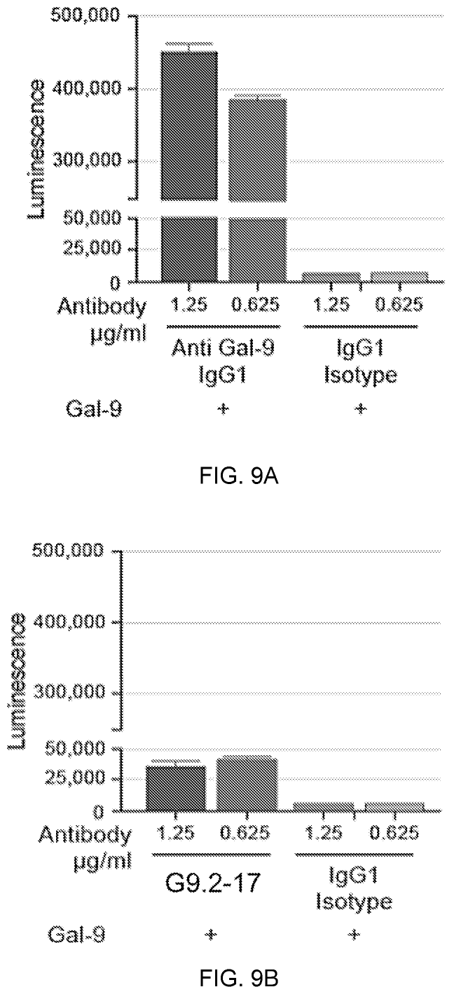

Studies to understand the mechanism of action included ADCC/ADCP (antibody dependent cell mediated cytotoxicity/antibody-dependent cellular phagocytosis) and blocking function assessment. As expected for a human IgG4 mAb, G9.2-17 does not mediate ADCC or ADCP (FIG. 9A). This was tested against the IgG1 human counterpart of G9.2-17 as a positive control, which mediates ADCC and ADCP, as expected (FIG. 9B).

Furthermore, blocking function of G9.2-17 was evaluated in a competition binding ELISA assay. G9.2-17 potently blocks binding of galectin-9 CRD2 domain to its binding partner CD206 human recombinant protein, confirming the intended mode of action for G9.2-17, which is to block galectin-9 activity. Moreover, we optimized a MOLM-13 T cell apoptosis assay where G9.2-17 proficiently rescues the cells from apoptosis caused by galectin-9 protein treatment (˜50% apoptosis with galectin-9 treatment and ˜10% apoptosis with galectin-9+G9.2-17 treatment).

Further extensive in vitro characterization has been done to compare binding and functional characteristics of G9.2-17 to the mouse IgG1 G9.2-17 mAb, which contains exactly the same CDR domains as G9.2-17, hence has the same binding epitope, i.e., CRD2 galectin-9 domain. mIgG1 G9.2-17 was developed for use in mouse syngeneic pharmacology efficacy studies, to avoid any potential development of immunogenicity with G9.2-17 itself. mIgG1 G9.2-17 has equivalent <1 nmol affinity across species, as well as the same cell based binding affinity to human cancer cell line, CRL-2134. mIgG1 G9.2-17 produces equivalent data in the MOLM-13 T cell apoptosis assay, as G9.2-17 itself.

### In Vivo Pharmacology

In vivo assays include syngeneic mouse models conducted using a mouse mAb-G9.2-17 binding epitope cloned into an IgG1 mouse backbone (G9.2-17 surrogate mAb for animal efficacy studies), which shares the cross reactivity and binding affinity characteristics of G9.2-17.

Syngeneic mouse models tested were:
Orthotopic pancreatic adenocarcinoma (KPC) mouse model (single agent and in combination with anti-PD-1): survival, tumor volume assessment and flow cytometry.
Subcutaneous melanoma B16F10 model (single agent and in combination with anti-PD-1): tumor volume assessment and flow cytometry.
Subcutaneous MC38 model (single agent and in combination with anti-PD-1): tumor volume assessment

Further, patient-derived tumor cultures ex vivo (organoids) treated with G9.2-17 are to be used for exploring mechanism of action of G9.2-17.

Mechanistically, G9.2-17 was found to have blocking activity and not ADCC/ADCP activity. Blocking of galectin-9 interactions with its binding receptors, such as CD206 on immunosuppressive macrophages, is observed. Functionally, in vivo studies demonstrated reduction of tumor growth in multiple syngeneic models treated with G9.2-17 mIgG1 surrogate antibody (orthotopic pancreatic KPC tumor growth and s.c. melanoma B16F10 model). In mouse tumors treated with single agent anti-galectin-9 mAb and in combination with anti-PD-1, G9.2-17 reactivates effector T cells and reduces levels of immunosuppressive cytokines. Combination studies with an anti-PD-1 mAb suggest higher intra-tumoral presence of effector T cells, supporting clinical testing of the combinatorial approach. Importantly, mechanistic effects of G9.2-17 have been investigated and demonstrated in patient derived tumor cultures (Jenkins et al., 2018) (tumor excisions from primary and metastatic sites from PDAC, CRC, CCA, HCC), where G9.2-17 induces reproducible and robust T cell reactivation, indicating reversal of galectin-9 imposed intra-tumoral immunosuppression ex vivo.

In order to assess relevance of combining anti-PD-1 and anti-galectin-9 mAbs, s.c. melanoma B16 model was treated with single agent anti-PD-1 and anti-galectin-9 as well as the combination. Intra-tumoral presence effector T cells were enhanced in the combination arm.

Significant increases in the level of cytotoxic T cells (CD8) are observed in treatments with anti-galectin-9 mIgG1 200 μg+anti-PD-1 (p<0.001) compared to that of anti-galectin-9 mIgG1 200 μg, and between anti-galectin-9 IgG1 200 μg+anti-PD-1 compared to anti-PD-1 alone (p<0.01). Such results suggest that anti-Gal9 antibody and anti-PD-1 antibody in combination would be expected to achieve superior therapeutic effects.

Table 18 below summarizes results from in vivo pharmacology studies.

TABLE 18
In Vivo Primary Pharmacodynamics

| Study Title | Test System | Key Results |
|---|---|---|
| Efficacy study assessing tumor | Orthotopic | Efficacy observed with single agent IgG1 |
| volume and flow cytometry of intra- | KPC model | mouse galectin-9 mAb, p = 0.05. Flow |
| tumoral immune cells in mice | | cytometry: CD8 T cells: Increase in CD8+ T |
| treated with IgG1 mouse anti- | | cell TNF alpha (p = 0.027), increase in |
| galectin-9 mAb at 150 μg/dose i.p. | | CD8+ T cell CD44 (p = 0.0008) and |
| | | reduction in CD8+ T cell IL10 (p = 0.0026). |
| | | Increase in CD4+ T Cell TNF alpha (p = |
| | | 0.0007). |
| Efficacy study assessing tumor | Orthotopic | Efficacy observed at 200 μg (p = 0.0005) and |
| volume and flow cytometry of intra- | KPC model | 400 μg (p = 0.01) dose levels of single agent |
| tumoral immune cells in mice | | anti-galectin-9 mIgG1 mAb. Flow |
| treated with IgG1 mouse anti- | | cytometry: CD8+ T cells: increase of CD44 |
| galectin-9 mAb at 200 and 400 μg/ | | (for dose levels 200 μg and 400 μg p = |
| dose i.p. | | 0.002). CD4+ T cells: Increase in CD44 (for |
| | | dose level 200 μg, p = 0.015 and for dose |
| | | level 400 μg p = 0.0003). |
| Efficacy study assessing tumor | orthotopic | Efficacy observed at both dose levels (p < |
| volume and flow cytometry of intra- | KPC model | 0.01). Flow cytometry: CD4+ T cells: |
| tumoral immune cells in mice | | increase in CD44 (p < 0.0001), PD-1 (for |
| treated with IgG1 mouse anti- | | dose level 100 μg p = 0.005 and for dose |
| galectin-9 mAb at 100 and 200 μg/ | | level 200 μg p = 0.001); CD8+ T cells: |
| dose i.p. | | increase in CD44 (p < 0.0001). |
| Efficacy study assessing tumor | Orthotopic | Efficacy observed at 50 μg (p < 0.05) and |
| volume in mice treated with IgG1 | KPC model | 100 μg (p < 0.0001) dose levels and no |
| mouse anti-galectin-9 mAb at 20, | | significant efficacy at 20 μg/dose. No |
| 50 and 100 μg/dose i.p. + 100 μg/ | | significant TV synergy effect with |
| dose IgG1 mouse anti-galectin-9 | | combination of 100 μg anti-galectin-9 mAb |
| mAb with anti-PD-1 | | and anti-PD-1 |
| Efficacy study assessing tumor | Sub cutaneous | Highest efficacy observed at 200 μg (p < |
| volume and flow cytometry in mice | B16F10 | 0.005) single agent mouse anti-galectin-9 |
| treated with IgG1 mouse anti- | model | mAb, superior to anti-PD-1 mAb. No |
| galectin-9 mAb at 200 and 400 μg/ | | significant TV synergy effect with |
| dose i.v. + anti-PD-1 mAb | | combination of 200 μg anti-galectin-9 mAb |
| | | and anti-PD-1 on tumor growth. However, |
| | | significant increase in cytotoxic CD8 T cell |
| | | levels were observed in mouse anti-galectin- |
| | | 9 mAb + anti-PD-1 mAb (p < 0.01). |
| Efficacy study assessing tumor | Sub cutaneous | Efficacy not superior to anti-PD-1 mAb in |
| volume in mice treated with IgG1 | MC38 model | this model. Combination with anti-PD-1 is |
| mouse anti-galectin-9 mAb at 200 | | equivalent to anti-PD-1 alone. Please refer to |
| and 400 μg/dose i.v. + anti-PD-1 | | CFCH001 for flow cytometry data |
| mAb | | explaining low expression of galectin-9 on |
| | | MC38 cells. |

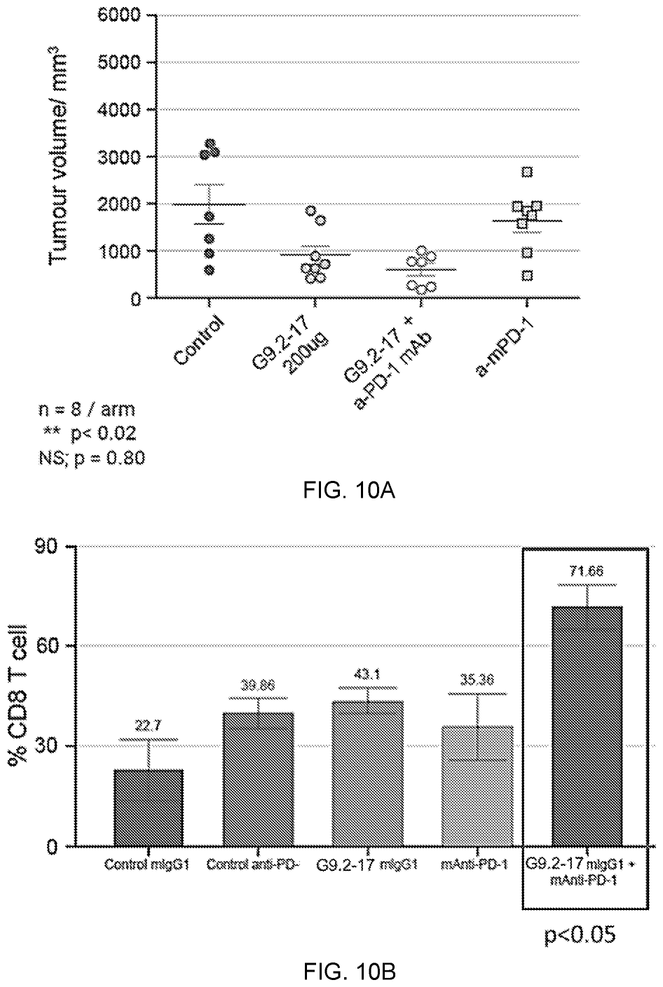

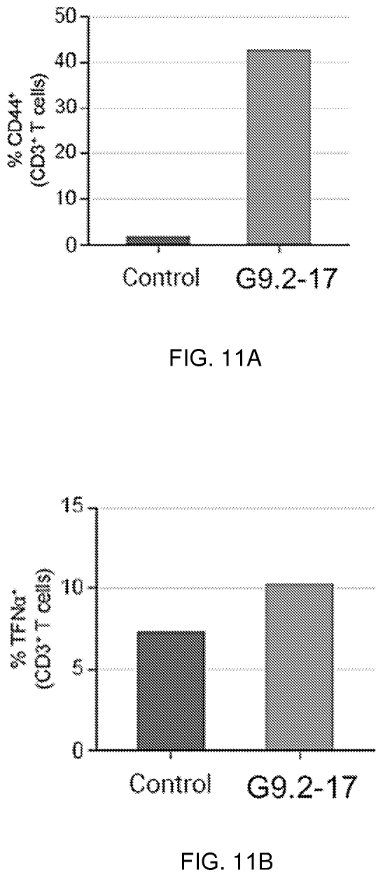

Further, tumor immune responses to treatment with G9.2-17 IgG1 mouse mAb (aka G9.2-17 mIgG), anti-PD-1 antibody, or a combination of the G9.2-17 IgG1 mouse mAb and anti-PD-1 antibody were investigated in the B16F10 subcutaneous syngeneic model described herein. As shown in FIG. 10A-10B, the G9.2-17 and anti-PD-1 combination showed synergistic effects in reducing tumor volume and in increasing CD8+ cells in the mouse model. FIGS. 11A-11B show that the G9.2-17 antibody increased CD44 and TNFa expression in intratumoral T cells.

### Example 13. A Non-GLP Single-Dose, Range-Finding Intravenous Toxicity Study in Male Sprague Dawley Rats with 1- and 3-Week Postdose Observation Periods

This study evaluated the anatomical endpoints of G9.2-17 IgG4 following a single intravenous bolus administration to Sprague Dawley rats followed by 1-week (terminal) and 3-week (recovery) necropsies on Days 8 and 22. All animals survived to the scheduled necropsies. There were no test article-related macroscopic findings, organ weight changes, or microscopic findings in either the terminal or recovery necropsy animals on this study.

The objective of this non-GLP exploratory, single-dose, range finding, intravenous toxicity study was to identify and characterize the acute toxicity G9.2-17 IgG4 following intravenous bolus administration over 2 minutes to Sprague Dawley rats followed by 1-week (terminal) and 3-week (recovery) postdose observation periods.

This non-GLP single dose toxicity study was conducted in 24 Sprague Dawley male rats to determine the toxicokinetics and potential toxicity of G9.2-17 IgG4. Animals were administered either vehicle or 10 mg/kg, 30 mg/kg or 70 mg/kg G9.2-17 IgG4 by slow bolus intravenous infusion for at least 2 minutes on Day 1 followed by either a 1-week (terminal, Day 8) or 3-week (recovery, Day 22) period after the dose. Study endpoints included mortality, clinical observations, body weights, and food consumption, clinical pathology (hematology, coagulation, clinical chemistry and urinalysis), toxicokinetic parameters, ADA evaluation and anatomic pathology (gross necropsy, organ weights, and histopathology). Summaries of the experimental design is provided in Table 19 below.

TABLE 19
Experimental Design

| Group | | Dosage Level | |
|---|---|---|---|
| Number | Treatment | (mg/kg) | Number of Males ^{a} |
| 1 | Vehicle ^{b} | 0 | 6 |
| 2 | G9.2-17 IgG4 | 10 | 6 |
| 3 | G9.2-17 IgG4 | 30 | 6 |
| 4 | G9.2-17 IgG4 | 70 | 6 |

^{a} 3 animals/sex/group were euthanized at the Day 8 terminal necropsy; the remaining 3 animals/sex/group were euthanized at the Day 22 recovery necropsy.
^{b} The vehicle was Formulation Buffer (20 mM Tris, 150 mM NaCl, pH 8.0 ± 0.05).

All surviving animals were submitted for necropsy on Day 8 or Day 22. Complete postmortem examinations were performed, and organ weights were collected. The organs were weighed from all animals at the terminal and recovery. Tissues required for microscopic evaluation were trimmed, processed routinely, embedded in paraffin, and stained with hematoxylin and eosin.

There were no unscheduled deaths during the course of this study. All animals survived to the terminal or recovery necropsies. Histological changes noted were considered to be incidental findings or related to some aspect of experimental manipulation other than administration of the test article. There was no test article related alteration in the prevalence, severity, or histologic character of those incidental tissue alterations. No G9.2-17 IgG4-related findings were noted in clinical observations, body weights, food consumption, clinical pathology or anatomic pathology. In conclusion, the single intravenous administration of 10, 30, and 70 mg/kg G9.2-17 IgG4 to Sprague Dawley rats was tolerated with no adverse findings. Therefore, under the conditions of this study the NOEL was 70 mg/kg.

### Example 14. A Non-GLP Single-Dose, Range-Finding Intravenous Infusion Toxicity Study of G9.2-17 IgG4 in Cynomolgus Monkeys with a 3-Week Post-Dose Observation Period

This non-GLP single-dose toxicity study was conducted in 8 cynomolgus monkeys to identify and characterize the acute toxicities of G9.2-17 IgG4. Animals (1 male [M]/1 female [F]/group) were administered either vehicle or 30 mg/kg, 100 mg/kg, or 200 mg/kg G9.2-17 IgG4 by 30-minute intravenous (IV) infusion followed by a 3-week post-dose observation period. Study endpoints included: mortality, clinical observations, body weights, and qualitative food consumption; clinical pathology (hematology, coagulation, clinical chemistry, immunophenotyping and galectin 9 expression on leukocyte subsets, and cytokine analysis); toxicokinetic parameters; serum collection for possible anti-drug antibody evaluation (ADA); and soluble galectin-9 analyses; and anatomic pathology (gross necropsy, organ weights, and histopathology).

No G9.2-17 IgG4-related findings were noted in clinical observations, body weights, food consumption, clinical pathology (hematology, clinical chemistry, coagulation, or cytokine analysis), immunophenotyping, galectin-9 expression on leukocyte subsets, soluble galectin-9 or anatomic pathology.

In conclusion, the single intravenous infusion administration of 30, 100, and 200 mg/kg G9.2-17 IgG4 to cynomolgus monkeys was tolerated with no adverse findings. Therefore, under the conditions of this study the No-observed-Adverse-Effect-Level (NOAEL) was 200 mg/kg, the highest dose level evaluated. The study design is shown in Table 20.

TABLE 20
Experimental Design

| | | | | | | Dose Volume | | Animal |
|---|---|---|---|---|---|---|---|---|
| | | | | Adjusted Dose | | (mL/kg) | | No. |
| Group | | Dose Level | | Concentration (mg/mL) | | Necropsy | | Necropsy |
| No. | Treatment | (mg/kg) | | | Males | Day | Females | Day |
| 1 | Vehicle | 0 | 0 | 20 | 1001 | 22 | 1501 | 22 |
| 2 | G9.2-17 | 30 | 1.5 | 20 | 2001 | 22 | 2501 | 22 |
| | IgG4 | | | | | | | |
| 3 | G9.2-17 | 100 | 5 | 20 | 3001 | 22 | 3501 | 22 |
| | IgG4 | | | | | | | |
| 4^{a} | G9.2-17 | 200 | 10 | 20 | 4001 | 22 | 4501 | 22 |
| | IgG4 | | | | | | | |

^{a}Group 4 was administered 1 week after administration of Groups 1 through 3.

| | | | Adjusted | | | | | |
|---|---|---|---|---|---|---|---|---|
| | | Dose | Dose | Dose | Animal No. | | | |
| Group | | Level | Concentration | Volume | | Necropsy | | Necropsy |
| No. | Treatment | (mg/kg) | (mg/mL) | (mL/kg) | Males | Day | Females | Day |
| 1 | Vehicle | 0 | 0 | 20 | 1001 | 22 | 1501 | 22 |
| 2 | G9.2-17 | 30 | 1.5 | 20 | 2001 | 22 | 2501 | 22 |
| | IgG4 | | | | | | | |
| 3 | G9.2-17 | 100 | 5 | 20 | 3001 | 22 | 3501 | 22 |
| | IgG4 | | | | | | | |
| 4^{a} | G9.2-17 | 200 | 10 | 20 | 4001 | 22 | 4501 | 22 |
| | IgG4 | | | | | | | |

^{a}Group 4 was administered 1 week after administration of Groups 1 through 3.

The vehicle and test article were administered once via IV infusion for 30 minutes during the study via a catheter percutaneously placed in the saphenous vein. The dose levels were 30, 100, and 200 mg/kg and administered at a dose volume of 20 mL/kg. The control group received the vehicle in the same manner as the treated groups.

The animals were placed in sling restraints during dosing. The vehicle or test article were based on the most recent body weights and administered using an infusion pump and sterile disposable syringes. The dosing syringes were filled with the appropriate volume of vehicle or test article (20 mL/kg with 2 mL extra). At the completion of dosing, the animals were removed from the infusion system. The weight of each dosing syringe was recorded prior to the start and end of each infusion to determine dose accountability.

### Detailed Clinical Observations

The animals were removed from the cage, and a detailed clinical examination of each animal was performed at 1 hour and 4.5 hours post-start of infusion (SOI) on Day 1 and once daily thereafter during the study. The animals were removed from the cage, and a detailed clinical examination of each animal was performed at 1 hour and 4.5 hours post-start of infusion (SOI) on Day 1 and once daily thereafter during the study. Body weights for all animals were measured and recorded at transfer, prior to randomization, on Day −1, and weekly during the study.

Clinical pathology evaluations (hematology, coagulation, and clinical chemistry) were conducted on all animal pretest and on Days 1 (prior to dosing), 3, 8, and 21. Additional samples for the determination of hematology parameters and peripheral blood lymphocyte and cytokine analysis samples were collected at 30 minutes (immediately after the end of infusion) and 4.5, 8.5, 24.5, and 72.5 hours post-SOI (relative to Day 1). Bone marrow smears were collected and preserved.

Blood samples (approximately 0.5 mL) were collected from all animals via the femoral vein for determination of the serum concentrations of the test article (see Table 21). The animals were not fasted prior to blood collection, with the exception of the intervals that coincided with fasting for clinical pathology collections.

TABLE 21
Bioanalysis Sample Collection Schedule

| | Sample Collection Time Points | | | | | | | | | | | | |
|---|---|---|---|---|---|---|---|---|---|---|---|---|---|
| | (Time Post-SOI) relative to Day 1 | | | | | | | | | | | | |
| | | | | | | | 24.5 | 48.5 | 72.5 | 120.5 | 168.5 | 360.5 | 504.5 |
| Group | Pre | 0.583 | 1 | 2.5 | 4.5 | 8.5 | hr | hr | hr | hr | hr | hr | hr |
| No. | dose | hr^{a} | hr | hr | hr | hr | (Day 2) | (Day 3) | (Day 4) | (Day 6) | (Day 8) | (Day 16) | (Day 22) |
| 1-4 | X | X | X | X | X | X | X | X | X | X | X | X | X |

X = Sample was collected.
^{a}Only the 0.583 hr post-SOI timepoint from Group 1 animals was analyzed for test article content.
Additional timepoints may be analyzed at the discretion of the Study Director.

For processing, blood samples were collected in non-additive barrier free microtubes and centrifuged at controlled room temperature within 1 hour of collection. The resulting serum was divided into 2 approximately equal aliquots in pre labeled cryovials. All aliquots were stored frozen at −60° C. to −90° ° C. within 2 hours of collection.

Postmortem study evaluations were performed on all animals euthanized at the scheduled necropsy.

Necropsy examinations were performed under procedures approved by a veterinary pathologist. The animals were examined carefully for external abnormalities including palpable masses. The skin was reflected from a ventral midline incision and any subcutaneous masses were identified and correlated with antemortem findings. The abdominal, thoracic, and cranial cavities were examined for abnormalities. The organs were removed, examined, and, where required, placed in fixative. All designated tissues were fixed in neutral buffered formalin (NBF), except for the eyes (including the optic nerve) and testes. The eyes (including the optic nerve) and testes were placed in a modified Davidson's fixative, and then transferred to 70% ethanol for up to three days prior to final placement in NBF. Formalin was infused into the lung via the trachea. A full complement of tissues and organs was collected from all animals.

Body weights and protocol-designated organ weights were recorded for all animals at the scheduled necropsy and appropriate organ weight ratios were calculated (relative to body and brain weights). Paired organs were weighed together. A combined weight for the thyroid and parathyroid glands was collected.

### Results

All animals survived to the scheduled necropsy on Day 22. No test article-related clinical or veterinary observations were noted in treated animals. No test article-related effects on body weight were observed in treated animals during the treatment or recovery period. There were no G9.2-17 IgG4-related effects on hematology endpoints in either sex at any dose level at any interval.

There were no G9.2-17 IgG4-related effects on coagulation times (i.e., activated partial thromboplastin times [APTT] and prothrombin times) or fibrinogen concentrations in either sex at any dose level at any interval. All fluctuations among individual coagulation values were considered sporadic, consistent with biologic and procedure-related variation, and/or negligible in magnitude, and not related to G9.2-17 IgG4 administration.

There were no G9.2-17 IgG4-related effects on clinical chemistry endpoints in either sex at any dose level at any interval. All fluctuations among individual clinical chemistry values were considered sporadic, consistent with biologic and procedure-related variation, and/or negligible in magnitude, and not related to G9.2-17 IgG4 administration.

There were no G9.2-17 IgG4-related effects on cytokine endpoints in either sex at any dose level at any interval. All fluctuations among individual cytokine values were considered sporadic, consistent with biologic and procedure-related variation, and/or negligible in magnitude, and not related to G9.2-17 IgG4 administration.

Review of the gross necropsy observations revealed no findings that were considered to be test article related. There were no organ weight alterations that were considered to be test article related. There were no test article-related changes.

In conclusion, the single intravenous infusion administration of 30, 100, and 200 mg/kg G9.2-17 IgG4 to cynomolgus monkeys was tolerated with no adverse findings. Therefore, under the conditions of this study the No-observed-Adverse-Effect-Level (NOAEL) was 200 mg/kg, the highest dose level evaluated.

The animals were removed from the cage, and a detailed clinical examination of each animal was performed at 1 hour and 4.5 hours post-start of infusion (SOI) on Day 1 and once daily thereafter during the study.

### Example 15: Intravenous Infusion Study of G9.2-17 in Cynomolgus Monkeys

The objective of this study was to further characterize the toxicity and toxicokinetics of the test article, G9.2-17 (a hIgG4 Monoclonal Antibody which binds to Galectin-9), following once weekly 30-minute intravenous (IV) infusion for 5 weeks in cynomolgus monkeys, and to evaluate the reversibility, progression, or delayed appearance of any observed changes following a 3-week recovery period.

### Experimental Design

Table 22 summarizes the study design.

TABLE 22
Experimental Design

| | | | Dose | Dose | Main Study | | Recovery Study | |
|---|---|---|---|---|---|---|---|---|
| Group | Test | Dose Level | Volume^{a} | Concentration | No. of | No. of | No. of | No. of |
| No. | Material | (mg/kg/dose) | (mL/kg) | (mg/mL) | Males | Females | Males | Females |
| 1 | Vehicle | 0 | 10 | 0 | 3 | 3 | 2 | 2 |
| 2 | G9.2-17 | 100 | 10 | 10 | 3 | 3 | 2 | 2 |
| 3 | G9.2-17 | 300 | 10 | 30 | 3 | 3 | 2 | 2 |

^{a}Based on the most recent practical body weight measurement.

Animals (cynomolgus monkeys) used in the study were assigned to study groups by a standard, by weight, randomization procedure designed to achieve similar group mean body weights. Males and females were randomized separately. Animals assigned to study had body weights within ±20% of the mean body weight for each sex.

The formulations lacking G9.2-17 (“vehicle”) or encompassing G9.2-17 (“test article”) were administered to the animals once weekly for 5 weeks (Days 1, 8, 15, 22, and 29) during the study via 30-minute IV infusion. The dose levels were 0, 100 and 300 mg/kg/dose and administered at a dose volume of 10 mL/kg. The control animals group received the vehicle in the same manner as the treated groups. Doses were administered via the saphenous vein via a percutaneously placed catheter and a new sterile disposable syringe was used for each dose. Dose accountability was measured and recorded prior to dosing and at the end of dosing on toxicokinetic sample collection days (Days 1, 15, and 29) to ensure a ±10% target dose was administered. Individual doses were based on the most recent body weights. The last dose site was marked for collection at the terminal and recovery necropsies. All doses were administered within 8 hours of test article preparation.

In-life procedures, observations, and measurements were performed on the animals as exemplified below.

Electrocardiographic examinations were performed on all animals. Insofar as possible, care was taken to avoid causing undue excitement of the animals before the recording of electrocardiograms (ECGs) in order to minimize extreme fluctuations or artifacts in these measurements. Standard ECGs (10 Lead) were recorded at 50 mm/sec. Using an appropriate lead, the RR, PR, and QT intervals, and QRS duration were measured, and heart rate was determined. Corrected QT (QTc) interval was calculated using a procedure based on the method described by Bazett (1920). All tracings were evaluated and reported by a consulting veterinary cardiologist.

To aid in continuity and reliability, functional observational battery (FOB) evaluations were conducted by two independent raters for all occasions and consisted of a detailed home cage and open area neurobehavioral evaluation (Gauvin and Baird, 2008). Each technician scored the monkey independently (without sharing the results with each other) for each home cage and out of cage observational score, and then the individual scores were assessed for agreement with their partner's score after the completion of the testing. FOB evaluations were conducted on each animal predose (on Day −9 or Day 8) to establish baseline differences and at 2 to 4 hours from the start of infusion on Days 1 and 15, and prior to the terminal and recovery necropsies. The observations included, but were not limited to, evaluation of activity level, posture, lacrimation, salivation, tremors, convulsions, fasciculations, stereotypic behavior, facial muscle movement, palpebral closure, pupil response, response to stimuli (visual, auditory, and food), body temperature, Chaddock and Babinski reflexes, proprioception, paresis, ataxia, dysmetria, and slope assessment, movement, and gait.

Blood pressure of each animal was measured and recorded and consisted of systolic, diastolic, and mean arterial pressure. Blood pressure measurements are reported using three readings that have the Mean Arterial Pressure (MAP) within 20 mmHg.

Respiratory rates of each animal were measured and recorded 3 times per animal/collection interval by visual assessment per Testing Facility SOP. The average of the 3 collections is the reported value.

Clinical pathology evaluations (e.g., immunophenotyping and cytokine evaluations) were conducted on all animals at predetermined intervals. Bone marrow smears were collected and preserved. Blood samples (approximately 0.5 mL) were collected from all animals via the femoral vein for determination of the serum concentrations of the test article. The animals were not fasted prior to blood collection, with the exception of the intervals that coincided with fasting for clinical pathology collections. At the conclusion of the study (day 36 or day 50), animals were euthanatized and tissues for histology processing and microscopic evaluation were collected.

Soluble galectin-9 was evaluated as follows. Blood samples (approximately 1 mL) were collected from all animals via the femoral vein for determination of the serum for soluble galectin 9 predose and 24 hours from the start of infusion on Days 1, 8, 15, and 29, and prior to the terminal and/or recovery necropsies. The animals were not fasted prior to blood collection, with the exception of the intervals that coincided with fasting for clinical pathology collections.

Soluble galectin-9 samples were processed as follows. Blood samples were collected in non-additive, barrier free tubes, allowed to clot at ambient temperature, and centrifuged at ambient temperature. The resulting serum was divided into 2 aliquots (100 μL in Aliquot 1 and remaining in Aliquot 2) in pre labeled cryovials. All aliquots were flash frozen on dry ice within 2 hours of collection and stored frozen at −60° C. to 90° C.

All results presented in the tables of the report were calculated using non-rounded values as per the raw data rounding procedure and may not be exactly reproduced from the individual data presented.

### Results

### Mortality

All animals survived to the scheduled terminal necropsy on Day 36 and recovery necropsy on Day 50.

### Detailed Clinical and Veterinary Observations

No test article-related clinical or veterinary observations were noted in treated animals during the treatment or recovery periods.

### Functional Observational Battery

No test article-related FOB observations were noted in treated animals during the treatment or recovery periods.

### Body Weight and Body Weight Gains

No test article-related effects in body weight and body weight gain were noted in treated animals during the treatment or recovery periods.

### Ophthalmology Examinations

No test article-related effects in ophthalmology examinations were noted in treated animals during the treatment or recovery periods.

### Blood Pressure Values

No test article-related effects in blood pressure values were noted in treated animals during the treatment or recovery periods.

### Respiratory Rate Values

No test article-related effects in respiratory rate values were noted in treated animals during the treatment or recovery periods.

### Electrocardiology

No test article-related effects in electrocardiographic examinations were noted in treated animals during the treatment or recovery periods.

### Hematology

There were no G9.2-17-related effects among hematology parameters in either sex at any dose level at any timepoint.

### Coagulation

There were no G9.2-17-related effects among coagulation parameters in either sex at any dose level at any timepoint.

### Clinical Chemistry

There were no G9.2-17-related effects among clinical chemistry parameters in either sex at any dose level at any timepoint.

### Urinalysis

No G9.2-17-related alterations were observed among urinalysis parameters in either sex at any dose level at the 13-week interim.

### Cytokine

No definitive G9.2-17-related effects on cytokines were seen at any dose level or timepoint.

### Peripheral Blood Leukocyte Analysis (PBLA)

There were no G9.2-17-related effects on PBLA endpoints in either sex at any dose level at any timepoint.

### Bioanalysis, Galectin-9, and Toxicokinetic Evaluation

G9.2-17 was quantifiable in all cynomolgus monkey samples from all G9.2-17-dosed animals after dose administration. No measurable amount of G9.2-17 was detected in control cynomolgus monkey samples. Soluble galectin-9 was quantifiable in all cynomolgus monkey samples from all animals. G9.2-17 serum concentrations were below the bioanalytical limit of quantitation (LLOQ<0.04 μg/mL) in all serum samples obtained predose from most G9.2-17 treated animals on Day 1 and from control animals on Days 1 and 29.

### Gross Pathology and Organ Weight

There were no definitive test article-related macroscopic observations in main study or recovery animals. There were also no test article-related organ weight changes for main study or recovery animals.

### Histopathology

There were no definitive test article-related microscopic observations.

In conclusion, once weekly intravenous infusion administration of 100 and 300 mg/kg of G9.2-17 for 5-weeks to cynomolgus monkeys was tolerated with no adverse findings.

### Example 16: Intravenous Infusion Study of G9.2-17 in Sprague Dawley Rats

The objective of this study was to evaluate potential toxicity of G9.2-17, an IgG4 human monoclonal antibody directed against galectin-9, when administered by intravenous infusion to Sprague Dawley Rats once weekly for 4 consecutive weeks followed by a 3-week post dose recovery period. In addition, the toxicokinetic characteristics of G9.2-17 were determined.

### Experimental Design

Table 23 summarizes the study design.

TABLE 23
Study Design

| | | Dose | Dose | Dose | | | | | | |
|---|---|---|---|---|---|---|---|---|---|---|
| | Test | Level | Concentration | Volume^{a} | Terminal | | Recovery | | TK/Gal-9/Cyto | |
| Group | Material | (mg/kg) | (mg/mL) | (mL/kg) | M | F | M | F | M | F |
| 1 | Control | 0 | 0 | 10 | 10 | 10 | 5 | 5 | 12 | 12 |
| 2 | G9.2-17 | 100 | 10 | 10 | 10 | 10 | 5 | 5 | 12 + 6^{b} | 12 + 6^{b} |
| 3 | G9.2-17 | 300 | 30 | 10 | 10 | 10 | 5 | 5 | 12 + 6^{b} | 12 + 6^{b} |

^{a}Individual dose volumes were calculated based on the most recent body weight.
^{b}SSD animals: 3 animals/sex/group for TK collections only following a single dose administration on Day 1.

One hundred eighty-six animals (Sprague Dawley rats) were assigned to treatment groups randomly by body weight. Control Article/Vehicle, Formulation Buffer for Test Article, and test article, G9.2-17, were administered via a single IV injection in a tail vein at dose levels of 0, 100, and 300 mg/kg once on Days 1, 8, 15, 22, and 29. Test article was administered at dose levels of 100 and 300 mg/kg once on Day 1 to animals assigned to the SSD subgroup.

Clinical observations were performed once daily prior to room cleaning in the morning, beginning on the second day of acclimation. A mortality check was conducted twice daily to assess general animal health and wellness. Food consumption was estimated by weighing the supplied and remaining amount of food in containers once weekly. The average gram (g)/animal/day was calculated from the weekly food consumption. Body weights were taken prior to randomization, on Day −1, then once weekly throughout the study, and on the day of each necropsy. Functional Observation Battery (FOB) observations were recorded for SSB animals approximately 24 hours post dose administrations on Days 1, 35 and 49. Urine was collected overnight using metabolic cages. Samples were obtained on Days 36 and 50.

Animals were fasted overnight prior to each series of collections that included specimens for serum chemistry. In these instances, associated clinical pathology evaluations were from fasted animals. Blood was collected from a jugular vein of restrained, conscious animals or from the vena cava of anesthetized animals at termination.

Parameters assessed during the In-life examinations of the study included clinical observations, food consumption, body weights, functional observational battery. Blood samples were collected at selected time points for clinical pathology (hematology, coagulation, and serum chemistry) analyses. Urine samples were collected for urinalysis. Blood samples were also collected at selected time points for toxicokinetic (TK), immunogenicity (e.g., anti-drug antibody or ADA), and cytokine analyses. Animals were necropsied on Days 36 and 50. At each necropsy, gross observations and organ weights were recorded, and tissues were collected for microscopic examination.

### Results

### In-Life Examinations

Mortality: There were no abnormal clinical observations or body weight changes noted for this animal during the study.

Clinical Observations: There were no G9.2-17-related clinical observations noted during the study.

Food Consumption/Body Weights: There were no G9.2-17-related changes in food consumption, body weights or body weight gain noted during the study.

Clinical Pathology: There were no G9.2-17-related changes noted in clinical pathology parameters.

Cytokine Analysis: There were no G9.2-17-related changed in serum concentrations of IL-2, IL-4, IFN-γ, IL-5, IL-6, IL-10, and/or TNF-α, MCP-1 and MIP-1b.

Gross Pathology: There were no G9.2-17-related gross observations. Further, were no G9.2-17-related changes in absolute or relative organ weights.

Histopathology: There were no G9.2-17-related histologic findings.

In conclusion, intravenous G9.2-17 administration to Sprague Dawley rats once weekly for a total of 5 doses was generally well tolerated. There were no G9.2-17-related changes in clinical observations, food consumption, body weights, FOB parameters, clinical pathology, cytokine, gross observations, or organ weights.

### Example 17: Inhibition of Polarization and Repolarization of M2 Macrophages

Macrophages play an indispensable role in the immune system with decisive functions in both innate and acquired immunity. M1 macrophages are generally considered potent effector cells which can kill tumor cells, while M2 polarized macrophages express a series of cytokines, chemokines, and proteases to promote angiogenesis, lymphangiogenesis, tumor growth, metastasis, and immunosuppression (Sica et al., 2008; Semin. Cancer Biol. 2008; 18:349-355). In M2 macrophages, production of anti-inflammatory cytokines, such as TGF-β and IL-10, is enhanced (Martinez et al., Front Biosci. 2008 Jan. 1; 13:453-61., Mantovani et al., Trends Immunol 2002 November; 23(11):549-55; Zhang et al., J Hematol Oncol 10, 58 (2017)). Given that macrophages comprise a key component of the host immune response, inhibition of polarization or repolarization of M2 macrophages is an important therapeutic consideration in oncological immunotherapy (Poh and Ernst, Front Oncol. 2018 Mar. 12; 8:49).

Whole blood from three healthy human donors was used to isolate CD14+ monocytes. The monocytes were allowed to differentiate to macrophages in X-VIVO-15 media (Lonza) in a 10 cm tissue culture dish for 7 days. The differentiated macrophages were either used directly for assessing inhibition of polarization, or they were cryopreserved and used at a later time or at any other clinically indicated time point for repolarization assays. Prior to use in an assay, the M0 macrophages were phenotyped.

Two different polarization cocktails were used to evaluate macrophage polarization: one with a mixture of IL-4 and IL-13, and a second containing only gal-9. The effect of G9.2-17 on M2 polarization was tested via its direct addition to one of these cocktails, and incubation with macrophages for 48 hours. The effect of G9.2-17 on repolarization of M2 macrophages was tested via addition to the M2-polarized macrophages.

The state of polarization was identified by the measurement of secretion of either IL-10 (repolarization) or TGF-beta1 (inhibition of polarization and repolarization). These factors were quantified in cell culture supernatants using CytoMetric Bead Arrays following the manufacturer's protocol.

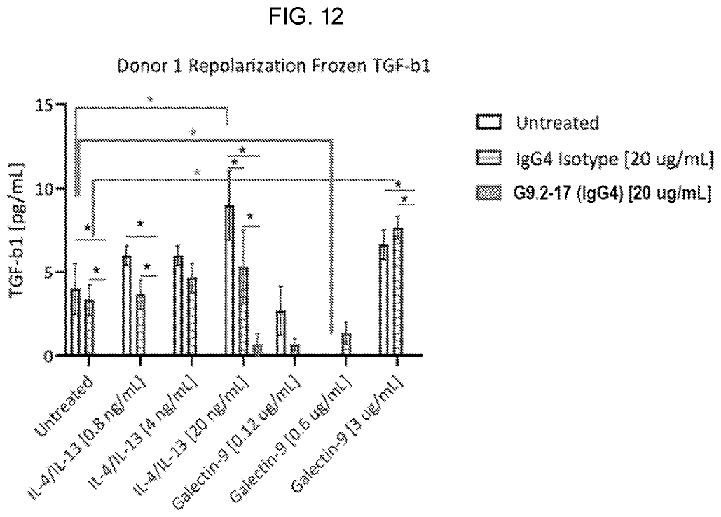

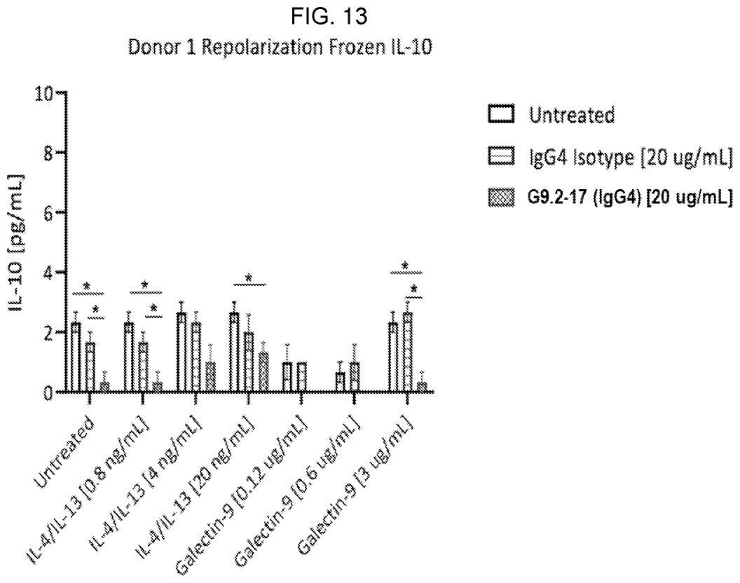

Representative data from one donor showing the effect of G9.2-17 on polarization of fresh monocyte-derived macrophages is in FIG. 12. All donor macrophages showed similar results, with a decrease in TGF-beta1 secretion following incubation with G9.2-17 compared to the isotype matched control or untreated cells. FIG. 12 shows the effect on TGF-beta1 secretion by previously frozen macrophages following incubation with G9.2-17 or an isotype matched control. Treatment with 20 ng/ml of polarization cocktail significantly induced TGF-β1 secretion, while G9.2-17 treatment abolished the IL-4/IL-13-dependent increase of TGF-β1 secretion. FIG. 13 shows the effects on IL-10 secretion on repolarization of cryopreserved macrophages. Treatment with G9.2-17 led to a reduction of secreted IL-10 and TGF-b1 levels in all donors compared to untreated and IgG4 isotype control antibody controls, in the presence of both types of polarization cocktails.

This assay confirms that G9.2-17 can potently inhibit TGF-beta1 and IL-10 at the concentration of 20 μg/ml.

### Example 18: Measurement of Biomarkers

A multiplex Immunofluorescence (mIF) technology is performed on clinical tissues from patients. The mIF assay consists of 10 rounds of staining with two biomarkers stained and imaged per round for a total of ten rounds. For every round, one antibody is conjugated to one of two fluorescent dyes that will allow imaging of the biomarker such that two biomarkers are imaged each round. Biomarkers are stained, imaged, and then the signal is quenched to allow for further staining and imaging rounds to occur without bleed-through of competing signal. When the staining and imaging of the entire 19-marker panel is complete, positivity of each biomarker on cells is classified by deep learning algorithms that are trained to detect positive signal. When analysis is complete, various data is generated, including density and raw counts of positive cells for each biomarker and co-expression of interest. Biomarkers include CD3, CD4, CD8, CD45RO, FoxP3, CD11b, CD14, CD15, CD16, CD33, CD68, CD163, HLA-DR, Arginase1, Granzyme B, Ki67, PD-1, PD-L1, F4/80, Ly6G/C and PanCK.

### Example 9. Evaluating Mouse Galectin-9 in Plasma by ELISA

This study evaluated galectin-9 in plasma of orthotopic pancreatic cancer xenograft model mPA6115 in female C57BL/6 mice. Mice were assigned to multiple groups and treated following the study design illustrated in Table 24 below. As per the protocol, plasma samples were collected from retro orbital sinus on day 3 before the 1st dose for all mice from Group 1-6 engrafted with tumors (pre-dose) and 10 non-tumor bearing mice in Group 7 (tumor implantation was on day 0) and by cardiac puncture at termination for euthanized mice which were moribund (post-dose).

TABLE 24
Study Dosing Schedule

| | | Dose | Dosing | Dosing | | Dosing |
|---|---|---|---|---|---|---|
| Group | | level | Solution | Volume | Route of | Frequency & |
| No. | Treatment | (mg/kg) | (mg/ml) | (μL/g) | Admin. | Duration |
| 1 | Untreated | — | — | — | — | — |
| 2 | Isotype IgG1 mouse | 200 | 2 | 100 | i.p. | Q4D*8 |
| | | μg/mouse | | μL/mouse | | |
| 3 | Anti-Gal9 mAb | 200 | 2 | 100 | i.p. | Q4D*14 |
| | | μg/mouse | | μL/mouse | | |

| | |
|---|---|
| 4 | 10 non-tumor bearing mice for blood sampling |

i.p. = intraperitoneal;
i.v. = intravenous;
QW = once a week;
Q4D = once every four days

Levels of galectin-9 in the plasma samples were analyzed by ELISA following the below procedure:
1. Brought all reagents and samples to room temperature (18-25° C.) before use. It was recommended that all standards and samples be run at least in duplicate.
2. Labeled removable 8-well strips as appropriate for your experiment.
3. Added 100 μl of each standard and prepared samples into appropriate wells. Covered wells and incubate for 2.5 hours at room temperature with gentle shaking.
4. Discarded the solution and wash 4 times with 1× Wash Solution. Wash by filling each well with Wash Buffer (300 μl) using a multi-channel Pipette or autowasher. Complete removal of liquid at each step was essential to good performance. After the last wash, removed any remaining Wash Buffer by aspirating or decanting. Inverted the plate and blot it against clean paper towels.
5. Added 100 μl of 1× prepared biotinylated antibody (Reagent Preparation step 3) to each well. Incubated for 1 hour at room temperature with gentle shaking.
6. Discarded the solution. Repeat the wash as in step 4.
7. Added 100 μl of prepared Streptavidin solution to each well. Incubated for 45 minutes at room temperature with gentle shaking.
8. Discarded the solution. Repeat the wash as in step 4.
9. Added 100 μl of TMB One-Step Substrate Reagent to each well. Incubated for 30 minutes at room temperature in the dark with gentle shaking.
10. Added 50 μl of Stop Solution to each well. Read at 450 nm immediately.

The results demonstrated that galectin-9 serum levels increased in the mPA6115 mouse model once tumors were orthotopically engrafted, which was aligned with observations in pancreatic adenocarcinoma cancer patients. This study demonstrated that galectin-9 serum levels increased significantly in animals where pancreatic ductal adenocarcinomas were growing orthotopically. This implies that the source of such galectin-9 is indeed the tumor tissue, further supporting the therapeutic approach of blocking galectin-9 in this disease context.

### EQUIVALENTS

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several inventive embodiments have been described and illustrated herein, those of ordinary skill in the art are readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art are readily appreciate that all parameters, dimensions, materials, and configurations described herein are meant to be exemplary and that the actual parameters, dimensions, materials, and/or configurations depend upon the specific application or applications for which the inventive teachings is/are used. Those skilled in the art are recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” are refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for treating a solid tumor, the method comprising administering to a subject in need thereof an effective amount of an antibody that binds human Galectin-9 (anti-Galectin-9 antibody), wherein the anti-Galectin-9 antibody comprises a light chain complementarity determining region 1 (CDR1) set forth as SEQ ID NO: 1, a light chain complementarity determining region 2 (CDR2) set forth as SEQ ID NO: 2, and a light chain complementarity determining region 3 (CDR3) set forth as SEQ ID NO: 3 and/or comprises a heavy chain complementarity determining region 1 (CDR1) set forth as SEQ ID NO: 4, a heavy chain complementarity determining region 2 (CDR2) set forth as SEQ ID NO: 5, and a heavy chain complementarity determining region 3 (CDR3) set forth as SEQ ID NO: 6 and wherein the anti-Galectin-9 antibody is administered to the subject at a dose of about 0.2 mg/kg to about 32 mg/kg,
wherein the subject has one or more of the following features:
(i) has no resectable cancer;
(ii) has no infection by SARS-COV-2;
(iii) has no active brain or leptomeningeal metastasis; and
(iv) has unresectable metastatic cancer, which is adenocarcinoma, optionally squamous cell carcinoma.

2. The method of claim 1, wherein the anti-Galectin-9 antibody is administered to the subject at a dose of about 3 mg/kg to about 15 mg/kg or about 0.2 mg/kg to about 16 mg/kg once every two weeks to once every six weeks, optionally once every two weeks.

3. The method of claim 2, wherein the anti-Galectin-9 antibody is administered to the subject at a dose of about 0.2 mg/kg, about 0.6 mg/kg, about 0.63 mg/kg, about 2 mg/kg, about 4 mg/kg, about 6 mg/kg, about 6.3 mg/kg, about 8 mg/kg, about 10 mg/kg, about 12 mg/kg, or about 16 mg/kg once every two weeks to once every six weeks, optionally once every two weeks.

4. The method of claim 1, wherein the anti-Galectin-9 antibody is administered to the subject at a dose of about 650 mg to about 1120 mg once every two weeks to once every six weeks, optionally once every two weeks.

5. The method of claim 4, wherein the anti-Galectin-9 antibody is administered to the subject at a dose of about 650 mg to about 700 mg once every two weeks to once every six weeks, optionally once every two weeks, or at a dose of about 1040 mg to about 1120 mg once every two weeks to once every six weeks, optionally once every two weeks.

6. A method for treating a solid tumor, the method comprising administering to a subject in need thereof an effective amount of an antibody that binds human Galectin-9 (anti-Galectin-9 antibody), wherein the anti-Galectin-9 antibody comprises:
(a) a light chain comprising a light chain variable region (VL), which comprises a light chain (LC) complementarity determining region 1 (CDR1) comprising the amino acid sequence of SEQ ID NO: 1, a LC complementarity determining region 2 (CDR2) comprising the amino acid sequence of SEQ ID NO: 2, and a LC complementarity determining region 3 (CDR3) comprising the amino acid sequence of SEQ ID NO: 3 and
(b) a heavy chain comprising a heavy chain variable region (VH), which comprises a heavy chain (HC) complementarity determining region 1 (CDR1) comprising the amino acid sequence of SEQ ID NO: 4, a HC complementarity determining region 2 (CDR2) comprising the amino acid sequence of SEQ ID NO: 5, and a HC complementarity determining region 3 (CDR3) comprising the amino acid sequence of SEQ ID NO: 6;
wherein the anti-Galectin-9 antibody is administered to the subject at a dose of about 0.2 mg/kg to about 32 mg/kg once every week.

7. The method of claim 6, wherein the anti-Galectin-9 antibody is administered to the subject at a dose of about 10 mg/kg to about 16 mg/kg once every week.

8. The method of claim 7, wherein the anti-Galectin-9 antibody is administered to the subject at a dose of 10 mg/kg or 16 mg/kg once every week.

9. The method of claim 6, wherein the anti-Galectin-9 antibody is administered to the subject at a dose of about 650 mg to about 1120 mg once every week.

10. The method of claim 9, wherein the anti-Galectin-9 antibody is administered to the subject at a dose of about 650 mg to about 700 mg once every week, or about 1040 to about 1120 mg once every week.

11. The method of claim 1, wherein the solid tumor is a metastatic solid tumor.

12. The method of claim 11, wherein the solid tumor is pancreatic ductal adenocarcinoma (PDAC), colorectal cancer (CRC), hepatocellular carcinoma (HCC), cholangiocarcinoma (CAA), renal cell carcinoma (RCC), urothelial cancer, head and neck cancer, breast cancer, lung cancer, or a gastrointestinal (GI) solid tumor.

13. The method of claim 1, wherein the anti-Galectin-9 antibody is administered to the subject by intravenous infusion.

14. The method of claim 1, wherein the V_{L} of the anti-Galectin-9 antibody comprises the amino acid sequence of SEQ ID NO: 8.

15. The method of claim 1, wherein the V_{H} of the anti-Galectin-9 antibody comprises the amino acid sequence of SEQ ID NO: 7.

16. The method of claim 1, wherein the anti-Galectin-9 antibody is a full-length antibody.

17. The method of claim 16, wherein the anti-Galectin-9 antibody is an IgG1 or IgG4 molecule.

18. The method of claim 17, wherein the anti-Galectin-9 antibody is a human IgG4 molecule having a modified Fc region relative to the wildtype human IgG4 counterpart.

19. The method of claim 18, wherein the modified Fc region comprises the amino acid sequence of SEQ ID NO: 14.

20. The method of claim 1, wherein the anti-Galectin-9 antibody comprises a heavy chain comprising the amino acid sequence of SEQ ID NO: 19 and a light chain comprising the amino acid sequence of SEQ ID NO: 15.

21. The method of claim 1, wherein the subject is free of other anti-cancer therapy concurrently with the treatment involving the anti-Galectin-9 antibody.

22. The method of claim 1, wherein the method further comprises administering to the subject an immune checkpoint inhibitor.

23. The method of claim 22, wherein the immune checkpoint inhibitor is an antibody that binds PD-1.

24. The method of claim 23, wherein the antibody that binds PD-1 is pembrolizumab, nivolumab, tislelizumab, dostarlimab, or cemiplimab.

25. The method of claim 22, wherein the subject is free of exposure to any anti-PD-1 or anti-PD-L1 agent in any prior lines of therapy, free of microstatellite instability (MSI-H) and/or deficient mismatch repair (dMMR), or a combination thereof.

26. The method of claim 24, wherein the antibody that binds PD-1 is nivolumab, which is administered to the subject at a dose of 240 mg once every two weeks.

27. The method of claim 22, wherein the checkpoint inhibitor is administered to the subject on a day when the subject receives the anti-Galectin 9 antibody.

28. The method of claim 22, wherein the checkpoint inhibitor and the anti-Galectin 9 antibody are administered to the subject on two consecutive days.

29. The method of claim 22, wherein the administration of the checkpoint inhibitor is performed prior to the administration of the anti-Galectin 9 antibody.

30. The method of claim 1, wherein the subject has undergone one or more prior anti-cancer therapies.

31. The method of claim 30, wherein the one or more prior anti-cancer therapies comprise chemotherapy, immunotherapy, radiation therapy, a therapy involving a biologic agent, or a combination thereof.

32. The method of claim 30, wherein the subject has progressed disease through the one or more prior anti-cancer therapies or is resistant to the one or more prior therapies.

33. The method of claim 1, wherein the subject is a human patient having an elevated level of Galectin-9 relative to a control value.

34. The method of claim 33, wherein the human patient has an elevated serum or plasma level of Galectin-9 relative to the control value.

35. The method of claim 1, wherein the human patient has cancer cells expressing Galectin-9.

36. The method of claim 1, wherein the human patient has immune cells expressing Galectin-9.

37. The method of claim 1, further comprising monitoring occurrence of adverse effects in the subject.

38. The method of claim 37, further comprising reducing the dose of the anti-Galectin-9 antibody, the dose of the checkpoint inhibitor, or both when an adverse effect is observed.

39. The method of claim 1, wherein the subject is administered multiple doses of the anti-Galectin9 antibody and a later dose is higher than an earlier dose.


SEQUENCE LISTING (35 sequences)

SEQ ID NO: 1 - protein, 11 aa, Artificial Sequence
  Synthetic
RASQSVSSAVA

SEQ ID NO: 2 - protein, 7 aa, Artificial Sequence
  Synthetic
SASSLYS

SEQ ID NO: 3 - protein, 9 aa, Artificial Sequence
  Synthetic
QQSSTDPIT

SEQ ID NO: 4 - protein, 9 aa, Artificial Sequence
  Synthetic
FTVSSSSIH

SEQ ID NO: 5 - protein, 17 aa, Artificial Sequence
  Synthetic
YISSSSGYTYYADSVKG

SEQ ID NO: 6 - protein, 15 aa, Artificial Sequence
  Synthetic
YWSYPSWWPYRGMDY

SEQ ID NO: 7 - protein, 124 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSS

SEQ ID NO: 8 - protein, 108 aa, Artificial Sequence
  Synthetic
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKR

SEQ ID NO: 9 - protein, 20 aa, Artificial Sequence
  Synthetic
MYRMQLLSCIALSLALVTNS

SEQ ID NO: 10 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 106 aa, Artificial Sequence
  Synthetic
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 14 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 15 - protein, 214 aa, Artificial Sequence
  Synthetic
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 454 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 454 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 451 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 19 - protein, 451 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 20 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 451 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 451 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 329 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 329 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 27 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 29 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - protein, 453 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 453 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 450 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 33 - protein, 450 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 34 - protein, 450 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 35 - protein, 450 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG